(19) United States
(12) Patent Application Publication
(10) Pub. No.: US 2011/0294778 A1
(43) Pub. Date: Dec. 1, 2011

(54) HEPATITIS C INHIBITOR COMPOUNDS

(75) Inventors: Montse LLINAS-BRUNET, Dollard-des-Ormeaux (CA); Josée BORDELEAU, Laval (CA); Cédrickx GODBOUT, Mascouche (CA); Mélissa LEBLANC, Laval (CA); Benoît MOREAU, Laval (CA); Jeffrey O'MEARA, Boisbriand (CA)

(73) Assignee: BOEHRINGER INGELHEIM INTERNATIONAL GMBH, Ingelheim am Rhein (DE)

(21) Appl. No.: 12/952,794

(22) Filed: Nov. 23, 2010

Related U.S. Application Data
(60) Provisional application No. 61/264,136, filed on Nov. 24, 2009.

(51) Int. Cl. A61K 31/4709; A61P 31/14; C07D 487/04

(52) U.S. Cl. 514/210.18; 540/460; 514/312

(57) ABSTRACT

Compounds of Formula (I)

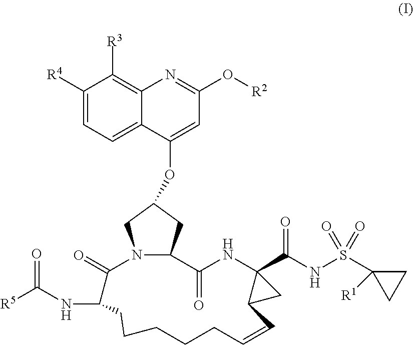

wherein R¹, R², R³, R⁴ and R⁵ are defined herein, maintain good activity against NS3 proteases containing clinically relevant genotype 1a R155K and genotype 1b D168V resistance mutations. The compounds are useful as inhibitors of HCV NS3 protease for the treatment of hepatitis C viral infection.

### FIELD OF THE INVENTION

The present invention relates to macrocycle peptide analogs and their use as inhibitors of hepatitis C virus (HCV) NS3 protease activity, pharmaceutical composition containing the same, and methods of using the same for the treatment of HCV infection.

### BACKGROUND OF THE INVENTION

It is estimated that at least 170 million persons worldwide are infected with the hepatitis C virus (HCV). Acute HCV infection progresses to chronic infection in a high number of cases, and, in some infected individuals, chronic infection leads to serious liver diseases such as cirrhosis and hepatocellular carcinoma.

HCV replicates to very high levels and the HCV polymerase is error-prone resulting in a wide variety of new sequence variants (Science 1998, 282, 103-107). Some new sequence variants confer resistance to drug candidates currently undergoing clinical trials. The emergence of such resistance mutations is one cause of treatment failure in HCV antiviral trials (New England Journal of Medicine 2009, 360, 1827-1838 and New England Journal of Medicine 2009, 360, 1839-1850). Resistance mutations observed in the clinical trials can also be selected for by in vitro experiments, with correlation between clinical resistance mutations and those from in vitro experiments (New England Journal of Medicine 2009, 360, 1827-1838).

The impact of emergence of any one resistance mutant on the outcome of therapy is determined not only by the effect of the particular resistance mutant on drug potency, but also by the fitness of the resulting virus variant. A resistance mutation which results in a virus with poor fitness will be more difficult to select under drug pressure, even if it results in a large decrease in potency for the drug. As a result not all resistance mutations observed are equally relevant to clinical therapy. (Antimicrobial Agents for Chemotherapy 2008, 52, 1101-1110)

There is a need for new antivirals with activity against known, relatively fit, resistance mutations for their target. In addition, a patient who has previously failed treatment with a HCV drug of a particular class (e.g. an HCV protease inhibitor) may be treated with another drug of that same class (e.g. another HCV protease inhibitor) if activity of the second drug is not affected by the resistance mutations selected on the earlier treatment. This has been demonstrated for HIV antiviral therapies (Journal of Medical Virology 2008, 80, 565-576).

HCV NS3 protease inhibitors currently in the clinic primarily target HCV genotype 1 infection. The vast majority of HCV genotype 1 infections are of either subtype 1a or subtype 1b (Clinics in Liver Disease 2003, 7, 45-66). The NS3 proteases from HCV-1a and HCV-1b subtypes have very similar but not identical sequences. HCV protease inhibitors currently in clinical trials can be divided into two classes based on their chemical structure, and these classes have distinct but overlapping resistance mutation profiles (Journal of Viral Hepatitis 2009, 16, 377-387). A first class, as exemplified by the inhibitors telaprevir and boceprevir, contain an α-ketoamide moiety as the active site binding group; characteristic mutations for these compounds result in substitutions at amino acids 36, 41, 54, 155, 156, and 170 of the NS3 protease. Compounds containing an acylsulfonamide in place of the α-ketoamide, for example ITMN-191, TMC435, and MK-7009, are recognized as being part of a second class.

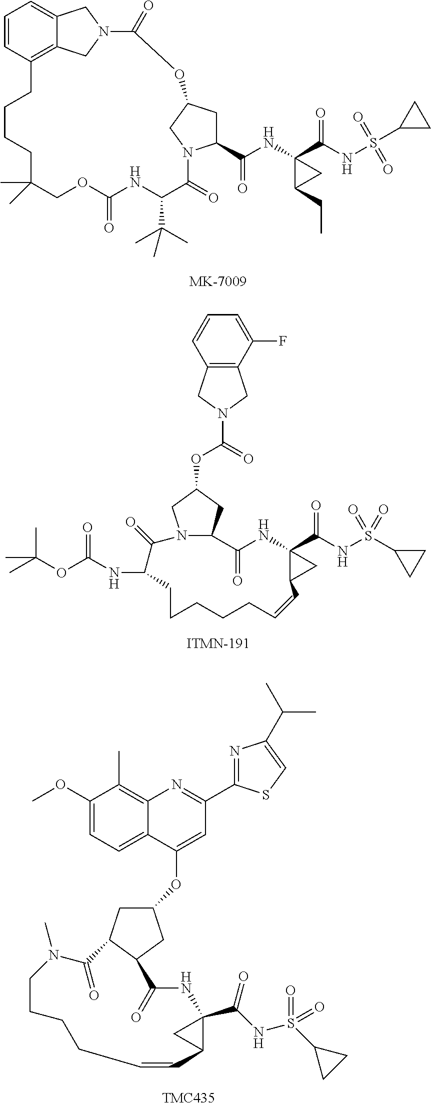

Resistance against this second class of protease inhibitors is primarily due to substitutions at amino acid 155, typically Arg to Lys (R155K), at amino acid 156, typically Ala to Val (A156V) or Ala to Thr (A156T), and at amino acid 168, typically Asp to Val (D168V) or Asp to Ala (D168A). Arg-155 and Ala-156 substitutions are observed for both classes (Antimicrobial Agents and Chemotherapy 2009, 53 (4) 1377-85, Antimicrobial Agents and Chemotherapy 2008, 52 (12) 4432-41, Antimicrobial Agents and Chemotherapy 2009, published online 19 Oct. 2009 doi:10.1128/AAC.00677-09, Journal of Viral Hepatitis 2009, 16, 377-387). There is increasing evidence that virus containing the R155K mutation is quite fit and can persist for long periods of time (Journal of Infectious Diseases 2009, 199:737-41). Virus containing the A156V or T mutations is not very fit and even though these are observed in the clinic they are transient and revert to wildtype in a short period of time. Virus containing the D168A mutation has relatively poor fitness. D168V appears to have intermediate fitness, though there is limited clinical data on the persistence of this variant in patients. (Expert Opinion on Investigational Drugs 2008 17(3):303-319, Antimicrobial Agents for Chemotherapy 2008, 52, 1101-1110, Hepatology 2007, 46, 631-639, Gastroenterology, 2007, 132, 1767-1777)

An R155K substitution appears in genotype 1a patients as the R155K mutation results from a single-base mutation but more rarely in genotype 1b patients which require a two-base mutation for the same substitution to occur. D168V can occur via a single-base mutation in either subtype 1a or 1b, but in clinical trials disclosed to date, it occurs more commonly in genotype 1b patients, (Marcellin et al., Antiviral activity and safety of TMC435 combined with peginterferon alpha-2A and ribavirin in patients with genotype 1 hepatitis C infection who failed previous IFN-based therapy, 44th Annual Meeting of the European Association for the Study of the Liver, Apr. 22-26, 2009, Copenhagen, Denmark, Lenz et al., In vitro resistance profile of the HCV NS3/4A inhibitor TMC435350, 15^{th} International Symposium on Hepatitis C Virus & Related Viruses, San Antonio, Tex., USA, Oct. 5-9, 2008) probably because for genotype 1a patients the more fit R155K is typically observed instead.

Accordingly, clinically relevant resistance mutations for the second class of HCV protease inhibitors are considered genotype 1a R155K and genotype 1b D168V.

Activity of HCV protease inhibitors is most effectively measured using the subgenomic replicon system, in which inhibition of the physiologically relevant HCV replication complex can be directly measured (Journal of Viral Hepatitis, 2007, 14 (Suppl. 1) 64-67). Inhibition in this system has translated into clinical efficacy as shown for all the clinical candidates described above (Antimicrobial Agents and Chemotherapy 2009, 53(4) 1377-85, Antimicrobial Agents and Chemotherapy 2008, 52(12) 4432-41, Antimicrobial Agents and Chemotherapy 2009, published online 19 Oct. 2009 doi:10.1128/AAC.00677-09).

Accordingly, there is a need to provide novel compounds as drug candidates that are active against clinically relevant resistance mutations as represented by genotype 1a R155K and genotype 1b D168V.

WO 2007/056120 describes macrocyclic peptides that are useful for inhibiting HCV.

### SUMMARY OF THE INVENTION

We have unexpectedly found that certain compounds of the invention maintain good activity against NS3 proteases containing clinically relevant resistance mutations for this class as represented by genotype 1a R155K and genotype 1b D168V resistance mutations.

Further objects of this invention arise for the one skilled in the art from the following description and the examples.

The invention provides a compound of Formula (I) or a racemate, diastereoisomer, optical isomer or salt thereof:

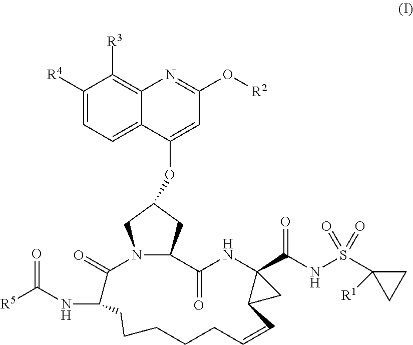

wherein:
R¹ is H or (C₁₋₆)alkyl;
R² is (C₁₋₆)alkyl optionally substituted 1-3 times with halo or (C₃₋₇)cycloalkyl;
R³ is (C₁₋₆)alkyl, —O—(C₁₋₆)alkyl or halo;
R⁴ is —O—(C₁₋₆)alkyl, —OH or halo;
R⁵ is Het optionally substituted 1-3 times with (C₁₋₆)alkyl, —(C₁₋₆)alkyl-C(═O)—N((C₁₋₆)alkyl)₂, —(C₁₋₆)alkyl-O—(C₁₋₆)alkyl or (C₁₋₆)haloalkyl; or

R⁵ is —N(R^{A})(R^{B}) wherein R^{A} and R^{B} are (C₁₋₆)alkyl or R^{A} and R^{B} are linked together with the N to which they are attached to form a 4- to 7-membered saturated ring, wherein said ring is optionally substituted 1-3 times with (C₁₋₆)alkyl, —O—(C₁₋₆)alkyl, —OH or halo.

The invention further provides a compound of Formula (I) or a racemate, diastereoisomer, optical isomer or salt thereof:

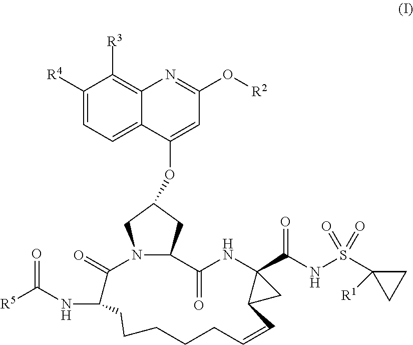

wherein:
R¹ is H or (C₁₋₆)alkyl;
R² is (C₁₋₆)alkyl optionally substituted 1-3 times with halo or (C₃₋₇)cycloalkyl;
R³ is (C₁₋₆)alkyl, —O—(C₁₋₆)alkyl or halo;
R⁴ is —O—(C₁₋₆)alkyl, —OH or halo;
R⁵ is Het optionally substituted 1-3 times with (C₁₋₆)alkyl, (C₁₋₆)alkyl-C(═O)—N((C₁₋₆)alkyl)₂, —(C₁₋₆)alkyl-O—(C₁₋₆)alkyl or (C₁₋₆)haloalkyl; or
R⁵ is —N(R^{A})(R^{B}) wherein R^{A} and R^{B} are (C₁₋₆)alkyl or R^{A} and R^{B} are linked together with the N to which they are attached to form a 4- to 7-membered saturated ring, wherein said ring is optionally substituted 1-3 times with (C₁₋₆)alkyl, —O—(C₁₋₆)alkyl, —OH or halo; with the proviso that the following compounds are excluded:
(2R,6S,12Z,13aS,14aR,16aS)-6-({[1-(2,2-difluoroethyl)-1H-pyrazol-3-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A1);
(2R,6S,12Z,13aS,14aR,16aS)-6-({[1-(2-methoxyethyl)-1H-pyrazol-3-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A2);
(2R,6S,12Z,13aS,14aR,16aS)-2-{[7-chloro-8-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A3);
(2R,6S,12Z,13aS,14aR,16aS)-6-({[1-(2-fluoroethyl)-1H-pyrazol-3-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A4);
(2R,6S,12Z,13aS,14aR,16aS)-2-{[7-methoxy-8-methyl-2-(propan-2-yloxy)quinolin-4-yl]oxy}-N-[(1-methylcyclopropyl)sulfonyl]-5,16-dioxo-6-({[1-(2,2,2-trifluoroethyl)-1H-pyrazol-3-yl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide (A5);
(2R,6S,12Z,13aS,14aR,16aS)-2-{[7-methoxy-8-methyl-2-(propan-2-yloxy)quinolin-4-yl]oxy}-N-[(1-methylcyclopropyl)sulfonyl]-6-{[(2-methyl-2H-1,2,3-triazol-4-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A6);
(2R,6S,12Z,13aS,14aR,16aS)-2-{[7-methoxy-8-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A7);
(2R,6S,12Z,13aS,14aR,16aS)-2-{[7-fluoro-8-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A8);
(2R,6S,12Z,13aS,14aR,16aS)—N-(cyclopropylsulfonyl)-2-{[7-methoxy-8-methyl-2-(propan-2-yloxy)quinolin-4-yl]oxy}-6-({[5-(methoxymethyl)thiophen-2-yl]carbonyl}amino)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A9);
(2R,6S,12Z,13aS,14aR,16aS)-6-({[2-(2,2-difluoroethyl)-2H-1,2,3-triazol-4-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A10);
(2R,6S,12Z,13aS,14aR,16aS)—N-(cyclopropylsulfonyl)-6-({[1-(2,2-difluoroethyl)-1H-pyrazol-3-yl]carbonyl}amino)-2-{[7-methoxy-8-methyl-2-(propan-2-yloxy)quinolin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A11);
(2R,6S,12Z,13aS,14aR,16aS)—N-(cyclopropylsulfonyl)-2-{[7-methoxy-8-methyl-2-(propan-2-yloxy)quinolin-4-yl]oxy}-5,16-dioxo-6-({[1-(2,2,2-trifluoroethyl)-1H-pyrazol-3-yl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide (A12);
(2R,6S,12Z,13aS,14aR,16aS)-2-{[7-fluoro-8-methoxy-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A13);
(2R,6S,12Z,13aS,14aR,16aS)—N-(cyclopropylsulfonyl)-6-({[5-(2-hydroxypropan-2-yl)thiophen-2-yl]carbonyl}amino)-2-{[7-methoxy-8-methyl-2-(propan-2-yloxy)quinolin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A14);
(2R,6S,12Z,13aS,14aR,16aS)-2-{[7-hydroxy-8-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A15);
(2R,6S,12Z,13aS,14aR,16aS)—N-(cyclopropylsulfonyl)-2-{[7-fluoro-8-methoxy-2-(propan-2-yloxy)quinolin-4-yl]oxy}-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A16);
(2R,6S,12Z,13aS,14aR,16aS)-6-[({1-[2-(dimethylamino)-2-oxoethyl]-1H-pyrazol-3-yl}carbonyl)amino]-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A17);
(2R,6S,12Z,13aS,14aR,16aS)—N-(cyclopropylsulfonyl)-2-{([7-methoxy-8-methyl-2-(propan-2-yloxy)quinolin-4-yl]oxy}-6-[({1-[2-(methylamino)-2-oxoethyl]-1H-pyrazol-3-yl}carbonyl)amino]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A18);
(2R,6S,12Z,13aS,14aR,16aS)-2-{[7-methoxy-8-methyl-2-(propan-2-yloxy)quinolin-4-yl]oxy}-6-[({1-[2-(methylamino)-2-oxoethyl]-1H-pyrazol-3-yl}carbonyl)amino]-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A19);
(2R,6S,12Z,13aS,14aR,16aS)-6-({[5-(2-hydroxypropan-2-yl)thiophen-2-yl]carbonyl}amino)-2-{([7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A20);
(2R,6S,12Z,13aS,14aR,16aS)-6-({[1-(2-fluoroethyl)-1H-pyrazol-3-yl]carbonyl}amino)-2-{[7-fluoro-8-methoxy-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A21);
(2R,6S,12Z,13aS,14aR,16aS)-6-({[1-(2,2-difluoroethyl)-1H-pyrazol-3-yl]carbonyl}amino)-2-{[7-fluoro-8-methoxy-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A22);
(2R,6S,12Z,13aS,14aR,16aS)-2-{[7-fluoro-8-methoxy-2-(propan-2-yloxy)quinolin-4-yl]oxy}-N-[(1-methylcyclopropyl)sulfonyl]-5,16-dioxo-6-({[1-(propan-2-yl)-1H-pyrazol-3-yl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide (A23);
(2R,6S,12Z,13aS,14aR,16aS)-2-{[8-bromo-7-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A24);
(2R,6S,12Z,13aS,14aR,16aS)-2-{[7,8-dichloro-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A25);
(2R,6S,12Z,13aS,14aR,16aS)-2-{[7-fluoro-8-methoxy-2-(propan-2-yloxy)quinolin-4-yl]oxy}-N-[(1-methylcyclopropyl)sulfonyl]-5,16-dioxo-6-({[1-(2,2,2-trifluoroethyl)-1H-pyrazol-3-yl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide (A26);
(2R,6S,12Z,13aS,14aR,16aS)-6-({[1-(difluoromethyl)-1H-pyrazol-3-yl]carbonyl}amino)-2-{[7-fluoro-8-methoxy-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A27);
(2R,6S,12Z,13aS,14aR,16aS)-2-{[7-fluoro-8-methoxy-2-(propan-2-yloxy)quinolin-4-yl]oxy}-N-[(1-methylcyclopropyl)sulfonyl]-6-{[(5-methylthiophen-2-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A28); and
(2R,6S,12Z,13aS,14aR,16aS)-2-{[7-methoxy-8-methyl-2-(2,2,2-trifluoroethoxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (A29).

Furthermore, the invention provides a compound or a salt thereof according to the following structures in Table 1 and Table 2:

TABLE 1

| Cmpd # | Structure | Name |
|---|---|---|
| 1001 | 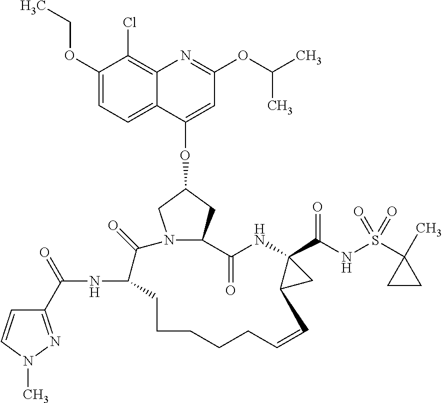 | (2R,6S,12Z,13aS,14aR,16aS)-2-{[8- chloro-7-ethoxy-2-(propan-2- yloxy)quinolin-4-yl]oxy}-N-[(1- methylcyclopropyl)sulfonyl]-6-{[(1- methyl-1H-pyrazol-3- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1002 | 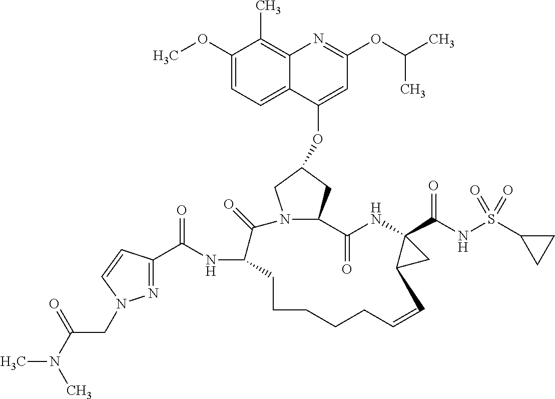 | (2R,6S,12Z,13aS,14aR,16aS)-N- (cyclopropylsulfonyl)-6-[({1-[2- (dimethylamino)-2-oxoethyl]-1H- pyrazol-3-yl}carbonyl)amino]-2-{[7- methoxy-8-methyl-2-(propan-2- yloxy)quinolin-4-yl]oxy}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1003 | 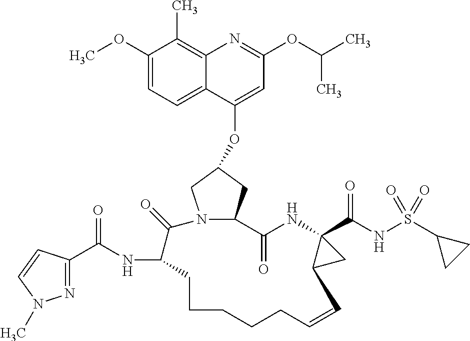 | (2R,6S,12Z,13aS,14aR,16aS)-N- (cyclopropylsulfonyl)-2-{[7-methoxy-8- methyl-2-(propan-2-yloxy)quinolin-4- yl]oxy}-6-{[(1-methyl-1H-pyrazol-3- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1004 | 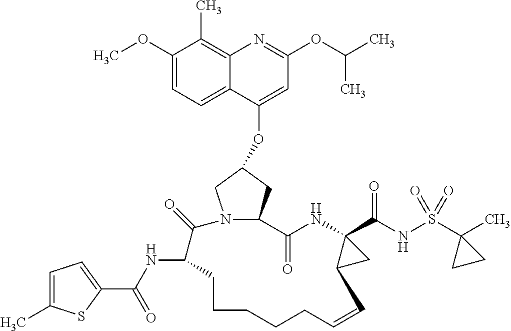 | (2R,6S,12Z,13aS,14aR,16aS)-2-{[7- methoxy-8-methyl-2-(propan-2- yloxy)quinolin-4-yl]oxy}-N-[(1- methylcyclopropyl)sulfonyl]-6-{[(5- methylthiophen-2-yl)carbonyl]amino}- 5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1005 | 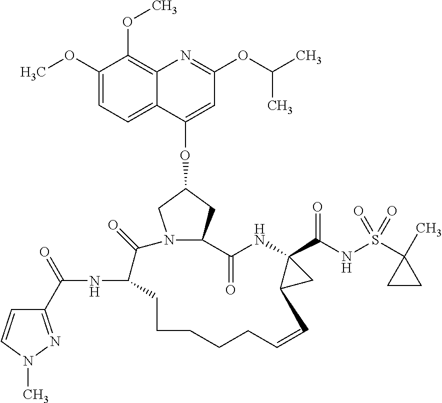 | (2R,6S,12Z,13aS,14aR,16aS)-2-{[7,8- dimethoxy-2-(propan-2-yloxy)quinolin- 4-yl]oxy}-N-[(1- methylcyclopropyl)sulfonyl]-6-{[(1- methyl-1H-pyrazol-3- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1006 | 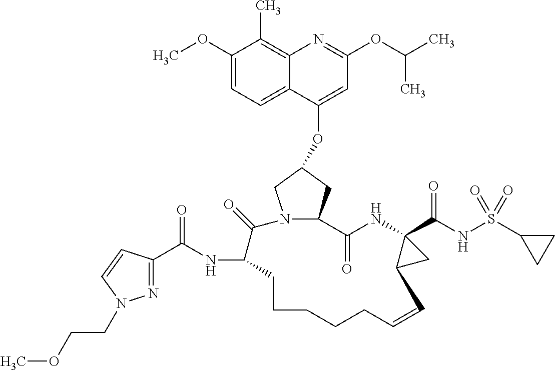 | (2R,6S,12Z,13aS,14aR,16aS)-N- (cyclopropylsulfonyl)-6-({[1-(2- methoxyethyl)-1H-pyrazol-3- yl]carbonyl}amino)-2-{[7-methoxy-8- methyl-2-(propan-2-yloxy)quinolin-4- yl]oxy}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1007 | 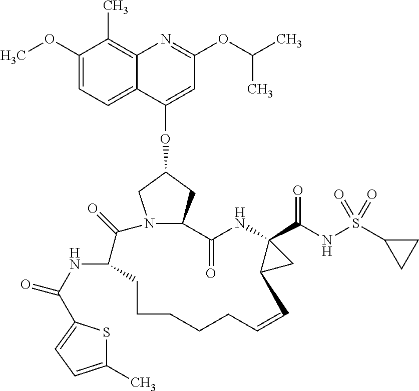 | (2R,6S,12Z,13aS,14aR,16aS)-N- (cyclopropylsulfonyl)-2-{[7-methoxy-8- methyl-2-(propan-2-yloxy)quinolin-4- yl]oxy}-6-{[(5-methylthiophen-2- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1008 | 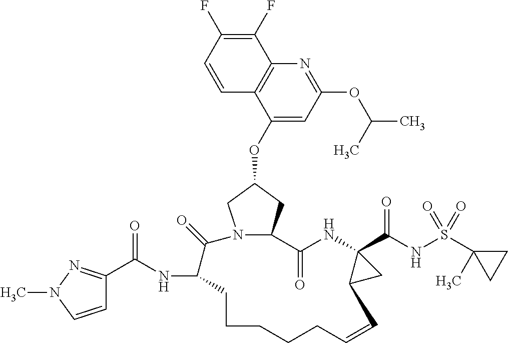 | (2R,6S,12Z,13aS,14aR,16aS)-2-{[7,8- difluoro-2-(propan-2-yloxy)quinolin-4- yl]oxy}-N-[(1- methylcyclopropyl)sulfonyl]-6-{[(1- methyl-1H-pyrazol-3- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1009 | 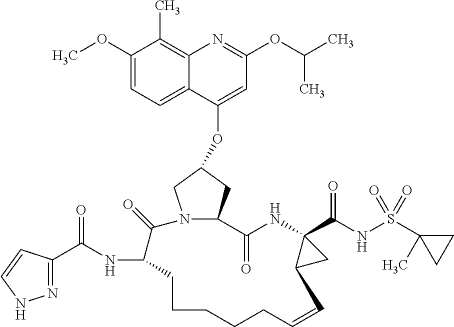 | (2R,6S,12Z,13aS,14aR,16aS)-2-{[7- methoxy-8-methyl-2-(propan-2- yloxy)quinolin-4-yl]oxy}-N-[(1- methylcyclopropyl)sulfonyl]-5,16-dioxo- 6-[(1H-pyrazol-3-ylcarbonyl)amino]- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1010 | 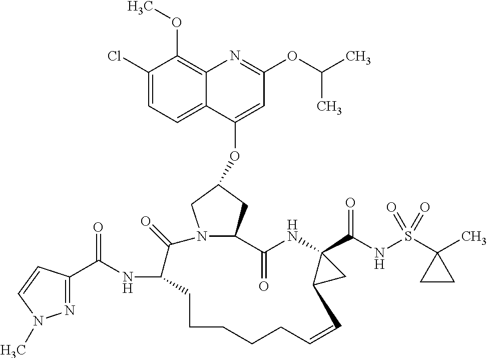 | (2R,6S,12Z,13aS,14aR,16aS)-2-{[7- chloro-8-methoxy-2-(propan-2- yloxy)quinolin-4-yl]oxy}-N-[(1- methylcyclopropyl)sulfonyl]-6-{[(1- methyl-1H-pyrazol-3- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1011 | 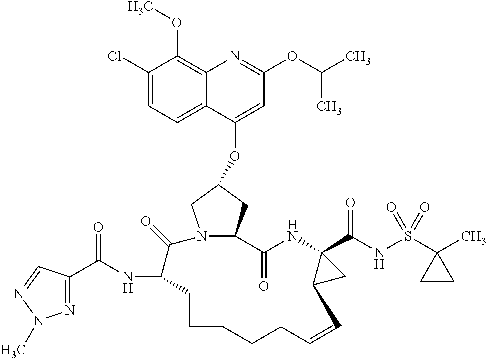 | (2R,6S,12Z,13aS,14aR,16aS)-2-{[7- chloro-8-methoxy-2-(propan-2- yloxy)quinolin-4-yl]oxy}-N-[(1- methylcyclopropyl)sulfonyl]-6-{[(2- methyl-2H-1,2,3-triazol-4- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1012 | 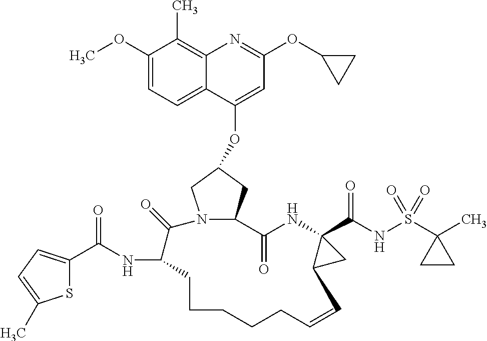 | (2R,6S,12Z,13aS,14aR,16aS)-2-{[2- (cyclopropyloxy)-7-methoxy-8- methylquinolin-4-yl]oxy}-N-[(1- methylcyclopropyl)sulfonyl]-6-{[(5- methylthiophen-2-yl)carbonyl]amino}- 5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1013 | 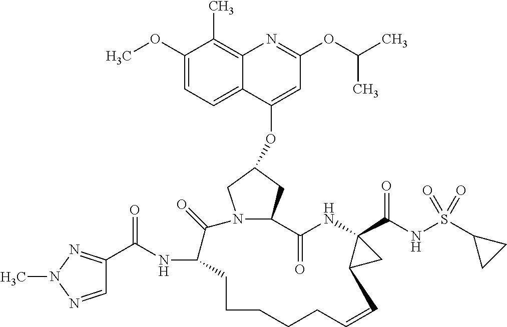 | (2R,6S,12Z,13aS,14aR,16aS)-N- (cyclopropylsulfonyl)-2-{[7-methoxy-8- methyl-2-(propan-2-yloxy)quinolin-4- yl]oxy}-6-{[(2-methyl-2H-1,2,3-triazol-4- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1014 | 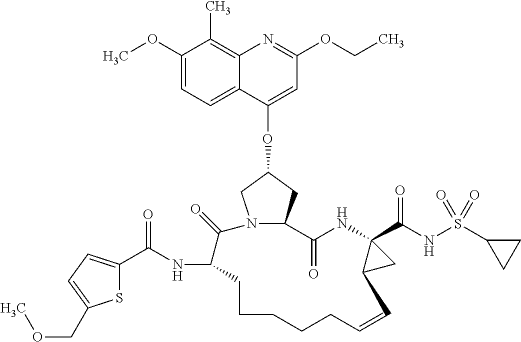 | (2R,6S,12Z,13aS,14aR,16aS)-N- (cyclopropylsulfonyl)-2-[(2-ethoxy-7- methoxy-8-methylquinolin-4-yl)oxy]-6- ({[5-(methoxymethyl)thiophen-2- yl]carbonyl}amino)-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1015 | 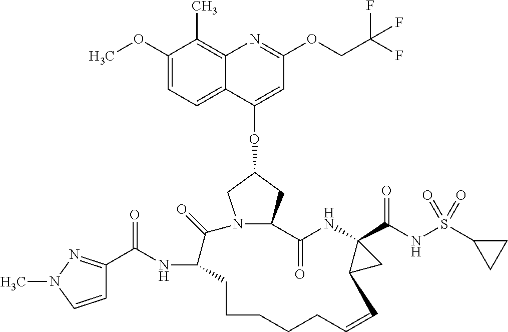 | (2R,6S,12Z,13aS,14aR,16aS)-N- (cyclopropylsulfonyl)-2-{[7-methoxy-8- methyl-2-(2,2,2-trifluoroethoxy)quinolin- 4-yl]oxy}-6-{[(1-methyl-1H-pyrazol-3- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1016 | 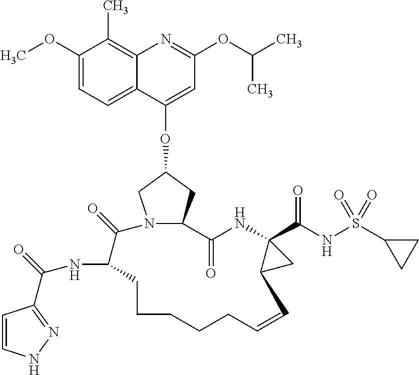 | (2R,6S,12Z,13aS,14aR,16aS)-N- (cyclopropylsulfonyl)-2-{[7-methoxy-8- methyl-2-(propan-2-yloxy)quinolin-4- yl]oxy}-5,16-dioxo-6-[(1H-pyrazol-3- ylcarbonyl)amino]- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1017 | 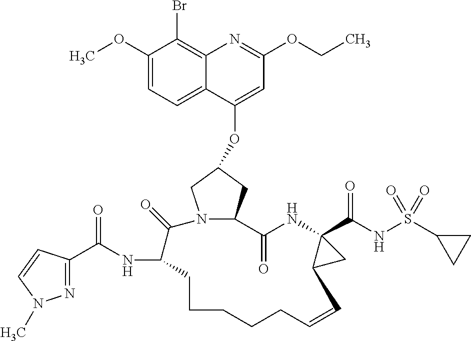 | (2R,6S,12Z,13aS,14aR,16aS)-2-[(8- bromo-2-ethoxy-7-methoxyquinolin-4- yl)oxy]-N-(cyclopropylsulfonyl)-6-{[(1- methyl-1H-pyrazol-3- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1018 | 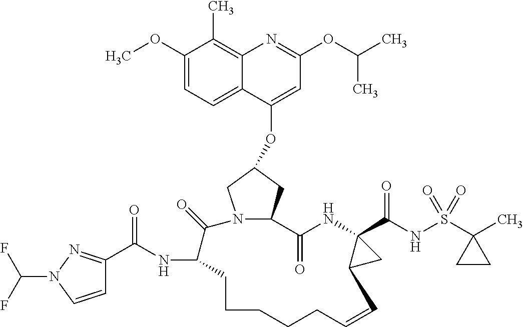 | (2R,6S,12Z,13aS,14aR,16aS)-6-({[1- (difluoromethyl)-1H-pyrazol-3- yl]carbonyl}amino)-2-{[7-methoxy-8- methyl-2-(propan-2-yloxy)quinolin-4- yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1019 | 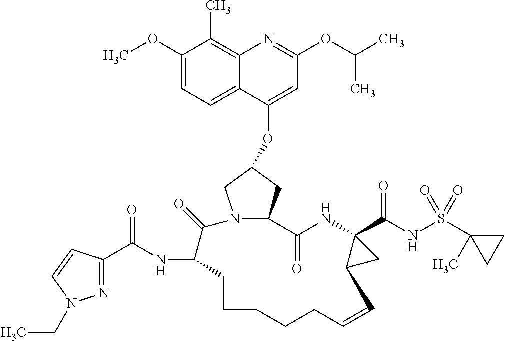 | (2R,6S,12Z,13aS,14aR,16aS)-6-{[(1- ethyl-1H-pyrazol-3-yl)carbonyl]amino}- 2-{[7-methoxy-8-methyl-2-(propan-2- yloxy)quinolin-4-yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1020 | 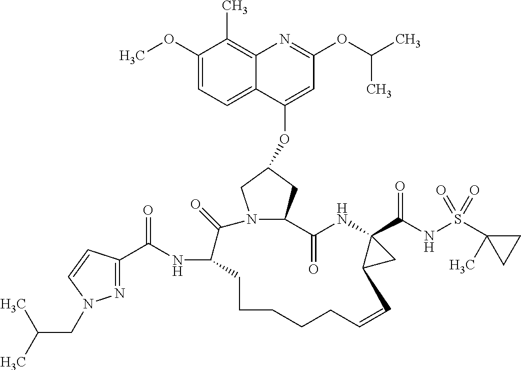 | (2R,6S,12Z,13aS,14aR,16aS)-2-{[7- methoxy-8-methyl-2-(propan-2- yloxy)quinolin-4-yl]oxy}-N-[(1- methylcyclopropyl)sulfonyl]-6-({[1-(2- methylpropyl)-1H-pyrazol-3- yl]carbonyl}amino)-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1021 | 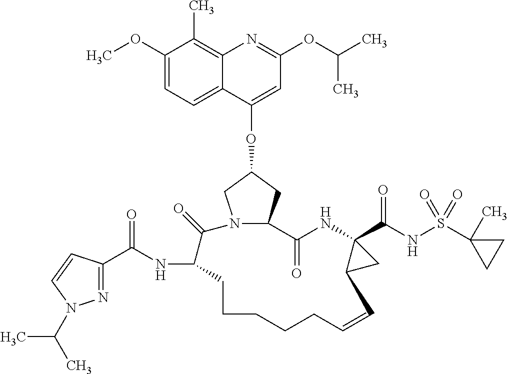 | (2R,6S,12Z,13aS,14aR,16aS)-2-{[7- methoxy-8-methyl-2-(propan-2- yloxy)quinolin-4-yl]oxy}-N-[(1- methylcyclopropyl)sulfonyl]-5,16-dioxo- 6-({[1-(propan-2-yl)-1H-pyrazol-3- yl]carbonyl}amino)- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1022 | 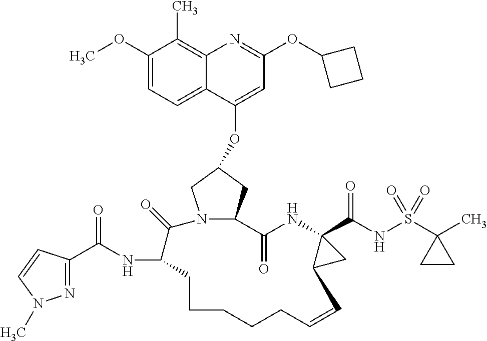 | (2R,6S,12Z,13aS,14aR,16aS)-2-{[2- (cyclobutyloxy)-7-methoxy-8- methylquinolin-4-yl]oxy}-N-[(1- methylcyclopropyl)sulfonyl]-6-{[(1- methyl-1H-pyrazol-3- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1023 | 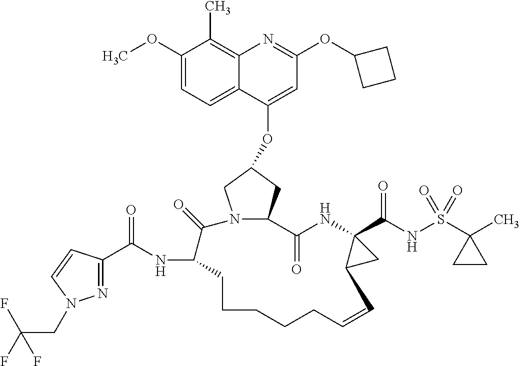 | (2R,6S,12Z,13aS,14aR,16aS)-2-{[2- (cyclobutyloxy)-7-methoxy-8- methylquinolin-4-yl]oxy}-N-[(1- methylcyclopropyl)sulfonyl]-5,16-dioxo- 6-({[1-(2,2,2-trifluoroethyl)-1H-pyrazol- 3-yl]carbonyl}amino)- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1024 | 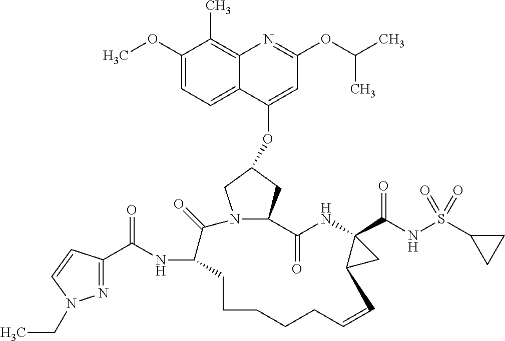 | (2R,6S,12Z,13aS,14aR,16aS)-N- (cyclopropylsulfonyl)-6-{[(1-ethyl-1H- pyrazol-3-yl)carbonyl]amino}-2-{[7- methoxy-8-methyl-2-(propan-2- yloxy)quinolin-4-yl]oxy}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1025 | 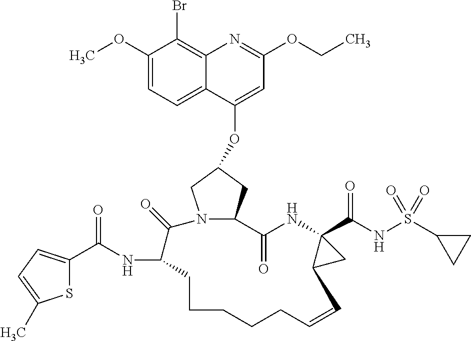 | (2R,6S,12Z,13aS,14aR,16aS)-2-[(8- bromo-2-ethoxy-7-methoxyquinolin-4- yl)oxy]-N-(cyclopropylsulfonyl)-6-{[(5- methylthiophen-2-yl)carbonyl]amino}- 5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1026 | 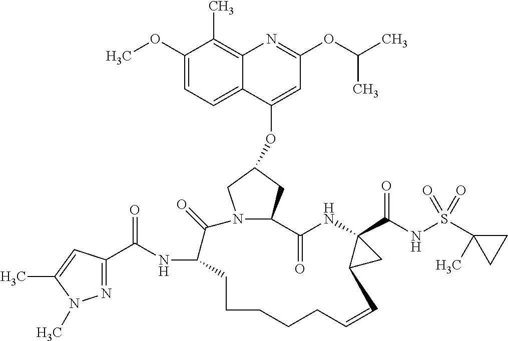 | (2R,6S,12Z,13aS,14aR,16aS)-6-{[(1,5- dimethyl-1H-pyrazol-3- yl)carbonyl]amino}-2-{[7-methoxy-8- methyl-2-(propan-2-yloxy)quinolin-4- yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1027 | 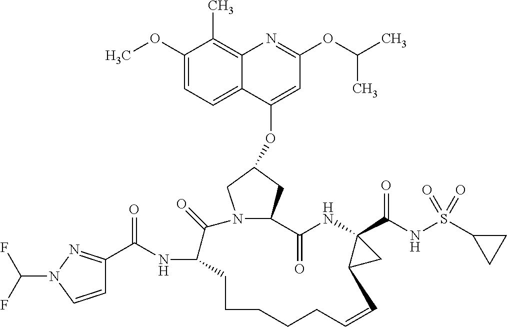 | (2R,6S,12Z,13aS,14aR,16aS)-N- (cyclopropylsulfonyl)-6-({[1- (difluoromethyl)-1H-pyrazol-3- yl]carbonyl}amino)-2-{[7-methoxy-8- methyl-2-(propan-2-yloxy)quinolin-4- yl]oxy}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1028 | 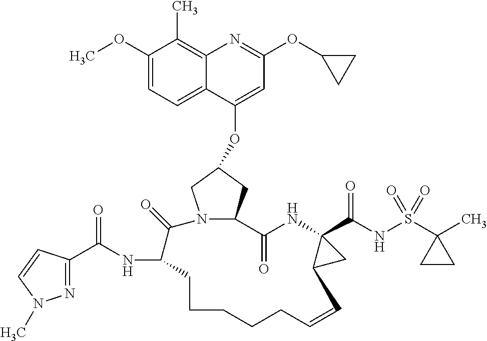 | (2R,6S,12Z,13aS,14aR,16aS)-2-{[2- (cyclopropyloxy)-7-methoxy-8- methylquinolin-4-yl]oxy}-N-[(1- methylcyclopropyl)sulfonyl]-6-{[(1- methyl-1H-pyrazol-3- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1029 | 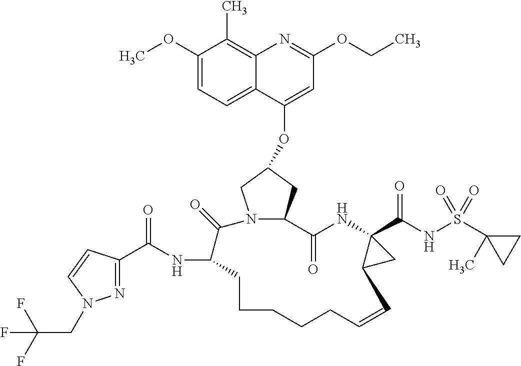 | (2R,6S,12Z,13aS,14aR,16aS)-2-[(2- ethoxy-7-methoxy-8-methylquinolin-4- yl)oxy]-N-[(1- methylcyclopropyl)sulfonyl]-5,16-dioxo- 6-({[1-(2,2,2-trifluoroethyl)-1H-pyrazol- 3-yl]carbonyl}amino)- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1030 | 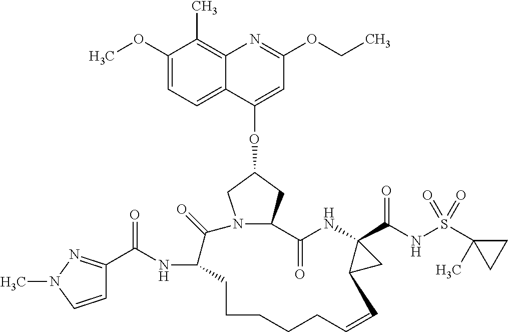 | (2R,6S,12Z,13aS,14aR,16aS)-2-[(2- ethoxy-7-methoxy-8-methylquinolin-4- yl)oxy]-N-[(1- methylcyclopropyl)sulfonyl]-6-{[(1- methyl-1H-pyrazol-3- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1031 | 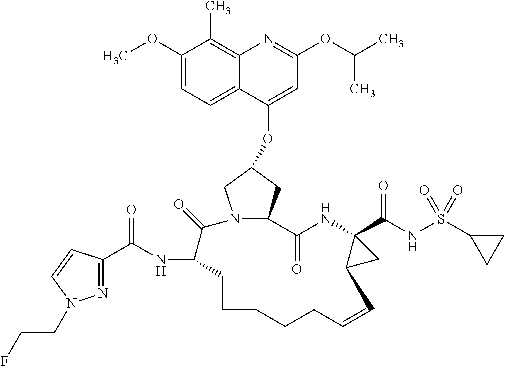 | (2R,6S,12Z,13aS,14aR,16aS)-N- (cyclopropylsulfonyl)-6-({[1-(2- fluoroethyl)-1H-pyrazol-3- yl]carbonyl}amino)-2-{[7-methoxy-8- methyl-2-(propan-2-yloxy)quinolin-4- yl]oxy}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1032 | 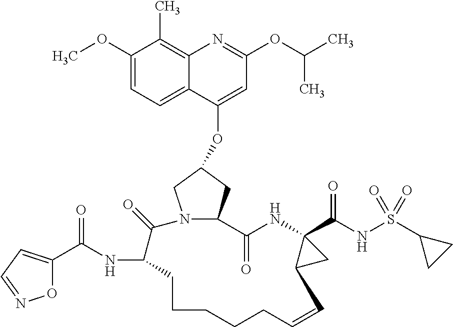 | (2R,6S,12Z,13aS,14aR,16aS)-N- (cyclopropylsulfonyl)-2-{[7-methoxy-8- methyl-2-(propan-2-yloxy)quinolin-4- yl]oxy}-6-[(1,2-oxazol-5- ylcarbonyl)amino]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1033 | 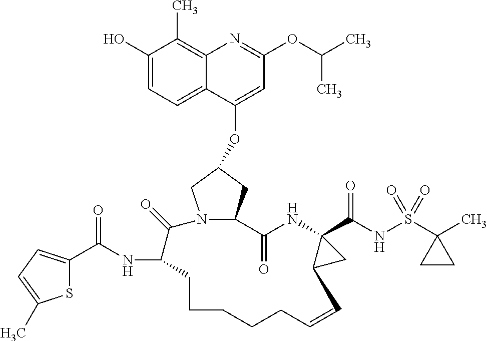 | (2R,6S,12Z,13aS,14aR,16aS)-2-{[7- hydroxy-8-methyl-2-(propan-2- yloxy)quinolin-4-yl]oxy}-N-[(1- methylcyclopropyl)sulfonyl]-6-{[(5- methylthiophen-2-yl)carbonyl]amino}- 5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1034 | 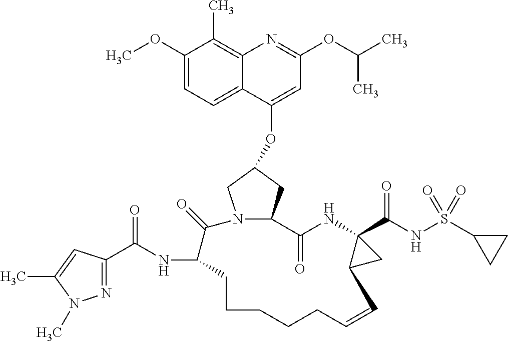 | (2R,6S,12Z,13aS,14aR,16aS)-N- (cyclopropylsulfonyl)-6-{[(1,5-dimethyl- 1H-pyrazol-3-yl)carbonyl]amino}-2-{[7- methoxy-8-methyl-2-(propan-2- yloxy)quinolin-4-yl]oxy}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1035 | 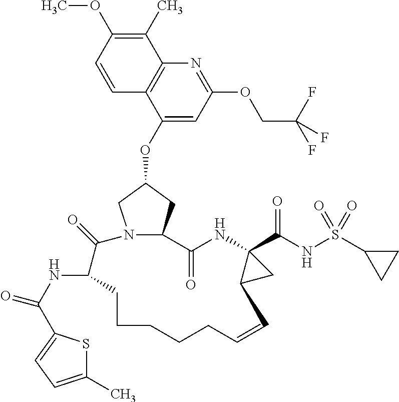 | (2R,6S,12Z,13aS,14aR,16aS)-N- (cyclopropylsulfonyl)-2-{[7-methoxy-8- methyl-2-(2,2,2-trifluoroethoxy)quinolin- 4-yl)oxy}-6-{[(5-methylthiophen-2- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1036 | 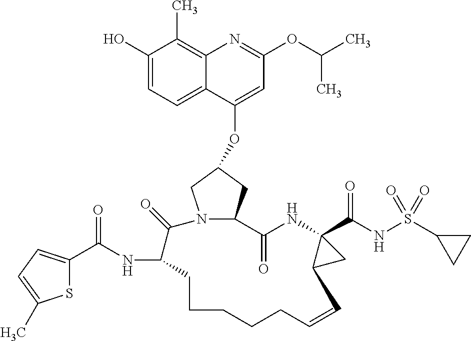 | (2R,6S,12Z,13aS,14aR,16aS)-N- (cyclopropylsulfonyl)-2-{[7-hydroxy-8- methyl-2-(propan-2-yloxy)quinolin-4- yl]oxy}-6-{[(5-methylthiophen-2- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 1037 | 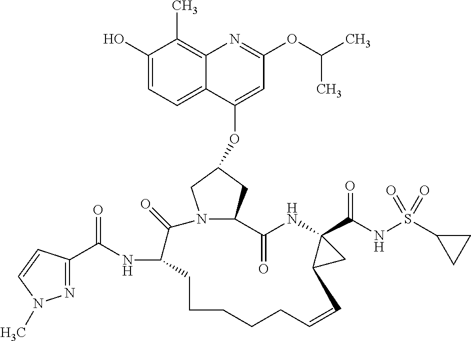 | (2R,6S,12Z,13aS,14aR,16aS)-N- (cyclopropylsulfonyl)-2-{[7-hydroxy-8- methyl-2-(propan-2-yloxy)quinolin-4- yl]oxy}-6-{[(1-methyl-1H-pyrazol-3- yl)carbonyl]amino}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |

TABLE 2

| Cmpd # | Structure | Name |
|---|---|---|
| 2001 | 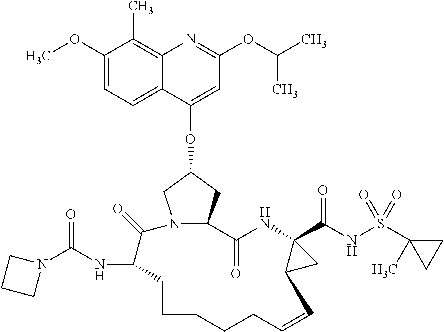 | (2R,6S,12Z,13aS,14aR,16aS)-6- [(azetidin-1-ylcarbonyl)amino]-2-{[7- methoxy-8-methyl-2-(propan-2- yloxy)quinolin-4-yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2002 | 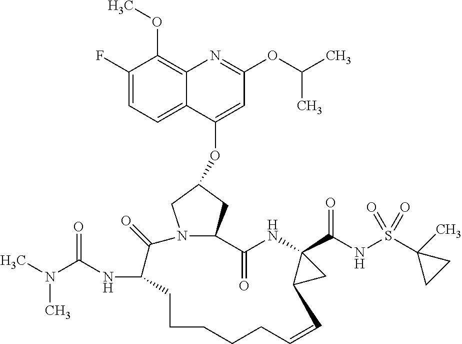 | (2R,6S,12Z,13aS,14aR,16aS)-6- [(dimethylcarbamoyl)amino]-2-{[7- fluoro-8-methoxy-2-(propan-2- yloxy)quinolin-4-yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2003 | 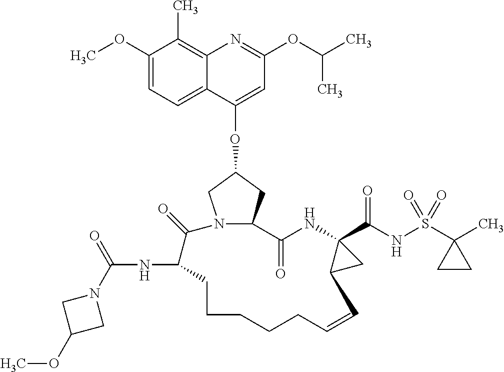 | (2R,6S,12Z,13aS,14aR,16aS)-6-{[(3- methoxyazetidin-1-yl)carbonyl]amino}- 2-{[7-methoxy-8-methyl-2-(propan-2- yloxy)quinolin-4-yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2004 | 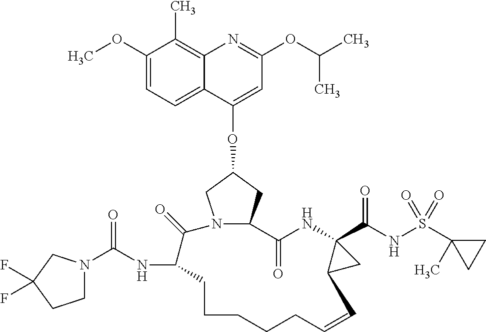 | (2R,6S,12Z,13aS,14aR,16aS)-6-{[(3,3- difluoropyrrolidin-1-yl)carbonyl]amino}- 2-{[7-methoxy-8-methyl-2-(propan-2- yloxy)quinolin-4-yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2005 | 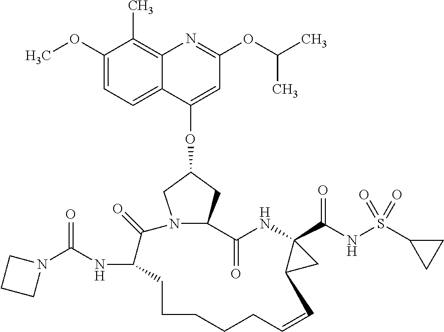 | (2R,6S,12Z,13aS,14aR,16aS)-6- [(azetidin-1-ylcarbonyl)amino]-N- (cyclopropylsulfonyl)-2-{[7-methoxy-8- methyl-2-(propan-2-yloxy)quinolin-4- yl]oxy}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2006 | 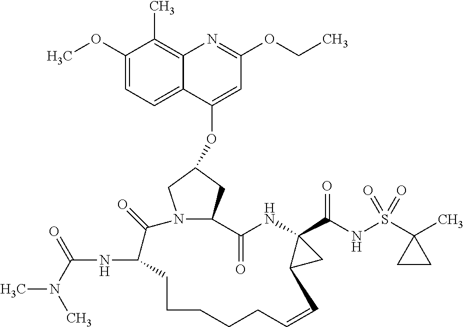 | (2R,6S,12Z,13aS,14aR,16aS)-6- [(dimethylcarbamoyl)amino]-2-[(2- ethoxy-7-methoxy-8-methylquinolin-4- yl)oxy]-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2007 | 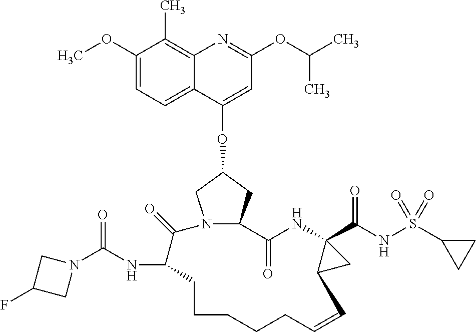 | (2R,6S,12Z,13aS,14aR,16aS)-N- (cyclopropylsulfonyl)-6-{[(3- fluoroazetidin-1-yl)carbonyl]amino}-2- {[7-methoxy-8-methyl-2-(propan-2- yloxy)quinolin-4-yl]oxy}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2008 | 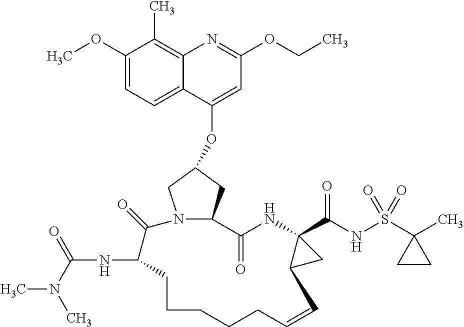 | (2R,6S,12Z,13aS,14aR,16aS)-6- [(dimethylcarbamoyl)amino]-2-{[7- methoxy-8-methyl-2-(propan-2- yloxy)quinolin-4-yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2009 | 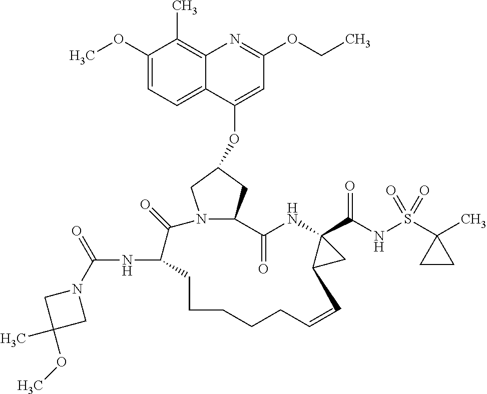 | (2R,6S,12Z,13aS,14aR,16aS)-6-{[(3- methoxy-3-methylazetidin-1- yl)carbonyl]amino}-2-{[7-methoxy-8- methyl-2-(propan-2-yloxy)quinolin-4- yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2010 | 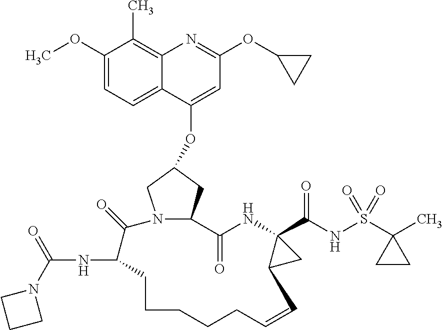 | (2R,6S,12Z,13aS,14aR,16aS)-6- [(azetidin-1-ylcarbonyl)amino]-2-{[2- (cyclopropyloxy)-7-methoxy-8- methylquinolin-4-yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2011 | 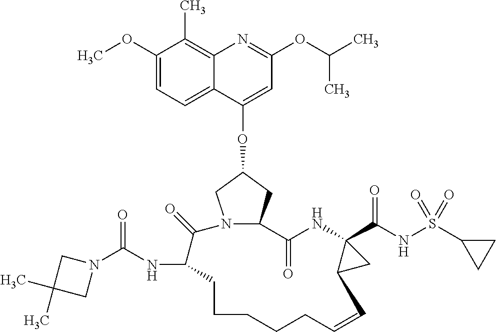 | (2R,6S,12Z,13aS,14aR,16aS)-N- (cyclopropylsulfonyl)-6-{[(3,3- dimethylazetidin-1-yl)carbonyl]amino}- 2-{[7-methoxy-8-methyl-2-(propan-2- yloxy)quinolin-4-yl]oxy}-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2012 | 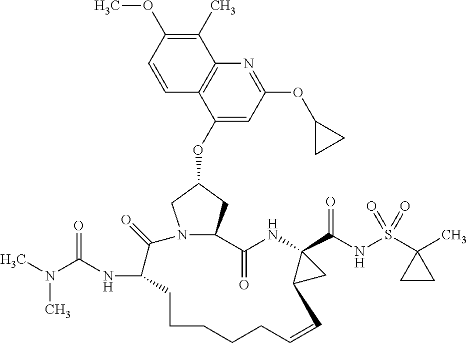 | (2R,6S,12Z,13aS,14aR,16aS)-2-{[2- (cyclopropyloxy)-7-methoxy-8- methylquinolin-4-yl]oxy}-6- [(dimethylcarbamoyl)amino]-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2013 | 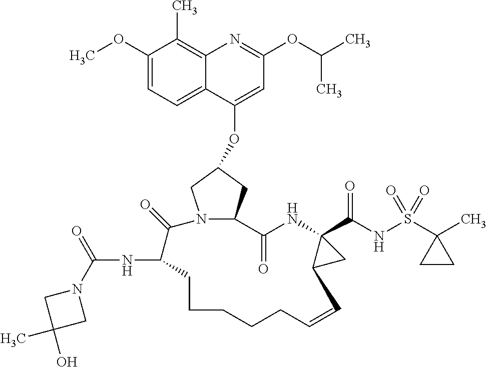 | (2R,6S,12Z,13aS,14aR,16aS)-6-{[(3- hydroxy-3-methylazetidin-1- yl)carbonyl]amino}-2-{[7-methoxy-8- methyl-2-(propan-2-yloxy)quinolin-4- yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2014 | 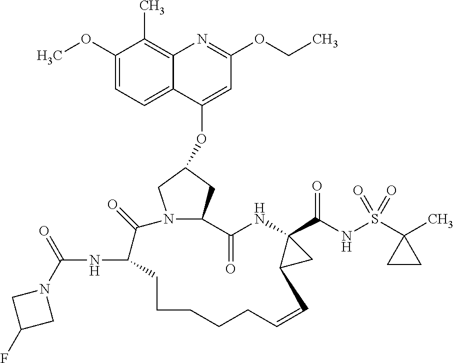 | (2R,6S,12Z,13aS,14aR,16aS)-2-[(2- ethoxy-7-methoxy-8-methylquinolin-4- yl)oxy]-6-{[(3-fluoroazetidin-1- yl)carbonyl]amino}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2015 | 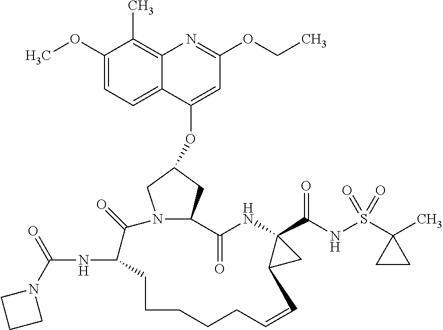 | (2R,6S,12Z,13aS,14aR,16aS)-6- [(azetidin-1-ylcarbonyl)amino]-2-[(2- ethoxy-7-methoxy-8-methylquinolin-4- yl)oxy]-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2016 | 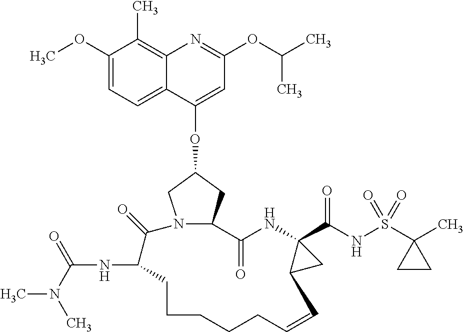 | (2R,6S,12Z,13aS,14aR,16aS)-2-{[8- bromo-7-methoxy-2-(propan-2- yloxy)quinolin-4-yl]oxy}-6- [(dimethylcarbamoyl)amino]-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2017 | 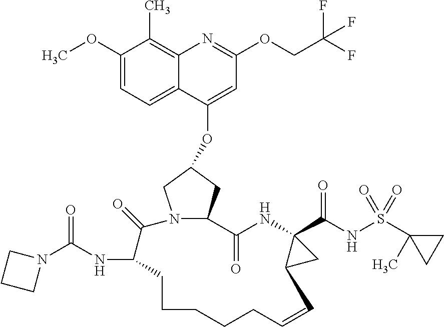 | (2R,6S,12Z,13aS,14aR,16aS)-6- [(azetidin-1-ylcarbonyl)amino]-2-{[7- methoxy-8-methyl-2-(2,2,2- trifluoroethoxy)quinolin-4-yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2018 | 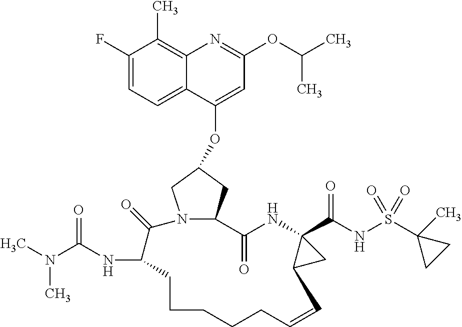 | (2R,6S,12Z,13aS,14aR,16aS)-6- [(dimethylcarbamoyl)amino]-2-{[7- fluoro-8-methyl-2-(propan-2- yloxy)quinolin-4-yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2019 | 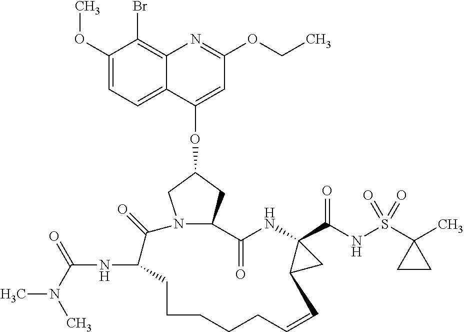 | (2R,6S,12Z,13aS,14aR,16aS)-2-[(8- bromo-2-ethoxy-7-methoxyquinolin-4- yl)oxy]-6-[(dimethylcarbamoyl)amino]- N-[(1-methylcyclopropyl)sulfonyl]-5,16- dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2020 | 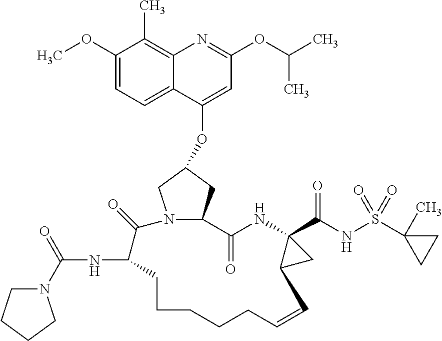 | (2R,6S,12Z,13aS,14aR,16aS)-2-{[7- methoxy-8-methyl-2-(propan-2- yloxy)quinolin-4-yl]oxy}-N-[(1- methylcyclopropyl)sulfonyl]-5,16-dioxo- 6-[(pyrrolidin-1-ylcarbonyl)amino]- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2021 | 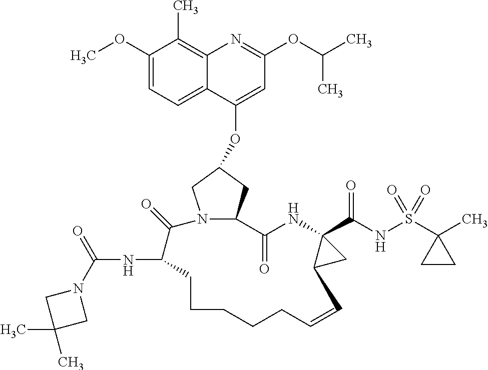 | (2R,6S,12Z,13aS,14aR,16aS)-6-{[(3,3- dimethylazetidin-1-yl)carbonyl]amino}- 2-{[7-methoxy-8-methyl-2-(propan-2- yloxy)quinolin-4-yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |
| 2022 | 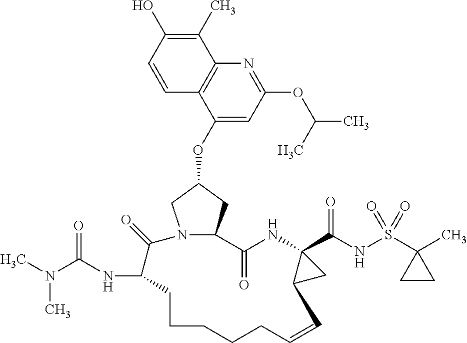 | (2R,6S,12Z,13aS,14aR,16aS)-6- [(dimethylcarbamoyl)amino]-2-{[7- hydroxy-8-methyl-2-(propan-2- yloxy)quinolin-4-yl]oxy}-N-[(1- methylcyclopropyl)sulfonyl]-5,16-dioxo- 1,2,3,6,7,8,9,10,11,13a,14,15,16,16a- tetradecahydrocyclopropa[e]pyrrolo[1,2- a][1,4]diazacyclopentadecine-14a(5H)- carboxamide |

Another aspect of this invention provides compounds of Formula (I) that exhibit unexpectedly good cell-based potency against genotype 1a R155K and genotype 1b D168V resistance mutations.

Furthermore, the invention provides compounds 1001-1037 and compounds 2001-2022 that exhibit unexpectedly good cell-based potency against genotype 1a R155K and genotype 1b D168V resistance mutations.

Furthermore, the invention provides any one of compounds 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 2001, 2002, 2003, 2004, 2005, 2006, 2007, 2008, 2009, 2010, 2011, 2012, 2013, 2014, 2015, 2016, 2017, 2018, 2019, 2020, 202, 2022 and their pharmaceutically acceptable salts forms that exhibit unexpectedly good cell-based potency against genotype 1a R155K and genotype 1b D168V resistance mutations.

Another aspect of this invention provides compounds of the invention, or a pharmaceutically acceptable salt thereof, as a medicament.

Included within the scope of this invention is a pharmaceutical composition comprising an anti-hepatitis C virally effective amount of a compound of the invention, or a pharmaceutically acceptable salt thereof, in admixture with at least one pharmaceutically acceptable carrier medium or auxiliary agent.

According to a further aspect of this embodiment the pharmaceutical composition according to this invention further comprises a therapeutically effective amount of at least one other antiviral agent.

The invention also provides the use of a pharmaceutical composition as described hereinabove for the treatment of a hepatitis C viral infection in a human being having or at risk of having the infection.

Another important aspect of the invention involves a method of treating or preventing a hepatitis C viral infection in a human being by administering to the human being an anti-hepatitis C virally effective amount of a compound of the invention, a pharmaceutically acceptable salt thereof, or a composition as described above, alone or in combination with at least one other antiviral agent, administered together or separately.

Also within the scope of this invention is the use of a compound of the invention, or a pharmaceutically acceptable salt thereof, as described herein, for the manufacture of a medicament for the treatment or prevention of hepatitis C viral infection in human being.

An additional aspect of this invention refers to an article of manufacture comprising a composition effective to treat a hepatitis C viral infection; and packaging material comprising a label which indicates that the composition can be used to treat infection by the hepatitis C virus; wherein the composition comprises a compound according to this invention or a pharmaceutically acceptable salt thereof.

Still another aspect of this invention relates to a method of inhibiting the replication of hepatitis C virus comprising exposing the virus to an effective amount of the compound of the invention, or a salt thereof, under conditions where replication of hepatitis C virus is inhibited.

Further included in the scope of the invention is the use of a compound of the invention, or a salt thereof, to inhibit the replication of hepatitis C virus.

Yet another aspect of this invention provides a method of inhibiting HCV NS3 protease activity in a human being by administering a compound of the invention, including a pharmaceutically acceptable salt thereof.

Another aspect of this invention provides a method of decreasing the NS3 protease activity of the hepatitis C virus infecting a human being by administering a compound of the invention, including a pharmaceutically acceptable salt thereof.

### DETAILED DESCRIPTION OF PREFERRED EMBODIMENTS

### Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general, for groups comprising two or more subgroups, the first named subgroup is the radical attachment point, for example, the substituent “—C₁₋₃-alkyl-aryl” means an aryl group which is bound to a C₁₋₃-alkyl-group, with the C₁₋₃-alky group bound to the core. Unless specifically stated otherwise, for groups comprising two or more subgroups, the substituent may be attached to either subgroup.

The following designation * is used in sub-formulas to indicate the bond which is connected to the rest of the molecule as defined.

Unless specifically indicated, throughout the specification and the appended claims, a given chemical formula or name shall encompass salts, including pharmaceutically acceptable salts thereof and solvates thereof, such as for instance hydrates, including solvates of the free compounds or solvates of a salt of the compound. For example, the compounds of the present invention can exist in unsolvated as well as solvated forms with pharmaceutically acceptable solvents such as water, ethanol, and the like. In general, the solvated forms are considered equivalent to the unsolvated forms for the purpose of the present invention.

Unless specifically indicated, throughout the specification and the appended claims, a given chemical formula or name shall encompass tautomers, and all stereo, optical and geometrical isomers (e.g. enantiomers, diastereomers, E/Z isomers) resulting from all possible steriochemistry at a chiral center for which specific steriochemistry is not otherwise decsribed, and racemates thereof, as well as mixtures in different proportions of the separate enantiomers, mixtures of diastereomers, or mixtures of any of the foregoing forms where such isomers and enantiomers exist.

One skilled in the art would know how to separate, enrich, or selectively prepare the enantiomers of the compounds of the present invention. Preparation of pure stereoisomers, e.g. enantiomers and diastereomers, or mixtures of desired enantiomeric excess (ee) or enantiomeric purity, are accomplished by one or more of the many methods of (a) separation or resolution of enantiomers, or (b) enantioselective synthesis known to those of skill in the art, or a combination thereof. These resolution methods generally rely on chiral recognition and include but not limited to chromatography using chiral stationary phases, enantioselective host-guest complexation, resolution or synthesis using chiral auxiliaries, enantioselective synthesis, enzymatic and nonenzymatic kinetic resolution, or spontaneous enantioselective crystallization. Such methods are disclosed generally in Chiral Separation Techniques: A Practical Approach (2nd Ed.), G. Subramanian (ed.), Wiley-VCH, 2000; T. E. Beesley and R. P. W. Scott, Chiral Chromatography, John Wiley & Sons, 1999; and Satinder Ahuja, Chiral Separations by Chromatography, Am. Chem. Soc., 2000. Furthermore, there are equally well-known methods for the quantitation of enantiomeric excess or purity, including but not limited to GC, HPLC, CE, or NMR, and assignment of absolute configuration and conformation, including but not limited to CD, ORD, X-ray crystallography, or NMR.

The term halo generally denotes fluorine, chlorine, bromine and iodine.

The term “C₁₋ₙ-alkyl”, wherein n is an integer from 2 to n, either alone or in combination with another radical denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—C(CH₃)₂—, H₃C—CH(CH₃)—CH₂—CH₂—, H₃C—CH₂—C(CH₃)₂—, H₃C—C(CH₃)₂—CH₂—, and H₃C—CH₂—CH(CH₂CH₃)—.

The term “C₃₋ₙ-cycloalkyl”, wherein n is an integer 4 to n, either alone or in combination with another radical denotes a cyclic, saturated, unbranched hydrocarbon radical with 3 to n C atoms. For example the term C₃₋₇-cycloalkyl includes cyclopropyl, cyclobutyl, cyclopentyl, cyclohexyl and cycloheptyl.

The term “Het” as used herein, either alone or in combination with another radical, is intended to mean a 4- to 7-membered saturated, unsaturated or aromatic heterocycle having 1 to 4 heteroatoms each independently selected from O, N and S; wherein each N heteroatom may, independently and where possible, exist in an oxidized state such that it is further bonded to an oxygen atom to form an N-oxide group and wherein each S heteroatom may, independently and where possible, exist in an oxidized state such that it is further bonded to one or two oxygen atoms to form the groups SO or SO₂, unless specified otherwise. When a Het group is substituted, it is understood that substituents may be attached to any carbon atom or heteroatom thereof which would otherwise bear a hydrogen atom, unless specified otherwise. Examples of Het include, but are not limited to,

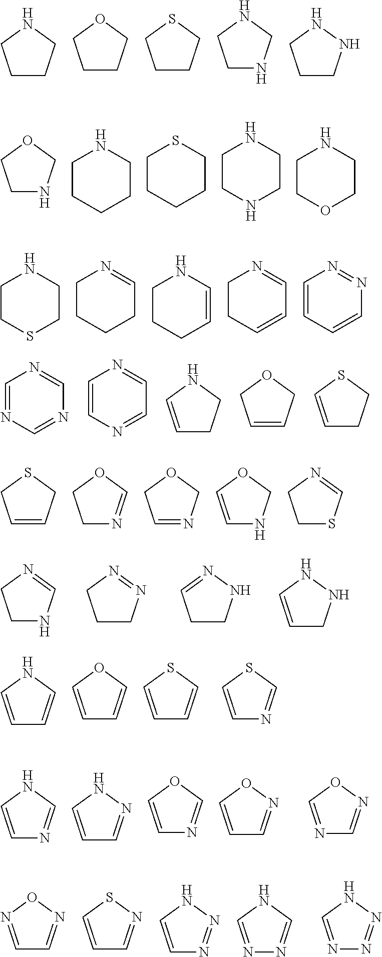

Unless specifically indicated, throughout the specification and the appended claims, a given chemical formula or name shall encompass salts, including pharmaceutically acceptable salts thereof. The term “salt thereof” as used herein is intended to mean any acid and/or base addition salt of a compound according to the invention, including but not limited to a pharmaceutically acceptable salt thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. For example, such salts include acetates, ascorbates, aspartates, benzenesulfonates, benzoates, besylates, bicarbonates, bitartrates, bromides/hydrobromides, Ca-edetates/edetates, camsylates, carbonates, chlorides/hydrochlorides, citrates, cyclamates, edisylates, ethane disulfonates, estolates, esylates, fumarates, gentisates (salt of 2,5-dihydroxy benzoic acid), gluceptates, gluconates, glutamates, glycinates, glycolates, glycollylarsnilates, hexylresorcinates, hydrabamines, hydroxymaleates, hydroxynaphthoates, iodides, isethionates, lactates, lactobionates, malates, maleates, malonates, mandelates, methanesulfonates, mesylates, methylbrom ides, methylnitrates, methylsulfates, mucates, napsylates, nitrates, oxalates, pamoates, pantothenates, phenylacetates, phosphates/diphosphates, polygalacturonates, propionates, saccharinates, salicylates, stearates subacetates, succinates, sulfamides, sulfates, tannates, tartrates, teoclates, toluenesulfonates, triethiodides, xinafoates (salt of 1-hydroxy-2-naphthoicacid), ammonium, arginine, benzathines, chloroprocaines, cholines, diethanolamines, ethylenediamines, lysine, meglumines, TRIS (C,C,C-tris(hydroxymethyl)-aminomethan or Trometamol) and procaines. Further pharmaceutically acceptable salts can be formed with cations from metals like aluminium, calcium, lithium, magnesium, potassium, sodium, zinc and the like. (also see Pharmaceutical salts, Birge, S. M. et al., J. Pharm. Sci., (1977), 66, 1-19).

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention also comprise a part of the invention.

The term “antiviral agent” as used herein means an agent (compound or biological) that is effective to inhibit the formation and/or replication of a virus in a human being. This includes agents that interfere with either host or viral mechanisms necessary for the formation and/or replication of a virus in a human being. Such agents can be selected from: another anti-HCV agent, HIV inhibitor, HAV inhibitor and HBV inhibitor.

As used herein, the term “treatment” means the administration of a compound or composition according to the present invention to alleviate or eliminate symptoms of the hepatitis C disease and/or to reduce viral load in a patient.

As used herein, the term “prevention” means the administration of a compound or composition according to the present invention post-exposure of the individual to the virus but before the appearance of symptoms of the disease, and/or prior to the detection of the virus in the blood, to prevent the appearance of symptoms of the disease.

The term “therapeutically effective amount” means an amount of a compound according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

### PREFERRED EMBODIMENTS

In the following preferred embodiments, groups and substituents of the compounds of Formula (I) according to this invention are described in detail.

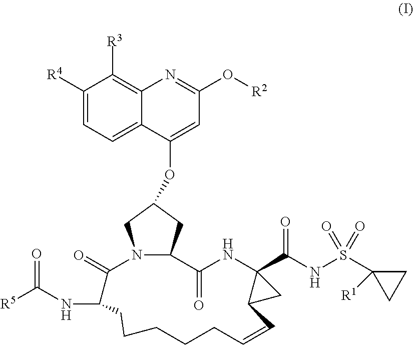

### R¹

R¹-A: R¹ is H or (C₁₋₆)alkyl.
R¹—B: R¹ is H or (C₁₋₃)alkyl.
R¹—C: R¹ is H or CH₃.

### R²

R²-A: R² is (C₁₋₆)alkyl optionally substituted 1-3 times with halo or (C₃₋₇)cycloalkyl.
R²—B: R² is (C₁₋₃)alkyl optionally substituted 1-3 times with halo or (C₃₋₄)cycloalkyl.
R²—C: R² is

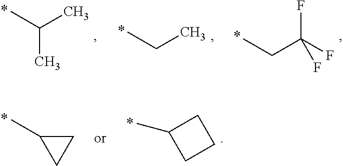

### R³

R³-A: R³ is (C₁₋₆)alkyl, —O—(C₁₋₆)alkyl or halo.
R³—B: R³ is C₁₋₃)alkyl, —O—(C₁₋₃)alkyl or halo.
R³—C: R³ is CH₃, —OCH₃, Cl, Br or F.

### R⁴

R⁴-A: R⁴ is —O—(C₁₋₆)alkyl, —OH or halo.
R⁴—B: R⁴ is —O—(C₁₋₃)alkyl, —OH or halo.
R⁴—C: R⁴ is —OCH₃, —OCH₂CH₃, —OH, F or Cl.

### R⁵

R⁵-A: R⁵ is Het optionally substituted 1-3 times with (C₁₋₆)alkyl, —(C₁₋₆)alkyl-C(═O)—N((C₁₋₆)alkyl)₂, —(C₁₋₆)alkyl-O—(C₁₋₆)alkyl or (C₁₋₆)haloalkyl; or
   R⁵ is —N(R^{A})(R^{B}) wherein R^{A} and R^{B} are (C₁₋₆)alkyl or R^{A} and R^{B} are linked together with the N to which they are attached to form a 4- to 7-membered saturated ring, wherein said ring is optionally substituted 1-3 times with (C₁₋₆)alkyl, —O—(C₁₋₆)alkyl,
   —OH or halo.
R⁵—B: R⁵ is a 5-membered aromatic Het optionally substituted 1-3 times with (C₁₋₄)alkyl, —(C₁₋₃)alkyl-C(═O)—N((C₁₋₃)alkyl)₂, —(C₁₋₃)alkyl-O—(C₁₋₃)alkyl or (C₁₋₃)haloalkyl; or
   R⁵ is —N(R^{A})(R^{B}) wherein R^{A} and R^{B} are (C₁₋₃)alkyl or R^{A} and R^{B} are linked together with the N to which they are attached to form a 4- to 5-membered saturated ring, wherein said ring is optionally substituted 1-3 times with (C₁₋₃)alkyl, —O—(C₁₋₃)alkyl, —OH or halo.
R⁵—C: R⁵ is

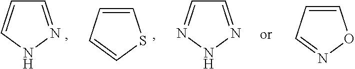

optionally substituted 1-3 times with (C₁₋₄)alkyl, —(C₁₋₃)alkyl-C(═O)—N((C₁₋₃)alkyl)₂, —(C₁₋₃)alkyl-O—(C₁₋₃)alkyl or (C₁₋₃)haloalkyl; or
R⁵ is —N(CH₃)₂,

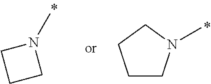

wherein said rings are optionally substituted 1-3 times with (C₁₋₃)alkyl, —O—(C₁₋₃)alkyl, —OH or halo.

Examples of preferred subgeneric embodiments of the present invention are set forth in the following table, wherein each substituent group of each embodiment is defined according to the definitions set forth above:

| | R¹ | R² | R³ | R⁴ | R⁵ |
|---|---|---|---|---|---|
| E-1 | R¹-A | R²-A | R³-A | R⁴-A | R⁵-B |
| E-2 | R¹-A | R²-A | R³-A | R⁴-A | R⁵-B |
| E-3 | R¹-B | R²-B | R³-A | R⁴-C | R⁵-B |
| E-4 | R¹-B | R²-B | R³-A | R⁴-B | R⁵-C |
| E-5 | R¹-B | R²-B | R³-A | R⁴-B | R⁵-B |
| E-6 | R¹-B | R²-B | R³-B | R⁴-B | R⁵-B |
| E-7 | R¹-C | R²-C | R³-B | R⁴-B | R⁵-C |
| E-8 | R¹-C | R²-B | R³-B | R⁴-B | R⁵-C |
| E-9 | R¹-C | R²-C | R³-B | R⁴-B | R⁵-B |
| E-10 | R¹-C | R²-B | R³-B | R⁴-B | R⁵-B |
| E-11 | R¹-C | R²-B | R³-A | R⁴-B | R⁵-C |
| E-12 | R¹-C | R²-A | R³-B | R⁴-B | R⁵-B |
| E-13 | R¹-C | R²-B | R³-A | R⁴-B | R⁵-B |
| E-14 | R¹-C | R²-C | R³-C | R⁴-C | R⁵-C |
| E-15 | R¹-C | R²-B | R³-C | R⁴-C | R⁵-C |
| E-16 | R¹-C | R²-C | R³-C | R⁴-C | R⁵-B |
| E-17 | R¹-C | R²-B | R³-C | R⁴-C | R⁵-B |
| E-18 | R¹-C | R²-B | R³-A | R⁴-C | R⁵-C |
| E-19 | R¹-C | R²-A | R³-C | R⁴-C | R⁵-B |
| E-20 | R¹-C | R²-B | R³-A | R⁴-C | R⁵-B |

In the aforementioned embodiments E1-E20, if applicable, the compounds A1-A29 are excluded.

Examples of most preferred compounds according to this invention are each single compound listed in Tables 1 to 2.

### Pharmaceutical Composition

Suitable preparations for administering the compounds of the invention will be apparent to those with ordinary skill in the art and include for example tablets, pills, capsules, suppositories, lozenges, troches, solutions, syrups, elixirs, sachets, injectables, inhalatives and powders. The content of the pharmaceutically active compound(s) should be in the range from 0.05 to 90 wt.-%, preferably 0.1 to 50 wt.-% of the composition as a whole.

Suitable tablets may be obtained, for example, by mixing one or more compounds according to the invention with known excipients, for example inert diluents, carriers, disintegrants, adjuvants, surfactants, binders and/or lubricants. The tablets may also consist of several layers.

According to an alternate embodiment, the pharmaceutical composition of this invention may additionally comprise at least one other anti-HCV agent.

The term “other anti-HCV agent” as used herein means those agents that are effective for diminishing or preventing the progression of hepatitis C related symptoms of disease. Such agents can be selected from: immunomodulatory agents, inhibitors of HCV NS3 protease, inhibitors of HCV polymerase or inhibitors of another target in the HCV life cycle. Examples of anti-HCV agents include, α-(alpha), β-(beta), δ-(delta), γ-(gamma), ω-(omega) or Σ-(tau) interferon, pegylated α-interferon, ribavirin, amantadine, taribavirin (Viramidine), Nitazoxannide and BMS-791325.

The term “immunomodulatory agent” as used herein includes those agents (compounds or biologicals) that are effective to enhance or potentiate the immune system response in a human being. Immunomodulatory agents include, but are not limited to, inosine monophosphate dehydrogenase inhibitors, class I interferons, class II interferons, consensus interferons, asialo-interferons pegylated interferons and conjugated interferons, including but not limited to interferons conjugated with other proteins including but not limited to human albumin. Class I interferons are a group of interferons that all bind to receptor type I, including both naturally and synthetically produced class I interferons, while class II interferons all bind to receptor type II. Examples of class I interferons include, but are not limited to, α-, β-, δ-, ω-, and τ-interferons, while examples of class II interferons include, but are not limited to, γ-interferons.

The term “inhibitor of HCV NS3 protease” as used herein means an agent (compound or biological) that is effective to inhibit the function of HCV NS3 protease in a human being. Inhibitors of HCV NS3 protease include, for example, those compounds described in WO 99/07733, WO 99/07734, WO 00/09558, WO 00/09543, WO 00/59929, WO 03/064416, WO 03/064455, WO 03/064456, WO 2004/039970, WO 2004/037855, WO 2004/039833, WO 2004/101602, WO 2004/101605, WO 2004/103996, WO 2005/028501, WO 2005/070955, WO 2006/000085, WO 2006/007700, WO 2006/007708, WO 2007/009227, WO 2004/093915, WO 2004/009121 (all by Boehringer Ingelheim), all of which are herein incorporated by reference; and the candidates ABT-450, ACH-1625, BMS-650032, PHX1766, VX-813, VX-950, AVL-181, SCH-503034, SCH-900518, ITMN-191, TMC 435350, MK7009 and BI 201335.

The term “inhibitor of HCV polymerase” as used herein means an agent (compound or biological) that is effective to inhibit the function of an HCV polymerase in a human being. This includes, for example, inhibitors of HCV NS5B polymerase. Inhibitors of HCV polymerase include for example, those compounds described in: WO 03/007945, WO 03/010140, WO 03/010141, U.S. Pat. No. 6,448,281, WO 02/04425, WO 2008/019477, WO 2007/087717, WO 2006/007693, WO 2005/080388, WO 2004/099241, WO 2004/065367, WO 2004/064925 (all by Boehringer Ingelheim), all of which are herein incorporated by reference. Specific examples of inhibitors of an HCV polymerase, include RG-7128, GS9190, IDX184, PSI-7851, MK-3281, PF868554, VCH-222, VCH-759, ANA598, ABT-333 and ABT-072.

The term “inhibitor of another target in the HCV life cycle” as used herein means an agent (compound or biological) that is effective to inhibit the formation and/or replication of HCV in a human being other than by inhibiting the function of the HCV NS3 protease. This includes agents that interfere with either host or HCV viral targets necessary for the HCV life cycle or agents which specifically inhibit in HCV cell culture assays through an undefined or incompletely defined mechanism. Inhibitors of another target in the HCV life cycle include, for example, agents that inhibit viral targets such as Core, E1, E2, p7, NS2/3 protease, NS3 helicase, NS4A, NS5A, NS5B polymerase, and internal ribosome entry site (IRES), or host targets such as cyclophilin B, phosphatidylinositol 4-kinase 111α, CD81, SR-B1, Claudin 1, VAP-A, VAP-B. Specific examples of inhibitors of another target in the HCV life cycle include SCY-635, ITX5061, NOV-205, AZD7295, BIT-225, NA808, MK-1220, PF-4878691, MX-3253, GS 9450, BMS-790052, ISIS-14803, GS9190, NIM-811, and DEBIO-025.

The term “HIV inhibitor” as used herein means an agent (compound or biological) that is effective to inhibit the formation and/or replication of HIV in a human being. This includes agents that interfere with either host or viral mechanisms necessary for the formation and/or replication of HIV in a human being. HIV inhibitors include, for example, nucleoside inhibitors, non-nucleoside inhibitors, protease inhibitors, fusion inhibitors and integrase inhibitors.

The term “HAV inhibitor” as used herein means an agent (compound or biological) that is effective to inhibit the formation and/or replication of HAV in a human being. This includes agents that interfere with either host or viral mechanisms necessary for the formation and/or replication of HAV in a human being. HAV inhibitors include Hepatitis A vaccines, for example, Havrix® (GlaxoSmithKline), VAQTA® (Merck) and Avaxim® (Aventis Pasteur).

The term “HBV inhibitor” as used herein means an agent (compound or biological) that is effective to inhibit the formation and/or replication of HBV in a human being. This includes agents that interfere with either host or viral mechanisms necessary for the formation and/or replication of HBV in a human being. HBV inhibitors include, for example, agents that inhibit HBV viral DNA polymerase or HBV vaccines. Specific examples of HBV inhibitors include Lamivudine (Epivir-HBV®), Adefovir Dipivoxil, Entecavir, FTC (Coviracil), DAPD (DXG), L-FMAU (Clevudine®), AM365 (Amrad), Ldt (Telbivudine), monoval-LdC (Valtorcitabine), ACH-126,443 (L-Fd4C) (Achillion), MCC478 (Eli Lilly), Racivir (RCV), Fluoro-L and D nucleosides, Robustaflavone, ICN 2001-3 (ICN), Bam 205 (Novelos), XTL-001 (XTL), Imino-Sugars (Nonyl-DNJ) (Synergy), HepBzyme; and immunomodulator products such as: interferon alpha 2b, HE2000 (Hollis-Eden), Theradigm (Epimmune), EHT899 (Enzo Biochem), Thymosin alpha-1 (Zadaxin®), HBV DNA vaccine (PowderJect), HBV DNA vaccine (Jefferon Center), HBV antigen (OraGen), BayHep B® (Bayer), Nabi-HB® (Nabi) and Anti-hepatitis B (Cangene); and HBV vaccine products such as the following: Engerix B, Recombivax HB, GenHevac B, Hepacare, Bio-Hep B, TwinRix, Comvax, Hexavac.

Specific preferred examples of some of these agents are listed below:
antiviral agents: ribavirin or amantadine;
immunomodulatory agents: class I interferons, class II interferons or pegylated forms thereof;
HCV polymerase inhibitors: nucleoside analogs or non-nucleosides;
inhibitor of another target in the HCV life cycle that inhibits a target selected from: NS3 helicase, NS2/3 protease, internal ribosome entry site (IRES), NS4A, NS5A, NS5B polymerase, or host targets such as cyclophilin A or B;
HIV inhibitors: nucleosidic inhibitors, non-nucleosidic inhibitors, protease inhibitors, fusion inhibitors or integrase inhibitors; or
HBV inhibitors: agents that inhibit viral DNA polymerase or is an HBV vaccine.

As discussed above, combination therapy is contemplated wherein a compound of the invention, or a pharmaceutically acceptable salt thereof, is co-administered with at least one additional agent selected from: an antiviral agent, an immunomodulatory agent, another inhibitor of HCV NS3 protease, an inhibitor of HCV polymerase, an inhibitor of another target in the HCV life cycle, an HIV inhibitor, an HAV inhibitor and an HBV inhibitor. These additional agents may be combined with the compounds of this invention to create a single pharmaceutical dosage form. Alternatively these additional agents may be separately administered to the patient as part of a multiple dosage form, for example, using a kit. Such additional agents may be administered to the patient prior to, concurrently with, or following the administration of a compound of the invention, or a pharmaceutically acceptable salt thereof.

According to another alternate embodiment, the pharmaceutical composition of this invention may additionally comprise at least one other inhibitor of HCV NS3 protease.

According to another alternate embodiment, the pharmaceutical composition of this invention may additionally comprise at least one inhibitor of HCV polymerase.

According to yet another alternate embodiment, the pharmaceutical composition of this invention may additionally comprise at least one inhibitor of other targets in the HCV life cycle, including but not limited to, helicase, NS5A protease, NS2/3 protease or internal ribosome entry site (IRES).

The dose range of the compounds of the invention applicable per day is usually from 0.01 to 100 mg/kg of body weight, preferably from 0.1 to 50 mg/kg of body weight. Each dosage unit may conveniently contain from 5% to 95% active compound (w/w). Preferably such preparations contain from 20% to 80% active compound.

The actual pharmaceutically effective amount or therapeutic dosage will of course depend on factors known by those skilled in the art such as age and weight of the patient, route of administration and severity of disease. In any case the combination will be administered at dosages and in a manner which allows a pharmaceutically effective amount to be delivered based upon patient's unique condition.

When the composition of this invention comprises a combination of a compound of the invention and one or more additional therapeutic or prophylactic agent, both the compound and the additional agent should be present at dosage levels of between about 10 to 100%, and more preferably between about 10 and 80% of the dosage normally administered in a monotherapy regimen.

### EXAMPLES

Temperatures are given in degrees Celsius. Solution percentages express a weight to volume relationship, and solution ratios express a volume to volume relationship, unless stated otherwise. Retention times (t_{R}) for each compound are measured using the standard analytical HPLC conditions described in the Examples. As is well known to one skilled in the art, retention time values are sensitive to the specific measurement conditions. Therefore, even if identical conditions of solvent, flow rate, linear gradient, and the like are used, the retention time values may vary when measured, for example, on different HPLC instruments. Even when measured on the same instrument, the values may vary when measured, for example, using different individual HPLC columns, or, when measured on the same instrument and the same individual column, the values may vary, for example, between individual measurements taken on different occasions.

Abbreviations used in the examples include:

Ac: acetyl; ACCA: 1-Aminocyclopropyl-carboxylic acid; BOC or Boc: tert-butyloxycarbonyl; DCM: dichloromethane; DIAD: diisopropylazodicarboxylate; DIPEA: diisopropylethylamine; DMF: N,N-dimethylformamide; DMSO: dimethylsulfoxide; equiv: equivalent; Et: ethyl; EtOAc: ethyl acetate; HATU: [0-7-azabenzotriazol-1-yl)-1,1,3,3-tetramethyluronium hexafluorophosphate]; hex: hexanes; Het: heterocycle; HPLC: high performance liquid chromatography; LiHMDS: lithium bis(trimethylsilyl)amide; M: mole/liter; Me: methyl; MeOH: methanol; mins: minutes; mmol: millimole; MS: mass spectrometry (FIA MS— flow injection analysis mass spectrometry HPLC: Ultraperformance Liquid Chromatography); NMP: N-methylpyrrolidinone; NMR: nuclear magnetic resonance; Ph: phenyl; Prep HPLC: preparative high performance liquid chromatography; RT: room temperature (18 to 22° C.); sat: saturated; SM: starting material; SNAr: Nucleophilic aromatic substitution; tert-butyl or t-butyl: 1,1-dimethylethyl; t-BME: tert-butyl methyl ether; TBTU: 2-(1H-benzotriazole-1-yl)-1,1,3,3-tetramethyl uronium tetrafluoroborate; TEA: triethylamine; TFA: trifluoroacetic acid; and THF: tetrahydrofuran.

NMR: Chemical shifts are reported in parts per million from tetramethylsilane with the solvent resonance as the internal standard. Data are reported as follows: chemical shift, multiplicity (s=singlet, d=doublet, t=triplet, q=quartet, qn=quintet, S=septuplet, m=multiplet and br=broad), integration and coupling constant in Hz. Flash chromatography is carried out on silica gel (SiO₂) according to Still's flash chromatography technique (W. C. Still et al., J. Org. Chem. 1978, 43, 2923). Alternatively compounds and intermediates can be purified on a Teledyne ISCO Combiflash Rf System at 254 nm using commercial normal phase silica 4-120 g RedisepRfor Silicycle columns eluting in 0-100% EtOAc/Hexane or 0-10% MeOH/DCM at a flow rate of 18-85 mL/min depending on column size. Mass spectral analyses are recorded using flow injection analysis mass spectrometry or Waters Acquity Ultraperformance LC System consisting of a sample organizer, PDA detector, column manager, sample manager, binary solvent manager and SQ detector. Analytical HPLC is carried out under standard conditions using a SunFire™ C₁₈ 3.5 μM reverse phase column, 4.6×30 mm and a linear gradient (0 to 100% over 8 mins with 2.5 mL/min) employing 0.1% TFA/acetonitrile and 0.1% TFA/water as solvents. Preparative chromatography purification is carried out using a Waters Autopurify Chromatography System consisting of the following components: 1) Sample Manager Model 2767; 2) Pump Model 2525 or 2545); 3) PDA Detector Model 2996 or 2998; 4) System Fluidics Organizer (SFO) or Column Fluidics Organizer (CFO) with or without the additional component 5) Mass Spec Model 3100.

### Purification Columns:

Sunfire Prep C₁₈ Column OBD; 19×50 mm, 5 μM (Part No. 186002566) using a 0-100% Gradient at 30 mL/min 0.1% TFA/Acetonitrile or Ammonium Formate/MeOH at pH 3.8 (for Ammonium Formate Gradient Conditions: see below).

X-Bridge Prep C₁₈ Column OBD; 19×50 mm, 5 μM (Part No. 186002977) using Ammonium Bicarbonate/MeOH at pH 10; (for Gradient Conditions: see below).

Gradient Program for Ammonium Formate or Ammonium Bicarbonate (A)/MeOH (B) are as follows:

At 30 mL/min:

| Time, min | % A | % B | Curve |
|---|---|---|---|
| 0 | 100 − X | X | — |
| 1.00 | 100 − X | X | 6 |
| 11.00 | 100 − (X + 20) | X + 20 | 6 |
| 11.10 | 0 | 100 | 6 |
| 13.10 | 0 | 100 | 6 |

where X is a predetermined value dependant on the obtained analytical HPLC retention time particular to each product.

### Methodology

### Methodology for compounds of Table 1

All compounds from Table 1 are prepared according to Scheme 1 and/or Scheme 2.

### General Scheme 1

In Scheme 1, the Boc protected amines Aa-n and Ba-n are deprotected under standard acidic conditions to provide Ca-n and Da-n. A coupling reaction with the corresponding heterocyclic acids or sodium salts R2a-o from FIG. 1 and appropriate reagents provides the desired products.

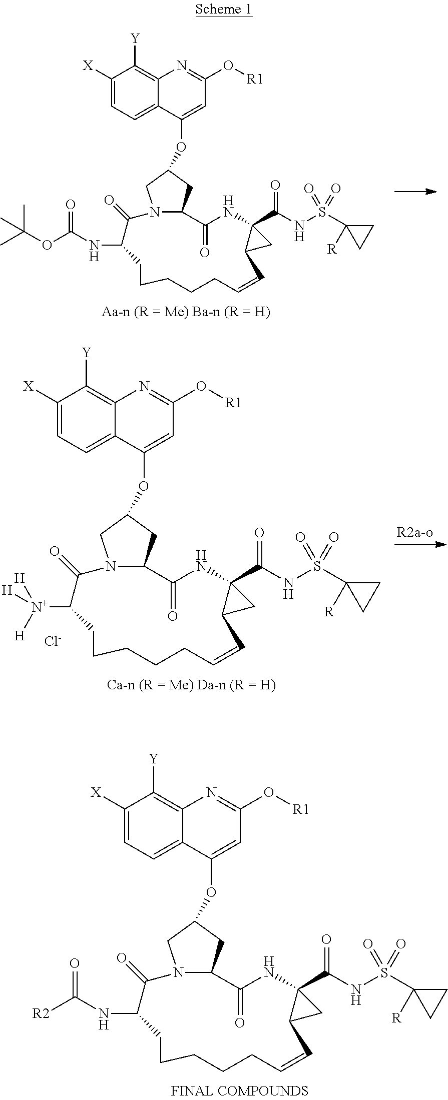

### General Scheme 2

Alternatively, in Scheme 2, the appropriate heterocyclic capping group (R2a-o from FIG. 1) is installed first on the macrocyclic brosylates (E and/or F) after removal of the Boc-protecting group under standard acidic conditions and then the appropriately substituted quinoline (Qa-n from FIG. 2) is installed via S_{N}Ar to provide the desired products.

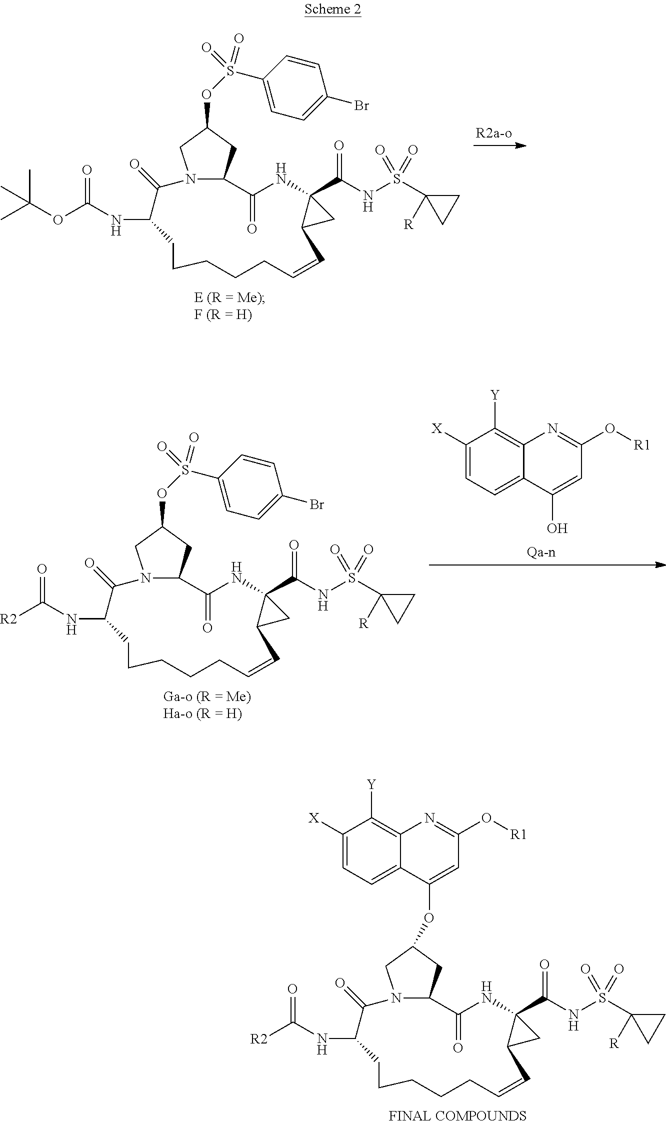

### Definition of Intermediates

Capping groups R

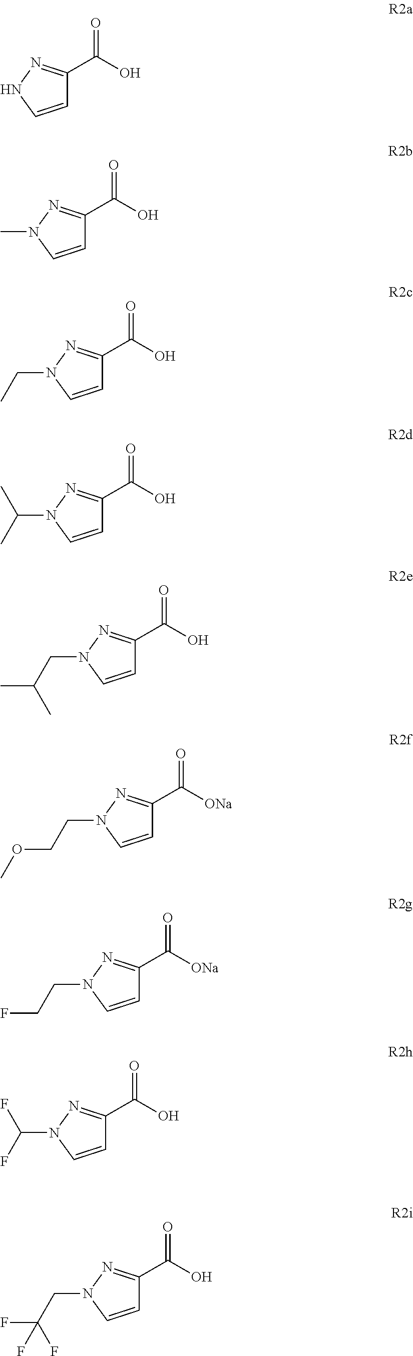

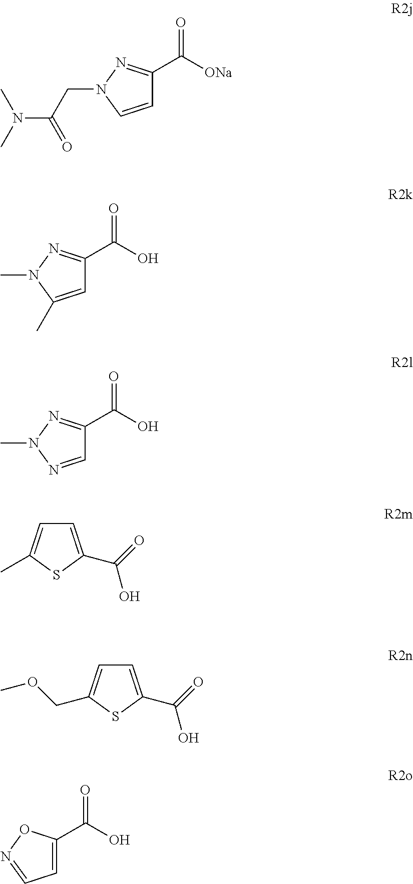

### Quinolines Q

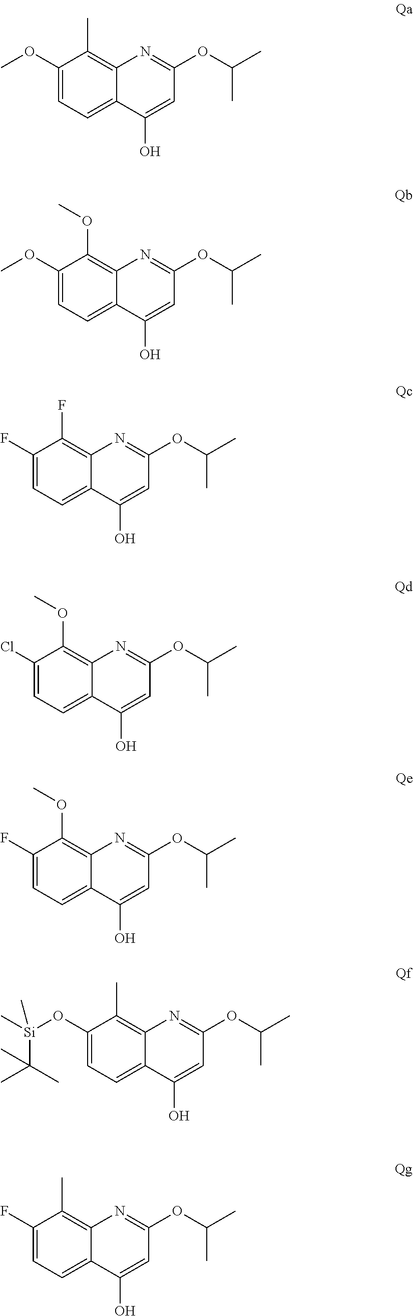

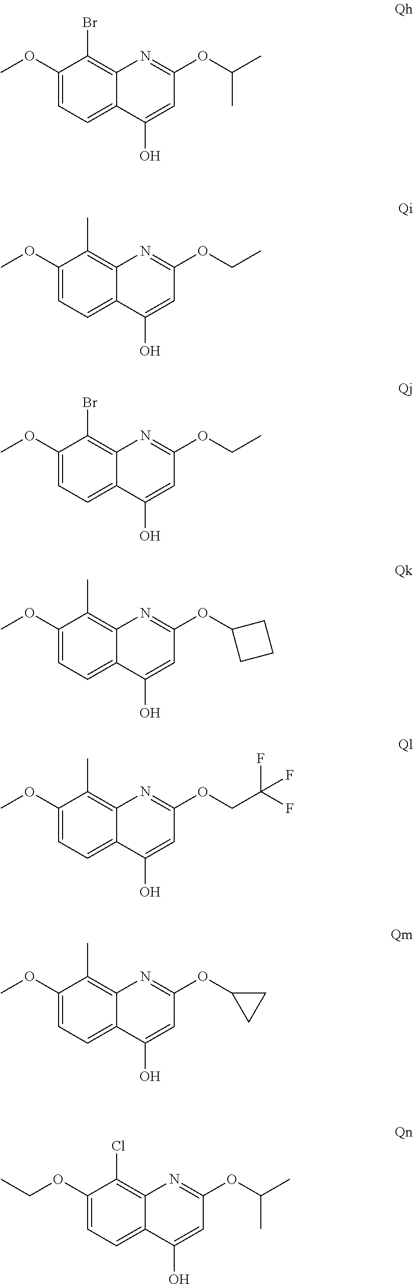

### Methodology for Compounds of Table 2

The compounds of Table 2 are prepared according to Scheme 3, 4 or 5.

In Scheme 3, the intermediates Ca-n or Da-n are converted to the corresponding isocyanate via an activated carbonyl species (carbonyl diimidazole or triphosgene) and reacted with the appropriate amines R3a-i to provide the targeted disubstituted ureas.

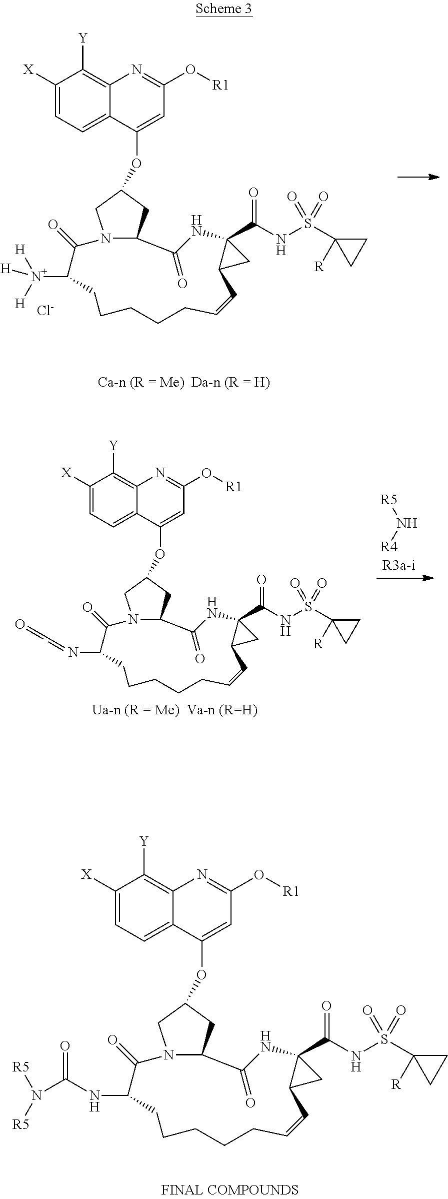

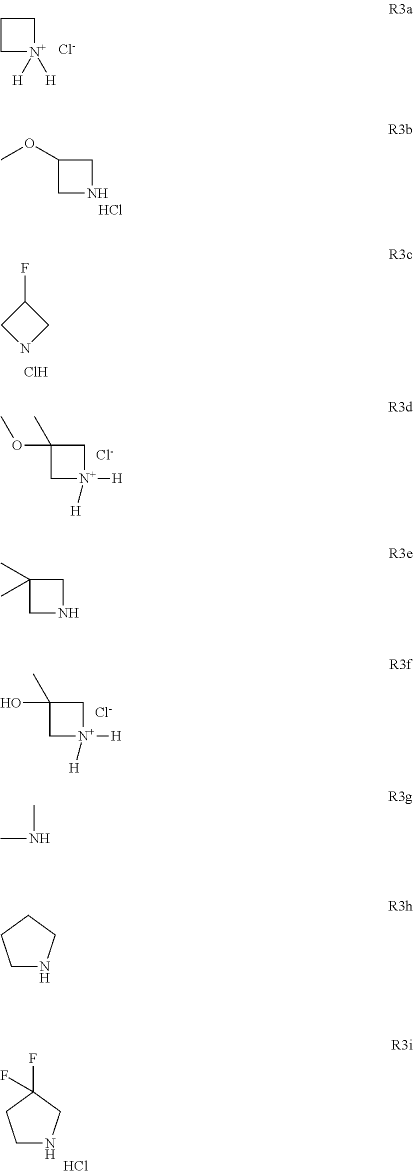

Alternatively, Scheme 4 shows that Ca-n or Da-n can be reacted with the dimethyl amidoyl chloride to give the final compounds containing a dimethyl urea.

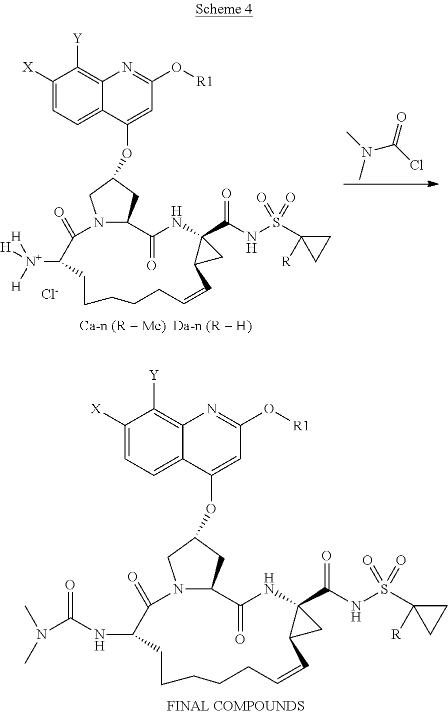

Scheme 5 shows that disubstituted ureas can be generated directly from the brosylates E or F and the quinoline is installed via S_{N}Ar as a final step towards the production of the final compounds.

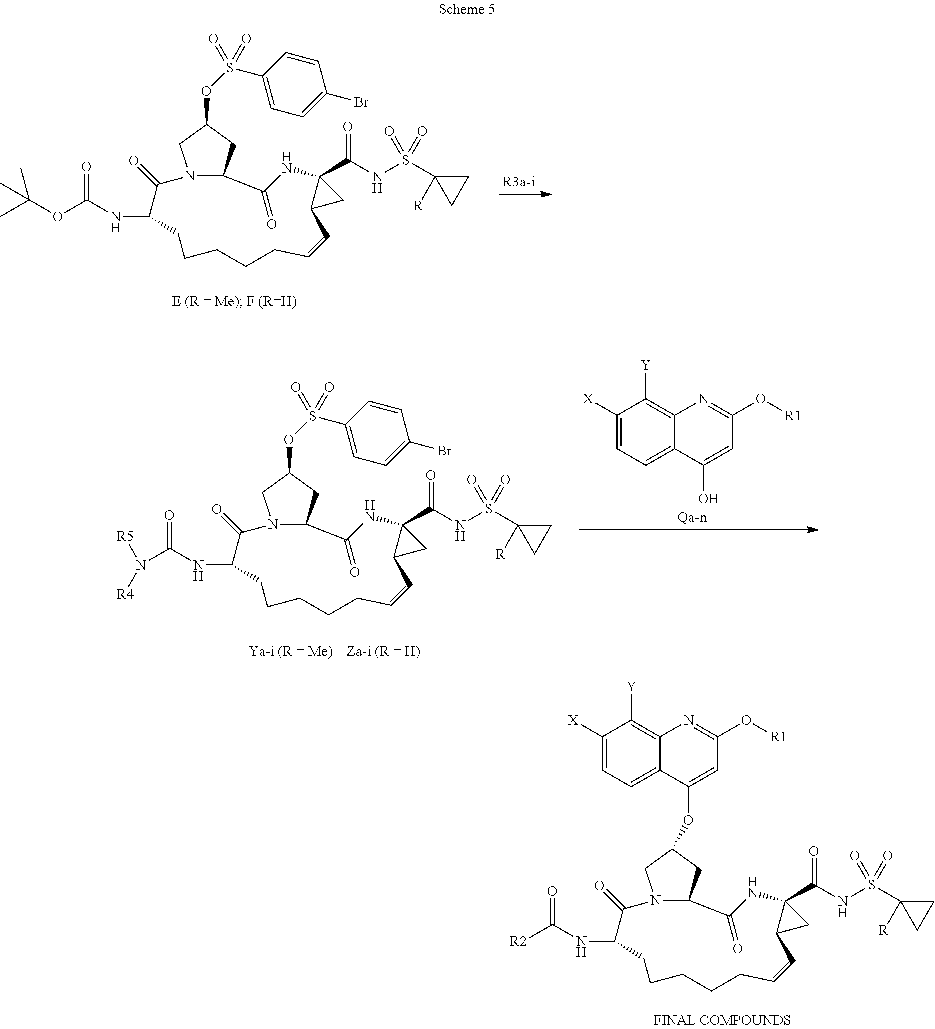

Macrocyclic Intermediates A and B

The synthesis of Aa-n and Ba-n is realized by utilizing the general procedures highlighted in either Scheme 6 or Scheme 7. In Scheme 6, the common intermediate I is submitted to SNAr conditions to incorporate the appropriately substituted quinolines (Qa-n). These intermediates are then converted to the corresponding acylsulfonamides Aa-n and Ba-n via the azalactones La-n through well documented procedures, such as WO 2006/000085, WO 2006/007700, WO 2006/007708, WO 2007/014922, Heterocycles 2009, 79, 985-1005, Synthesis 2009, 4, 620-626 and European Journal of Medicinal Chemistry 2009, 44(2), 891-900).

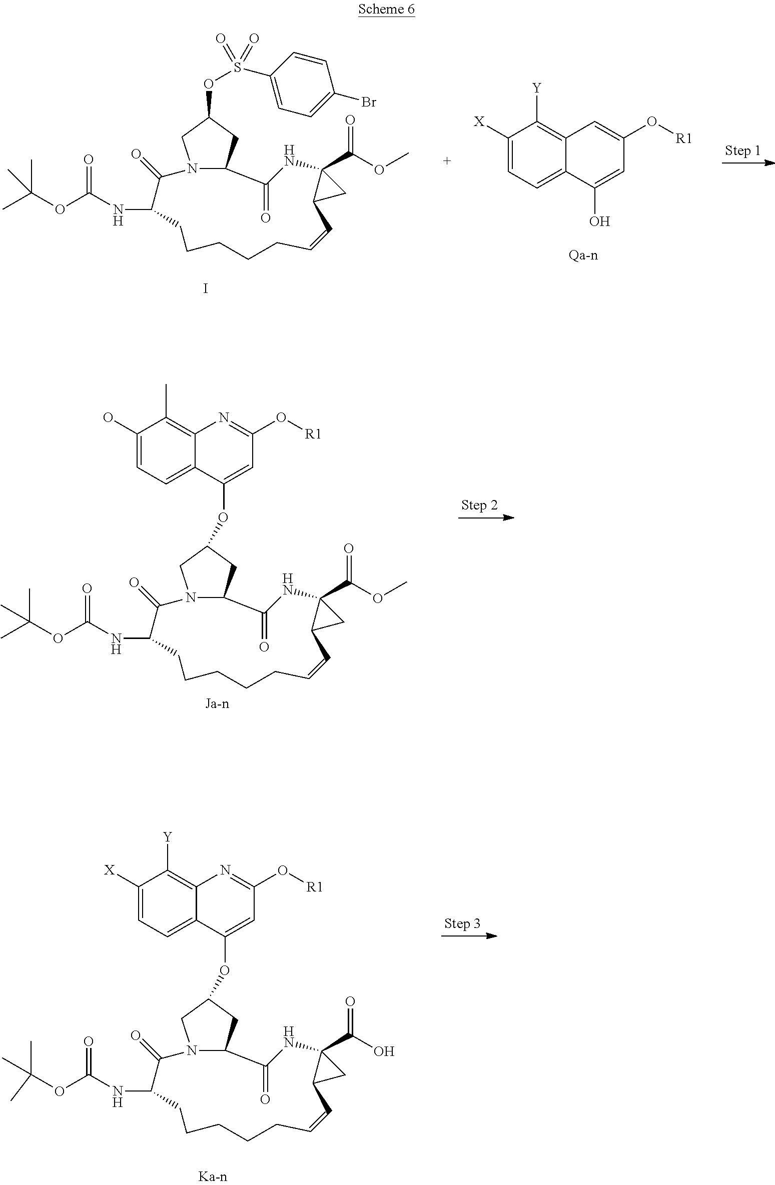

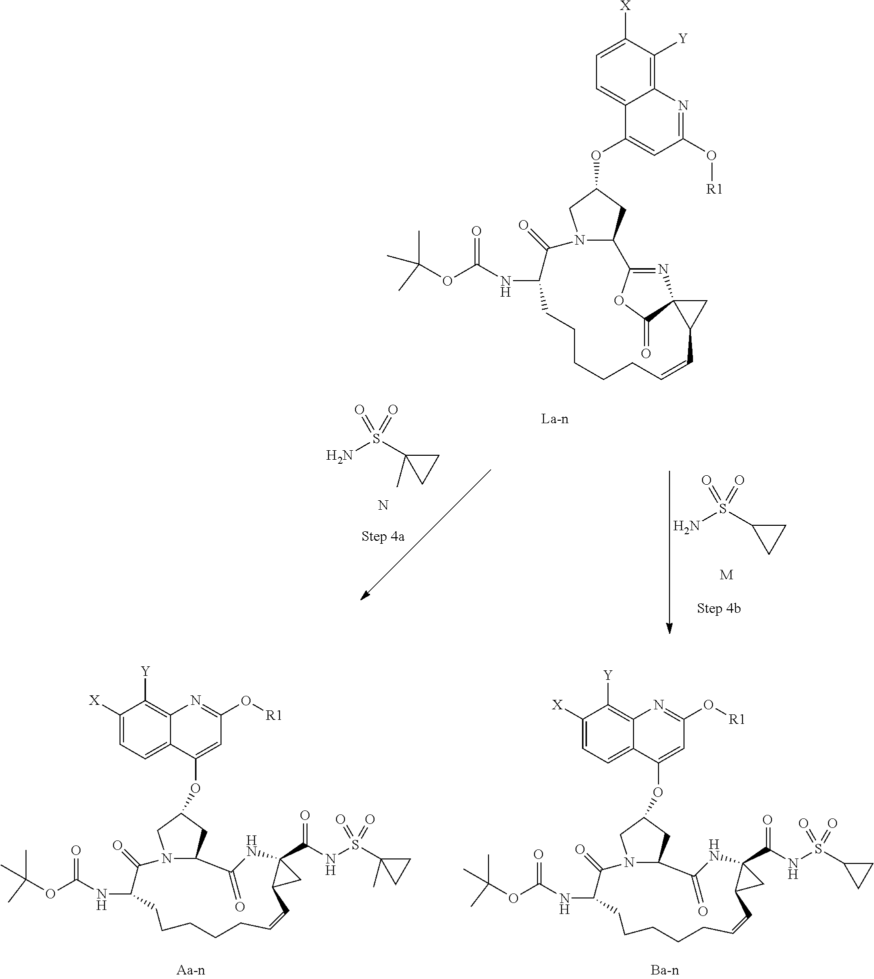

Alternatively, in Scheme 7, the azalactone O is formed first from the brosylate intermediate I which is then reacted with sulfonamides M or N to form intermediates E or F followed by incorporation of the appropriately substituted hydroxyquinoline (Qa-Qn) via S_{N}Ar to provide the desired products.

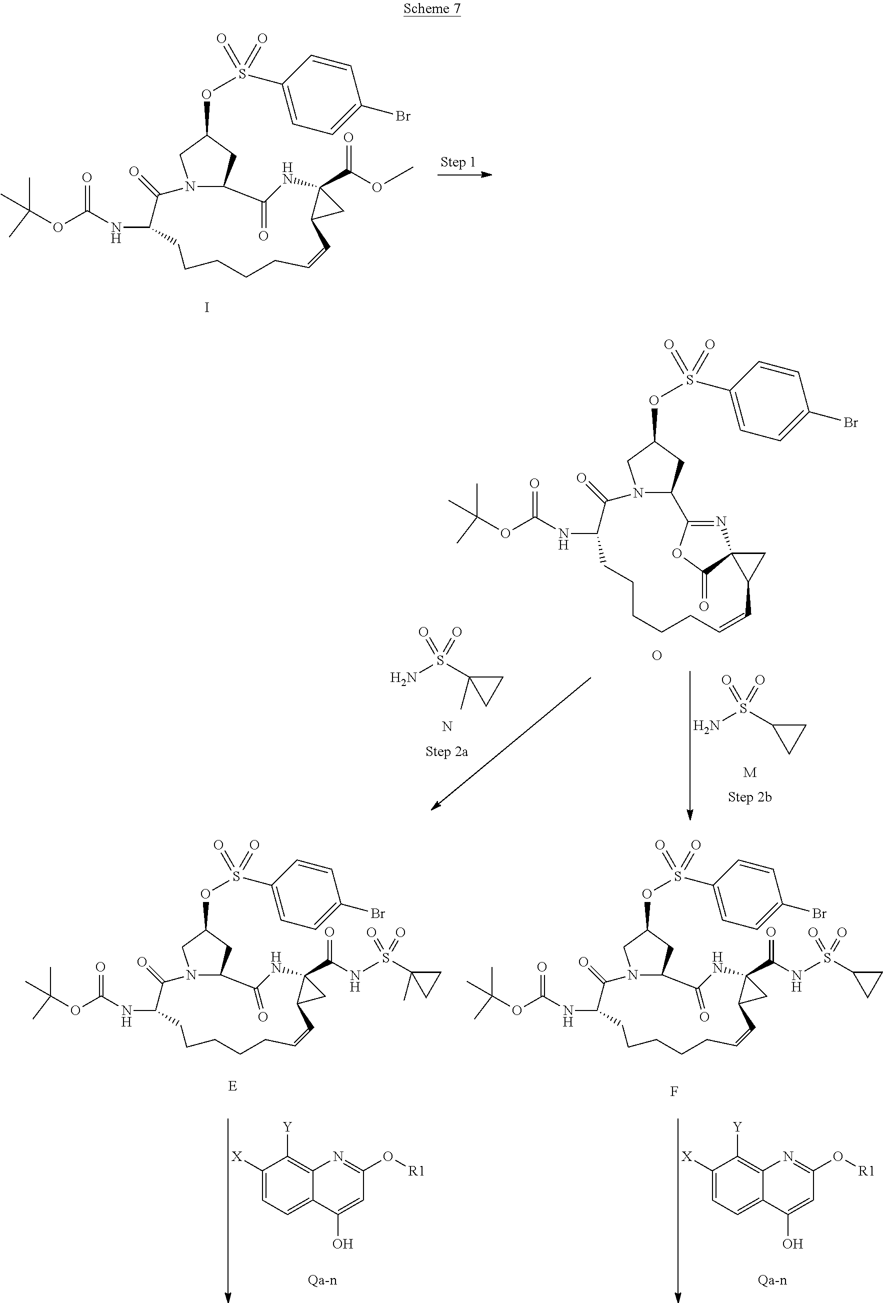

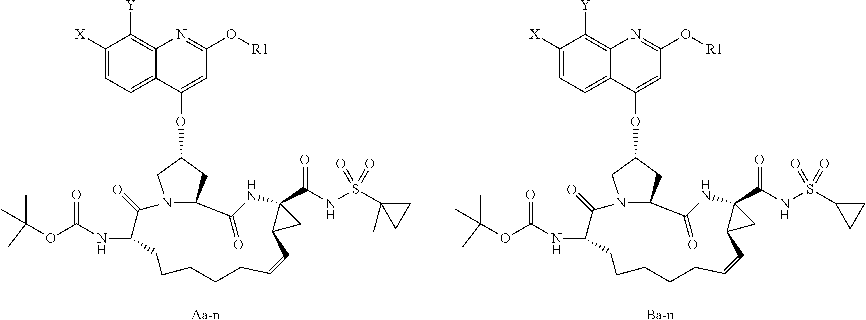

### Synthesis of Intermediates

Synthesis of Capping groups R2a-R2o (from FIG. 1):

R2a, R2b, R2c, R2d, R2e, R2h, R2i, R2k, R2m, R2n and R2o are available from commercial sources and are used as received without further purification (Commercial sources: R2a: Oakwood; R2b, R2n: Chembridge BB; R2c, R2d, R2e, R2h: Art-Chem-BB; R2i: Akos; R2k: Maybridge-Int; R2m, R2o: Aldrich)

### Synthesis of R2f, R2g

The synthesis is done as described in scheme 8:

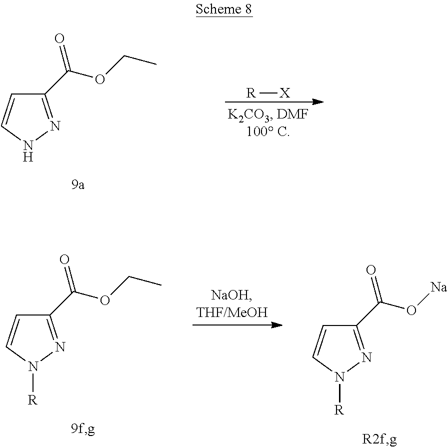

### Synthesis of Sodium 1-(2-fluoro-ethyl)-1H-pyrazole-3-carboxylate (R2g)

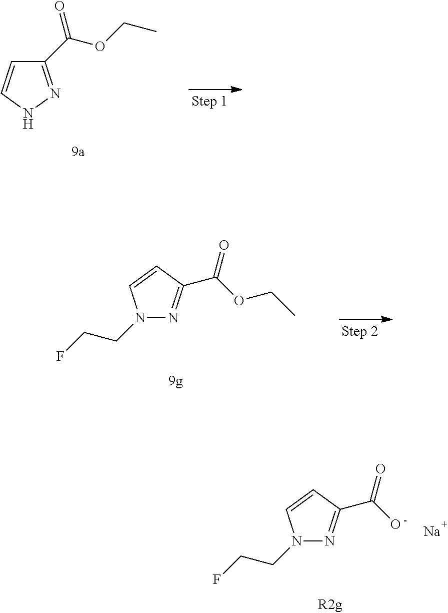

### Step 1:

To a solution of 1H-pyrazole-3-carboxylic acid ethyl ester 9a (500 mg, 3.57 mmol) in DMF (6.5 mL) is added K₂CO₃ (542 mg, 3.93 mmol, 1.10 equiv), NaI (1.07 g, 7.14 mmol, 2.00 equiv) and 1-bromo-2-fluoroethane (928 mg, 7.14 mmol, 2.00 equiv). The reaction mixture is stirred at 100° C. for 48 h in a closed vial. The reaction is quenched with HCl 1 N (pH ˜5-6), water is added and the mixture is extracted with EtOAc (5×). The organics are washed with brine, dried with anhydrous MgSO₄, filtered and concentrated. The crude mixture containing the 2 regioisomers is purified by prep HPLC. The appropriate fractions are combined, frozen and lyophilized to give 9g.

UPLC-MS: 186.8 (M+H)⁺

¹H NMR (400 MHz, DMSO-d₆) δ (ppm): 7.90 (d, 1H, J=2.4 Hz), 6.76 (d, 1H, J=2.4 Hz), 4.79 (dt, 2H, J=47.3, 4.7 Hz), 4.53 (dt, 2H, J=27.8, 4.7 Hz), 4.26 (q, 2H, J=7.1 Hz), 1.28 (t, 3H, J=7.1 Hz).

### Step 2:

1-(2-Fluoro-ethyl)-1H-pyrazole-3-carboxylic acid ethyl ester (9g) (337 mg, 1.81 mmol) is dissolved in THF/MeOH (6 mL, 3:1 mixture). NaOH 1 M (1.99 mL, 1.99 mmol, 1.10 equiv) is added and the reaction is stirred at RT for ˜12 h. The crude mixture is evaporated to dryness, diluted in H₂O/MeCN, frozen and lyophilized to give R2g.

UPLC-MS: 158.9 (M+H)⁺ (for the Corresponding Acid)

¹H NMR (400 MHz, DMSO-d₆) δ (ppm): 7.58 (d, 1H, J=1.9 Hz), 6.33 (d, 1H, J=1.9 Hz), 4.75 (dt, 2H, J=47.3, 4.7 Hz), 4.41 (dt, 2H, J=27.4, 4.7 Hz).

### Synthesis of Sodium 1-(2-methoxy-ethyl)-1H-pyrazole-3-carboxylate (R2f)

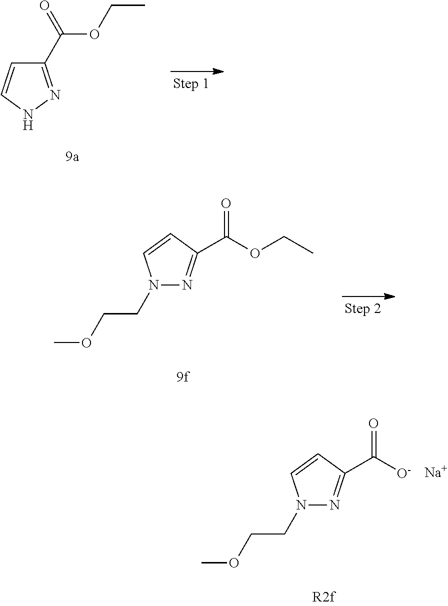

### Step 1:

Compound 9f is synthesized analogously to the procedure used for the preparation of 1-(2-Fluoro-ethyl)-1H-pyrazole-3-carboxylic acid ethyl ester (9g) using 1H-pyrazole-3-carboxylic acid ethyl ester 9a (500 mg, 3.57 mmol) and 2-bromomethyl methyl ether (685 μL, 7.28 mmol, 2.00 equiv) as the alkylating agent. The crude mixture containing the 2 regioisomers is purified by prep HPLC. The appropriate fractions are combined, frozen and lyophilized to give 9f.

UPLC-MS: 199.4 (M+H)⁺

¹H NMR (400 MHz, DMSO-d₆) δ (ppm): 7.83 (d, 1H, J=2.3 Hz), 6.72 (d, 1H, J=2.3 Hz), 4.34 (t, 2H, J=5.3 Hz), 4.26 (q, 2H, J=7.1 Hz), 3.69 (t, 2H, J=5.3 Hz), 3.22 (s, 3H), 1.28 (t, 3H, J=7.1 Hz).

### Step 2:

Compound R2f is synthesized analogously to the procedure for the hydrolysis of sodium 1-(2-fluoro-ethyl)-1H-pyrazole-3-carboxylate (R2g) using 426 mg (2.15 mmol) of 9f.

UPLC-MS: 171.1 (M+H)⁺ (for the corresponding acid)

¹H NMR (400 MHz, DMSO-d₆) δ (ppm): 7.52 (d, 1H, J=2.4 Hz), 6.30 (d, 1H, J=2.4 Hz), 4.21 (t, 2H, J=5.5 Hz), 3.65 (t, 2H, J=5.5 Hz), 3.21 (s, 3H).

### Synthesis of R2j

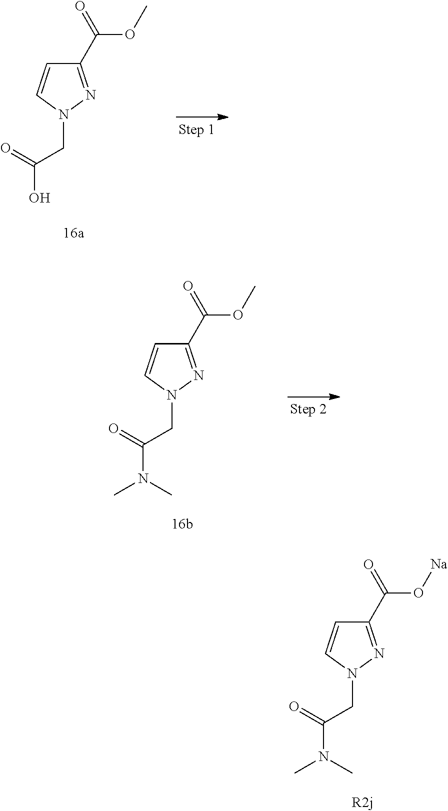

To a solution of 1-carboxymethyl-1H-pyrazole-3-carboxylic acid methyl ester 16a (200 mg, 1.09 mmol) in DCM (3.6 mL) at 0° C. is added DMF (10 μL, 0.13 mmol, 0.12 equiv) followed by oxalyl chloride 2 M in DCM (0.71 mL, 1.41 mmol, 1.3 equiv). The reaction is stirred at RT for 2 h. The reaction mixture is evaporated to dryness and the acyl chloride is used as such for the next step. To the acyl chloride (220 mg, 1.09 mmol) in DCM (3.6 mL) is added dimethylamine 2 M in THF (0.81 mL, 1.63 mmol, 1.5 equiv) and triethylamine (0.45 mL, 3.26 mmol, 3.00 equiv). The reaction is stirred at RT for 12 h. The crude mixture is evaporated to dryness and purified by flash chromatography to give 16b.

UPLC-MS: 212.1 (M+H)⁺

¹H NMR (400 MHz, DMSO-d₆) δ (ppm): 7.76 (d, 1H, J=2.3 Hz), 6.75 (d, 1H, J=2.3 Hz), 5.23 (s, 2H), 3.78 (s, 3H), 3.03 (s, 3H), 2.85 (s, 3H).

### Step 2:

Compound R2j is made analogously to the procedure used for the hydrolysis of sodium 1-(2-fluoro-ethyl)-1H-pyrazole-3-carboxylate (R2g) using 179 mg (0.85 mmol) of 16b.

UPLC-MS: 197.9 (M+H)⁺ (for the corresponding acid)

¹H NMR (400 MHz, DMSO-d₆) δ (ppm): 7.43 (d, 1H, J=2.2 Hz), 6.30 (d, 1H, J=2.2 Hz), 5.04 (s, 2H), 3.02 (s, 3H), 2.85 (s, 3H).

### Synthesis of 2-methyl 2H-1,2,3-triazole 4-carboxylic acid (R2l)

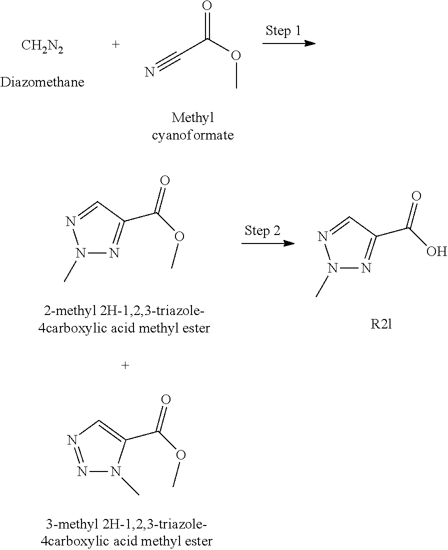

### Step 1:

Methyl cyanoformate (1.00 g, 11.7 mmol) is charged in a flask, dissolved in THF (40 mL), then a 0.6 M diazomethane solution in Et₂O (58.8 mL, 35.3 mmol, 3.0 equiv) is added. This solution is stirred at RT for 16 h. Water (40 mL) and EtOAc (40 mL) are added and then the layers are separated. The solvent is evaporated and purification is performed on Combiflash (20-100% hexane) to provide the 3,4-regioisomer and the desired 2,4-regioisomer methyl esters.

2,4-regioisomer (434 mg, 26% yield): FIA M.S. (electrospray): 142.2 (M+H)⁺

¹H NMR (400 MHz, CDCl₃): δ 8.05 (s, 1H), 4.28 (s, 3H) 3.96 (s, 3H).

3,4-regioisomer (654 mg, 39% yield): ¹H NMR (400 MHz, CDCl₃): δ 8.14 (s, 1H), 4.35 (s, 3H) 3.95 (s, 3H).

### Step 2:

2-methyl 2H-1,2,3-triazole-4-carboxylic acid methyl ester (263 mg, 1.86 mmol) is charged in a round-bottom flask, then THF (15 mL), 1 M solution NaOH (9.3 mL, 9.3 mmol, 5.0 equiv) and MeOH (5 mL) are measured and mixed in a graduated cylinder, then added to flask. Solution is stirred at room temperature. After 4 h, 1 M HCl is added (10 mL) and solvent is evaporated. EtOAc is added and layers are separated. Solvent is evaporated. Desired product R2l is obtained as a white solid (215 mg, 91%).

¹H NMR (400 MHz, DMSO-d6): δ 8.17 (s, 1H), 4.22 (s, 3H).

### Synthesis of Intermediates R3a-R3i

R3a, R3e, R3g, R3h and R3i are available from commercial sources and are used as received without further purification (Commercial sources: R3a: Oakwood; R3e: Princeton; R2g, R2h: Aldrich; R2i: Akos).

### Synthesis of R3b

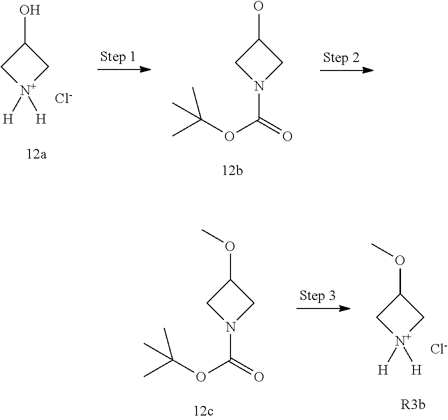

### Step 1:

Compound 12a (Oakwood, 250 mg, 2.28 mmol) is dissolved in THF (4.0 mL), NaHCO₃ (sat) (4.0 mL) and Boc₂O (1 M solution in THF, 2.6 mL, 2.6 mmol) are added. The reaction mixture is stirred overnight. The reaction is poured into H₂O, extracted with EtOAc (2×), washed with brine, dried over MgSO₄, filtered and concentrated in vacuo. The material is purified by flash chromatography using 0-7% MeOH/DCM as the eluent to provide 12b.

### Step 2:

12b (372 mg, 2.15 mmol) is dissolved in THF (7 mL) and cooled to 0° C. NaH (128.9 mg of 60% suspension in oil, 3.22 mmol) is added and the mixture is stirred for 30 min. MeI (0.40 mL, 6.4 mmol) is added and the reaction mixture is capped, allowed to warm to RT and is stirred overnight. The reaction is poured into H₂O, extracted with EtOAc (2×), washed with brine, dried over MgSO₄, filtered and concentrated in vacuo. The material is purified by flash chromatography using 0-50% EtOAc/hexanes as the eluent to provide 12c.

¹H NMR (400 MHz, CDCl₃): δ 4.15-4.10 (m, 1H), 4.09-4.04 (m, 2H), 3.82 (dd, 2H, J=10.2, 3.9 Hz), 3.28 (s, 3H), 1.44 (s, 9H).

### Step 3:

12c (339 mg) is dissolved in 1.0 N HCl/dioxane (10 mL), stirred for 1 h, then concentrated in vacuo to give compound R3b which is used as such without further purification.

### Synthesis of R3c

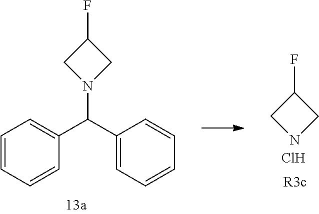

In a vial is incorporated the protected azetidine 13a (100 mg, 0.414 mmol). A mixture of MeOH (2 mL) and EtOAc (3 mL) is used to dissolve the starting material. The mixture is equally divided in four fractions and loaded on the H-Cube hydrogenation system (40 bar H₂, 40° C.). The mixture collected is concentrated to afford R3c.

### Synthesis of R3d and R3f

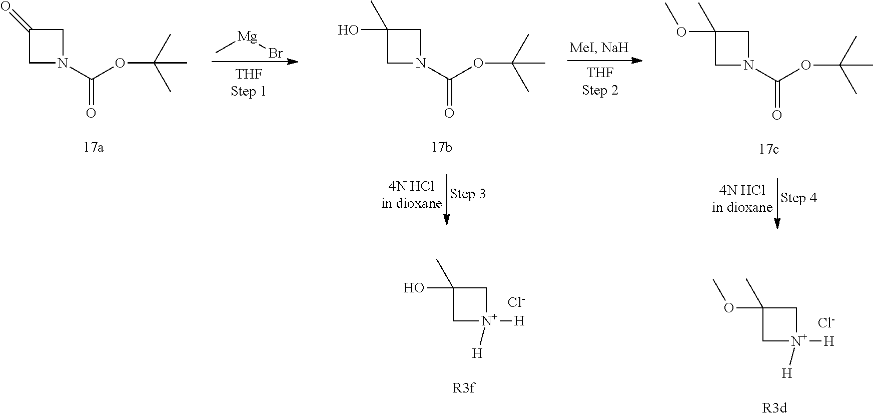

### Step 1:

Oxoazetidine 17a (400 mg, 2.34 mmol) is charged in a flask and diluted in dry THF (5 mL). The solution is cooled to 0° C., then a MeMgBr solution (1.95 mL, 3.0 M) is added under nitrogen. The reaction is allowed to reach RT and is stirred for 2 h. The reaction is quenched with a saturated solution of NH₄Cl. EtOAc is added for the extraction. The organic phase is separated, combined, dried (MgSO₄) and concentrated to give 17b (438 mg) which is used in the next step without further purification.

¹H NMR (400 MHz, CDCl₃): δ 3.85 (q, 4H, J=9.1 Hz), 2.35 (bs, 1H), 1.52 (s, 3H), 1.45 (s, 9H).

### Step 2:

Hydroxyazetidine 17b (100 mg, 0.53 mmol) is charged in a flask and diluted in dry THF (2 mL). Then NaH (51.3 mg, 2.14 mmol) is added and stirred for 2 mins. MeI (0.2 mL, 3.21 mmol) is added at RT and the reaction is stirred until the starting material is consumed. The reaction is quenched with water and then EtOAc is added for the extraction. The organic phase is separated, combined, dried (MgSO₄) and concentrated to give 17c (108 mg) which is used in the next step without further purification.

¹H NMR (400 MHz, CDCl₃): δ3.91 (d, 2H, J=9.0 Hz), 3.67 (dd, 2H, J=9.0, 0.8 Hz), 3.24 (s, 3H), 1.46 (s, 3H), 1.45 (s, 9H).

### Steps 3 and 4:

Methoxyazetidine 17c (108 mg, 0.53 mmol) is charged in a flask followed by the addition of 4 M HCl in dioxane (3 mL, 12 mmol). The reaction is stirred for 1.5 h and then concentrated to give R3d which is used in the next step without further purification.

R3f is prepared in an analogous manner by treatment of 17b with 4 M HCl/dioxane.

Synthesis of Quinolines (Qa-Qn) from FIG. 2:

Quinolines (Qa-Qn) are synthesized according to scheme 9

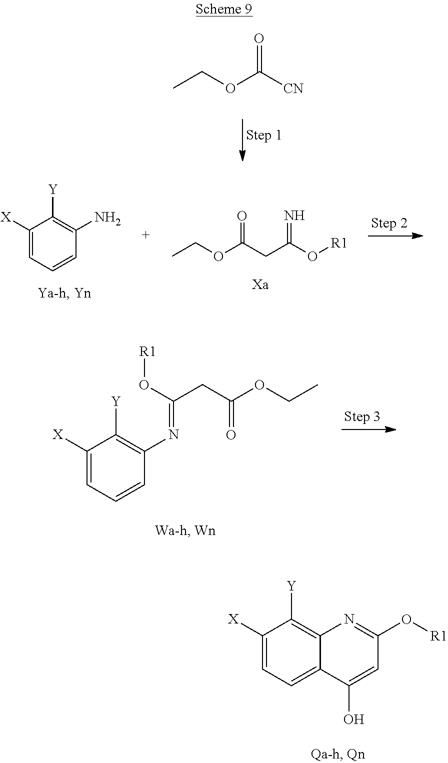

The synthesis of quinolines Qa to Qh and Qn requires the following anilines Ya-h, Yn:

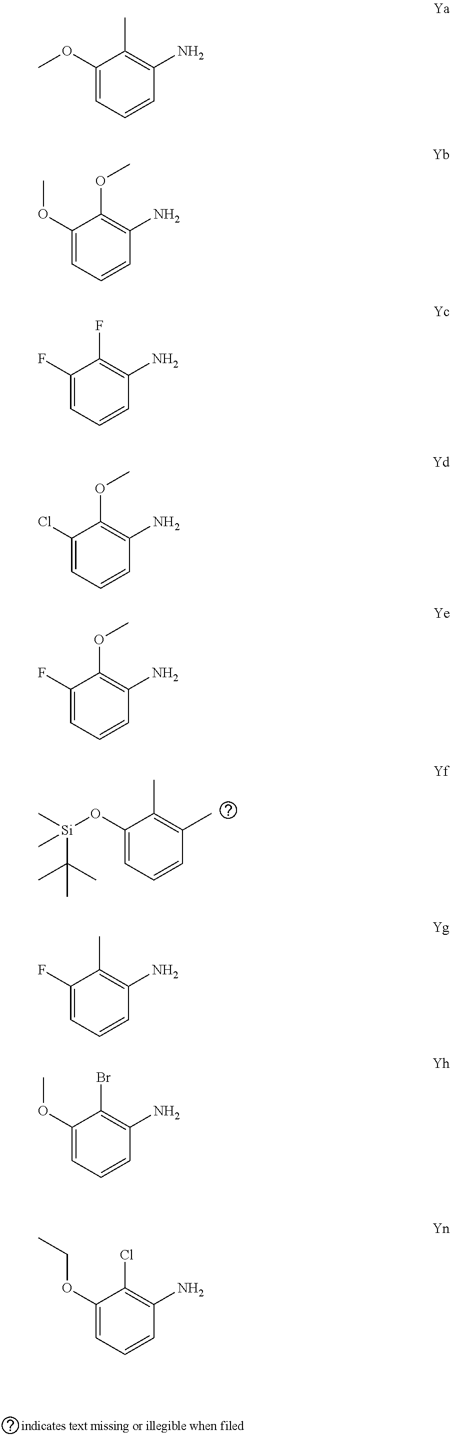

The following anilines are commercially available: Ya, Yb, Yc, Yd, Ye, Yg, and Yn. Commercial suppliers are as follows (Ya—TCl-US; Yb—TCl-JP; Yc, Yg—Aldrich; Yd—Apollo International; Ye—Chontech; Yn—Sinochem)

Aniline Yh is prepared according to procedure in WO 2007/053755;

### Synthesis of Aniline Yf

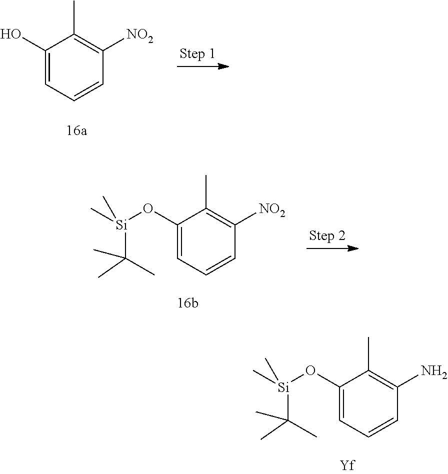

### Step 1:

To a solution of the 2-methyl-3-nitrophenol 16a (5.00 g, 32.6 mmol) in DCM (60 mL) and DMF (15 mL) is added imidazole (4.45 g, 65.3 mmol) followed by tert-butyldimethylchlorosilane (6.40 g, 42.4 mmol) slowly. The solution is left to stir at room temperature for the night. DCM is removed under vacuum. The solution is taken up in EtOAc, washed with 0.1 N HCl, saturated NaHCO₃ and brine (2×). The organic phase is dried over MgSO₄, filtered and concentrated. The crude is purified by combiflash with 80 g column starting with 1% EtOAc/hexane to 10% over 20 minutes to afford 16b.

### Step 2:

16b (5.3 g, 19.82 mmol) is dissolved in EtOH and the flask is purged with nitrogen. Palladium on carbon (400 mg) is added and the flask is evacuated and backfilled with hydrogen (3×). The reaction mixture is stirred at RT for 16 h. The flask is then evacuated and backfilled with nitrogen (3×). The product is filtered through a celite pad, rinsed with EtOAc and MeOH. The solution is evaporated to dryness to obtain an oil which is passed trough a silica pad on a fritted funnel with 50% EtOAc/hexane to obtain Yf.

Retention time (min)=3.51 min

¹H NMR (400 MHz, CDCl₃): δ 6.88 (t, 1H, J=7.8 Hz), 6.34 (d, 1H, J=8.3 Hz), 6.29 (d, 1H, J=8.2 Hz), 3.60, (bs, 2H), 2.05 (s, 3H), 1.03 (s, 9H), 0.22 (s, 6H).

### Synthesis of Quinoline Qa

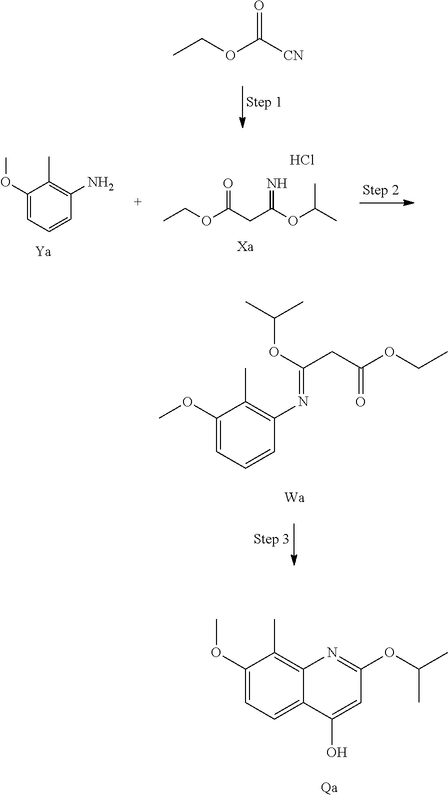

Step 1: Synthesis of imidate Xa

A solution of ethyl cyanoacetate (120 g, 1.06 mol) and isopropanol (70.1 g, 1.16 mol, 1.1 equiv) in anhydrous diethyl ether (1 L) is cooled to 0° C. This solution is purged with HCl gas for 45 mins and then the reaction mixture is warmed to ambient temperature and stirred for 19.5 h. The solvent is removed in vacuo. The residue is triturated with hexanes, collected by suction filtration and dried in vacuo to yield imidate hydrochloride Xa. This intermediate is used in the next synthetic step without further purification.

### Step 2:

In a 2 L round bottom flask, 3-methoxy-2-methyl aniline (53.0 g, 386 mmol), intermediate Xa (81.0 g, 386 mmol, 1 equiv) and isopropanol (800 mL) are stirred at 40° C. for 3.5 h. The solvent is removed in vacuo, and the remaining residue is dissolved in EtOAc (1.5 L) and washed with brine (500 mL). The organic phase is dried over anhydrous sodium sulfate and concentrated to give Wa (128 g) which is used in the next step without further purification.

### Step 3: Cyclization

In a 1 L round bottom flask, Wa (128 g) is dissolved in diphenyl ether (600 mL), and this mixture is quickly heated up (heating mantle) to 230° C. The temperature is kept between 230° C. and 245° C. for 8 mins. The reaction mixture is then cooled to RT, passed through a pad of silica gel (˜1 kg) and washed with hexanes to remove the diphenyl ether. The column is then eluted with a 20% to 80% EtOAc in hexanes to afford compound Qa.

¹H NMR (400 MHz, DMSO-d₆) δ (ppm): 11.07 (s, 1H), 7.84 (d, 1H, J=9.2 Hz), 7.14 (d, 1H, J=9.2 Hz), 6.04 (s, 1H), 5.44 (S, 1H, J=6.3 Hz), 3.89 (s, 3H), 2.41 (s, 3H), 1.34 (d, 6H, J=6.3 Hz).

### Synthesis of Quinoline Qb

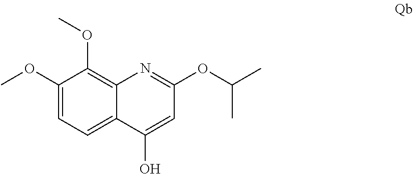

Quinoline Qb is prepared analogously to Qa but starting with the aniline Yb.

¹H NMR (400 MHz, CDCl₃) δ (ppm): 8.24-8.07 (m, 1H), 7.99 (d, 1H, J=9.0 Hz), 6.93 (d, 1H, J=9.0 Hz), 5.72-5.66 (m, 1H), 4.70 (S, 1H, J=6.1 Hz), 4.00 (s, 3H), 3.98 (s, 3H), 1.43 (d, 6H, J=6.1 Hz).

### Synthesis of Quinoline Qc

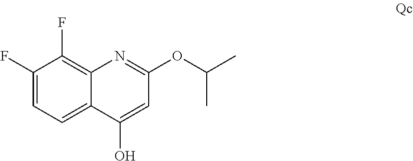

Quinoline Qc is prepared analogously to Qa but starting with the aniline Yc.

¹H NMR (400 MHz, DMSO-d₆) δ (ppm): 11.78-11.88 (m, 1H), 7.80 (ddd, 1H, J=9.2, 5.5, 2.3 Hz), 7.14 (ddd, 1H, J=9.9, 9.2, 7.1 Hz), 6.22 (s, 1H), 5.47 (S, 1H, J=6.2 Hz), 1.33 (d, 6H, J=6.2 Hz).

### Synthesis of Quinoline Qd

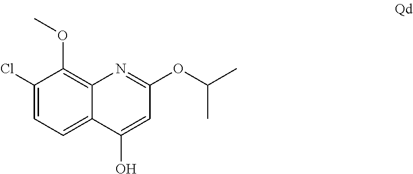

Quinoline Qd is prepared analogously to Qa but starting with the aniline Yd.

¹H NMR (400 MHz, CDCl₃) δ (ppm): 8.19-8.12 (m, 1H), 7.95 (d, 1H, J=9.0 Hz), 7.22 (d, 1H, J=9.0 Hz), 5.74 (d, 1H, J=2.3 Hz), 4.71 (S, 1H, J=6.3 Hz), 4.04 (s, 3H), 1.44 (d, 6H, J=6.3 Hz).

### Synthesis of Quinoline Qe

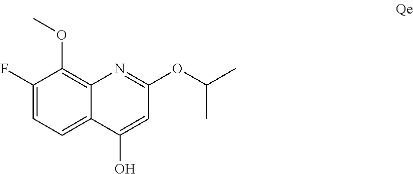

Quinoline Qe is prepared analogously to Qa but starting with the aniline Ye.

### Synthesis of Quinoline Qf

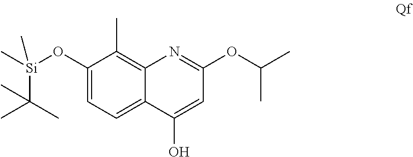

Quinoline Qf is prepared analogously to Qa but starting with the aniline Yf

¹H NMR (400 MHz, CDCl₃) δ (ppm): 8.06 (d, 1H, J=9.2 Hz), 7.61-7.52 (m, 1H), 6.78 (d, 1H, J=9.2 Hz), 5.69 (d, 1H, J=1.6 Hz), 4.69 (S, 1H, J=6.3 Hz), 2.27 (s, 3H), 1.42 (d, 6H, J=6.3 Hz), 1.04 (s, 9H), 0.26 (s, 6H).

### Synthesis of Quinoline Qg

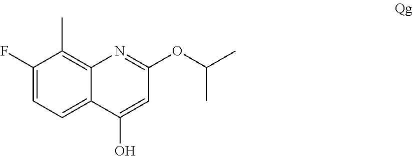

Quinoline Qg is prepared analogously to Qa but starting with the aniline Yg

¹H NMR (400 MHz, DMSO-d₆) δ (ppm): 11.44-11.25 (m, 1H), 7.85 (dd, 1H, J=9.3, 6.8 Hz), 7.13 (t, 1H, J=9.3 Hz), 6.16 (s, 1H), 5.44 (S, 1H, J=6.2 Hz), 2.44 (d, 3H, J=2.2 Hz), 1.33 (d, 6H, J=6.2 Hz).

### Synthesis of Quinoline Qh

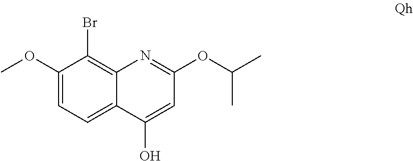

Quinoline Qh is prepared analogously to Qa but starting with the aniline Yh

¹H NMR (400 MHz, DMSO-d₆) δ (ppm): 11.68-11.59 (m, 1H), 8.15 (d, 1H, J=8.9 Hz), 7.41 (d, 1H, J=8.9 Hz), 6.28 (s, 1H), 5.66 (S, 1H, J=6.3 Hz), 4.14 (s, 3H), 1.53 (d, 6H, J=6.3 Hz).

### Synthesis of Quinoline Qn

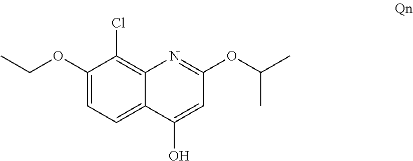

Quinoline Qn is prepared analogously to Qa but starting with the aniline Yn

¹H NMR (400 MHz, CDCl₃) δ (ppm): 8.15 (d, 1H, J=8.8 Hz), 6.93 (d, 1H, J=9.2 Hz), 5.84-5.71 (m, 1H), 4.89-4.64 (m, 1H) 4.23 (q, 1H, J=6.9 Hz), 1.50 (t, 3H, J=6.9 Hz), 1.41 (d, 6H, J=6.3 Hz).

### Synthesis of Quinoline Qi

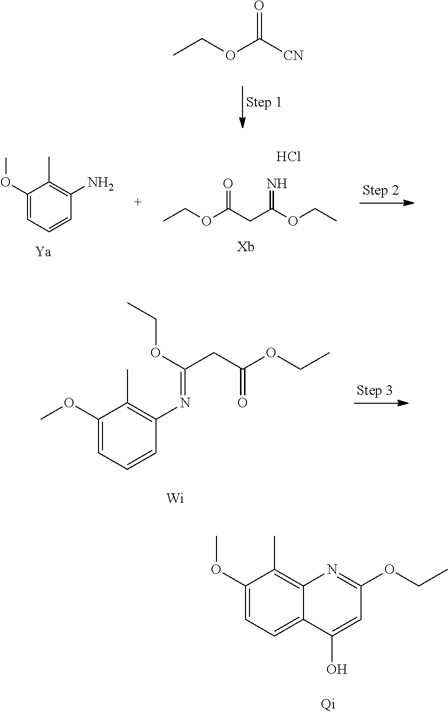

Qi is prepared analogously to Qa by substituting iPrOH for EtOH in Step 1 so that imidate Xb is obtained. Steps 2 and 3 are the same as for the synthesis of Qa.

¹H NMR (400 MHz, DMSO-d₆) δ (ppm): 11.10 (s, 1H), 7.85 (d, 1H, J=9.2 Hz), 7.15 (d, 1H, J=9.2 Hz), 6.09 (s, 1H), 4.42 (q, 2H, J=7.0 Hz), 3.89 (s, 3H), 2.42 (s, 3H), 1.34 (t, 3H, J=7.0 Hz).

### Synthesis of Quinoline Qj

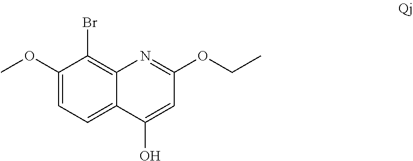

Qj is prepared analogously to Qi but starting with the aniline Yh

¹H NMR (400 MHz, DMSO-d₆) δ (ppm): 11.48 (s, 1H), 8.00 (d, 1H, J=9.2 Hz), 7.26 (d, 1H, J=9.2 Hz), 6.16 (s, 1H), 4.47 (q, 2H, J=7 Hz), 3.97 (s, 3H), 1.37 (t, 3H, J=7 Hz).

### Synthesis of Quinoline Qk

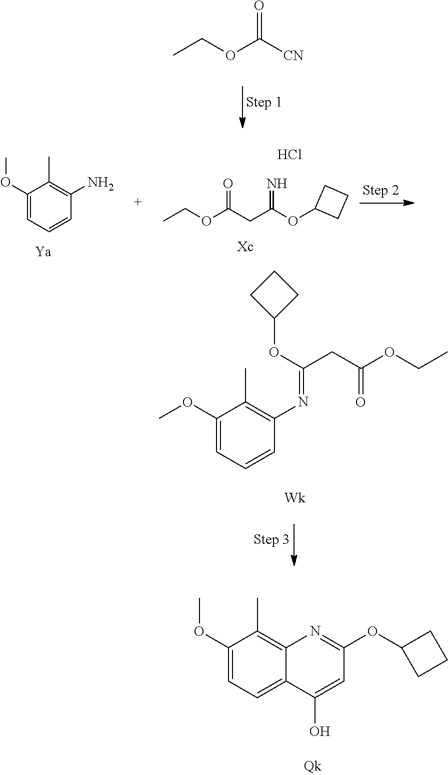

Qk is prepared analogously to Qa by substituting iPrOH for cyclobutanol in Step 1 so that imidate Xc is obtained. Steps 2 and 3 are the same as for the synthesis of Qa.

¹H NMR (400 MHz, DMSO-d₆) δ (ppm): 11.12 (s, 1H), 7.84 (d, 1H, J=9.0 Hz), 7.14 (d, 1H, J=9.0 Hz), 6.07 (s, 1H), 5.26 (q, 1H, J=7.5 Hz), 3.89 (s, 3H), 2.49-2.42 (m, 2H), 2.42 (s, 3H), 2.13-2.02, 1.84-1.63 (m, 2H).

### Synthesis of Quinoline Ql

Ql is prepared via an SNAr reaction with previously published intermediate 10a (WO 2009/14730).

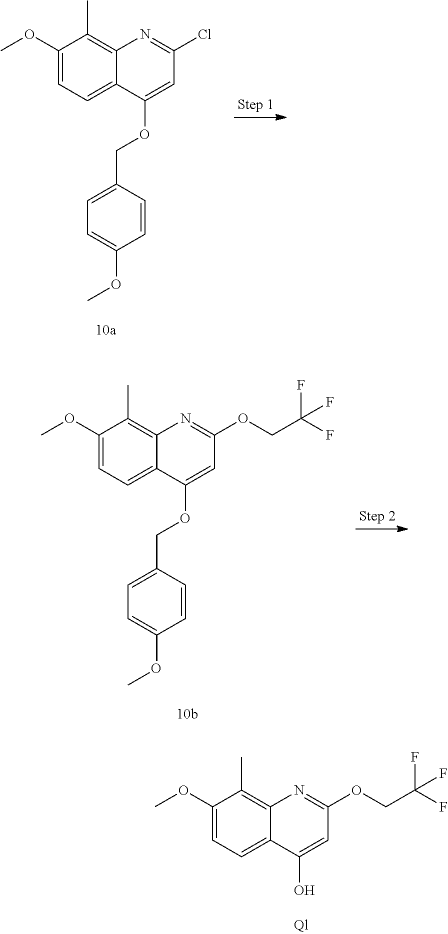

### Step 1:

2,2,2-trifluoroethanol (2.182 g, 21.82 mmol) is added dropwise to NaH powder 60% (872 mg, 218 mmol, 10.0 equiv) as a suspension in DMF (5 mL) at 0° C. The mixture is stirred 1 h at RT, then cooled to 0° C. 10a (750 mg, 2.18 mmol) is added in DMF (5 mL). The resulting mixture is stirred at 60° C. overnight. EtOAc is added and the organic phase is washed with NaHCO₃ (sat.), H₂O and brine; dried over MgSO₄, filtered and concentrated under reduced pressure. The crude material is purified by Combiflash (silica gel 40 g, 2-10% EtOAc/hexanes) to give 10b.

### Step 2:

10b (774 mg) is dissolved in CH₂Cl₂ (5 mL) and treated with TFA (3 mL) for 1 h. The reaction mixture is concentrated in vacuo to provide Ql.

¹H NMR (400 MHz, DMSO-d₆) δ (ppm): 11.48 (s, 1H), 7.91 (d, 1H, J=9.0 Hz), 7.23 (d, 1H, J=9.0 Hz), 6.21 (s, 1H), 5.10 (q, 2H, J=8.7 Hz), 3.91 (s, 3H), 2.43 (s, 3H).

### Synthesis of Quinoline Qm

Qm is prepared via an SNAr reaction with previously published intermediate 10a (WO 2009/14730).

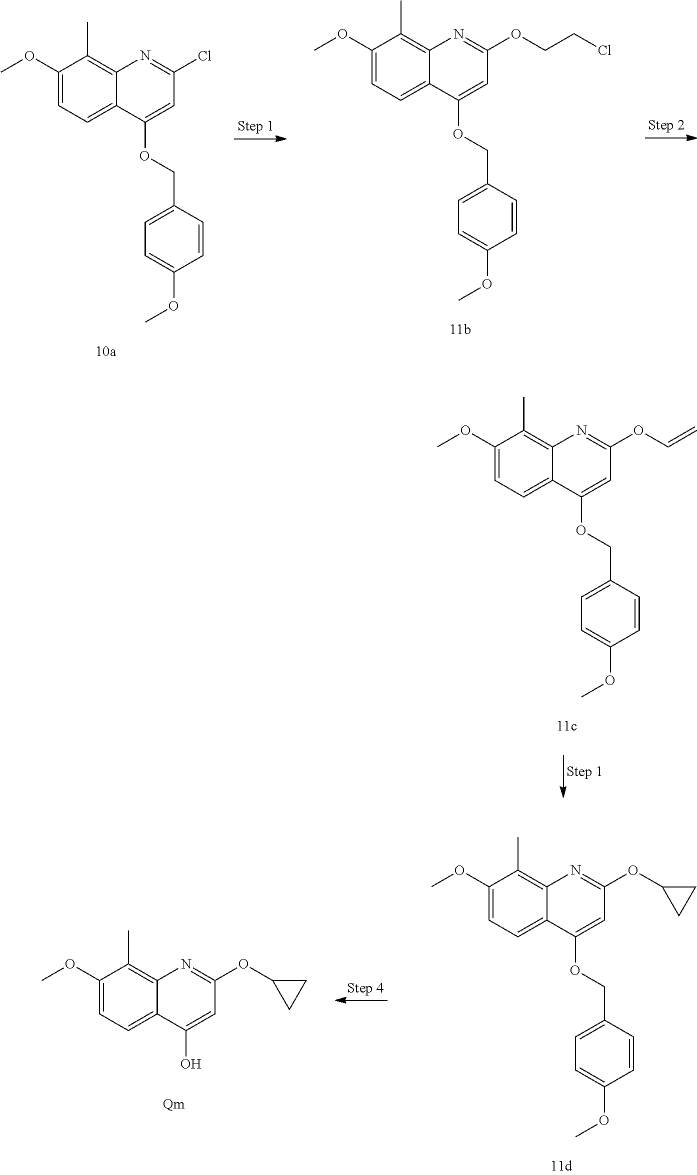

### Step 1:

A 20 mL micowave tube is charged with chloroquinoline 10a (250 mg, 0.727 mmol), cesium carbonate (592 mg, 1.82 mmol), ligand 2-dicyclohexylphosphino-2′,4′,6′-tri-1-propyl-1,1′-biphenyl (45 mg, 0.095 mmol) and palladium acetate (16.3 mg, 0.076 mmol). The tube is sealed and the reaction is evacuated and backfilled with Ar (3×).

Purged toluene is added followed by 2-chloroethanol (98 μL, 1.45 mmol). The tube is again evacuated and backfilled with Ar. The tube is heated for 10 mins at 70° C. in the microwave. CHCl₃, MeOH and EtOAc are added and the crude reaction mixture is concentrated in vacuo to give crude 11b which is used as such in the next synthetic step.

### Step 2:

To a solution of the crude 11b (280 mg, 0.722 mmol) in DMF (7.5 mL) is added LiHMDS 1 M in THF (1.4 mL, 1.4 mmol) and the resulting mixture is stirred at RT for 1 h. Additional LiHMDS 1 M in THF (0.72 mL, 0.72 mmol) is added and the resulting solution is stirred at RT for 30 mins. The reaction mixture is concentrated. The resulting product is dissolved in CHCl₃ then silica gel is added, followed by concentration. Purification by combiflash (24 g column starting at 1% EtOAc/hexane for 1.5 min, then 10% EtOAc/hexane for 10 mins and 100% EtOAc for 4 mins) gives after concentration of the appropriate fraction 11c.

UPLC-MS (electrospray): Retention time=2.4 min, (M+H)⁺ 352.3

Retention Time (min)=4.7 min

¹H NMR (400 MHz, DMSO-d₆): 8, 7.92-7.87 (m, 2H), 7.47 (dd, 2H, J=8.7, 2.0 Hz), 7.26 (d, 1H, J=9.2 Hz), 7.00 (dd, 2H, J=8.8, 2.1 Hz), 6.55 (s, 1H), 5.29 (s, 2H), 4.90 (dd, 1H, J=14.1, 1.4 Hz), 4.61 (dd, 1H, J=6.3, 1.4 Hz), 3.91 (s, 3H), 3.77 (s, 3H), 2.44 (s, 3H).

### Step 3:

To the alkene 11c (370 mg, 1.05 mmol) in dried DCM (10.5 mL) at −20° C. is added chloroiodomethane (461 μL, 6.32 mmol) followed by a slow addition of diethylzinc 1 M in THF (3.16 mL, 3.16 mmol). The mixture is stirred at −20° C. for 60 mins then is slowly increased to −5° C. and kept at −5° C. for 4 h. Chloroiodomethane (231 μL, 3.16 mmol) is added followed by a slow addition of diethylzinc (1.58 mL, 1.58 mmol). This mixture is stirred at 0° C. for 2 h. The reaction mixture is quenched with saturated NH₄Cl and the layers are separated. The aqueous layer is extracted with DCM and the combined organic layers are dried by passing through a phase cartridge separator to give, after concentration, a mixture of 11c and 11d. This mixture is redissolved in anhydrous THF (3 mL) and a 1 M solution of BH₃ in THF (1.26 mL, 1.26 mmol) is added. The solution is stirred for 15 mins then silica gel is added followed by concentration. The crude product is purified by combiflash (12g column starting with 1% EtOAc/hexane for 12 mins then 100% EtOAc for 4 mins). Concentration of the appropriate fraction gives 11d.

UPLC-MS (electrospray): Retention time=2.4 min, (M+H)⁺ 366.3

Retention time (min)=3.8 min

¹H NMR (400 MHz, DMSO-d₆): 8, 7.85 (d, 1H, J=9.2 Hz), 7.47 (dd, 2H, J=6.7, 2.0 Hz), 7.20 (d, 1H, J=9.2 Hz), 6.98 (dd, 2H, J=6.6, 2.1 Hz), 6.38 (s, 1H), 5.24 (s, 2H), 4.48 (tt, 1H, J=6.3, 6.1 Hz), 3.90 (s, 3H), 3.77 (s, 3H), 2.47 (s, 3H), 0.83-0.78 (m, 2H), 0.72-0.69 (m, 2H).

### Step 4:

To a solution of p-methoxybenzyl ether 11d (145 mg, 0.397 mmol) in DCM (2 mL) at RT is added trifluoroacetic acid (0.93 mL, 12.07 mmol) and the mixture is stirred for 30 mins. The mixture is concentrated under vacuum. DCM is added and the resulting solution is then concentrated on the vacuum pump for 1.5 h to give Qm that is used without further purification.

UPLC-MS (electrospray): Retention time=1.5 min, (M+H)⁺ 246.2,

Retention time (min)=2.7 min

¹H NMR (400 MHz, CDCl₃) δ (ppm): 8.09 (d, 1H, J=9.2 Hz), 7.11 (s, 1H), 7.09 (d, 1H, J=9.2 Hz), 4.16 (tt, 1H, J=6.1, 6.0 Hz), 4.01 (s, 3H), 3.79 (bs, 1H), 2.37 (s, 3H), 1.05-1.00 (m, 2H), 0.98-0.94 (m, 2H).

Synthesis of Macrocyclic Intermediate I from Schemes 6 and 7

The synthesis is done as described in Scheme 10

The synthesis of a closely related analog (cyclopentyl carbamate instead of t-butyl carbamate) has been previously described in several patent applications and literature: (see for example: WO 2007030656; Tsantrizos et al., J. Organometallic Chem. 2006, 691, 5163-5171; Yee et al., J. Org. Chem. 2006, 71, 7133-7145). In particular the synthesis of intermediate 1a and 1c has been extensively described in the literature. See above and references within.

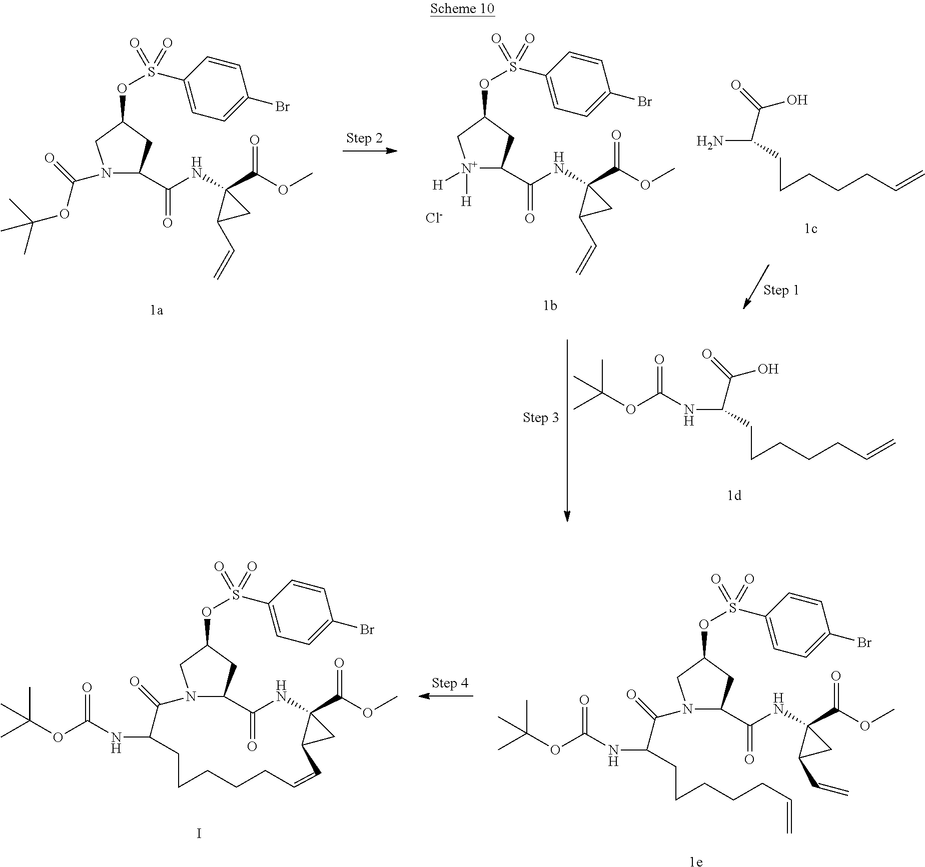

### Step 1:

To a suspension of 1c (50.0 g, 292 mmol) in dioxane (300 mL) and water (200 mL) is added NaOH (12.8 g, 321 mmol) as a solution in water (150 mL). Boc₂O (76.6 g, 218 mmol) is dissolved in dioxane (50 mL) and added dropwise over a period of 30 mins. Addition of the reagent causes a suspension to form and there is a slight exotherm which is controlled with the use of a RT water bath. The reaction is left stirring overnight. The reaction mixture is transferred to a 2 L round-bottom flask using water (250 mL) for the transfer. The dioxane is evaporated at 40° C. Water is added to bring the volume to 1 L and 1 M NaOH (aq., ˜50 mL) is added to adjust the pH to ˜12. Any remaining solids are filtered and discarded. The aqueous solution is washed with a 50/50 mixture of t-BME/hexane (200 mL, 2×). The organic portions are discarded and the aqueous portion is transferred to a 2 L Erlenmeyer flask. t-BME (600 mL) is added and the mixture is cooled in an ice/water bath. 4 M HCl is added slowly until the pH is approximately 3. During the addition, a solid forms which causes the mixture to become an emulsion. The solids are filtered over a glass fiber filter disc and discarded. The filtrate is collected and the aqueous portion removed and extracted with t-BME (200 mL). The organic portions are combined and washed with 0.2 M KHSO₄ (200 mL, 2×), brine (200 mL), dried over Na₂SO₄, filtered and evaporated to give 1d.

### Step 2:

Intermediate 1a (50.2 g, 87.5 mmol, 1.00 equiv) is suspended in EtOAc (225 mL). To the resulting slurry is added a 4 M HCl/dioxane solution (90 mL, 360 mmol, 4.0 equiv) slowly from an addition funnel over about 30 mins, with vigorous stirring. The reaction is only very slightly exothermic—the temperature of the reaction mixture rose from 20° C. to 25° C. (no cooling bath). At the end of the addition, all solids are dissolved. The reaction mixture is stirred for 3 h. The reaction mixture is concentrated in vacuo. The residual viscous liquid is diluted with EtOAc (500 mL) and re-concentrated. The residue is re-dissolved in EtOAc (500 mL), and then further diluted with Et₂O (500 mL). The solution is stored in the fridge (+5° C.) overnight. The precipitate which forms is collected using a sintered glass funnel and rinsed with EtOAc (500 mL, 2×) to give 1b.

### Step 3:

The carbamate 1d (24.5 g, 90.3 mmol, 1.00 equiv) and HBTU (41.1 g, 108 mmol) are suspended in DCM (220 mL) and the suspension is stirred rapidly. DIPEA (15.7 mL, 90.4 mmol, 1.00 equiv) is added at ambient temperature and after 20 mins, a cloudy solution forms. A solution of 1b (47.9 g, 93.9 mmol, 1.04 equiv) in anhydrous dichloromethane (330 mL) containing DIPEA (16.36 mL, 93.9 mmol 1.04 equiv) is then poured into the reaction. The resulting solution is allowed to stir for 16 h. The solvent is then evaporated yielding a syrup which is taken up in EtOAc (1.2 L) and washed with 0.05 N HCl (2×500 mL), saturated Na₂CO₃ (800 mL) and brine (500 mL). The combined extracts are dried over NaSO₄ filtered and concentrated in vacuo. The material is purified by flash chromatography to yield the tripeptide 1e.

### Step 4:

The tripeptide 1e (25.0 g, 34.4 mmol, 1.00 equiv) is dissolved in toluene (2.1 L). The reaction is heated to 80° C. While the mixture is heated, Ar is bubbled through the solution for 1 h. The catalyst (Hoveyda-Grubbs 2^{nd} generation catalyst from Aldrich, 0.3 g×4) is added in 4 equal portions, 30 mins apart. After complete addition, HPLC indicates that the ratio of product to starting material is about 35-40 to 1. The reaction is cooled to 50° C. and a solution of trihydroxymethyl phosphine (see below) is added and the mixture stirred at this temperature for 1 h. The mixture is cooled to RT and silica gel (21 g) is added and the mixture stirred a further 30 mins. The solids are filtered and washed with EtOAc, the filtrate and washings are combined, then washed with 0.5 M KHSO₄ (500 mL), saturated NaHCO₃ (500 mL), water (500 mL) and brine (500 mL). The organic portion is dried over a combination of MgSO₄, silica gel and activated charcoal with stirring for 30 mins. The solids are filtered through a bed of celite and silica, and washed with small portions of EtOAc. The filtrate and washings are combined and evaporated. The residue is co-evaporated with t-BME (300 mL). t-BME (108 mL) is added, followed by the rapid addition of hexane (320 mL). A gummy solid forms which is stirred for about 48 h. A suspension forms. This is further diluted with t-BME (40 mL) and the volume is adjusted to 800 mL with hexanes. The suspension is stirred for 30 mins. The solids are collected and washed with hexanes and air-dried to obtain macrocycle I.

### Trihydroxymethyl Phosphine Preparation

13 g of Tetrakishydroxymethylphosphoniumchloride (80% w/w H₂O) is dissolved in i-PrOH (21.30 mL) under a nitrogen atmosphere. 6.5 g of 45% w/w KOH in water (8 mL) is then added dropwise at RT. After stirring the suspension for 30 mins under nitrogen, the mixture is filtered and the solids washed with degassed i-PrOH (20 mL). The solids are discarded, and the filtrate and washings are combined and stored under nitrogen until used.

### Synthesis of Macrocyclic Intermediates

### Synthesis of Macrocyclic Intermediates Aa and Ba via Scheme 6

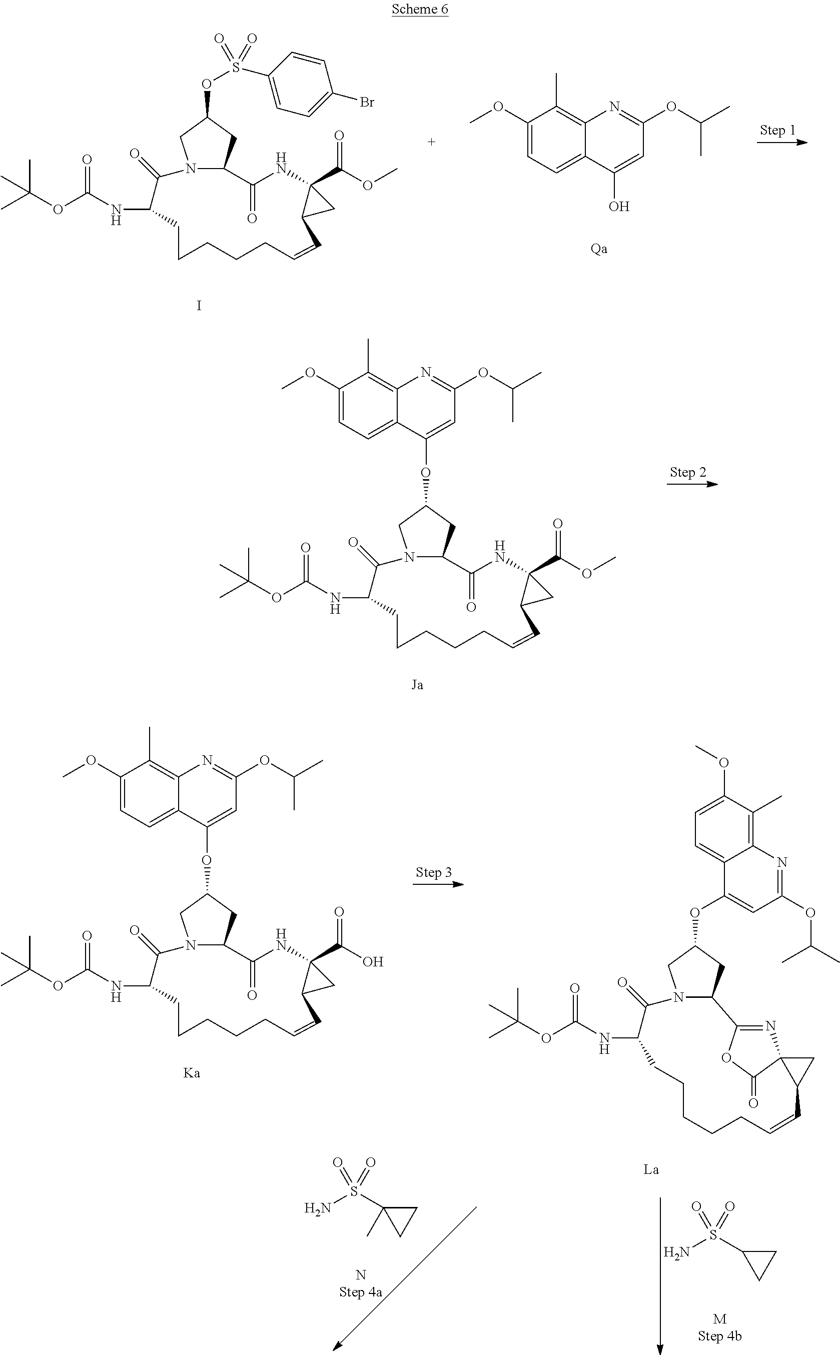

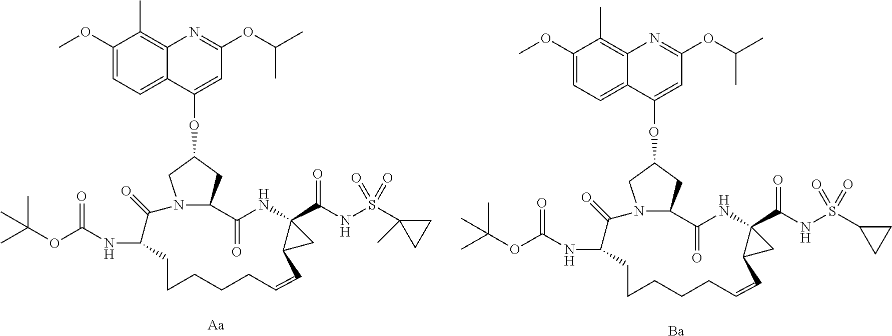

### Step 1: Brosylate Displacement

Brosylate I (10.0 g, 14.31 mmol) and hydroxy quinoline Qa (3.9 g, 15.75 mmol) are dissolved in NMP (150 mL). Cs₂CO₃ (9.33 g, 28.6 mmol) is added and the mixture is heated to 70° C. for 8 h. The solution is cooled to RT and stirred an additional 8 h. The mixture is diluted with EtOAc and washed with H₂O (3×), NaHCO₃ (sat.) (2×), 1.0 N NaOH (1×), H₂O (2×) and brine (1×). The organics are dried over MgSO₄, filtered and concentrated in vacuo. The material is purified by flash chromatography using 30-40% EtOAc/hexanes as the eluent. The product containing fractions are combined and concentrated in vacuo to give Ja.

### Step 2: Hydrolysis

Ja (5.94 g, 8.38 mmol) is dissolved in THF/MeOH (2/1-120 mL) and 1 N NaOH (67 mL, 67 mmol) is added. The reaction mixture is stirred overnight at RT and then concentrated to dryness. The residue is then taken up in EtOAc/H₂O. The two phase mixture is acidified to pH ˜5 with 10% citric acid. The aqueous phase is extracted with EtOAc (3×) and the combined organics are washed with H₂O (3×), brine (1×), dried over MgSO₄, filtered and concentrated in vacuo to give the acid Ka which is used without further purification.

### Step 3: Azalactone Formation

The acid Ka (5.9 g, 8.38 mmol) is dissolved in DCM (55 mL). Triethylamine (3.85 mL, 27.65 mmol) is added and the solution is cooled to 0° C. in an ice bath. Isobutylchlorformate (1.63 mL, 12.57 mmol) is added dropwise and the mixture is stirred at 0° C. for 1 h and then allowed to warm slowly to RT and stirred overnight. The mixture is concentrated in vacuo and the residue taken up in THF/Et₂O (1:1, 100 mL). The mixture is filtered through Celite to remove the salts. The mother liquor is concentrated to dryness to provide azalactone La which is used as such without further purification.

Step 4a: Acyl Sulfonamide Formation with Sulfonamide N

Sulfonamide N (2.4 g, 25.1 mmol) is dissolved in THF (100 mL) and cooled to −20° C. LiHMDS (1.0 N solution in THF, 21.8 mL, 21.8 mmol) is added all at once. The reaction is stirred at −20° C. for 5 min and then allowed to warm to RT for 20 mins. The mixture is then recooled to −20° C. The azalactone La (5.67 g, 8.38 mmol) is dissolved in THF (40 mL) and added dropwise over 1 h to the sulfonamide anion solution. After the addition is complete, the mixture is allowed to warm to RT and the solution stirred overnight. Glacial HOAc (2.0 mL) is added and the reaction mixture is concentrated to dryness. The material is purified by flash chromatography using 25-55% EtOAc/hexanes as the eluent. The pure fractions are combined and concentrated in vacuo to provide Aa.

Step 4b: Synthesis of Ba-Acyl Sulfonamide formation with sulfonamide M

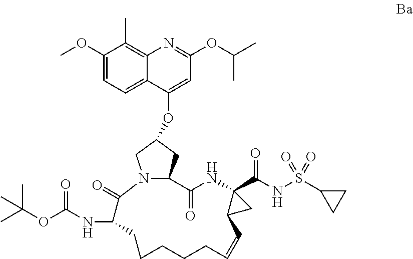

Intermediate Ba is prepared analogously to that of Aa by substituting sulfonamide M in place of sulfonamide N in step 4a of Scheme 5.

### Synthesis of Af

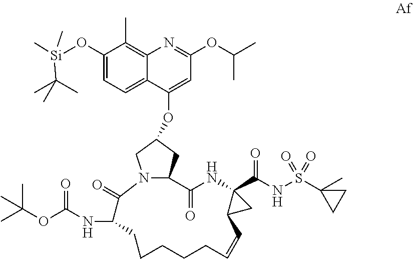

Intermediate Af is prepared analogously to Aa but substituting hydroxyquinoline Qa for Qf in step 1.

### Synthesis of Bf

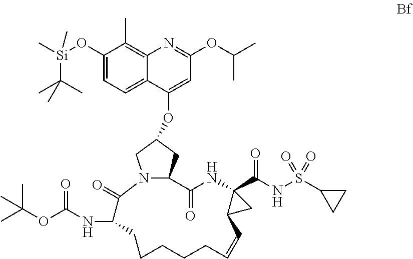

Intermediate Bf is prepared analogously to Ba but substituting hydroxyquinoline Qa for Qf in step 1.

### Synthesis of Ai

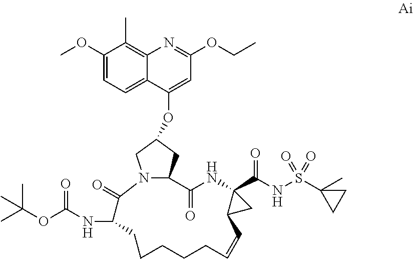

Intermediate Ai is prepared analogously to Aa but substituting hydroxyquinoline Qa for Qi in step 1.

### Synthesis of Bi

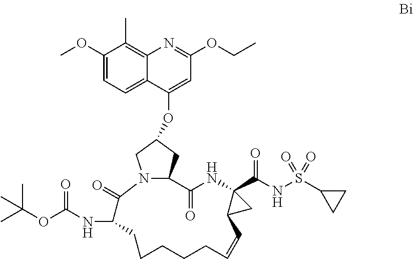

Intermediate Bi is prepared analogously to Ba but substituting hydroxyquinoline Qa for Qi in step 1.

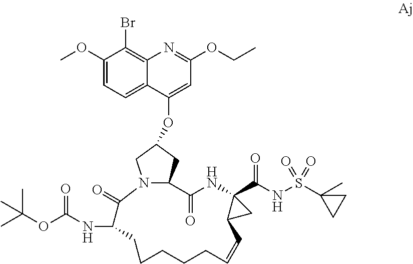

### Synthesis of Aj

Intermediate Ai is prepared analogously to Aa but substituting hydroxyquinoline Qa for Qj in step 1.

### Synthesis of Ak

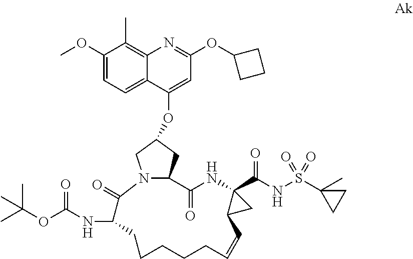

Intermediate Ak is prepared analogously to Aa but substituting hydroxyquinoline Qa for Qk in step 1.

### Synthesis of Al

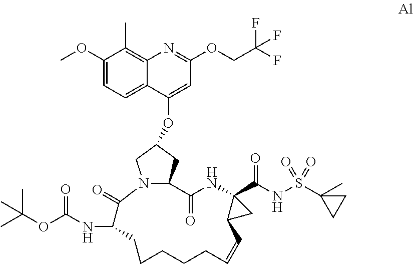

Intermediate Al is prepared analogously to Aa but substituting hydroxyquinoline Qa for Ql in step 1.

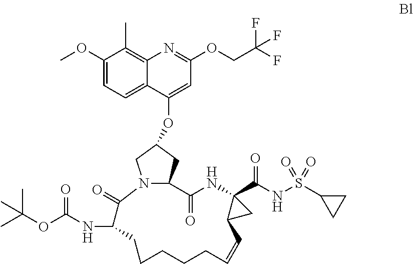

### Synthesis of Bl

Intermediate Bl is prepared analogously to Aa but substituting hydroxyquinoline Qa for Ql in step 1.

Synthesis of macrocyclic intermediate Ae via Scheme 7

### Synthesis of Ae

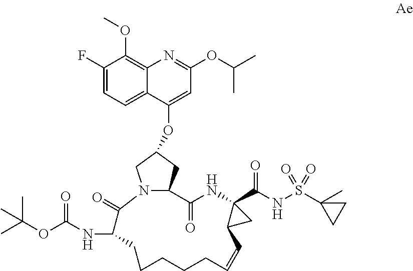

### Example

### Synthesis of Ae via Scheme 7

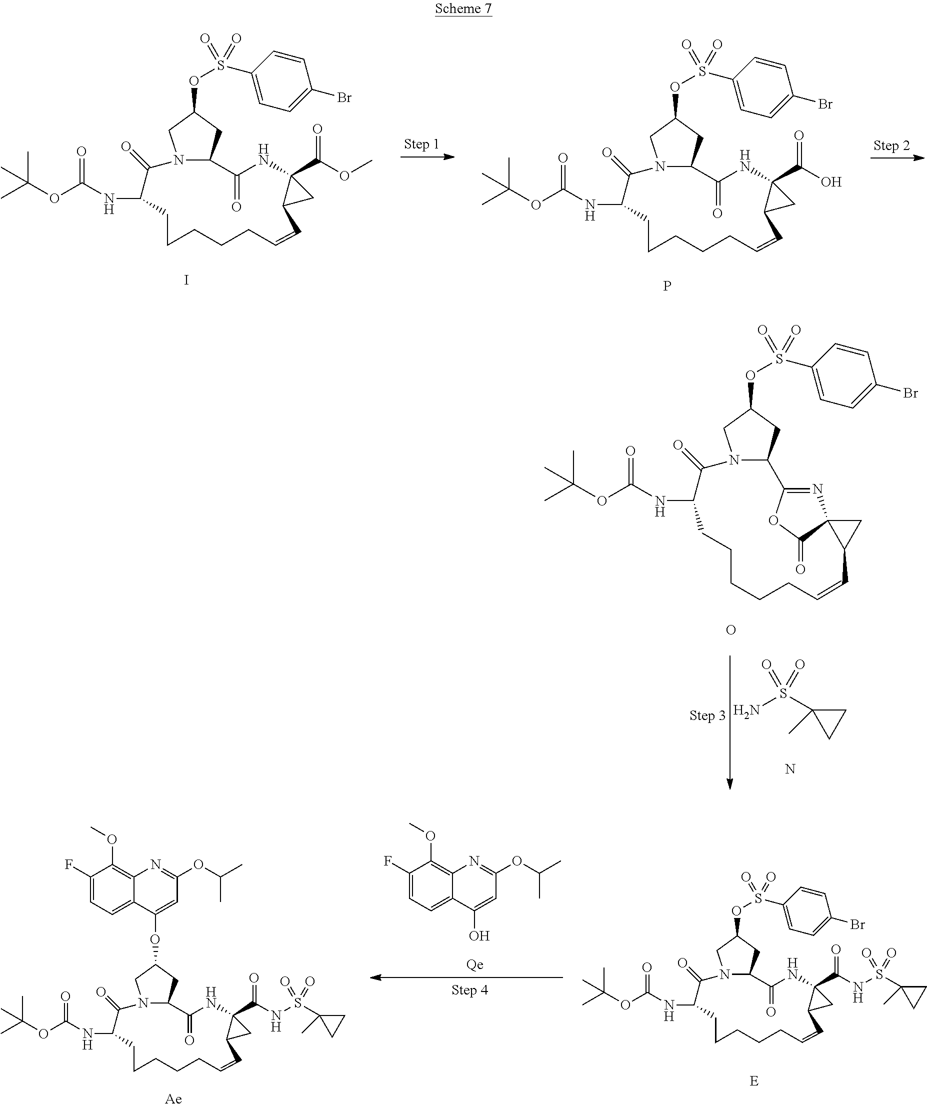

### Step 1: Hydrolysis

Intermediate I (3.0 g, 4.3 mmol) is dissolved in THF/MeOH (3/1, 28 mL) and 1.0 N NaOH (12.9 mL, 12.9 mmol) is added and the reaction is stirred overnight at RT. The reaction mixture is concentrated in vacuo, acidified with 10% citric acid to pH ˜6 and extracted with EtOAc (3×). The combined organic extracts are washed with H₂O (3×), brine (1×), dried over MgSO₄, filtered and concentrated in vacuo to yield carboxylic acid P (2.94 g).

### Step 2: Azalactone Formation

Carboxylic acid P (11.5 g, 16.8 mmol) is dissolved in DCM (160 mL) and triethylamine (7.73 mL, 55.4 mmol) is added. The reaction mixture is cooled to 0° C. in an ice bath. Isobutylchloroformate (3.70 mL, 28.6 mmol) is added dropwise and the mixture is stirred at 0° C. for 1 h and then allowed to warm slowly to RT and stirred overnight. The mixture is concentrated in vacuo and purified by flash chromatography using 35-100% EtOAc/hexanes as the eluent, the pure fractions are combined and concentrated in vacuo to give azalactone O.

### Step 3: Acylsulfonamide Formation

Sulfonamide N (2.96 g, 21.9 mmol) is dissolved in THF (80 mL) and cooled to −20° C. LiHMDS (1.0 N solution in THF, 18.8 mL, 18.8 mmol) is added all at once. The reaction is stirred at −20° C. for 5 mins and then allowed to warm to RT for 20 mins. The mixture is then recooled to −20° C. The azalactone Q (5.66 g, 8.49 mmol) is dissolved in THF (40 mL) and added dropwise over 1 h to the sulfonamide anion solution. After the addition is complete, the mixture is allowed to warm to RT and the solution stirred overnight. Glacial AcOH (2.0 mL) is added and the reaction mixture is concentrated to dryness. The material is purified by flash chromatography using 30-85% EtOAc/hexanes as the eluent. The pure fractions are combined and concentrated in vacuo to provide E.

Step 4: Brosylate Displacement with Hydroxy Quinoline (Qe)

Intermediate E (1.34 g, 1.67 mmol), and hydroxy quinoline Qe (400 mg, 1.59 mmol) are dissolved in NMP (10 mL), Cs₂CO₃ (2.08 g, 6.37 mmol) is added and the mixture is heated to 70° C. for 16 h. The solution is cooled to RT and the mixture is diluted with EtOAc and washed with H₂O (2×), 10% citric acid (1×) and brine (1×). The organics are dried over MgSO₄, filtered and concentrated in vacuo. The material is purified by flash chromatography using 20-60% EtOAc/hexanes as the eluent. The product containing fractions are combined and concentrated in vacuo to give Ae.

### Synthesis of Ac

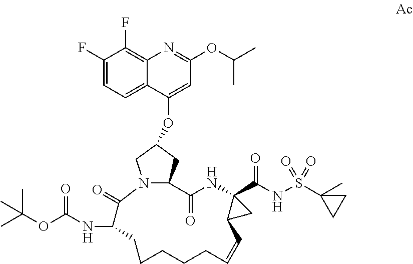

Intermediate Ac is prepared analogously to Ae but substituting hydroxyquinoline Qe for Qc in step 4.

### Synthesis of Ad

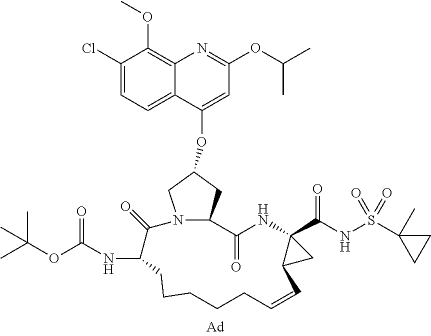

Intermediate Ad is prepared analogously to Ae but substituting hydroxyquinoline Qe for Qd in step 4.

### Synthesis of Ag

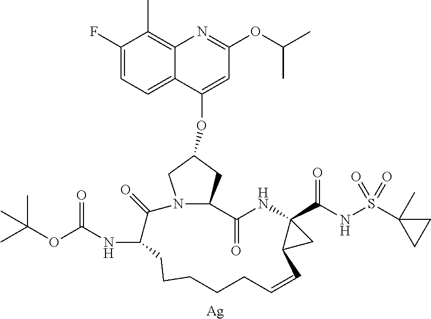

Intermediate Ag is prepared analogously to Ae but substituting hydroxyquinoline Qa for Qg in step 4.

### Synthesis of Am

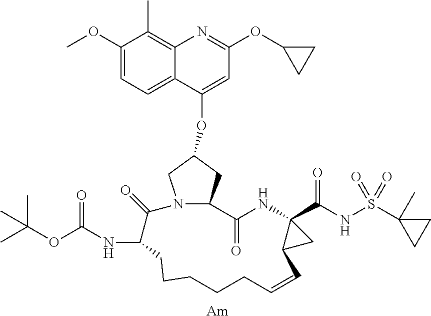

Intermediate Am is prepared analogously to Ae but substituting hydroxyquinoline Qe for Qm in step 4.

### Synthesis of Compounds from Table 1 Using Intermediate Aa

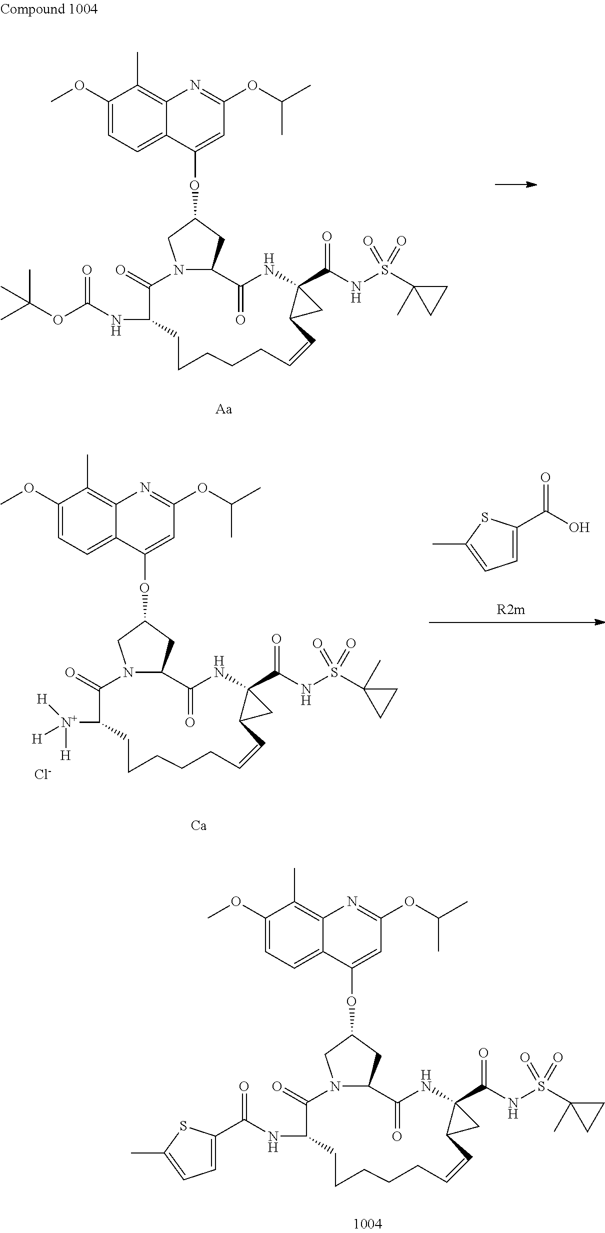

Ca is prepared by dissolving Aa in 4 N HCl/dioxane for 1 h followed by concentration in vacuo.

5-Methyl-2-thiophene carboxilic acid R2m (9.9 mg, 0.069 mmol) is dissolved in DMF (1 mL), then Et₃N (37 μL, 0.267 mmol) is added followed by TBTU (20.6 mg, 0.064 mmol). The solution is stirred for 15 mins, after which the amine hydrochloride Ca (40 mg, 0.053 mmol) is added in DMF (1 mL) and the solution is stirred at RT for 16 h. AcOH is added and the resulting solution is filtered through a Millex filter and purified by prep HPLC (Sunfire column, 0.1% TFA). The pure fractions are combined, frozen and lyophilized to provide compound 1004.

FIA M.S. (electrospray): 836.3 (M+H)⁺

Retention Time (min)=6.2 min

¹H NMR (400 MHz, DMSO-d₆): δ 10.83 (s, 1H), 8.88 (s, 1H), 8.59 (d, 1H, J=6.7 Hz), 7.91 (d, 1H, J=9.0 Hz), 7.68 (d, 1H, J=3.9 Hz), 7.06 (d, 1H, J=9.0 Hz), 6.82 (d, 1H, J=2.7 Hz), 6.35 (s, 1H), 5.65-5.58 (m 1H), 5.49 (S, 1H, J=6.3 Hz), 5.44 (m, 1H), 5.07 (dd, 1H J=10.5, 9.1 Hz), 4.68 (d, 1H, J=10.9 Hz), 4.51-4.43 (m, 1H), 4.34 (dd, 1H, J=7.0, 6.7 Hz), 3.97-3.93 (m, 1H), 3.88 (s, 3H), 2.68-2.58 (m, 2H), 2.45 (s, 3H), 2.43 (s, 3H), 2.40-2.28 (m, 2H), 2.05-1.96 (m, 1H), 1.79-1.69 (m, 1H), 1.57-1.40 (m, 7H), 1.39-1.36 (m, 9H), 1.34-1.22 (m, 4H) 0.89-0.85 (m, 2H).

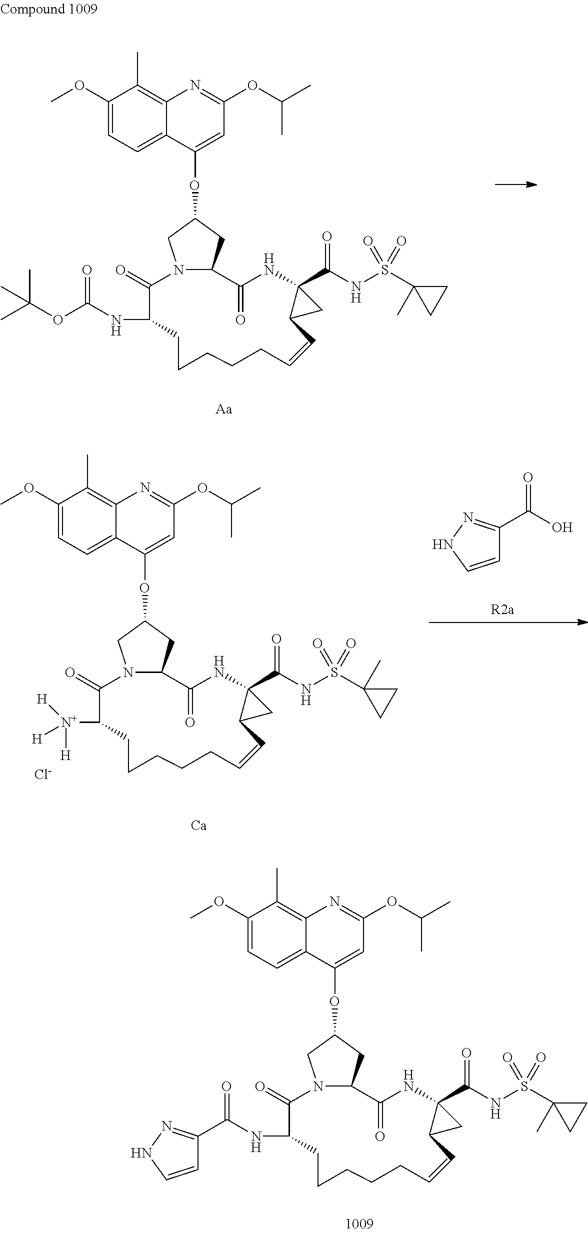

Compound 1009 is synthesized analogously to the procedure used for the preparation of compound 1004 using 40 mg (0.053 mmol) of Aa and R2a (15 mg, 0.090 mmol) as the coupling partner.

FIA M.S. (electrospray): 806.4 (M+H)⁺

Retention Time (min)=5.4 min

¹H NMR (400 MHz, DMSO-d₆): δ 10.81 (s, 1H), 8.86 (s, 1H), 7.86-7.75 (m, 2H), 7.18-7.01 (m, 1H), 7.07 (d, 1H, J=9.4 Hz), 6.62 (s, 1H), 6.36 (s, 1H), 5.59 (b, 1H), 5.49 (p, 1H, J=6.1 Hz), 5.45 (m, 1H), 5.10 (b, 1H), 4.63-4.50 (m, 2H), 4.43-4.35 (m, 1H), 4.06 (m, 1H), 3.92-3.92 (m, 1H), 3.87 (s, 3H), 2.68-2.66 (m, 1H), 2.43 (s, 3H), 2.38-2.32 (m, 2H), 1.99-1.83 (m, 2H), 1.58-1.23 (m, 15H), 1.39 (d, 3H, J=6.1 Hz), 1.37 (d, 3H, J=6.1 Hz), 0.87-0.81 (m, 2H).

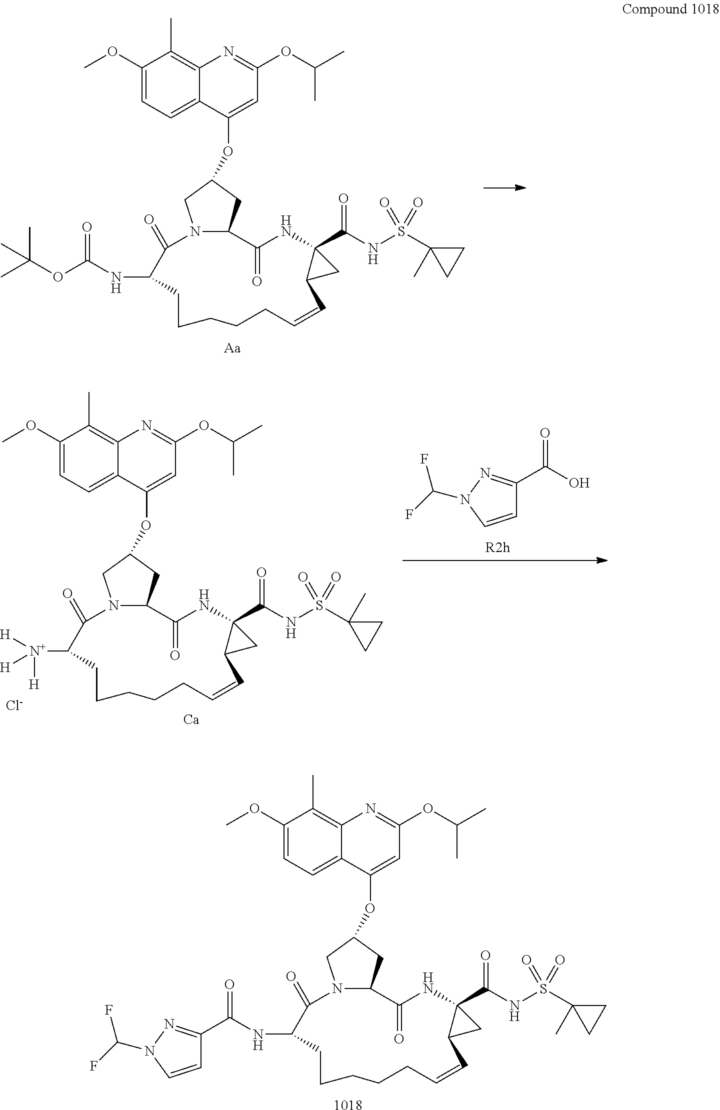

Boc protected macrocyclic amine Aa (75 mg, 0.092 mmol) is charged in a vial with a 4 M solution of HCl in dioxane (2 mL). The solution is stirred at RT for 1 h, after which the solution is evaporated to dryness. 1-difluoromethyl-1H-pyrazole-3-carboxylic acid R2h (17.9 mg, 0.111 mmol, 1.2 equiv) is dissolved in DMF (2 mL) and TEA (51.5 μL; 0.396 mmol, 4 equiv) and TBTU (35.6 mg; 0.111 mmol, 1.2 equiv) are added. The mixture stirred for 15 mins. The Boc de-protected macrocyclic amine hydrochloride Ca is dissolved in DMF (1.0 mL) and added to the acid solution. The reaction is stirred at RT overnight. The resulting solution is filtered through a Millex filter and purified by prep HPLC (X-Bridge, Ammonium Bicarbonate/MeOH) The pure fractions are combined, concentrated, frozen and lyophilized to provide Compound 1018.

FIA M.S. (electrospray): 854.5 (M−H)⁻, 856.4 (M+H)⁺

Retention Time (min)=6.0 min

¹H NMR (400 MHz, DMSO-d₆): δ 10.82 (bs, 1H), 8.91 (bs, 1H), 8.33 (d, 1H, J=Hz), 8.39-8.2 (bs, 1H), 7.87 (s, 1H), 7.84 (d, 1H, J=9.0 Hz), 7.07 (d, 1H, J=9.0 Hz), 6.86 (d, 1H, J=2.4 Hz), 6.35 (s, 1H), 5.70-5.60 (m, 1H), 5.52-5.44 (p, 1H, J=5.9 Hz), 5.44 (s, 1H), 5.20-4.93 (m, 1H), 4.75-4.47 (m, 2H), 4.46-3.15 (m, 1H), 4.14-3.97 (m, 1H), 3.75 (s, 3H), 2.68-2.51 (m, 1H), 2.43 (s, 3H), 2.39-2.22 (m, 2H), 2.12-1.92 (m, 1H), 1.91-1.71 (m, 1H), 1.68-1.49 (m, 4H), 1.48-1.33 (m, 13H), 1.32-1.07 (m, 4H), 0.87 (bs, 2H).

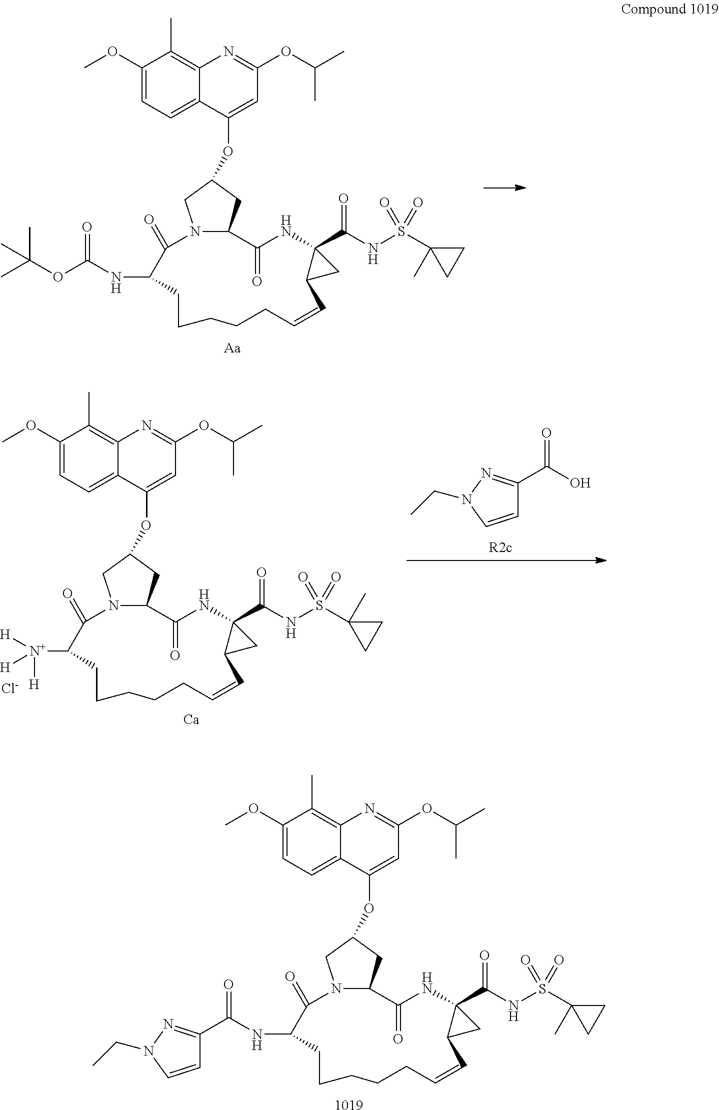

Compound 1019 is synthesized analogously to the procedure described for the preparation of compound 1004 using 46 mg (0.057 mmol) of Aa and R2c (15 mg, 0.090 mmol) as the coupling partner.

FIA M.S. (electrospray): 834.5 (M+H)⁺

Retention time (min)=5.9 min

¹H NMR (400 MHz, DMSO-d₆): δ 10.80 (s, 1H), 8.91 (s, 1H), 7.82 (d, 1H, J=9.2 Hz), 7.80 (d, 1H, J=2.2 Hz), 7.74 (d, 1H, J=6.9 Hz), 7.07 (d, 1H, J=9.2 Hz), 6.59 (d, 1H, J=2.2 Hz), 6.35 (s, 1H), 5.64-5.56 (1H, m), 5.49 (p, 1H, J=6.2 Hz), 5.44 (m, 1H), 5.11-5.04 (m, 1H), 4.65-4.58 (m, 1H), 4.53-4.48 (m, 1H), 4.42 (dd, 1H, J=9.0, 7.7 Hz), 4.18 (q, 2H, J=7.2 Hz), 4.07-4.01 (m, 1H), 3.87 (s, 3H), 2.68-2.59 (m, 1H), 2.43 (s, 3H), 2.38-2.30 (m, 2H), 2.00-1.83 (m, 2H), 1.59-1.47 (m, 4H), 1.41-1.37 (m, 16H), 1.31-1.23 (m, 4H), 0.90-0.83 (m, 2H).

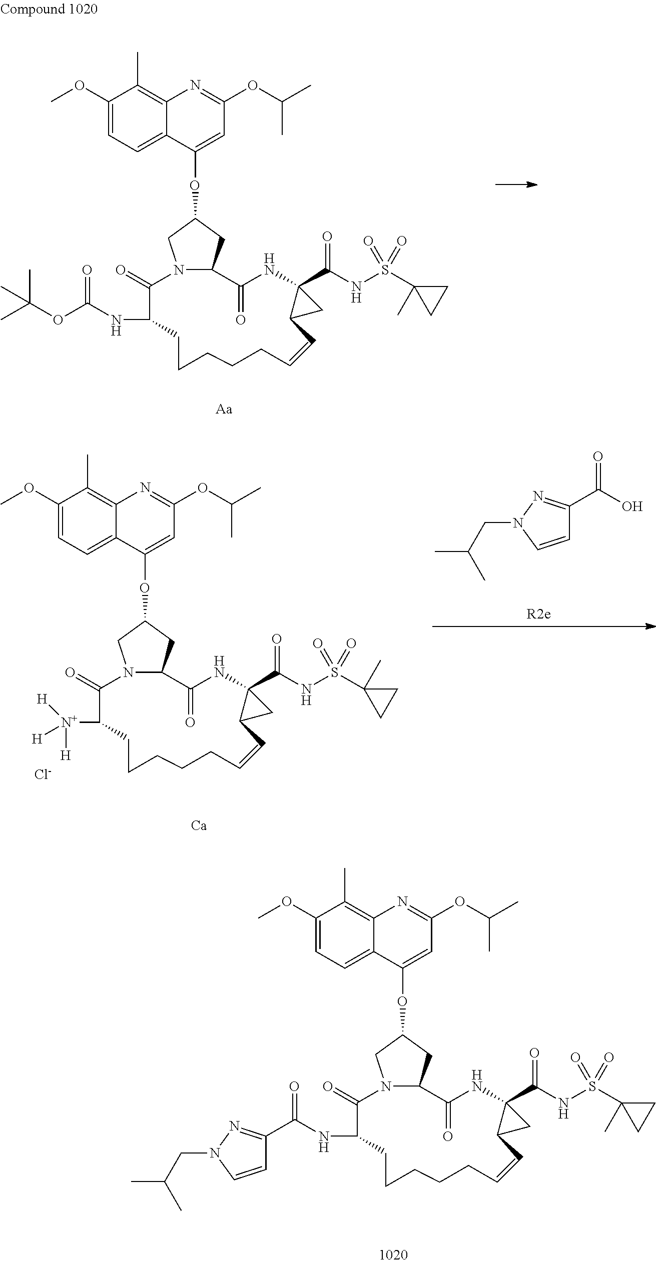

Compound 1020 is synthesized analogously to the procedure described for the preparation of compound 1004 using 45 mg (0.055 mmol) of Aa and R2e (15 mg, 0.087 mmol) as the coupling partner.

FIA M.S. (electrospray): 862.5 (M+H)⁺

Retention time (min)=6.3 min

¹H NMR (400 MHz, DMSO-d₆): δ 10.82 (s, 1H), 8.92 (s, 1H), 7.85-7.78 (m, 3H), 7.06 (d, 1H, J=9.0 Hz), 6.61 (d, 1H, J=2.4 Hz), 7.07, 6.35 (s, 1H), 5.65-5.58 (1H, m), 5.48 (p, 1H, J=6.2 Hz), 5.45 (m, 1H), 5.09-5.04 (m, 1H), 4.61-4.51 (m, 2H), 4.42-4.38 (m, 1H), 4.03-4.01 (m, 1H), 3.95 (d, 2H, J=7.4 Hz), 3.87 (s, 3H), 2.68-2.58 (m, 1H), 2.43 (s, 3H), 2.36-2.31 (m, 2H), 2.19-2.08 (m, 1H), 2.00-1.91 (m, 1H), 1.85-1.78 (m, 1H), 1.59-1.10 (m, 15H), 1.38 (d, 3H, J=6.2 Hz), 1.37 (d, 3H, J=6.2 Hz), 0.88 (m, 2H), 0.83 (d, 3H, J=6.5 Hz), 0.82 (d, 3H, J=6.5 Hz).

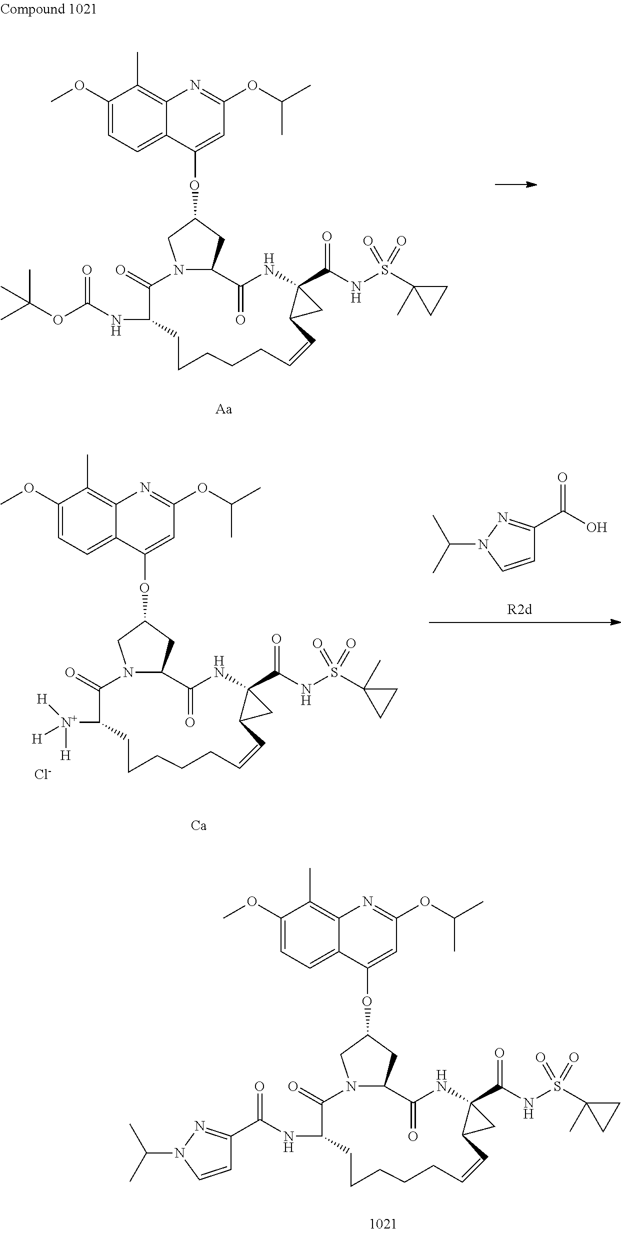

Boc protected macrocyclic amine Aa (60 mg, 0.074 mmol) is charged in a vial, dissolved in DCM (500 μL), then a 4 M solution of HCl in dioxane (2 mL) is added. The solution is stirred at RT for 2 h, after which the solution is evaporated to dryness. The Boc de-protected macrocyclic amine hydrochloride Ca is dissolved in DMF (1.0 mL), then, diisopropylethylamine (64 μL; 0.369 mmol), the 1-isopropyl-1H-pyrazole-3-carboxylic acid R2d (13 mg; 0.084 mmol) and HATU (33.7 mg; 0.089 mmol) are added. The reaction is stirred at RT overnight. The resulting solution is filtered through a Millex filter and purified by prep HPLC (X-Bridge column, Ammonium Bicarbonate pH10: MeOH). The pure fractions are combined, concentrated, frozen and lyophilized to provide compound 1021.

FIA M.S. (electrospray): 846.5 (M−H)−, 848.5 (M+H)⁺

Retention Time (min)=6.1 min

¹H NMR (400 MHz, DMSO-d₆): δ 10.79 (s, 1H), 8.91 (s, 1H), 7.86-7.71 (m, 2H), 7.74 (d, 1H, J=5.7 Hz), 7.06 (d, 1H, J=9.2 Hz), 6.59 (d, 1H, J=2.3 Hz), 6.35 (s, 1H), 5.74-5.50 (m, 1H), 5.55-5.45 (m, 1H), 5.44 (bs, 1H), 5.13-5.02 (m, 1H), 4.65-4.48 (m, 3H), 4.48-4.37 (m, 1H), 4.11-3.95 (m, 1H), 3.87 (s, 3H), 2.74-2.55 (m, 1H), 2.43 (s, 3H), 2.40-2.28 (m, 2H), 2.08-1.91 (m, 1H), 1.91-1.78 (m, 1H), 1.61-1.45 (m, 3H), 1.44 (d, 3H, J=1.7 Hz), 1.43 (d, 3H, J=1.8 Hz), 1.39 (d, 3H, J=4.8 Hz), 1.38 (d, 3H, J=6.1 Hz), 1.46-1.35 (m, 7H), 1.35-1.20 (m, 5H), 0.93-0.82 (m, 2H).

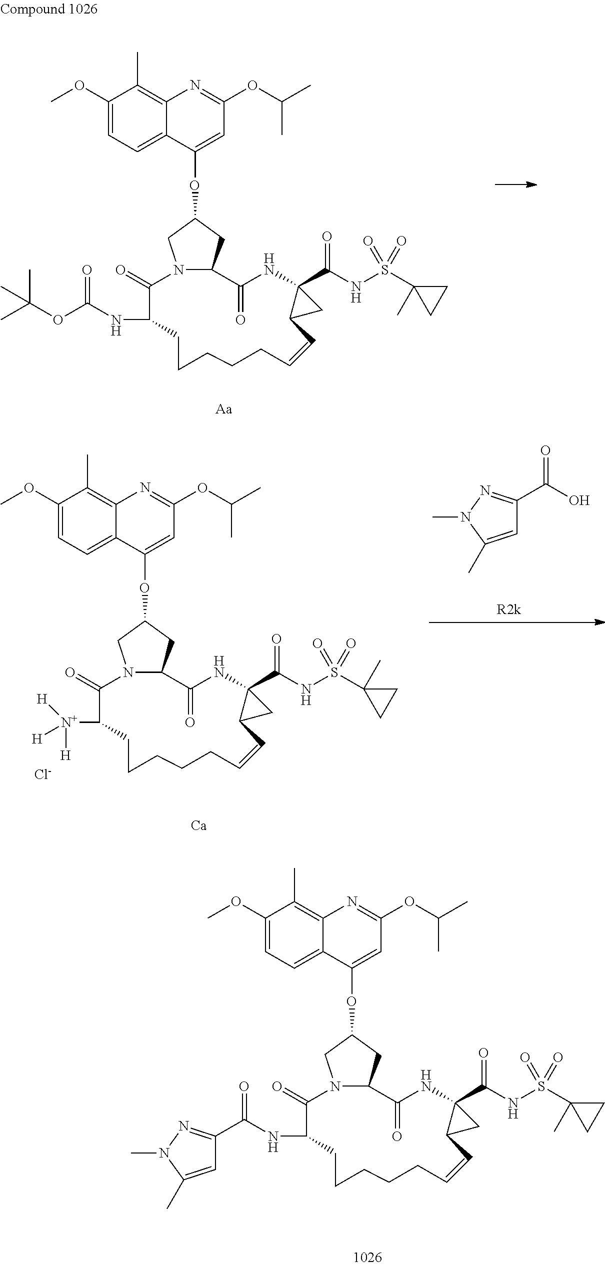

Compound 1026 is synthesized analogously to the procedure described for the preparation of compound 1004 using 40 mg (0.053 mmol) of Aa and R2k (15 mg, 0.085 mmol) as the coupling partner.

FIA M.S. (electrospray): 834.5 (M+H)⁺

Retention Time (min)=5.8 min

¹H NMR (400 MHz, DMSO-d₆): δ 10.83 (s, 1H), 8.92 (s, 1H), 7.81 (d, 1H, J=9.0 Hz), 7.66 (d, 1H, J=6.7 Hz), 7.07 (d, 1H, J=9.0 Hz), 6.36 (s, 1H), 6.35 (s, 1H), 5.63-5.58 (m, 1H), 5.48 (p, 1H, J=6.2 Hz), 5.44 (m, 1H), 5.09-5.04 (m, 1H), 4.61-4.57 (m, 1H), 4.52-4.47 (m, 1H), 4.42-4.38 (m, 1H), 4.05-3.98 (m, 1H), 3.87 (s, 3H), 3.76 (s, 3H), 2.67-2.59 (m, 1H), 2.43 (s, 3H), 2.38-2.30 (m, 2H), 2.25 (s, 3H), 1.97-1.78 (m, 2H), 1.58-1.50 (m, 3H), 1.44-1.23 (m, 12H), 1.39 (d, 3H, J=6.2 Hz), 1.37 (d, 3H, J=6.2 Hz), 0.91-0.85 (m, 2H).

### Synthesis of Compounds from Table 2 Using Intermediate Ca

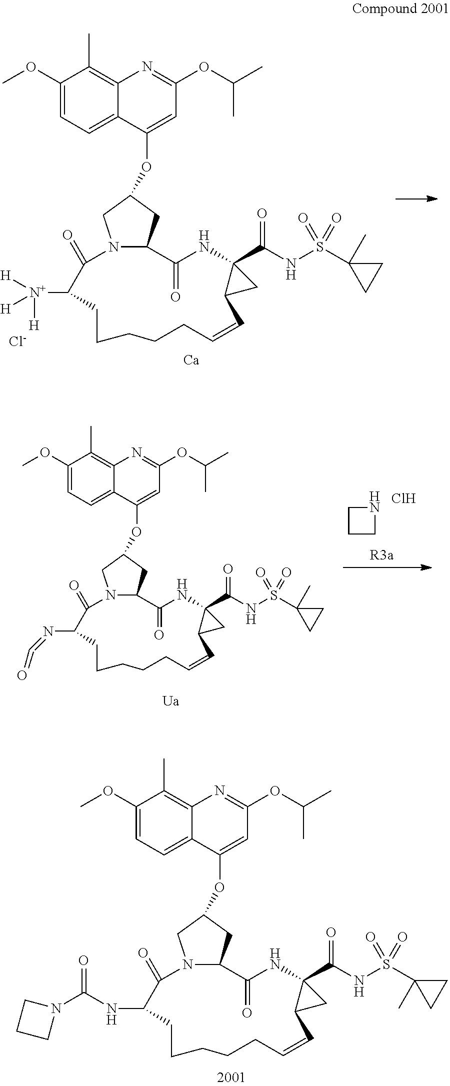

Amine hydrochloride salt Ca (168 mg, 0.23 mmol) is dissolved in DCM (2 mL) and then diisopropylethylamine (79 μL, 0.45 mmol) is added. This mixture is cooled at 0° C., and then triphosgene (33.4 mg, 0.12 mmol) is slowly added via a syringe as a DCM (1 mL) solution. This mixture of crude Ua is stirred at this temperature for 25 mins and is kept as a stock solution for the next step.

In another vial, azetidine hydrochloride R3a (14.1 mg, 0.15 mmol) is charged in 0.2 mL DCM along with diisopropylethylamine (26 μL, 0.15 mmol). This mixture is cooled at 0° C. Using the stock solution of Ua previously prepared, 1 mL (assuming 55.4 mg, 0.075 mmol) is transferred and this mixture is stirred overnight at RT. The resulting mixture is concentrated, redissolved in a minimal amount of MeCN and filtered through a Millex filter prior to being purified by prep HPLC (Sunfire column, 0.1% TFA). The pure fractions are combined, concentrated, frozen and lyophilized to provide compound 2001.

FIA M.S. (electrospray): 795.3 (M+H)⁺, 793.5 (M−H)⁺

Retention time (min)=5.5 min

¹H NMR (400 MHz, DMSO-d₆): δ10.83 (s, 1H), 8.88 (s, 1H), 7.89 (d, 1H, J=9.0 Hz), 7.12 (d, 1H, J=9.0 Hz), 6.40 (bs, 1H), 6.33 (s, 1H), 5.64-5.58 (m, 1H), 5.49 (S, 1H, J=6.2 Hz), 5.43-5.40 (m 1H), 5.05 (dd, 1H, J=9.6, 9.4 Hz), 4.54 (d, 1H, J=11.4 Hz), 4.37 (dd, 1H, J=10.0, 7.0 Hz), 4.22-4.16 (m, 1H), 3.93 (d, 1H, J=7.8, 3.7 Hz), 3.90 (s, 3H), 3.69 (t, 4H, J=7.7 Hz), 2.61-2.55 (m, 2H), 2.44 (s, 3H), 2.42-2.27 (m, 2H), 2.07-1.99 (m, 2H), 1.87-1.70 (m, 2H), 1.58 (dd, 1H, J=8.2, 5.1 Hz), 1.51 (dd, 1H, J=9.4, 4.9 Hz), 1.46-1.34 (m, 5H), 1.39 (s, 3H), 1.39 (d, 3H, J=6.2 Hz), 1.38 (d, 3, J=6.2 Hz), 1.32-1.17 (m, 4H), 0.91-0.86 (m, 2H).

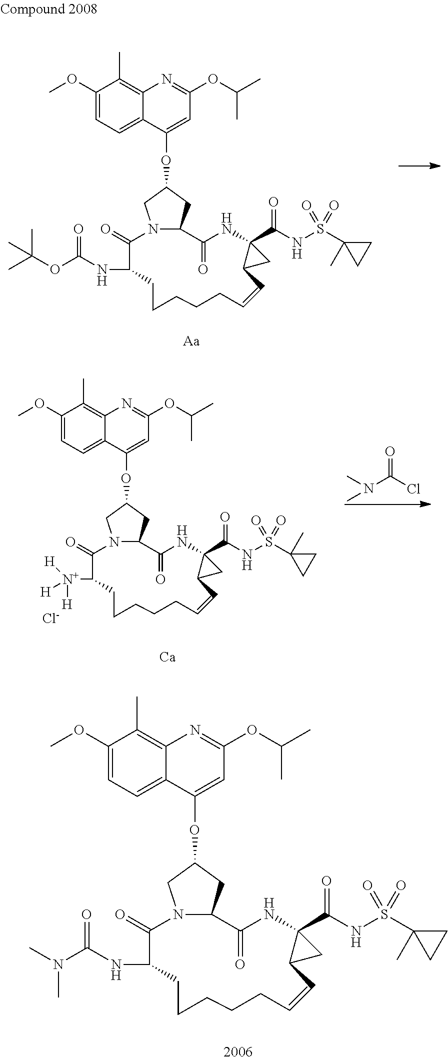

Boc protected amine Aa (2.52 g, 3.10 mmol) is charged in a vial, then a 4 M solution of HCl in dioxane (40 mL, 160 mmol) is added. The solution is stirred at RT for 2 h, after which a precipitate forms. The solution is evaporated to dryness to give intermediate Ca. Amine hydrochloride Ca is redissolved in DCM (40 mL), then Et₃N (2.16 mL, 15.5 mmol, 5.0 equiv) is added. Dimethylcarbamyl chloride (400 mg, 3.72 mmol, 1.20 equiv) is dissolved in DCM (10 mL), then added into the amine solution. This solution is stirred at RT. The reaction is completed after 48 h. Water (25 mL) is added and the organic layer is extracted with DCM (3×50 mL). The solution is concentrated and purified on CombiFlash (50-100% EtOAc/Hexane). The pure fractions are combined, concentrated, frozen and lyophilized to provide compound 2008.

FIA M.S. (electrospray): 783.4 (M+H)⁺

Retention time (min)=5.6 min

¹H NMR (400 MHz, DMSO-d₆): δ 10.81 (s, 1H), 8.85 (s, 1H), 7.96 (d, 1H, J=8.8 Hz), 7.07 (d, 1H, J=9.2 Hz), 6.33 (s, 1H), 6.26 (d, 1H, J=7.0 Hz), 5.67-5.57 (m 1H), 5.49 (S, 1H, J=6.0 Hz), 5.43-5.38 (m, 1H), 5.05 (dd, 1H, J=9.5, 9.4 Hz), 4.67 (d, 1H, J=11.3 Hz), 4.36 (dd, 1H, J=10.1, 7.0 Hz), 4.23-4.13 (m, 1H), 3.94-3.89 (m, 1H), 3.89 (s, 3H), 2.74 (s, 6H), 2.65-2.54 (m, 2H), 2.44 (s, 3H), 2.41-2.27 (m, 2H), 1.93-1.82 (m, 1H), 1.81-1.69 (m, 1H), 1.58 (dd, 1H, J=8.2, 5.1 Hz), 1.51 (dd, 1H, J=9.3, 5 Hz), 1.47-1.33 (m, 14H), 1.33-1.18 (m, 4H), 0.93-0.83 (m, 2H).

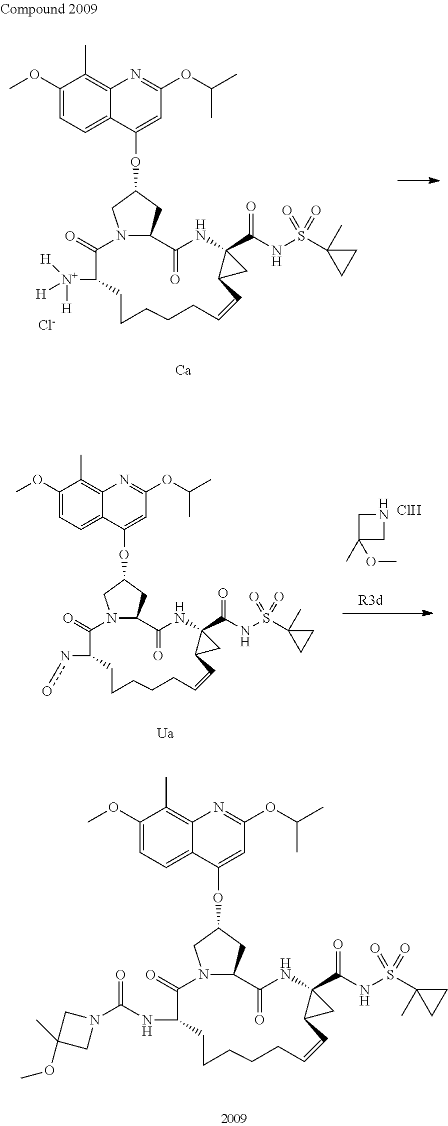

Amine hydrochloride Ca (55 mg, 0.073 mmol) is dissolved in DCM (1 mL), then Et₃N (31 μL, 0.22 mmol, 5.0 equiv) is added, followed by carbonyl diimidazole (14.3 mg, 0.088 mmol, 1.2 equiv). This solution is stirred at RT. The azetidine hydrochloride R3d is dissolved in DCM (1 mL) and then added into the solution of Ua. The reaction is completed after 16 h. The solution is concentrated and purified on prep HPLC (MeCN:H₂O, 0.1% TFA). The pure fractions are combined, concentrated, frozen and lyophilized to provide compound 2009.

FIA M.S. (electrospray): 839.3 (M+H)⁺; 837.4 (M−H)⁺

Retention time (min)=5.6 min

¹H NMR (400 MHz, DMSO-d₆): δ 10.81 (s, 1H), 8.87 (s, 1H), 7.87 (d, 1H, J=9.2 Hz), 7.11 (d, 1H, J=9.2 Hz), 6.60-6.51 (m, 1H), 6.33 (s, 1H), 5.65-5.56 (m 1H), 5.48 (S, 1H, J=6.2 Hz), 5.42-5.39 (m, 1H), 5.04 (dd, 1H, J=10.1, 8.9 Hz), 4.57 (d, 1H, J=11.3 Hz), 4.36 (dd, 1H, J=10.1, 6.9 Hz), 4.24-4.17 (m, 1H), 3.93 (dd, 1H, J=11.6, 3.6 Hz), 3.89 (s, 3H), 3.68 (d, 1H, J=8.8 Hz), 3.64 (d, 1H, J=8.7 Hz), 3.52 (d, 1H, J=8.6 Hz), 3.48 (d, 1H, J=8.5 Hz), 3.10 (s, 3H), 2.64-2.56 (m, 2H), 2.43 (s, 3H), 2.42-2.26 (m, 2H), 1.91-1.69 (m, 2H), 1.57 (dd, 1H, J=8.2, 5.1 Hz), 1.50 (dd, 1H, J=9.3, 5 Hz), 1.47-1.14 (m, 21H), 0.92-0.82 (m, 2H).

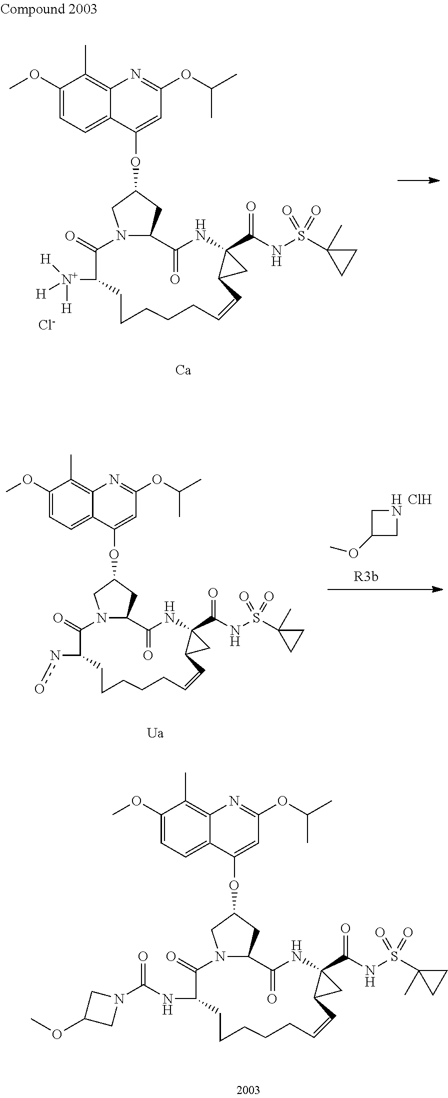

The crude amine-HCl macrocycle Ca (55 mg; 0.073 mmol) is dissolved in DCM (1 mL), carbonyl diimidazole (14.3 mg; 0.88 mmol) is added and the mixture is allowed to stir at RT for 1 h. Meanwhile, the t-butyl-3-methoxyazetidine-1-carboxylate (16.5 mg; 0.88 mmol) is charged in a vial with a 4 M solution of HCl in dioxane (3 mL). The solution is stirred at RT for 1.5 h, after which the solution is evaporated to dryness.

This deprotected 3-methoxyazetidine-1-carboxylate. HCl R3b is dissolved in DCM (1 mL) and is added to the activated macrocyclic component and stirred at RT overnight. The mixture is evaporated to dryness and subsequentlydissolved in a mixture of CH₃CN/DMSO/HOAc, filtered through a Millex filter and purified by prep HPLC (Sunfire column; 0.1% TFA/CH₃CN: 0.1% TFA/H₂O). The pure fractions are combined, concentrated, frozen and lyophilized to provide compound 2003.

FIA M.S. (electrospray): 823.4 (M−H)−, 825.3 (M+H)⁺

Retention time (min)=5.5 min

¹H NMR (400 MHz, DMSO-d₆): δ 10.81 (s, 1H), 8.87 (s, 1H), 7.88 (d, 1H, J=9.2 Hz), 7.12 (d, 1H, J=9.2 Hz), 6.55 (d, 1H, J=5.3 Hz), 6.33 (s, 1H), 5.66-5.56 (m, 1H), 5.53-5.44 (m, 1H), 5.47 (bs, 1H), 5.04 (dd, 1H, J=8.8, 18.8 Hz), 4.56 (d, 1H, J=10.9 Hz), 4.364 (dd, 1H, J=7, 17 Hz),), 4.23-4.16 (m, 1H), 4.05-3.99 (m, 1H), 3.95-2.82 (m, 2H), 3.89 (s, 3H), 3.58-3.49 (m, 2H), 3.16 (s, 3H), 2.63-2.52 (m, 2H), 2.43 (s, 3H), 2.43-2.25 (m, 2H), 1.89-1.70 (m, 2H), 1.60-1.54 (m, 1H), 1.53-1.47 (m, 1H), 1.44-1.15 (m, 19H), 0.93-0.81 (m, 2H).

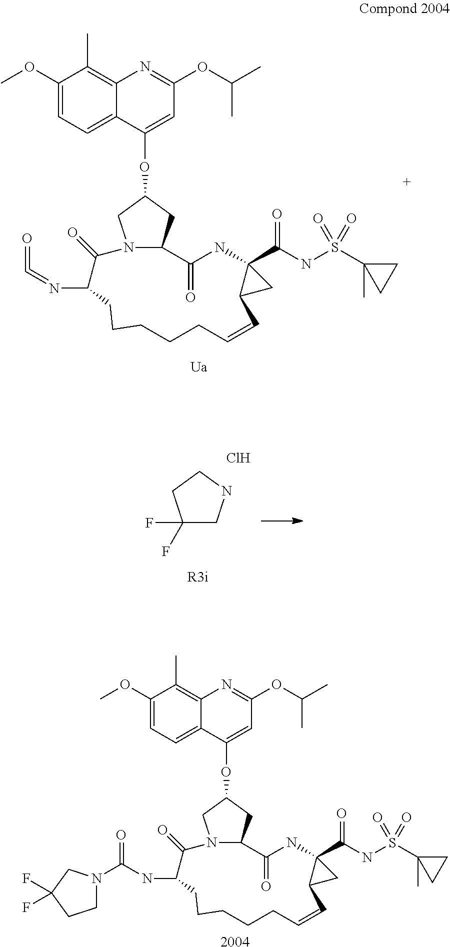

Compound 2004 is prepared analogously to the procedure described for compound 2003 using 3,3-difluoropyrrolidine hydrochloride R3i (21.6 mg, 0.15 mmol) in the presence of isocyanate Ua (55.4 mg, 0.075 mmol).

FIA M.S. (electrospray): 845.3 (M+H)⁺, 843.4 (M−H)⁺

Retention time (min)=5.9 min

¹H NMR (400 MHz, DMSO-d₆): δ10.82 (s, 1H), 8.87 (s, 1H), 7.89 (d, 1H, J=9.0 Hz), 7.09 (d, 1H, J=9.0 Hz), 6.51 (d, 1H, J=7.0 Hz), 6.34 (s, 1H), 5.65-5.58 (m, 1H), 5.49 (S, 1H, J=6.2 Hz), 5.43-5.41 (m, 1H), 5.05 (dd, 1H, J=9.6, 9.2 Hz), 4.63 (d, 1H, J=11.5 Hz), 4.37 (dd, 1H, J=9.9, 6.9 Hz), 4.24-4.18 (m, 1H), 3.92 (d, 1H, J=3.7 Hz), 3.89 (s, 3H), 3.63-3.45 (m, 4H), 2.63-2.55 (m, 2H), 2.43 (s, 3H), 2.40-2.27 (m, 4H), 1.93-1.84 (m, 1H), 1.79-1.70 (m, 1H), 1.58 (dd, 1H, J=8.1, 5.2 Hz), 1.51 (dd, 1H, J=9.4, 4.9 Hz), 1.46-1.37 (m, 5H), 1.39 (s, 3H), 1.39 (d, 3H, J=6.2 Hz), 1.38 (d, 3H, J=6.1 Hz), 1.33-1.20 (m, 4H), 0.92-0.84 (m, 2H).

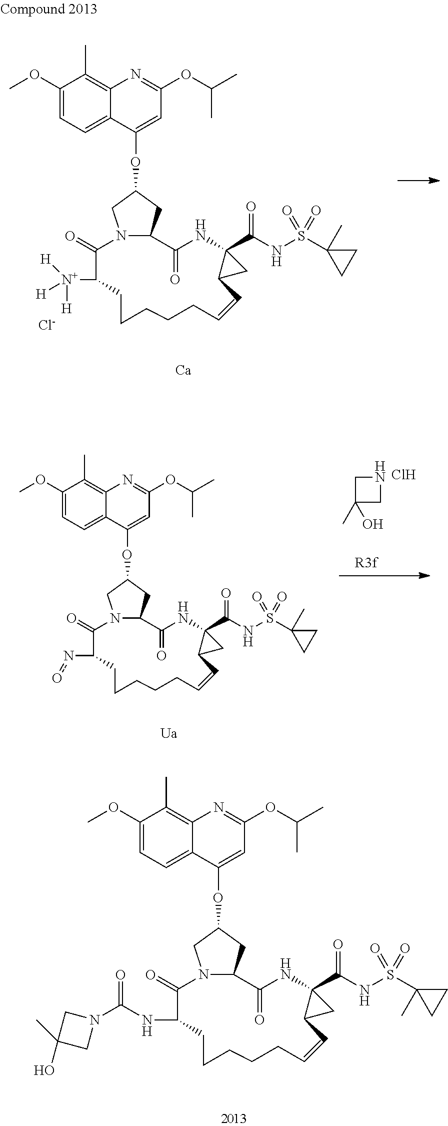

To the crude deprotected macrocyclic amine Ca (50 mg, 0.067 mmol) in DCM (1 mL) is added carbonyl diimidazole (13 mg, 0.08 mmol) and Et₃N (37 μl, 0267 mmol). The reaction mixture is stirred at RT for 60 mins to give a stock solution of Ua. To the azetidine hydrochloride R3f (13 mg, 0.08 mmol) in solution in DCM (1 mL) is added the stock solution of Ua. The reaction mixture is stirred at RT for 16 h. The resulting solution is concentrated in vacuo. To the crude solid is added a mixture of MeCN, DMSO, acetic acid and water. The resulting solution is filtered through a Millex filter and purified by prep HPLC (Sunfire column, 0.1% TFA). The pure fractions are combined, frozen and lyophilized to provide compound 2013.

FIA M.S. (electrospray): 825.4 (M+H)⁺

Retention time (min)=5.1 min

¹H NMR (400 MHz, DMSO-d₆): δ 10.83 (s, 1H), 8.86 (s, 1H), 7.87 (d, 1H, J=9.0 Hz), 7.12 (d, 1H, J=9.4 Hz), 6.49 (bs, 1H), 6.33 (s, 1H), 5.64-5.57 (m 1H), 5.48 (S, 1H, J=6.3 Hz), 5.40 (m, 1H), 5.05 (dd, 1H, J=9.4, 9.4 Hz), 4.57 (d, 1H, J=11.4 Hz), 4.33 (dd, 1H, J=7.0, 6.7 Hz), 4.24-4.19 (m, 1H), 3.95-3.91 (m, 1H), 3.89 (s, 3H), 3.63-3.53 (m, 4H), 2.60-2.57 (m, 1H), 2.43 (s, 3H), 2.41-2.30 (m, 3H), 1.89-1.80 (m, 1H), 1.78-1.68 (m, 1H), 1.57 (dd, 1H, J=8.2, 5.1 Hz), 1.50 (dd, 1H, J=9.4, 5.1 Hz), 1.43-1.41 (m, 2H), 1.39-1.36 (m, 12H), 1.35-1.31 (m, 2H), 1.29 (s, 3H), 1.27-1.20 (m, 3H), 0.91-0.82 (m, 2H).

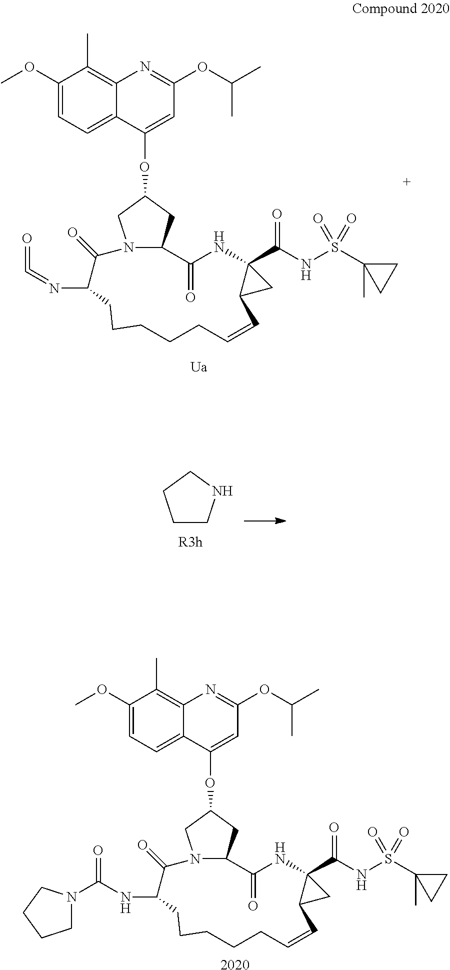

Ua (89 mg, 0.22 mmol) is prepared as previously described in the synthesis of compound 2004 by treating Ca with carbonyl diimidazole under basic conditions. Ua is charged in a vial in DMF (1 mL), then TEA (77 μL, 0.55 mmol) and pyrrolidine R3h (18 μL, 0.22 mmol) are added. The solution is stirred at RT for 1 h, and then it is purified directly by prep HPLC (MeOH, pH 10). The appropriate fractions are combined, frozen and lyophilized to give compound 2020.

UPLC M.S. (electrospray): 809.5 (M+H)⁺, 807.4 (M−H)⁺

Retention time (min)=5.7 min

¹H NMR (400 MHz, DMSO-d₆): δ 10.85 (bs, 1H), 8.86 (bs, 1H), 7.91 (d, 1H, J=9.0 Hz), 7.08 (d, 1H, J=9.0 Hz), 6.33 (s, 1H), 6.08 (bs, 1H), 5.67-5.55 (m, 1H), 5.48 (S, 1H, J=6.3 Hz), 5.41-5.38 (m, 1H), 5.05 (bs, 1H), 4.62 (bs, 1H), 4.34 (dd, 1H, J=9.6, 7.2 Hz), 4.22 (bs, 1H), 3.93-3.89 (m, 1H), 3.88 (s, 3H), 3.14-3.08 (m, 5H), 2.60-2.45 (m, 2H), 2.42 (s, 3H), 2.37-2.26 (m, 2H), 1.90-1.80 (m, 2H), 1.74-1.70 (m, 6H), 1.56 (dd, 1H, J=8.0, 5.3 Hz), 1.54-1.47 (m, 1H), 1.38 (d, 3H, J=6.3 Hz), 1.37 (d, 3H, J=5.9 Hz), 1.38-1.17 (m, 9H), 0.91-0.80 (m, 2H).

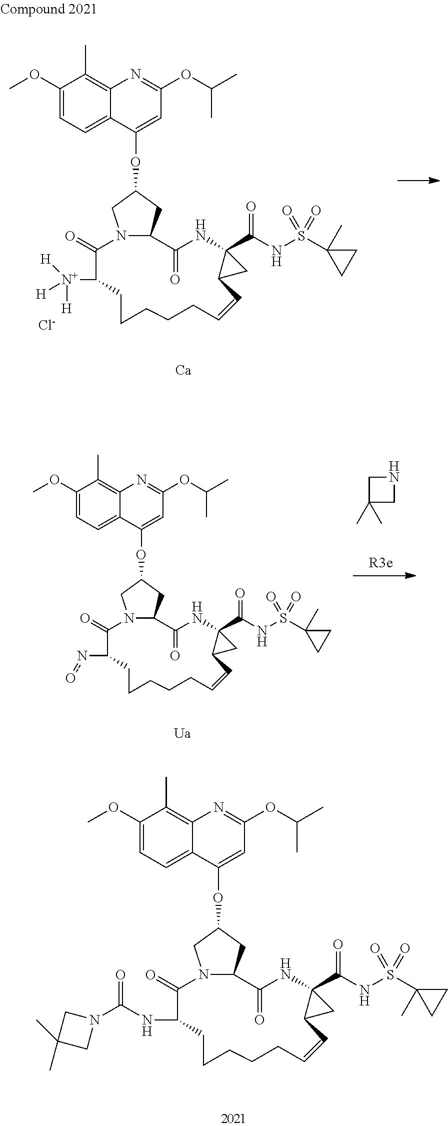

Amine hydrochloride Ca (46 mg, 0.062 mmol) is dissolved in DCM (1 mL), then TEA (26 μL, 0.19 mmol, 3.0 equiv) is added, followed by carbonyl diimidazole (12 mg, 0.074 mmol, 1.2 equiv). The solution is stirred at RT for 1 h. The azetidine R3e is dissolved in DCM (1 mL) and then added into the carbamyl imidazole solution. The reaction is completed after 16 h. The solution is then concentrated and purified on prep HPLC (MeCN:H₂O, 0.1% TFA). The pure fractions are combined, concentrated, frozen and lyophilized to provide compound 2021.

FIA M.S. (electrospray): 823.4 (M+H)⁺

Retention time (min)=5.9 min

¹H NMR (400 MHz, DMSO-d₆): δ 10.88-10.73 (m, 1H), 8.95-8.81 (m, 1H), 7.86 (d, 1H, J=8.9 Hz), 7.10 (d, 1H, J=8.9 Hz), 6.43-6.34 (b, 1H), 6.32 (s, 1H), 5.65-5.55 (m, 1H), 5.49 (S, 1H, J=6.2 Hz), 5.43-5.37 (m, 1H), 5.14-4.98 (m, 1H), 4.61-4.47 (m, 1H), 4.40 (dd, 1H, J=9.7, 7.1 Hz), 4.29-4.17 (m, 1H), 3.99-3.88 (m, 5H), 2.66-2.57 (m, 1H), 2.47-2.39 (m, 5H), 1.91-1.75 (m, 2H), 1.61-1.48 (m, 2H), 1.48-1.24 (m, 18H), 1.19-1.12 (m, 10H), 0.95-0.80 (m, 2H).

### Synthesis of Compounds from Table 1 Using Intermediate Ba

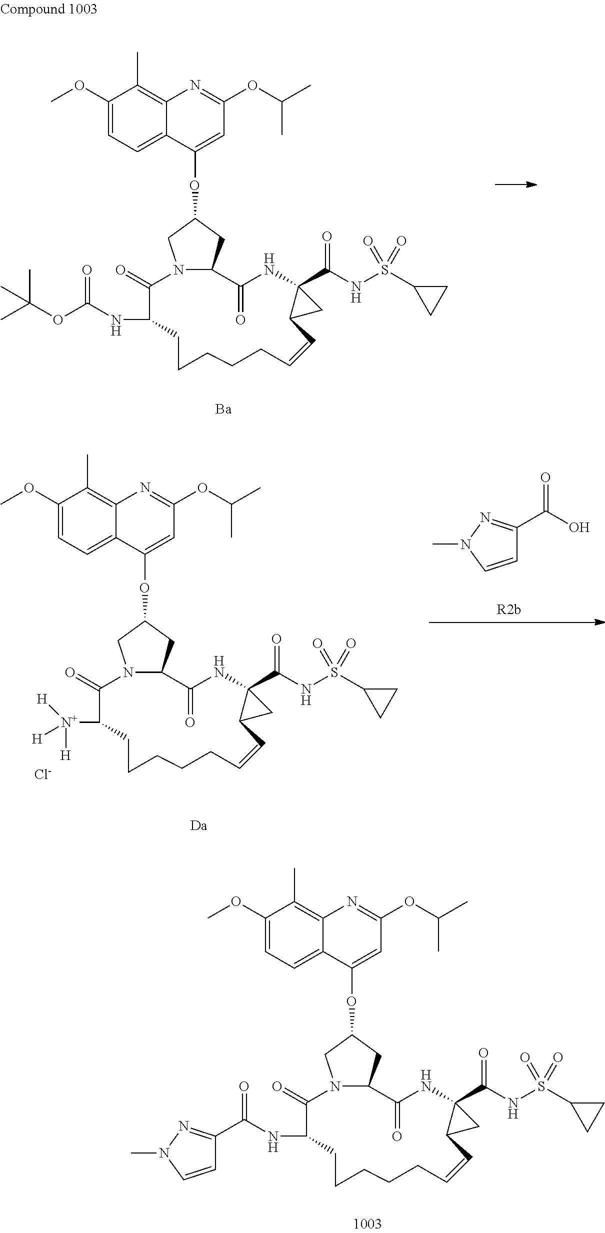

Acid R2b (8.2 mg, 0.065 mmol, 1.3 equiv) is dissolved in DMF (0.5 mL), then TEA (35 μL, 0.25 mmol, 5.0 equiv) is added followed by TBTU (19 mg, 0.060 mmol, 1.2 equiv). The solution is stirred for 15 mins, after which the amine hydrochloride Da (37 mg, 0.050 mmol) is added in DMF (0.5 mL). The solution is stirred at RT for 16 h. Water (2 mL) is added and the organic layer is extracted with EtOAc (3×5 mL). The solvent is then evaporated and purified on prep HPLC (MeCN:H₂O, 0.1% TFA). The pure fractions are combined, concentrated, frozen and lyophilized to provide compound 1003.

FIA M.S. (electrospray): 806.4 (M+H)⁺

Retention time (min)=5.5 min

¹H NMR (400 MHz, DMSO-d₆): δ 11.03 (s, 1H), 8.81 (s, 1H), 7.83 (d, 1H, J=8.8 Hz), 7.78-7.74 (m, 2H), 7.08 (d, 1H, J=9.3 Hz), 6.60 (d, 1H, J=2.3 Hz), 6.37 (s, 1H), 5.67-5.58 (m, 1H), 5.53-5.42 (m, 2H), 5.14 (dd, 1H, J=9.5, 9.2 Hz), 4.64-4.57 (m, 1H), 4.51 (d, 1H, J=11.6 Hz), 4.38 (dd, 1H, J=9.4, 7.0 Hz), 4.01 (dd, 1H, J=11.8, 3.5 Hz), 3.89 (s, 3H), 3.88 (s, 3H), 2.96-2.88 (m, 1H), 2.65-2.57 (m, 1H), 2.44 (s, 3H), 2.40-2.28 (m, 2H), 2.00-1.89 (m, 1H), 1.88-1.77 (m, 1H), 1.62-1.51 (m, 3H), 1.50-1.33 (m, 11H), 1.31-1.19 (m, 2H), 1.14-0.98 (m, 4H).

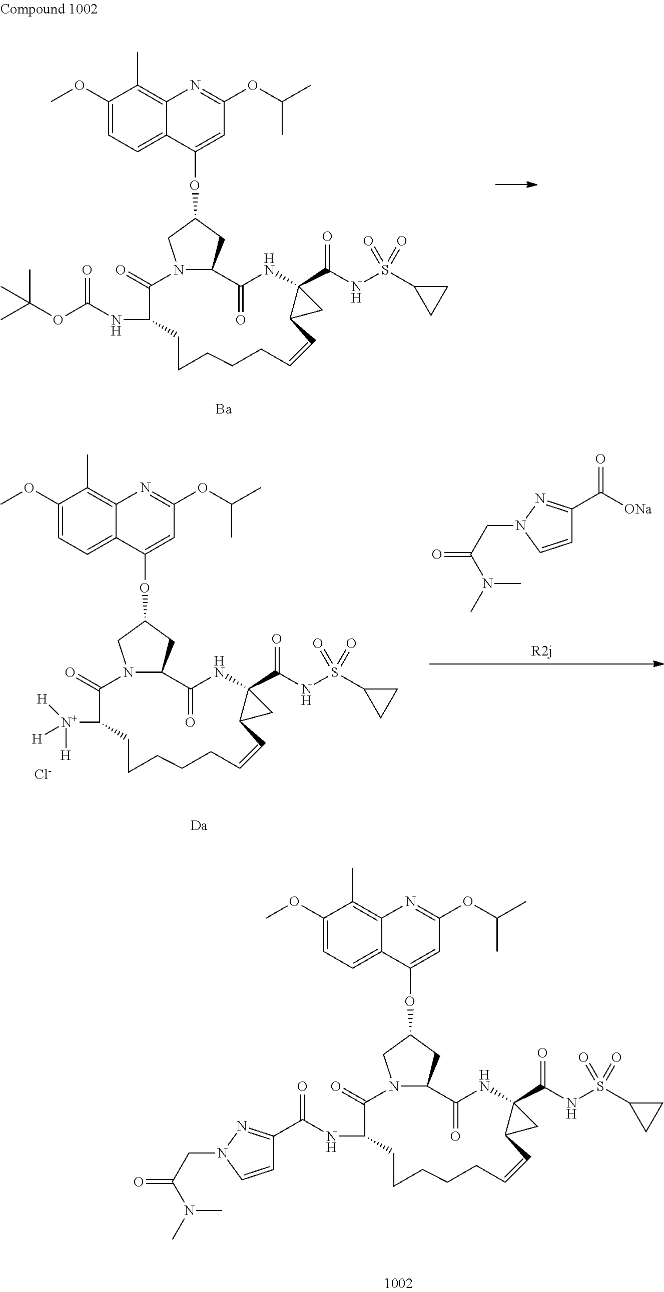

Compound 1002 is synthesized analogously to the procedure described for compound 1003 using 50 mg (0.063 mmol) of Ba and R2j (12 mg, 0.075 mmol) as the coupling partner.

FIA M.S. (electrospray): 877.5 (M+H)⁺

Retention time (min)=5.2 min

¹H NMR (400 MHz, DMSO-d₆): δ 11.04 (s, 1H), 8.77 (s, 1H), 7.81 (d, 1H, J=9.0 Hz), 7.74-7.69 (m, 1H), 7.71 (d, 1H, J=2.3 Hz), 7.09 (d, 1H, J=9.0 Hz), 6.64 (d, 1H, J=2.3 Hz), 6.35 (s, 1H), 5.65-5.54 (m, 1H), 5.48 (p, 1H, J=6.0 Hz), 5.44 (m, 1H), 5.22-5.13 (m, 1H), 5.20 (s, 2H), 4.67-4.60 (m, 1H), 4.55-4.47 (m, 1H), 4.41-4.32 (m, 1H), 4.07-3.98 (m, 1H), 3.87 (s, 3H), 3.02 (s, 3H), 2.92-2.83 (m, 1H), 2.85 (s, 3H), 2.63-2.54 (m, 1H), 2.42 (s, 3H), 2.39-2.27 (m, 2H), 1.98-1.89 (m, 1H), 1.85-1.75 (m, 1H), 1.59-1.52 (m, 3H), 1.45-1.19 (m, 7H), 1.38 (d, 3H, J=6.0 Hz), 1.37 (d, 3H, J=6.0 Hz), 1.09-0.95 (m, 4H).

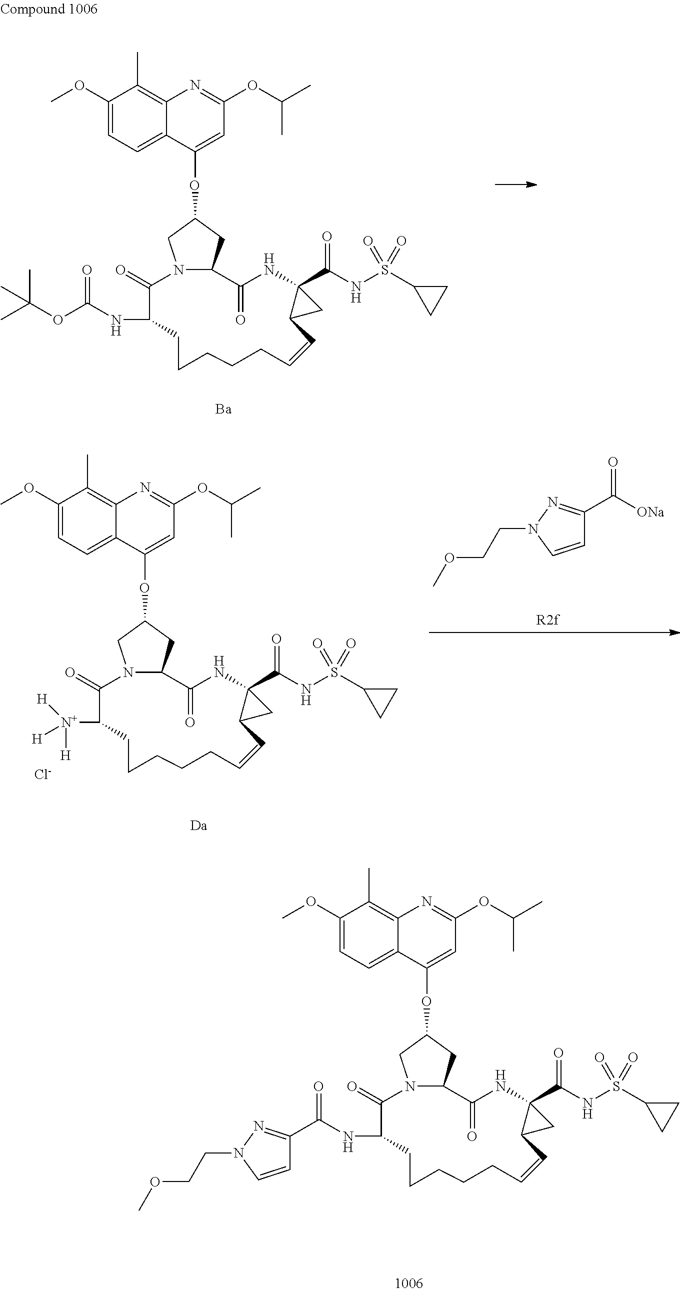

Compound 1006 is synthesized analogously to the procedure described for compound 1003 using the macrocyclic amine hydrochloride salt Da (52.9 mg, 0.072 mmol) in the presence of the crude R2f (27.7 mg, 0.14 mmol).

FIA M.S. (electrospray): 850.4 (M+H)⁺, 848.5 (M−H)⁺

Retention time (min)=5.5 min

¹H NMR (400 MHz, DMSO-d₆): δ 11.05 (bs, 1H), 8.79 (bs, 1H), 7.83 (d, 1H, J=9.4 Hz), 7.82 (bs, 1H), 7.78 (d, 1H, J=2.4 Hz), 7.08 (d, 1H, J=9.4 Hz), 6.61 (d, 1H, J=2.4 Hz), 6.35 (bs, 1H), 5.65-5.56 (m, 1H), 5.49 (S, 1H, J=6.1 Hz), 5.43 (bs, 1H), 5.17-5.10 (m, 1H), 4.63-4.55 (m, 2H), 4.39-4.33 (m, 1H), 4.31 (t, 2H, J=5.3 Hz), 4.05-3.97 (m, 1H), 3.87 (s, 3H), 3.70 (t, 2H, J=5.3 Hz), 3.22 (s, 3H), 2.94-2.87 (m, 1H), 2.63-2.54 (m, 2H), 2.43 (s, 3H), 2.38-2.29 (m, 2H), 2.00-1.89 (m, 1H), 1.85-1.74 (m, 1H), 1.61-1.51 (m, 3H), 1.46-1.38 (m, 4H), 1.39 (d, 3H, J=6.1 Hz), 1.37 (d, 3H, J=6.1 Hz), 1.30-1.19 (m, 2H), 1.12-0.98 (m, 4H).

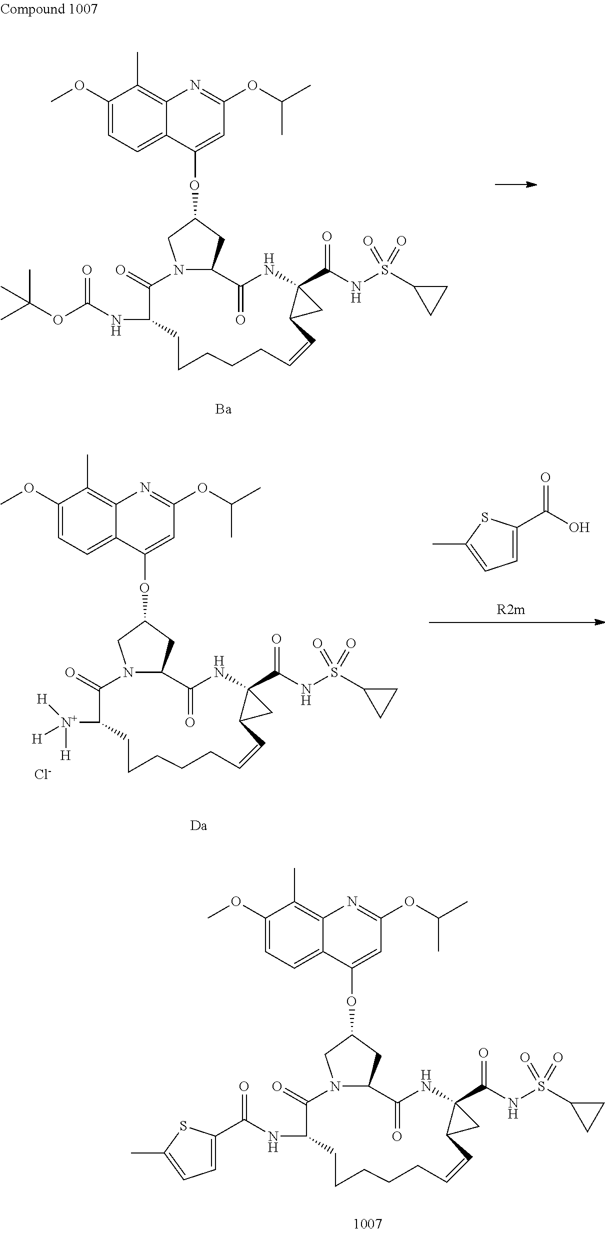

Boc protected macrocyclic amine Ba (100 mg, 0.136 mmol) is charged in a vial with a 4 M solution of HCl in dioxane (3 mL). The solution is stirred at RT for 1 h, after which the solution is evaporated to dryness. 5-methyl-2-thiophene carboxylic acid R2m (23.2 mg; 0.163 mmol, 1.20 equiv) is dissolved in DMF (2 mL) and TEA (75.9 μL; 0.545 mmol, 4.00 equiv) and HATU (62.1 mg; 0.163 mmol, 1.20 equiv) are added and the mixture stirred for 15 mins. The Boc de-protected macrocyclic amine hydrochloride Da is dissolved in DMF (2.0 mL) and added to the acid solution. The reaction is stirred at RT overnight. The resulting solution is filtered through a Millex filter and purified by prep HPLC (X-Bridge column, Ammonium Bicarbonate pH 10: MeOH) The pure fractions are combined, concentrated, frozen and lyophilized to provide compound 1007.

FIA M.S. (electrospray): 820.3 (M−H)−, 822.3 (M+H)⁺

Retention time (min)=6.1 min

¹H NMR (400 MHz, DMSO-d₆): δ 11.05 (s, 1H), 8.75 (s, 1H), 8.59 (d, 1H, J=6.6 Hz), 7.91 (d, 1H, J=9 Hz), 7.68 (d, 1H, J=3.9 Hz), 7.06 (d, 1H, J=9.4 Hz), 6.83 (d, 1H, J=3.5 Hz), 6.35 (s, 1H), 5.61 (dd, 1H, J=8.6, 8.6 Hz), 5.49 (p, 1H, J=6.2 Hz), 5.13 (dd, 2H, J=10.2, 8.2 Hz), 4.68 (d, 1H, J=10.9 Hz), 4.49-4.44 (m, 1H), 4.29 (dd, 1H, J=9.98, 6.85 Hz), 3.93 (dd, 1H, J=11.35, 3.52 Hz), 3.89 (s, 3H), 2.93-2.87 (m, 1H), 2.68-2.57 (m, 2H), 2.44 (d, 6H, J=7.83 Hz), 2.40-2.29 (m, 2H), 2.01-1.98 (m, 1H), 1.75-1.71 (m, 1H), 1.57-1.33 (m, 13H), 1.31-1.24 (m, 2H), 1.10-0.97 (m, 4H).

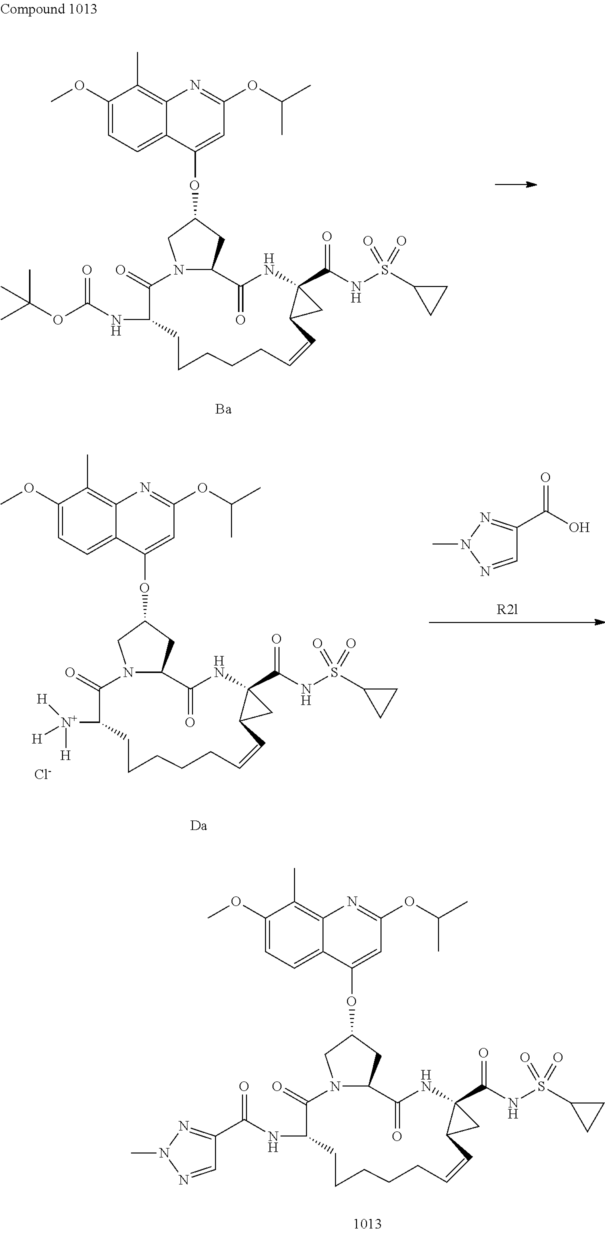

Boc protected amine Ba (50 mg, 0.063 mmol) is charged in a vial, then a 4 M solution of HCl in dioxane (1 mL, 4 mmol) is added. The solution is stirred at RT for 2 h, after which a precipitate forms. The solution is evaporated to dryness. Acid R2l (9.6 mg, 0.075 mmol, 1.2 equiv) is dissolved in DMF (0.3 mL), then TEA (44 μL, 0.31 mmol, 5.0 equiv) is added followed by TBTU (28 mg, 0.075 mmol, 1.2 equiv). This solution is stirred for 15 mins, after which the amine hydrochloride Da is added in DMF (0.4 mL). The solution is stirred at RT for 16 h. Water (2 mL) is added and the organic layer is extracted with EtOAc (3×5 mL). The solvent is evaporated and the residue is purified on prep HPLC (MeCN:H₂O, 0.06% TFA). The pure fractions are combined, concentrated, frozen and lyophilized to provide compound 1013.

FIA M.S. (electrospray): 807.4 (M+H)⁺

Retention time (min)=5.5 min

¹H NMR (400 MHz, DMSO-d₆): δ 8.09 (s, 1H), 7.92 (d, 1H, J=6.6 Hz), 7.79 (d, 1H, J=9.0 Hz), 7.68 (s, 1H), 7.09 (d, 1H, J=9.0 Hz), 6.29 (s, 1H), 5.53-5.44 (m, 2H), 5.39-5.33 (m, 1H), 5.30-5.22 (m, 1H), 4.84 (dt, 1H, J=7.0, 2.9 Hz), 4.52 (dd, 1H, J=8.1, 6.8 Hz), 4.33-4.27 (m, 1H), 4.21 (s, 3H), 4.19-4.17 (m, 1H), 3.87 (s, 3H), 3.20-3.16 (m, 1H), 2.74-2.66 (m, 1H), 2.45-2.32 (m, 4H), 2.23-2.12 (m, 1H), 2.03-1.78 (m, 6H), 1.68 (dd, 1H, J=9.2, 4.1 Hz), 1.55 (dd, 1H, J=7.8, 4.1 Hz), 1.47-1.41 (m, 2H), 1.39 (dd, 6H, J=6.3, 2.3 Hz), 1.26-1.08 (m, 3H), 0.79-0.73 (m, 2H), 0.63-0.56 (m, 2H).

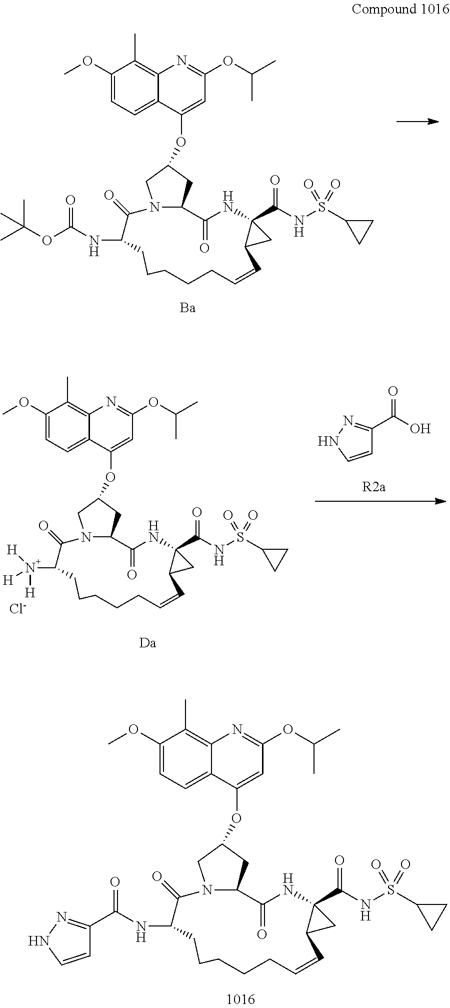

Boc protected macrocyclic amine Ba (100 mg, 0.136 mmol) is charged in a vial with a 4 M solution of HCl in dioxane (3 mL). The solution is stirred at RT for 1 h, after which the solution is evaporated to dryness. 1H-pyrazole-3-carboxylic acid R2a (18.3 mg; 0.163 mmol, 1.20 equiv) is dissolved in DMF (2 mL) and TEA (75.9 μL; 0.545 mmol, 4.00 equiv) and HATU (62.1 mg; 0.163 mmol, 1.20 equiv) are added. The mixture is stirred for 15 mins. The Boc de-protected macrocyclic amine hydrochloride Da is dissolved in DMF (2.0 mL) and added to the acid solution. The reaction is stirred at RT overnight. The resulting solution is filtered through a Millex filter and purified by prep HPLC (X-Bridge column, Ammonium Bicarbonate pH10: MeOH). The pure fractions are combined, concentrated, frozen and lyophilized to provide compound 1016.

FIA M.S. (electrospray): 790.1 (M−H)−, 792.2 (M+H)⁺

Retention time (min)=5.2 min

¹H NMR (400 MHz, DMSO-d₆): δ 11.05 (s, 1H), 8.79 (s, 1H), 8.04 (bs, 1H), 7.82 (d, 1H, J=9 Hz), 7.74 (d, 1H, J=2.0 Hz), 7.07 (d, 1H, J=9.0 Hz), 6.71 (s, 1H), 6.37 (s, 1H), 5.61 (dd, 1H, J=18.4, 8.3 Hz), 5.49 (p, 1H, J=5.8 Hz), 5.45 (bs, 1H), 5.14 (dd, 2H, J=10.2, 9 Hz), 4.62-4.54 (m, 2H), 4.34 (dd, 1H, J=9.8, 7.0 Hz), 4.02 (dd, 1H, J=11.3, 3.5 Hz), 3.87 (s, 3H), 2.94-2.88 (m, 1H), 2.62-2.56 (m, 2H), 2.43 (s, 3H), 2.38-2.31 (m, 2H), 2.01-1.92 (m, 1H), 1.80-1.75 (m, 1H), 1.58-1.52 (m, 3H), 1.44-1.36 (m, 10H), 1.31-1.20 (m, 2H), 1.11-0.97 (m, 4H).

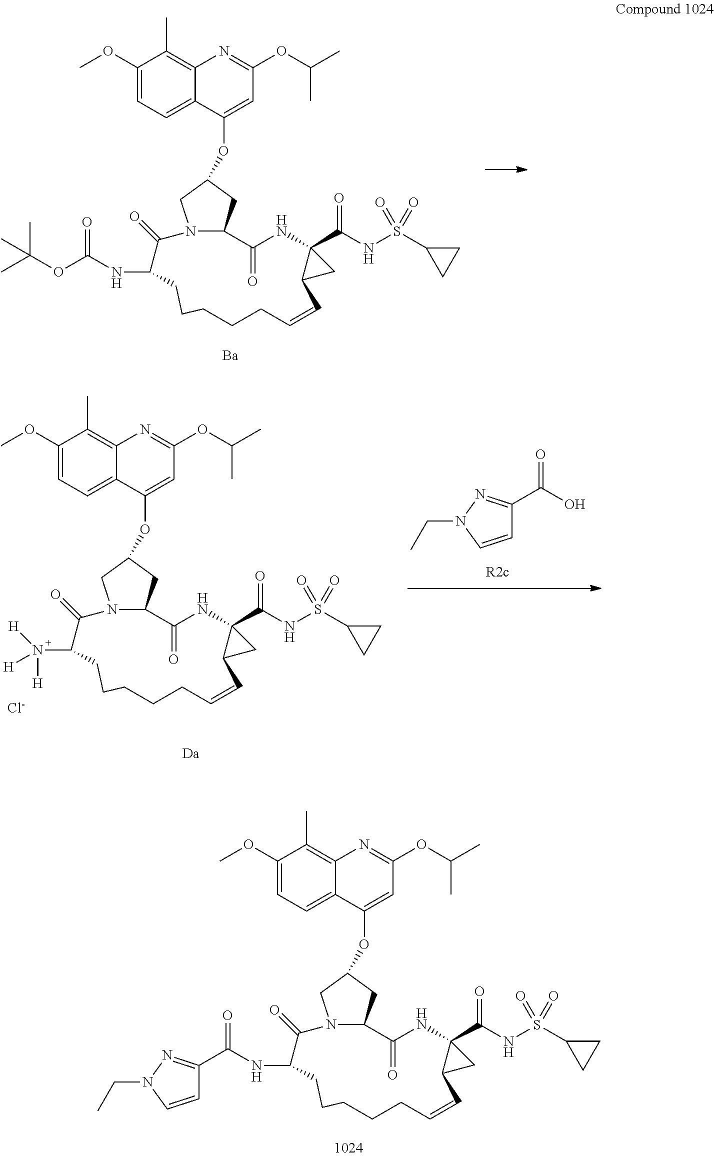

Compound 1024 is synthesized analogously to the procedure described for the preparation of compound 1016 using Ba (46 mg, 0.058 mmol) and R2c (0.015, 0.09 mmol) as the coupling partner.

FIA M.S. (electrospray): 820.5 (M+H)⁺

Retention time (min)=5.7 min

¹H NMR (400 MHz, DMSO-d₆): δ 11.01 (s, 1H), 8.78 (s, 1H), 7.82 (d, 1H, J=9.0 Hz), 7.80 (d, 1H, J=2.1 Hz), 7.72 (d, 1H, J=6.1 Hz), 7.07 (d, 1H, J=9.2 Hz), 6.60 (d, 1H, J=2.1 Hz), 6.34 (s, 1H), 5.63-5.56 (1H, m), 5.49 (p, 1H, J=6.2 Hz), 5.43 (m, 1H), 5.17-5.12 (m, 1H), 4.65-4.58 (m, 1H), 4.52-4.47 (m, 1H), 4.42-4.37 (m, 1H), 4.18 (q, 2H, J=7.2 Hz), 4.06-4.01 (m, 1H), 3.87 (s, 3H), 2.92-2.86 (m, 1H), 2.68-2.59 (m, 1H), 2.43 (s, 3H), 2.38-2.30 (m, 2H), 1.98-1.78 (m, 2H), 1.58-1.56 (m, 3H), 1.44-1.23 (m, 16H), 1.09-0.97 (m, 4H).

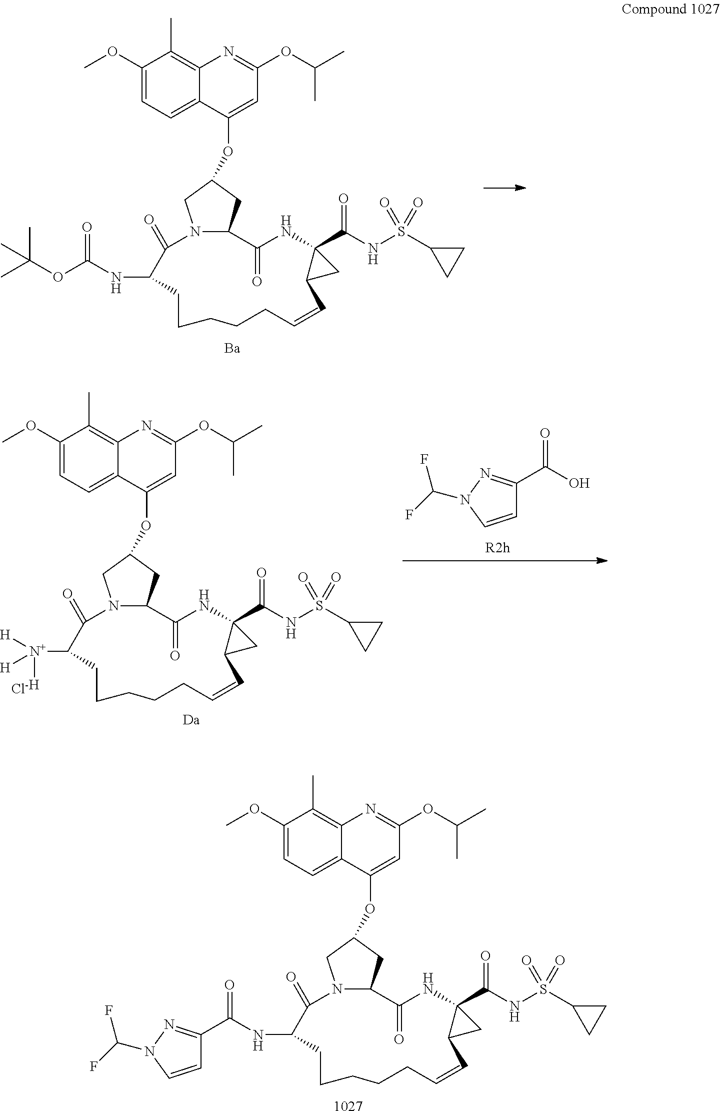

Boc protected macrocyclic amine Ba (75 mg, 0.094 mmol) is charged in a vial with a 4 M solution of HCl in dioxane (2 mL). The solution is stirred at RT for 1 h, after which the solution is evaporated to dryness. 1-Difluoromethyl-1H-pyrazole-3-carboxylic acid R2h (18.3 mg; 0.113 mmol) is dissolved in DMF (2 mL) and TEA (52.4 μL; 0.376 mmol) and TBTU (36.2 mg; 0.113 mmol) are added. The mixture is stirred for 15 mins. The Boc de-protected macrocyclic amine hydrochloride Da is dissolved in DMF (1.0 mL) and added to the acid solution. This solution is stirred at RT overnight. The resulting solution is filtered through a Millex filter and purified by prep HPLC (X-Bridge column, Ammonium Bicarbonate pH10: MeOH). The pure fractions are combined, concentrated, frozen and lyophilized to provide compound 1027.

FIA M.S. (electrospray): 840.5 (M−H)−, 842.4 (M+H)⁺

Retention time (min)=5.9 min

¹H NMR (400 MHz, DMSO-d₆): δ 11.05 (s, 1H), 8.79 (s, 1H), 8.40-8.23 (m, 1H), 8.33 (d, 1H, J=2.7 Hz), 7.87 (s, 1H), 7.84 (d, 1H, J=9 Hz), 7.07 (d, 1H, J=9.4 Hz), 6.87 (d, 1H, J=2.7 Hz), 6.35 (s, 1H), 5.72-5.55 (m, 1H), 5.55-5.37 (m, 2H), 5.25-5.07 (m, 1H), 4.62-4.45 (m, 2H), 4.45-4.31 (m, 1H), 4.09-3.95 (m, 1H), 3.87 (s, 3H), 2.95-2.86 (m, 1H), 2.64-2.52 (m, 1H), 2.43 (s, 3H), 2.42-2.26 (m, 2H), 2.05-1.92 (m, 1H), 1.92-1.70 (m, 1H), 1.64-1.50 (m, 3H), 1.50-1.32 (m, 10H), 1.32-1.15 (m, 3H), 1.13-0.92 (m, 4H).

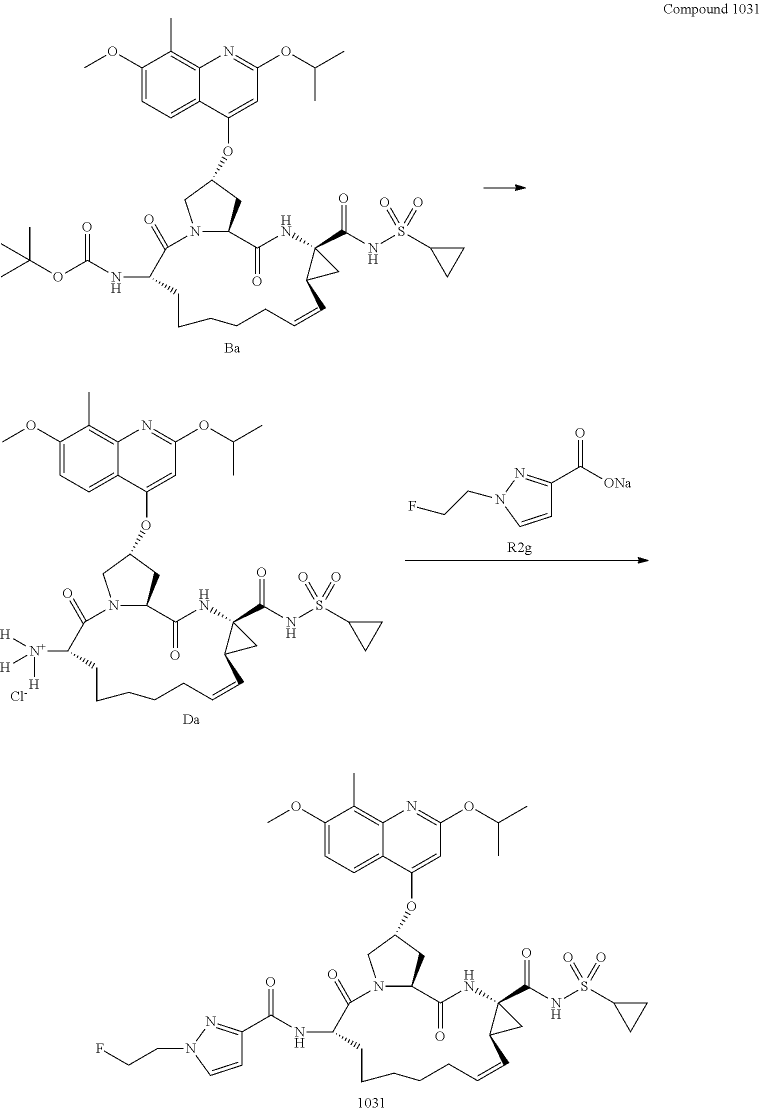

The acid component R2g (24.5 mg; 0.136 mmol) is dissolved in DMF (1 mL) and DIPEA (71 μL; 0.409 mmol) and HATU (51.8 mg; 0.136 mmol) are added. The mixture is stirred for 15 mins. The Boc de-protected macrocyclic amine hydrochloride Da (50 mg; 0.068 mmol) is dissolved in DMF (1.0 mL) and added to the acid solution. The reaction is stirred at RT overnight. The resulting solution is filtered through a Millex filter and purified by prep HPLC (Sunfire column, Ammonium Formate pH 3.8: MeOH). The pure fractions are combined, concentrated, frozen and lyophilized to provide compound 1031.

FIA M.S. (electrospray): 836.4 (M−H)−, 838.3 (M+H)⁺

Retention time (min)=5.6 min

¹H NMR (400 MHz, DMSO-d₆): δ 11.05 (s, 1H), 8.79 (s, 1H), 7.90-7.80 (m, 2H), 7.85 (d, 1H, J=2.3 Hz), 7.07 (d, 1H, J=9.4 Hz), 6.65 (d, 1H, J=2.4 Hz), 6.35 (s, 1H), 5.66-5.54 (m, 1H), 5.53-5.45 (m, 1H), 5.45-5.40 (m, 1H), 5.18-5.09 (m, 1H), 4.88-4.83 (m, 1H), 4.76-4.72 (m, 1H), 3.65-3.30 (m, 5H), 4.06-3.97 (m, 1H), 3.87 (s, 3H), 2.95-2.86 (m, 1H), 2.64-2.55 (m, 1H), 2.43 (s, 3H), 2.40-2.28 (m, 2H), 2.02-1.87 (m, 1H), 1.87-1.71 (m, 1H), 1.61-1.50 (m, 3H), 1.50-1.16 (m, 13H), 1.16-0.93 (m, 4H).

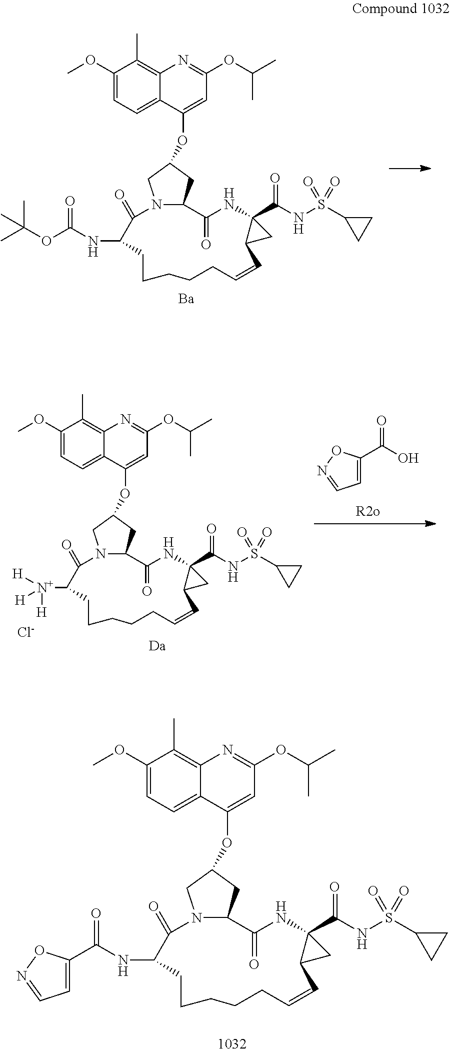

Boc protected amine Ba (35 mg, 0.044 mmol) is charged in a vial, then a 4 M solution of HCl in dioxane (1 mL, 4 mmol) is added. The solution is stirred at RT for 2 h, after which a precipitate forms. The solution is evaporated to dryness. Acid R2o (6.5 mg, 0.057 mmol, 1.30 equiv) is dissolved in DMF (0.5 mL), then TEA (30 μL, 0.22 mmol, 5.0 equiv) is added followed by TBTU (17 mg, 0.53 mmol, 1.2 equiv). The solution is stirred for 15 mins, after which the amine hydrochloride Da is added in DMF (0.5 mL). This solution is stirred at RT for 16 h. Water (2 mL) is added and the organic layer is extracted with EtOAc (3×5 mL). The solvent is evaporated and the residue is purified on prep HPLC (MeCN:H₂O, 0.1% TFA). The pure fractions are combined, concentrated, frozen and lyophilized to provide compound 1032.

FIA M.S. (electrospray): 793.4 (M+H)⁺

Retention time (min)=5.6 min

¹H NMR (400 MHz, DMSO-d₆): δ 11.03 (s, 1H), 9.13 (d, 1H, J=6.6 Hz), 8.80 (s, 1H), 8.72 (d, 1H, J=2.1 Hz), 7.86 (d, 1H, J=9.1 Hz), 7.11 (d, 1H J=2 Hz), 7.10 (d, 1H, J=9.2 Hz), 6.36 (s, 1H), 5.67-5.59 (m, 1H), 5.55-5.44 (m, 2H), 5.14 (dd, 1H, J=10.1, 8.7 Hz), 4.60-4.51 (m, 2H), 4.36 (dd, 1H, J=9.9, 7.0 Hz), 4.01 (dd, 1H, J=11.7, 3.5 Hz), 3.89 (s, 3H), 2.94-2.87 (m, 1H), 2.69-2.56 (m, 2H), 2.44 (s, 3H), 2.41-2.31 (m, 2H), 2.07-1.95 (m, 1H), 1.83-1.71 (m, 1H), 1.60-1.35 (m, 13H), 1.33-1.19 (m, 2H), 1.12-0.99 (m, 4H).

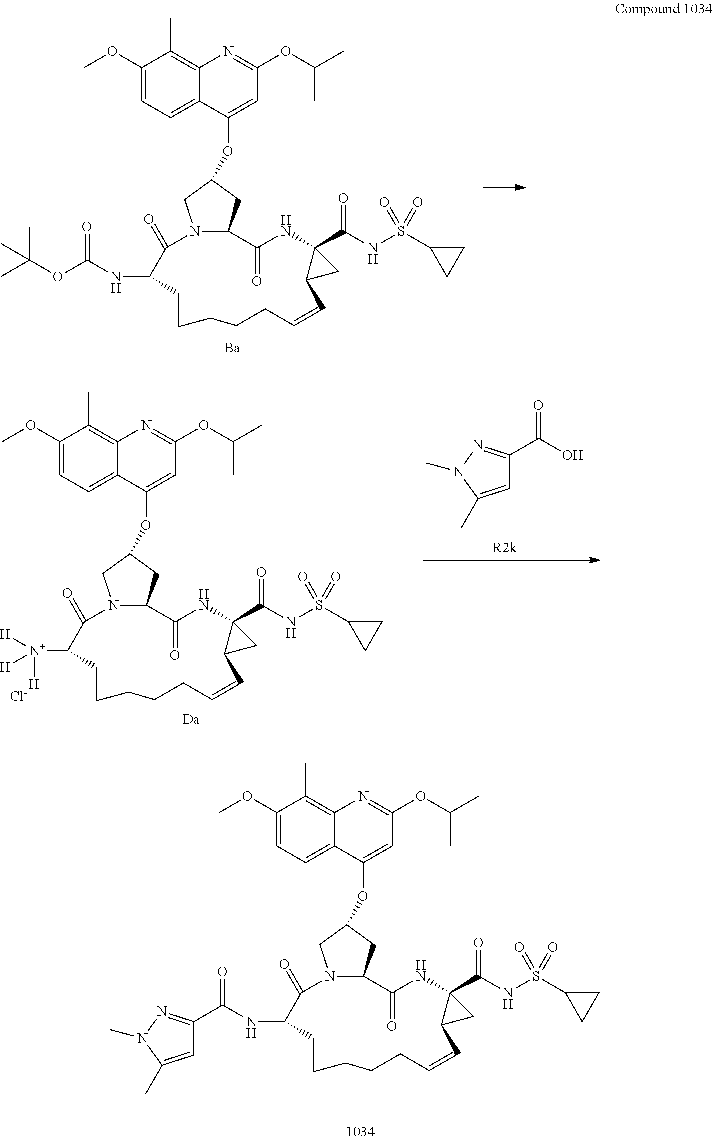

Compound 1034 is synthesized analogously to the procedure described for the preparation of compound 1032 using Ba (45 mg, 0.056 mmol) and R2k (10 mg, 0.070 mmol) as the coupling partner.

FIA M.S. (electrospray): 820.5 (M+H)⁺

Retention time (min)=5.6 min

¹H NMR (400 MHz, DMSO-d₆): δ 11.06 (s, 1H), 8.80 (s, 1H), 7.81 (d, 1H, J=9.0 Hz), 7.66 (d, 1H, J=6.7 Hz), 7.08 (d, 1H, J=9.4 Hz), 6.37 (s, 1H), 6.35 (s, 1H), 5.64-5.58 (m, 1H), 5.48 (p, 1H, J=6.2 Hz), 5.43 (m, 1H), 5.16-4.11 (m, 1H), 4.60-4.48 (m, 2H), 4.37-4.33 (m, 1H), 4.00-3.98 (m, 1H), 3.87 (s, 3H), 3.76 (s, 3H), 2.94-2.88 (m, 1H), 2.67-2.59 (m, 1H), 2.43 (s, 3H), 2.38-2.30 (m, 2H), 2.25 (s, 3H), 1.97-1.78 (m, 2H), 1.59-1.53 (m, 3H), 1.44-1.23 (m, 7H), 1.38 (d, 3H, J=6.2 Hz), 1.37 (d, 3H, J=6.2 Hz), 1.09-1.00 (m, 4H).

### Synthesis of Compounds from Table 2 Using Intermediate Va

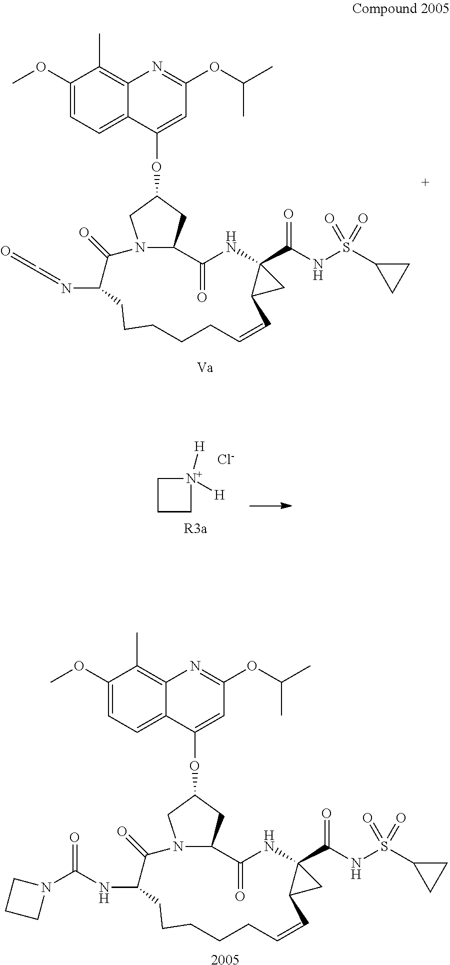

Azetidine hydrochloride R3a (10.5 mg; 0.112 mmol) is suspended in DCM (0.4 mL), DIPEA (29 μL; 0.169 mmol) is added and the mixture is heated slightly with a heat gun. The mixture is left to stir for 2 mins after which it is cooled to 0° C. The macrocyclic isocyanate Va (40.7 mg; 0.056 mmol) is added and reaction is stirred at RT overnight. The mixture is dissolved in MeOH/DMSO, filtered through a Millex filter and purified by prep HPLC (Sunfire column; 0.1% TFA/CH₃CN: 0.1% TFA/H₂O). The pure fractions are combined, concentrated, frozen and lyophilized to provide compound 2005.

FIA M.S. (electrospray): 779.4 (M−H)−, 781.3 (M+H)⁺

Retention time (min)=5.3 min

¹H NMR (400 MHz, DMSO-d₆): δ 11.04 (s, 1H), 8.75 (s, 1H), 7.89 (d, 1H, J=9.2 Hz), 7.12 (d, 1H, J=9.2 Hz), 6.38 (d, 1H, J=7.4 Hz), 6.33 (s, 1H), 5.67-5.55 (m, 1H), 5.55-5.43 (m, 1H), 5.43-5.36 (m, 1H, 5.13-5.07 (m, 1H), 4.53 (d, 1H, J=11.8 Hz), 4.37-4.27 (m, 1H), 4.23-4.12 (m, 1H), 3.93-3.90 (m, 1H), 3.89 (s, 3H), 3.75-3.65 (m, 4H), 2.94-2.85 (m, 1H), 2.70-2.55 (m, 1H), 2.43 (s, 3H), 2.43-2.27 (m, 2H), 2.07-1.98 (m, 2H), 1.89-1.63 (m, 2H), 1.60-1.50 (m, 2H), 1.47-0.95 (m, 18H).

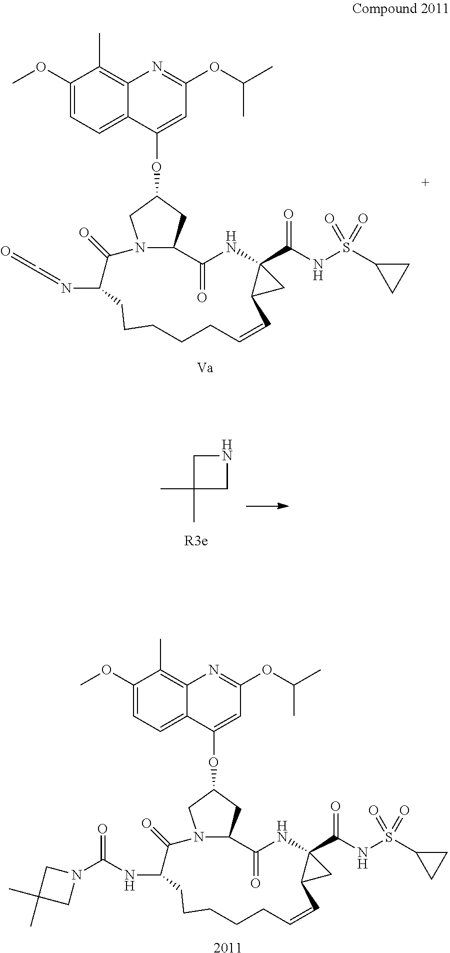

Compound 2011 is synthesized analogously to the procedure described for compound 2001 using 3,3-dimethylazetidine (13.4 mg, 0.16 mmol) in the presence of isocyanate Va (45.4 mg, 0.063 mmol).

3,3,-Dimethylazetidine is prepared according to a literature procedure (J. Org. Chem. 1981, 46, 4907-4911).

FIA M.S. (electrospray): 809.3 (M+H)⁺, 807.4 (M−H)⁺

Retention time (min)=5.9 min

¹H NMR (400 MHz, DMSO-d₆): δ 11.04 (s, 1H), 8.76 (s, 1H), 7.86 (d, 1H, J=9.0 Hz), 7.10 (d, 1H, J=9.0 Hz), 6.39 (bs, 1H), 6.32 (s, 1H), 5.65-5.57 (m, 1H), 5.49 (S, 1H, J=6.2 Hz), 5.42-5.38 (m, 1H), 5.14-5.08 (m, 1H), 4.58-4.53 (m, 1H), 4.36-4.30 (m, 1H), 4.24-4.18 (m, 1H), 3.95-3.90 (m, 1H), 3.90 (s, 3H), 3.43-3.337 (m, 4H), 2.62-2.54 (m, 2H), 2.44 (s, 3H), 2.46-2.38 (m, 3H), 1.86-1.70 (m, 2H), 1.60-1.51 (m, 2H), 1.45-1.32 (m, 5H), 1.39 (d, 3H, J=6.2 Hz), 1.38 (d, 3H, J=6.1 Hz), 1.25-0.98 (m, 6H), 1.14 (s, 6H).

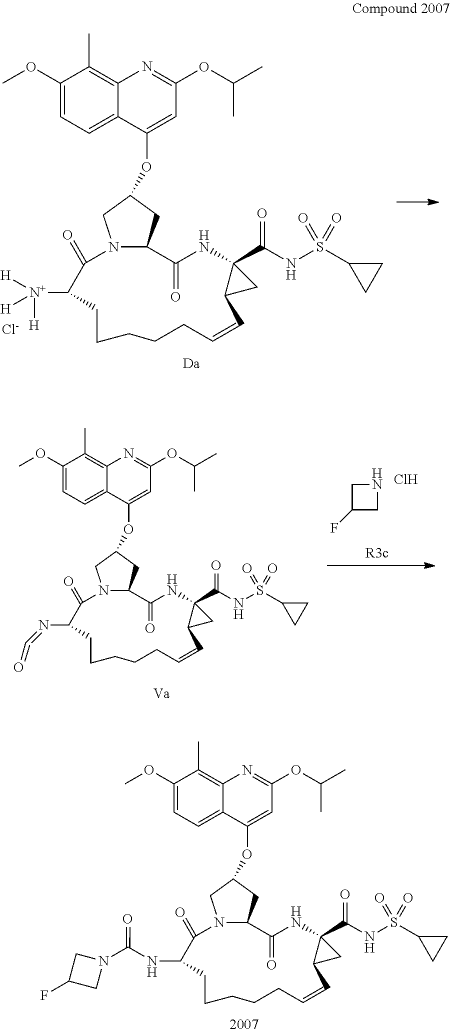

Macrocyclic amine salt Da (184 mg, 0.251 mmol) is charged in a vial and dissolved in DCM (0.8 mL). Diisopropylethylamine (87 μL, 0.501 mmol) is added and the solution is cooled to 0° C. A solution of triphosgene (37.2 mg, 0.125 mmol) in DCM (0.2 mL) is then added. The solution is stirred at RT for 25 min, and then used as such for the next step.

In another vial, azetidine R3c is dissolved in DCM (0.2 mL), diisopropylethylamine (0.02 mL, 0.125 mmol) is added and the solution is stirred for 2 mins. The azetidine solution is cooled to 0° C. after which the solution containing Va (45.4 mg, 0.063 mmol) is added to the reaction. The reaction is stirred overnight at RT. The resulting solution is filtered through a Millex filter and purified by prep HPLC. The pure fractions are combined, concentrated, frozen and lyophilized to provide compound 2007.

FIA M.S. (electrospray): 799.3 (M+H)⁺

Retention time (min)=5.5 min

¹H NMR (400 MHz, DMSO-d₆): δ 11.03 (s, 1H), 8.76 (s, 1H), 7.88 (d, 1H, J=9.0 Hz), 7.14 (d, 1H, J=9.2 Hz), 6.72 (br.s., 1H), 6.33 (s, 1H), 5.64-5.57 (m, 1H), 5.52 (S, 1H, J=5.9 Hz), 5.42-5.38 (m, 1H), 5.36 (ttd, 1H, ¹J_{H-F}=56.0 Hz, J=7.3, 7.1 Hz), 5.14 (dd, 1H, J=8.9, 8.4 Hz), 4.55 (d, 1H, J=10.9 Hz), 4.35 (dd, 1H, J=9.9, 7.1 Hz), 4.23-4.18 (m, 1H), 4.08-3.99 (m, 2H), 3.90 (s, 3H), 3.85-3.71 (m, 2H), 2.93-2.87 (m, 1H), 2.60-2.56 (m, 2H), 2.44 (s, 3H), 2.40-2.38 (m, 1H), 2.36-2.28 (m, 1H), 1.85-1.75 (m, 2H), 1.59-1.54 (m, 2H), 1.39-1.37 (m, 12H), 1.26-1.18 (m, 2H), 1.09-1.00 (m, 4H).

### Synthesis of Compounds from Table 1 Using Intermediate Ac

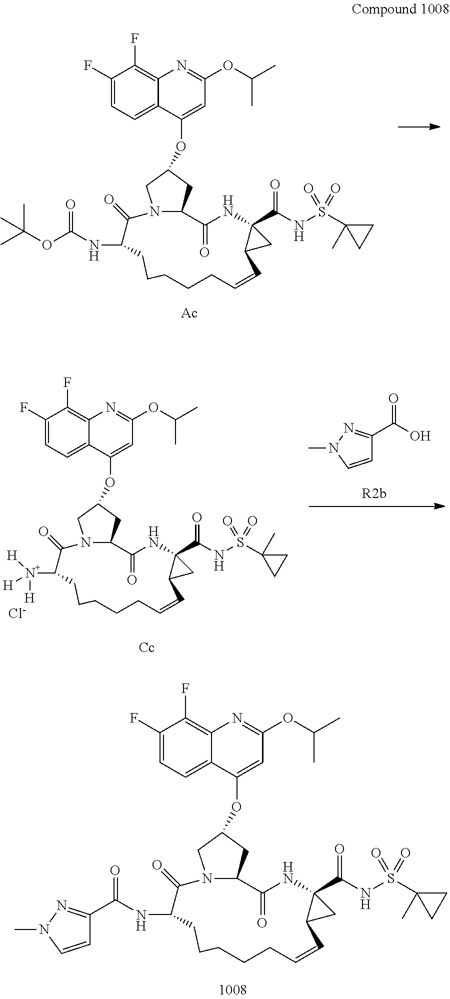

Boc protected macrocyclic amine Ac (65 mg, 0.081 mmol) is charged in a vial with a 4 M solution of HCl in dioxane (3 mL). The solution is stirred at RT for 1 h, after which the solution is evaporated to dryness. 1-methyl-1H-pyrazole-3-carboxylic acid R2b (12.2 mg; 0.097 mmol, 1.2 equiv) is dissolved in DMF (2 mL) and TEA (45.1 μL; 0.323 mmol, 4 equiv) and TBTU (29.9 mg; 0.097 mmol, 1.2 equiv) are added. The mixture is stirred for 15 mins. The Boc de-protected macrocyclic amine hydrochloride Cc is dissolved in DMF (1.0 mL) and added to the acid solution. The reaction is stirred at RT overnight. The resulting solution is filtered through a Millex filter and purified by prep HPLC (X-Bridge column, Ammonium Bicarbonate pH10: MeOH). The pure fractions are combined, concentrated, frozen and lyophilized to provide compound 1008.

FIA M.S. (electrospray): 812.3 (M+H)⁺

Retention time (min)=6.8 min

¹H NMR (400 MHz, DMSO-d₆): δ 10.83 (s, 1H), 8.91 (s, 1H), 7.88-7.77 (m, 2H), 7.75 (d, 1H, J=2.4 Hz), 7.36-7.30 (m, 1H), 6.62 (s, 1H), 6.55 (d, 1H, J=2 Hz), 5.65-5.57 (m, 1H), 5.57-5.45 (m, 2H), 5.15-5.03 (m, 1H), 4.64-4.50 (m, 2H), 4.49-4.37 (m, 1H), 4.03-3.88 (m, 1H), 3.88 (s, 3H), 2.67-2.55 (m, 1H), 2.38-2.24 (m, 2H), 2.00-1.87 (m, 1H), 1.87-1.72 (m, 1H), 1.61-1.47 (m, 3H), 1.47-1.17 (m, 18H), 0.95-0.78 (m, 2H).

### Synthesis of Compounds from Table 1 Using Intermediate Ad

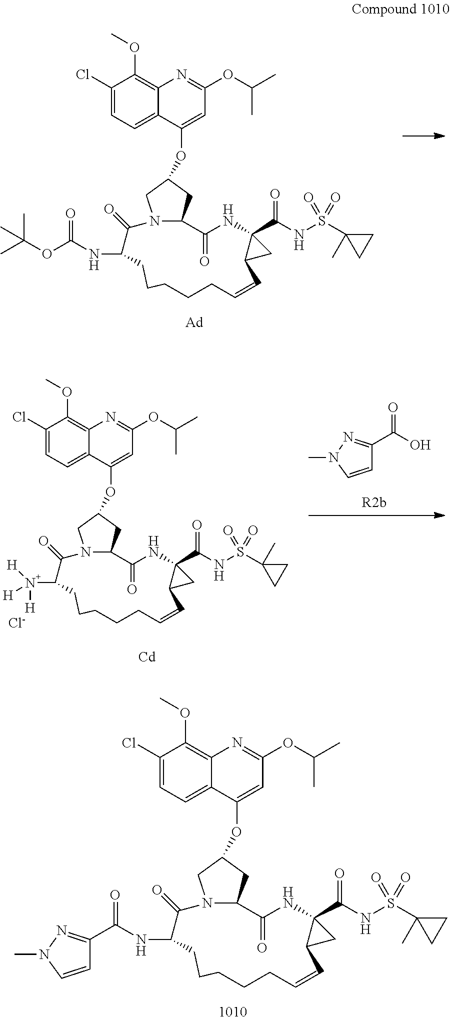

Boc protected amine Ad (85 mg, 0.102 mmol) is charged in a flask, then a 4 M solution of HCl in dioxane (1 mL, 4 mmol) is added. The solution is stirred at RT for 1.5 h, after which a precipitate forms. The solution is evaporated to dryness. 1-methyl-1H-pyrazole-3-carboxylic Acid R2b (15.4 mg, 0.123 mmol) is dissolved in DCM (1.5 mL), then TEA (57 μL, 0.408 mmol) is added followed by TBTU (37.7 mg, 0.117 mmol). The solution is stirred for 15 mins, after which it is added to the amine hydrochloride Cd in solution in DCM (0.5 mL). This solution is stirred at RT for 16 h and then concentrated. The residual is dissolved in DMSO. The resulting solution is filtered through a Millex filter and purified by prep HPLC (Sunfire column, ammonium formate and MeOH). The pure fractions are combined, concentrated, redissolved in MeCN and water, frozen and lyophilized to provide compound 1010.

FIA M.S. (electrospray): 838.4, 840.4 (M−H)⁻

Retention time (min)=6.5 min

¹H NMR (400 MHz, DMSO-d₆): δ 8.14 (bs, 1H), 7.76 (d, 1H, J=1.9 Hz), 7.72 (d, 2H, J=9 Hz), 7.30 (d, 1H, J=8.6 Hz), 6.57 (s, 1H), 6.56 (d, 1H, J=2.3 Hz), 5.52-5.24 (m 4H), 4.75-4.63 (m, 1H), 4.45 (dd, 1H, J=8.3, 8.2 Hz), 4.42-4.33 (m, 1H), 4.18-4.08 (m, 1H), 4.05 (s, 3H), 3.89 (s, 3H), 2.62-2.55 (m, 1H), 2.11-1.87 (m, 3H), 1.78-1.63 (m, 1H), 1.56. 1.54 (m, 2H), 1.45-1.35 (m, 15H), 1.31-1.14 (m, 5H), 0.86-0.53 (m, 2H).

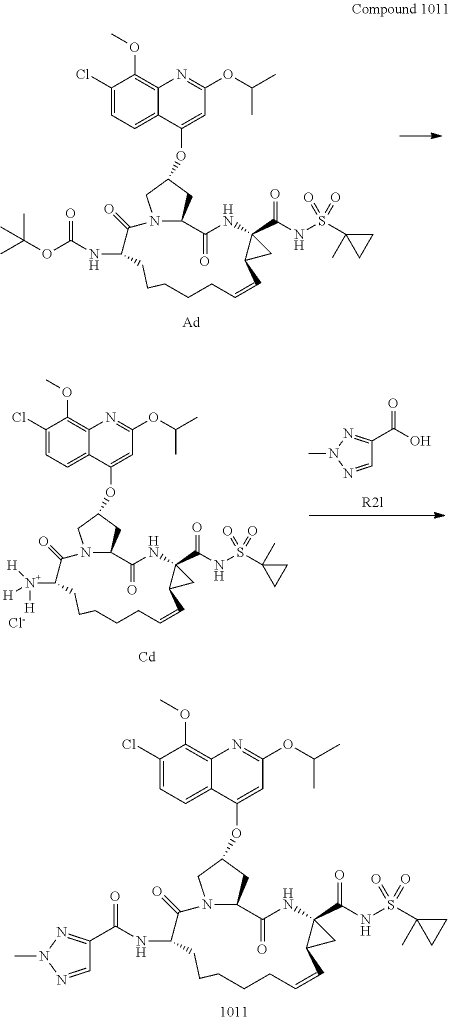

Boc protected amine Ad (85 mg, 0.102 mmol) is charged in a vial, then a 4 M solution of HCl in dioxane (1 mL, 4 mmol) is added. The solution is stirred at RT for 2 h, after which a precipitate forms. The solution is evaporated to dryness. Acid R2l (14.9 mg, 0.117 mmol, 1.15 equiv) is dissolved in DCM (2 mL), then TEA (57 μL, 0.408 mmol, 4.0 equiv) is added followed by TBTU (37.7 mg, 0.117 mmol, 1.15 equiv). The solution is stirred for 15 mins, after which the amine hydrochloride is added in DCM (1 mL) and solution is stirred at RT for 16 h. Water (2 mL) is added and the organic layer is extracted with EtOAc (3×5 mL). The solvent is evaporated and the residue is purified on prep HPLC (MeCN:H₂O, 0.1% TFA). The pure fractions are combined, concentrated, frozen and lyophilized to provide compound 1011.

FIA M.S. (electrospray): 839.4 (M−H)⁺

Retention time (min)=6.5 min

¹H NMR (400 MHz, DMSO-d₆): δ 10.77-10.42 (m, 1H), 8.23-8.13 (m, 1H), 8.00 (s, 1H), 7.96-7.91 (m, 1H), 7.81-7.71 (m, 1H), 7.63 (d, 1H, J=8.8 Hz), 7.24 (d, 1H, J=8.8 Hz), 6.48 (s, 1H), 5.46-5.34 (m, 2H), 5.27-5.18 (m, 1H), 4.74-4.68 (m, 1H), 4.41 (t, 1H, J=7.7 Hz), 4.25-4.14 (m, 2H), 4.13 (s, 3H), 3.98 (s, 3H), 2.47 (s, 3H), 2.42-2.36 (m, 1H), 2.07-1.97 (m, 2H), 1.95-1.65 (m, 4H), 1.54-1.49 (m, 1H), 1.47-1.43 (m, 1H), 1.40-1.23 (m, 10H), 1.19-1.13 (m, 1H), 1.09-1.02 (m, 2H), 0.93-0.88 (t, 1H, J=7.2 Hz), 0.43-0.36 (m, 2H).

### Synthesis of Using Intermediate Ae

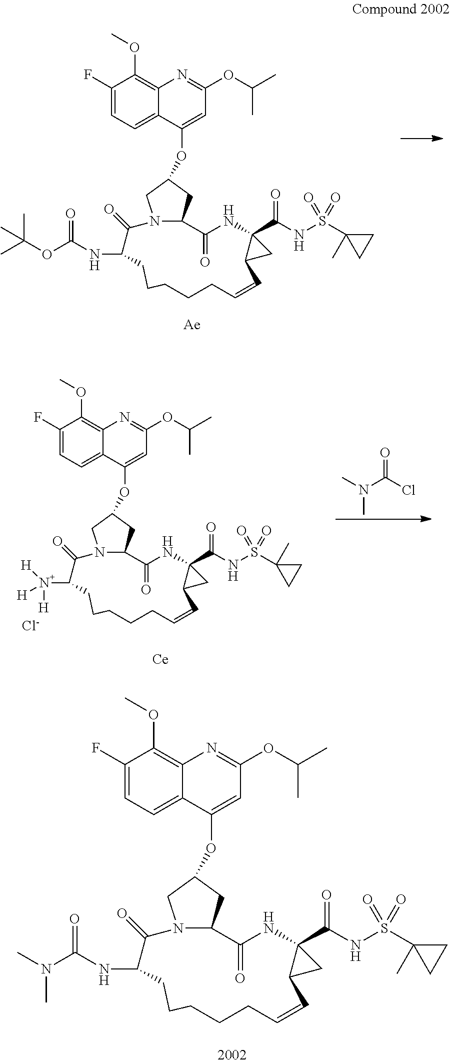

Boc protected macrocyclic amine Ae (84 mg, 0.103 mmol) is charged in a vial, dissolved in DCM (500 μL), then a 4 M solution of HCl in dioxane (3 mL) is added. The solution is stirred at RT for 1 h, after which the solution is evaporated to dryness. The Boc de-protected macrocyclic amine hydrochloride Ce is dissolved in DCM (4.0 mL), then, TEA (57.4 μL; 0.412 mmol, 4 equiv) and the dimethyl carbamyl chloride (13.3 mg; 0.124 mmol, 1.2 equiv) in DCM (1 mL) are added. The reaction is stirred at RT overnight. The resulting solution is filtered through a Millex filter and purified by prep HPLC (X-Bridge column, Ammonium Bicarbonate pH10: MeOH). The pure fractions are combined, concentrated, frozen and lyophilized to provide compound 2002.

FIA M.S. (electrospray): 785.4 (M−H)−, 787.3 (M+H)⁺

Retention time (min)=5.4 min

¹H NMR (400 MHz, DMSO-d₆): δ 10.82 (s, 1H), 8.83 (s, 1H), 7.85 (d, 1H, J=12.1 Hz), 7.23 (d, 1H, J=7.8 Hz), 6.43 (s, 1H), 6.32 (bs, 1H), 5.66-5.52 (m, 1H), 5.54-5.42 (m, 1H), 5.39 (bs, 1H), 5.17-5.00 (m, 1H), 4.76-4.63 (m, 1H), 4.35-4.27 (m, 1H), 4.20-4.08 (m, 1H), 3.94 (s, 3H), 3.88-3.78 (m, 1H), 5.86 (s, 6H), 2.65-2.58 (m, 2H), 2.33-2.15 (m, 2H), 1.90-1.79 (m, 1H), 1.79-1.60 (m, 1H), 1.58-1.50 (m, 2H), 1.50-1.31 (m, 15H), 1.31-1.14 (m, 3H), 0.94-0.70 (m, 2H).

### Synthesis of Compounds from Tables 1 and 2 Using Intermediates Af and Bf

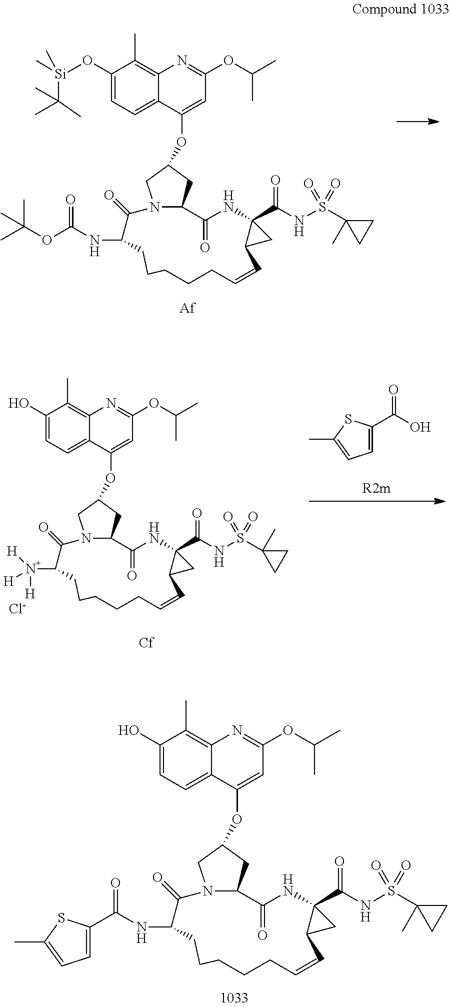

5-methyl-2-thiophene carboxilic Acid R2m (11.6 mg, 0.082 mmol) is dissolved in DCM (2 mL), then TEA (47 μL, 0.340 mmol) is added followed by TBTU (25.1 mg, 0.078 mmol). The solution is stirred for 15 mins, after which the amine hydrochloride Cf (50 mg, 0.068 mmol) is added. The solution is stirred at RT for 16 h and then concentrated. The residue is redissolved in DMSO and the resulting solution is filtered through a Millex filter and purified by prep HPLC (Sunfire column, ammonium formate and MeOH). The pure fractions are combined, concentrated redissolved in MeCN and water, frozen and lyophilized to provide compound 1033.

FIA M.S. (electrospray): 822.4 (M+H)⁺

Retention time (min)=5.6 min

¹H NMR (400 MHz, DMSO-d₆): δ 10.82 (bs, 1H), 9.62 (s, 1H), 8.84 (bs, 1H), 8.58 (bs, 1H), 7.80 (d, 1H, J=8.6 Hz), 7.70 (d, 1H, J=3.5 Hz), 6.91 (d, 1H, J=9.0 Hz), 6.83 (d, 1H, J=2.7 Hz), 6.27 (s, 1H), 5.65-5.53 (m, 1H), 5.47 (S, 1H, J=6.3 Hz), 5.41-5.35 (m, 1H), 5.17-5.01 (m, 1H), 4.73-4.60 (m 1H), 4.54-4.43 (m, 1H), 4.32 (dd, 1H, J=9, 7.4 Hz), 4.02-3.91 (m, 1H), 2.67-2.56 (m, 1H),), 2.48 (s, 3H), 2.39 (s, 3H), 2.36-2.24 (m, 2H), 2.04-1.90 (m, 1H), 1.85-1.67 (m, 1H), 1.59-1.15 (m, 21H), 0.92-0.77 (m, 2H).

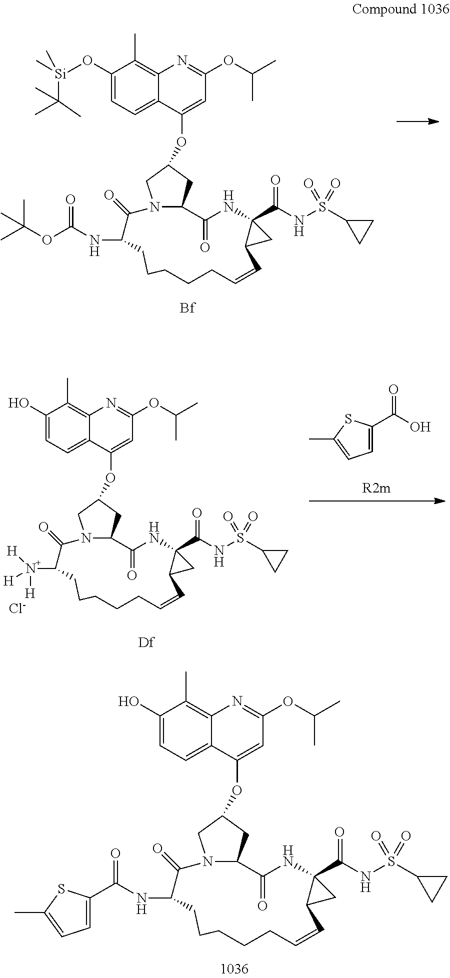

5-methyl-2-thiophene carboxilic Acid R2m (11.8 mg, 0.083 mmol) is dissolved in DCM (2 mL), then TEA (48 μL, 0.347 mmol) is added followed by TBTU (25.6 mg, 0.080 mmol). The solution is stirred for 15 mins, after which the amine hydrochloride Df (50 mg, 0.069 mmol) is added. The solution is stirred at RT for 16 h, concentrated and then the residual is dissolved in DMSO. The resulting solution is filtered through a Millex filter and purified by prep HPLC (Sunfire column, ammonium formate and MeOH). The pure fractions are combined, concentrated redissolved in MeCN and water, frozen and lyophilized to provide compound 1036.

FIA M.S. (electrospray): 808.4 (M+H)⁺

Retention time (min)=5.4 min

¹H NMR (400 MHz, DMSO-d₆): δ 11.08 (bs, 1H), 9.62 (s, 1H), 8.74-8.63 (m, 1H), 8.58 (bs, 1H), 7.80 (d, 1H, J=9 Hz), 7.70 (d, 1H, J=3.5 Hz), 6.91 (d, 1H, J=9.0 Hz), 6.83 (d, 1H, J=2.7 Hz), 6.26 (s, 1H), 5.65-5.52 (m, 1H), 5.48 (S, 1H, J=6.3 Hz), 5.40-5.33 (m, 1H), 5.25-5.09 (m, 1H), 4.73-4.60 (m 1H), 4.52-4.41 (m, 1H), 4.28 (dd, 1H, J=8.6, 7.8 Hz), 4.01-3.88 (m, 1H), 2.92-2.82 (m, 1H),), 2.71-2.58 (m, 2H), 2.48 (s, 3H), 2.39 (s, 3H), 2.34-2.22 (m, 2H), 2.02-1.65 (m, 2H), 1.60-1.50 (m, 3H), 1.50-1.41 (m, 4H), 1.37 (d, 3H, J=5.1 Hz), 1.36 (d, 3H, J=5.8 Hz), 1.31-1.15 (m, 2H), 1.11-0.96 (m, 4H).

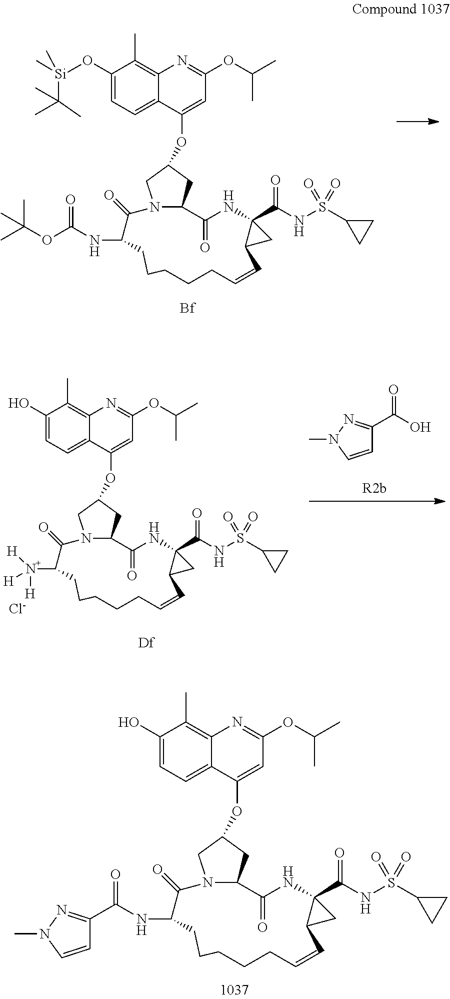

1-methyl-1H-pyrazole-3-carboxylic Acid R2b (10.9 mg, 0.087 mmol) is dissolved in DCM (2 mL), then TEA (48 μL, 0.347 mmol) is added followed by TBTU (25.6 mg, 0.080 mmol). The solution is stirred for 15 mins, after which the amine hydrochloride Df (50 mg, 0.069 mmol) is added and the solution is stirred at RT for 16 h. The reaction mixture is concentrated then the residue is redissolved in DMSO. The resulting solution is filtered through a Millex filter and purified by prep HPLC (Sunfire column, ammonium formate and MeOH). The pure fractions are combined, concentrated redissolved in MeCN and water, frozen and lyophilized to provide compound 1037.

FIA M.S. (electrospray): 792.4 (M+H)⁺

Retention time (min)=5.0 min

¹H NMR (400 MHz, DMSO-d₆): δ 11.05 (bs, 1H), 9.62 (s, 1H), 8.78 (bs, 1H), 7.81-7.78 (m, 1H) 7.76 (d, 1H, J=2.4 Hz), 7.67 (d, 1H, J=9 Hz), 6.87 (d, 1H, J=8.6 Hz), 6.60 (d, 1H, J=2.3 Hz), 6.28 (s, 1H), 5.65-5.58 (m, 1H) 5.47 (S, 1H, J=5.9 Hz), 5.43-5.37 (m, 1H), 5.17-5.09 (m, 1H), 4.62-4.54 (m 1H), 4.51-4.48 (m, 1H), 4.35-4.32 (m, 1H), 3.89 (s, 3H), 2.94-2.86 (m, 1H),), 2.60-2.55 (m, 2H), 2.38 (s, 3H), 2.33-2.28 (m, 2H), 1.99-1.73 (m, 3H), 1.59-1.53 (m, 3H), 1.48-1.37 (m, 4H), 1.37 (d, 3H, J=5.9 Hz), 1.36 (d, 3H, J=5.8 Hz) 1.31-1.15 (m, 2H), 1.11-0.96 (m, 4H).

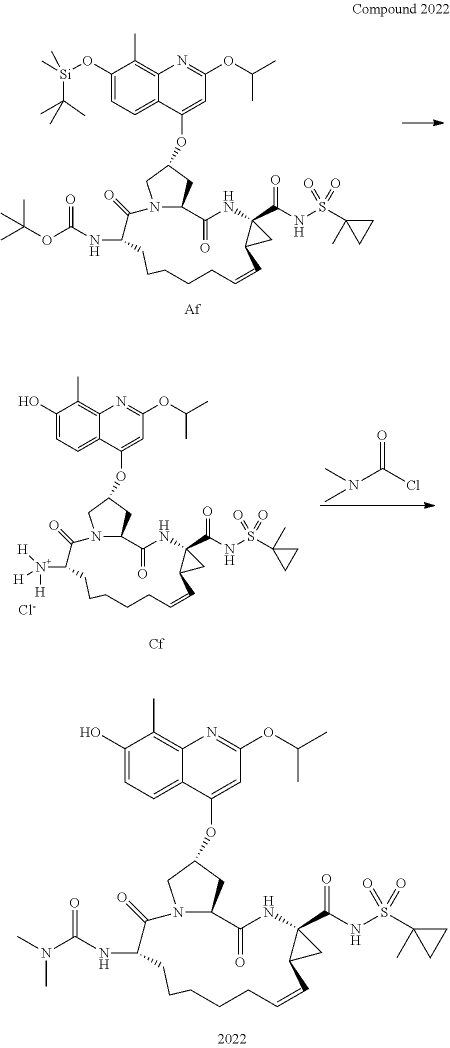

Treatment of Af with 4 N HCl in dioxane for 2 h followed by concentration in vacuo provides amine hydrochloride Cf. Amine hydrochloride Cf (45 mg, 0.061 mmol) is dissolved in DCM (1 mL), then TEA (25 μL, 0.184 mmol) is added. Dimethylcarbamyl chloride (6.7 μL, 0.074 mmol) is dissolved in DCM (1 mL) and added into the amine solution. This solution is stirred at RT, concentrated in vacuo and redissolved in DMSO. The resulting solution is filtered through a Millex filter and purified by prep HPLC (Sunfire column, ammonium formate and MeOH). The pure fractions are combined, concentrated, redissolved in MeCN and water, frozen and lyophilized to provide compound 2022.

FIA M.S. (electrospray): 767.5 (M−H)⁻

Retention time (min)=5.0 min

¹H NMR (400 MHz, DMSO-d₆): δ 10.83 (bs, 1H), 9.57 (s, 1H), 8.84 (bs, 1H), 7.83 (d, 1H, J=8.2 Hz), 6.87 (d, 1H, J=8.8 Hz), 6.37-6.29 (m, 1H), 6.26 (s, 1H), 5.65-5.56 (m, 1H), 5.47 (S, 1H, J=6.1 Hz), 5.37-5.33 (m, 1H), 5.05 (dd, 1H, J=9.2 Hz), 4.66 (d, 1H, J=10.4 Hz), 4.31 (dd, 1H, J=9.2, 6.9 Hz), 4.20-4.12 (m, 1H), 3.90-3.83 (m, 1H), 2.76 (s, 6H), 2.55-2.52 (m, 1H), 2.37 (s, 3H), 2.34-2.24 (m, 2H), 1.91-1.64 (m, 2H), 1.57-1.46 (m, 3H), 1.43-1.12 (m, 18H), 0.90-0.83 (m, 2H).

### Synthesis of Compound from Table 2 Using Intermediate Ag

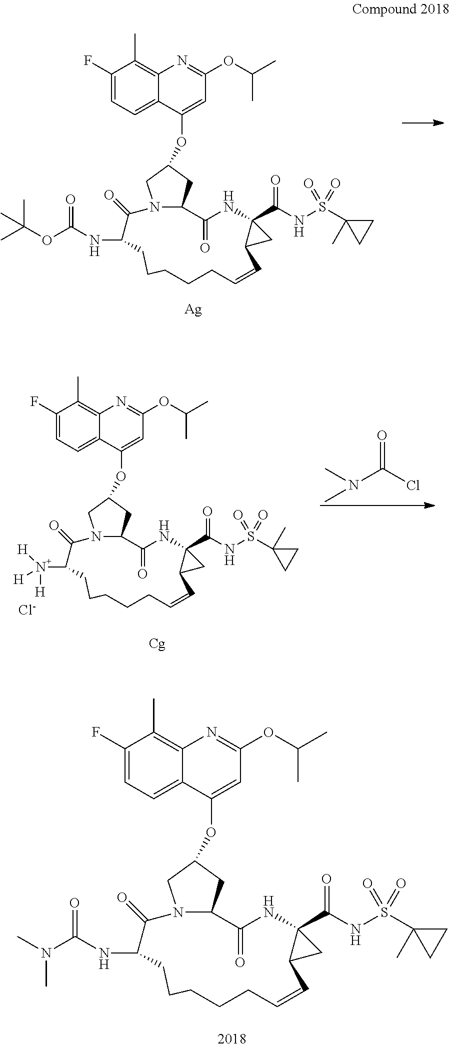

Boc protected macrocyclic amine Ag (74 mg, 0.093 mmol) is charged in a vial and a 4 M solution of HCl in dioxane (3 mL) is added. The solution is stirred at RT for 1 h, after which the solution is evaporated to dryness to provide Cg. The Boc de-protected macrocyclic amine hydrochloride Cg is dissolved in DCM (4.0 mL), then, TEA (51.6 μL; 0.370 mmol, 4.00 equiv) and the dimethylcarbamyl chloride (11.9 mg; 0.111 mmol, 1.20 equiv) in DCM (1 mL) are added. The reaction is stirred at RT overnight. The resulting solution is filtered through a Millex filter and purified by prepHPLC (X-Bridge column, Ammonium Bicarbonate pH10: MeOH). The pure fractions are combined, concentrated, frozen and lyophilized to provide compound 2018.

FIA M.S. (electrospray): 769.5 (M−H)−, 771.4 (M+H)⁺

Retention time (min)=6.7 min

¹H NMR (400 MHz, DMSO-d₆): δ 10.83 (s, 1H), 8.85 (s, 1H), 8.04 (dd, 1H, J=8.6, 7.1 Hz), 7.11 (dd, 1H, J=9.4, 9.4 Hz), 6.49 (s, 1H), 6.31 (bs 1H), 5.6 (bs, 1H), 5.53-5.47 (m, 1H), 5.43 (bs, 1H), 5.06 (bs, 1H), 4.71-4.69 (m, 1H), 4.35-4.31 (dd, 1H, J=7.4, 7 Hz), 4.14 (bs, 1H), 3.88-3.85 (m, 1H), 2.79 (s, 6H), 2.63-2.54 (m, 2H), 2.48 (s, 3H), 2.23-2.28 (m, 2H), 1.89-1.71 (m, 2H), 1.57-1.53 (m, 2H), 1.40-1.37 (m, 15H), 1.28-1.22 (m, 3H); 0.85 (bs, 2H).

### Synthesis of Compounds from Tables 1 and 2 Using Intermediate Ai

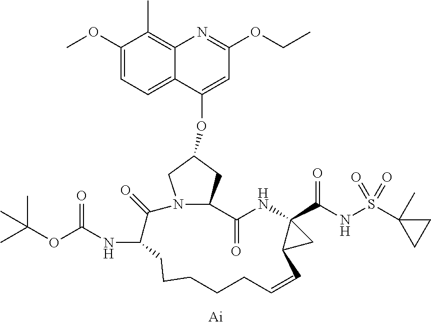

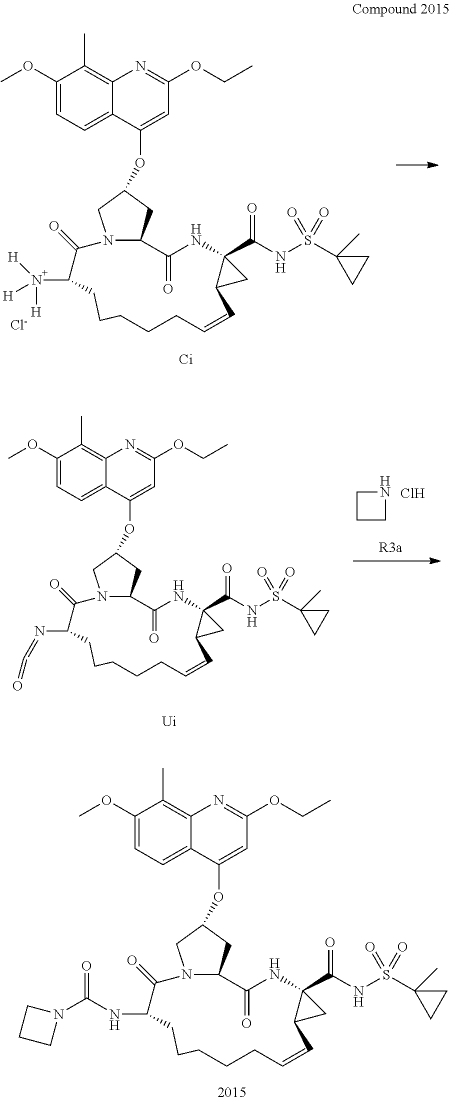

Ci is prepared by treating Ai with 4 N HCl in dioxane for 2 h followed by concentration in vacuo. To the crude deprotected macrocyclic amine Ci (50 mg, 0.067 mmol) in DCM (1 mL) is added carbonyl diimidazole (13 mg, 0.082 mmol) and TEA (37 μl, 0.267 mmol). The reaction mixture is stirred at RT for 60 mins. Azetidine hydrochloride salt (12.7 mg, 0.136 mmol) in solution in DCM (1 mL) is added to the activated macrocyclic amine Ui. The reaction mixture is stirred at RT for 16 h. The reaction is incomplete so azetidine hydrochloride salt R3a (12.7 mg, 0.136 mmol) and TEA (28 μl, 0.276 mmol) is added. The resulting solution is stirred at RT for 40 h and concentrated. To the solid is added DMSO and a few drops of acetic acid. The resulting solution is filtered through a Millex filter and purified by prep HPLC (Sunfire column, ammonium formate and MeOH). The pure fractions are combined concentrated, frozen and lyophilized to provide compound 2015.

FIA M.S. (electrospray): 781.4 (M+H)⁺

Retention time (min)=5.3 min

¹H NMR (400 MHz, DMSO-d₆): δ 10.82 (bs, 1H), 8.89 (bs, 1H), 7.89 (d, 1H, J=9.0 Hz), 7.12 (d, 1H, J=9.2 Hz), 6.37 (s, 2H), 5.65-5.55 (m 1H), 5.41 (bs, 1H), 5.12-4.99 (m, 1H), 4.58-4.51 (m, 1H), 4.47 (q, 2H, J=6.8), 4.37 (dd, 1H, J=7.4, 7.4 Hz), 4.25-4.16 (m, 1H), 3.99-3.91 (m, 1H), 3.89 (s, 3H), 3.67 (dd, 4H, J=7.6, 7.4 Hz), 2.61-2.59 (m, 1H), 2.56-2.54 (m, 1H), 2.43 (s, 3H), 2.34-2.31 (m, 1H), 2.06-1.97 (m, 2H), 1.87-1.71 (m, 2H), 1.59-1.48 (m, 2H), 1.45-1.10 (m, 16H), 0.92-0.80 (m, 2H).

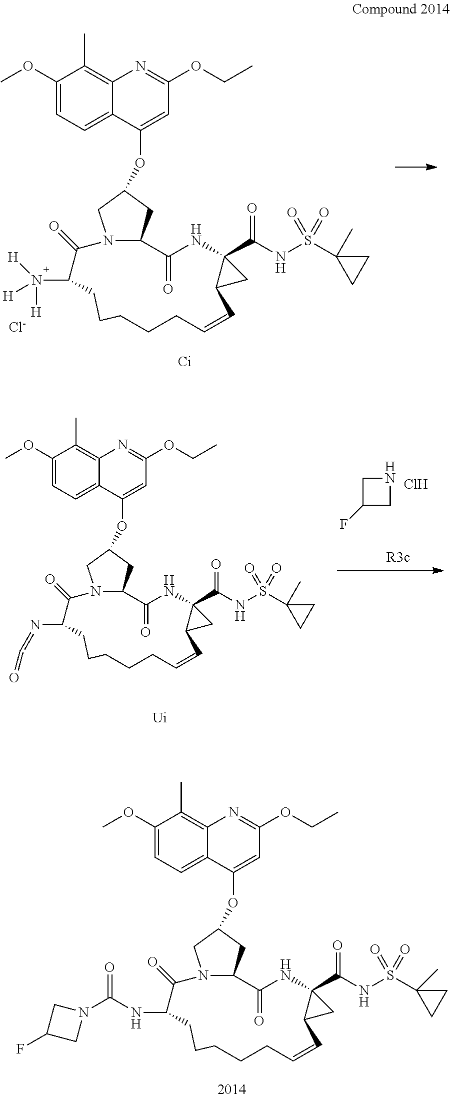

To the crude deprotected macrocyclic amine Ci (60 mg, 0.082 mmol) in DCM (1 mL) is added carbonyl diimidazole (16 mg, 0.098 mmol) and TEA (68 μl, 0.488 mmol). The reaction mixture is stirred at RT for 60 mins. Azetidine hydrochloride R3c (13.6 mg, 0.123 mmol) in solution in DCM (1 mL) is added to the solution activated macrocyclic amine Ui. The reaction mixture is stirred at RT for 16 h. The reaction is incomplete so azetidine R3c (13.6 mg, 0.123 mmol) is added. The resulting solution is stirred at RT for 24 h and then concentrated. To the solid is added DMSO. The resulting solution is filtered through a Millex filter and purified by prep HPLC (Sunfire column, ammonium formate MeOH). The pure fractions are combined concentrated, frozen and lyophilized to provide compound 2014.

FIA M.S. (electrospray): 799.4 (M+H)⁺

Retention time (min)=5.4 min

¹H NMR (400 MHz, DMSO-d₆): δ 10.85 (bs, 1H), 8.88 (bs, 1H), 7.88 (d, 1H, J=9.0 Hz), 7.13 (d, 1H, J=9.0 Hz), 6.76 (bs, 1H), 6.38 (s, 1H), 5.63-5.53 (m 1H), 5.41 (m, 1H), 5.25 (ttd, 1H, ¹J_{H-F}=59.6 Hz, J=6.0, 3.0 Hz), 5.10-5.01 (m, 1H), 4.57-4.50 (m, 1H), 4.47 (q, 2H, J=7.0 Hz), 4.36 (dd, 1H, J=7.4, 7.1 Hz), 4.24-4.17 (m, 1H), 4.07-3.93 (m, 3H), 3.89 (s, 3H), 3.84-3.69 (m, 1H), 2.62-2.57 (m, 1H), 2.43 (s, 3H), 2.40-2.31 (m, 2H), 1.89-1.69 (m, 2H), 1.58-1.47 (m, 2H), 1.43-1.32 (m, 13H), 1.30-1.14 (m, 4H), 0.91-0.78 (m, 2H).

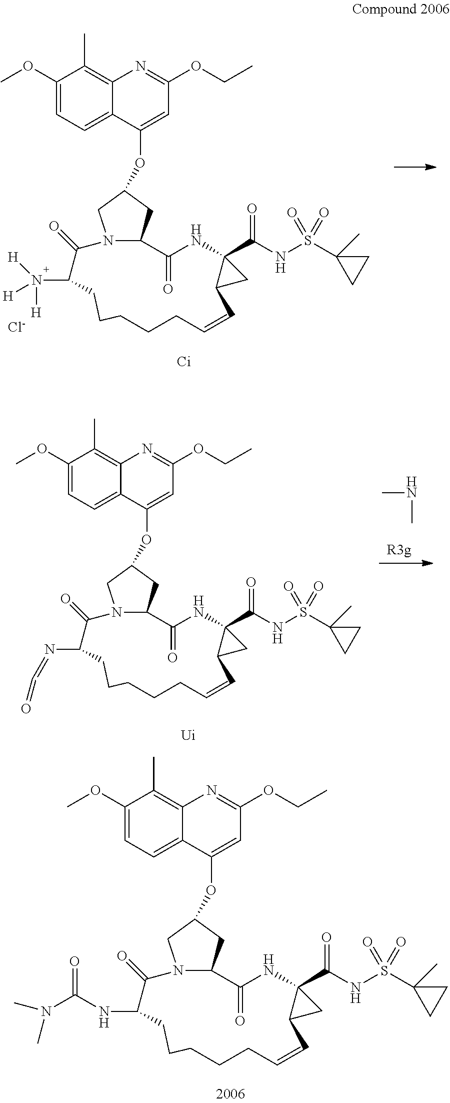

Compound 2006 is synthesized analogously to the procedure described for compound 2014 starting from the macrocyclic intermediate Ci (50 mg, 0.068 mmol) and using excess dimethyl amine R3g as a solution in THF as the amine nucleophile.

FIA M.S. (electrospray): 769.4 (M+H)⁺, 767.5 (M−H)⁻

Retention time (min)=5.3 min

¹H NMR (400 MHz, DMSO-d₆): δ 10.84 (bs, 1H), 8.87 (s, 1H), 7.96 (d, 1H, J=9.4 Hz), 7.08 (d, 1H, J=9.0 Hz), 6.38 (s, 1H), 6.30 (d, 1H, J=7.0 Hz), 5.61 (q, 1H, J=8.9 Hz), 5.41 (bs, 1H), 5.03 (t, 1H, J=9.4), 4.68 (d, 1H, J=11.0 Hz), 4.47 (q, 2H, J=6.5 Hz), 4.34 (dd, 1H, J=10.0, 6.9 Hz), 4.18-4.13 (m, 1H), 3.91-3.86 (m, 1H), 3.88 (s, 3H), 2.72 (s, 6H), 2.61-2.53 (m, 2H), 2.46-2.43 (m, 1H), 2.43 (s, 3H), 2.39-2.27 (m, 2H), 1.91-1.82 (m, 1H), 1.76-1.68 (m, 1H), 1.57 (dd, 1H, J=8.0, 4.9 Hz), 1.49 (dd, 1H, J=9.4, 5.1 Hz), 1.45-1.33 (m, 5H), 1.38 (t, 3H, J=6.5 Hz), 1.37 (s, 3H), 1.32-1.18 (m, 3H), 0.91-0.82 (m, 2H).

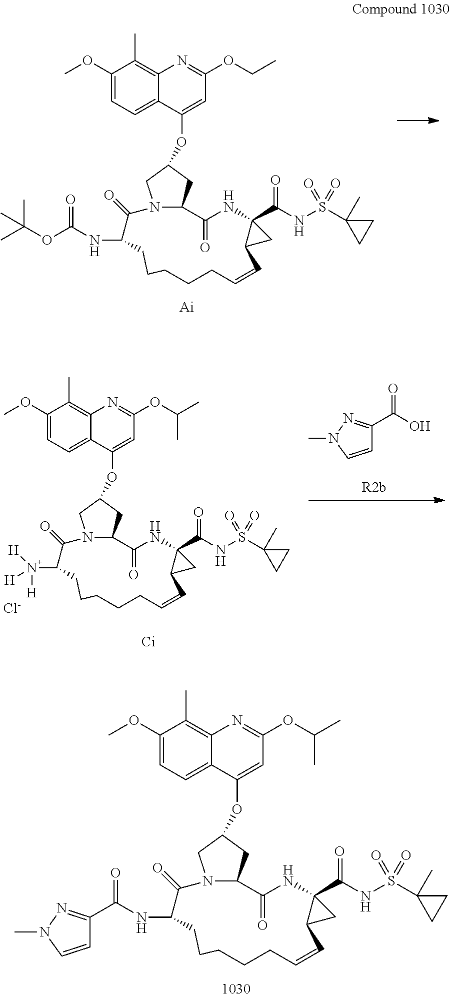

Boc protected amine Ai (40 mg, 0.050 mmol) is charged in a vial, then a 4 M solution of HCl in dioxane (2 mL, 8 mmol) is added. The solution is stirred at RT for 2 h, after which a precipitate forms. The solution is evaporated to dryness to give Ci. Acid R2b (7.6 mg, 0.060 mmol, 1.2 equiv) is dissolved in DMF (2 mL), then TEA (35 μL, 0.25 mmol, 5.0 equiv) is added followed by TBTU (22.9 mg, 0.060 mmol, 1.2 equiv). The solution is stirred for 15 mins, after which the amine hydrochloride Ci is added in DMF (1 mL). This solution is stirred at RT for 16 h. Water (2 mL) is added and the organic layer is extracted with EtOAc (3×5 mL). The solvent is evaporated and the residue is purified on prep HPLC (MeCN:H₂O, 0.1% TFA). The pure fractions are combined, concentrated, frozen and lyophilized to provide compound 1030.

FIA M.S. (electrospray): 806.2 (M+H)⁺; 804.3 (M−H)⁺

Retention time (min)=5.5 min

¹H NMR (400 MHz, DMSO-d₆): δ 10.84 (s, 1H), 8.94 (s, 1H), 7.83 (d, 1H, J=9.4 Hz), 7.79 (d, 1H, J=7.1 Hz), 7.76 (d, 1H, J=2.3 Hz), 7.08 (d, 1H, J=8.9 Hz), 6.58 (d, 1H, J=2.3 Hz), 6.41 (s, 1H), 5.66-5.57 (m, 1H), 5.47-5.43 (m, 1H), 5.06 (dd, 1H, J=9.5, 9.2 Hz), 4.62-4.56 (m, 1H), 4.55-4.38 (m, 5H), 4.05-3.99 (m, 1H), 3.88 (s, 3H), 3.87 (s, 3H), 2.65-2.58 (m, 1H), 2.43 (s, 3H), 2.39-2.28 (m, 2H), 2.01-1.89 (m, 1H), 1.87-1.76 (m, 1H), 1.61-1.54 (m, 1H), 1.53-1.48 (m, 1H), 1.46-1.34 (m, 11H), 1.32-1.18 (m, 4H), 0.93-0.84 (m, 2H).

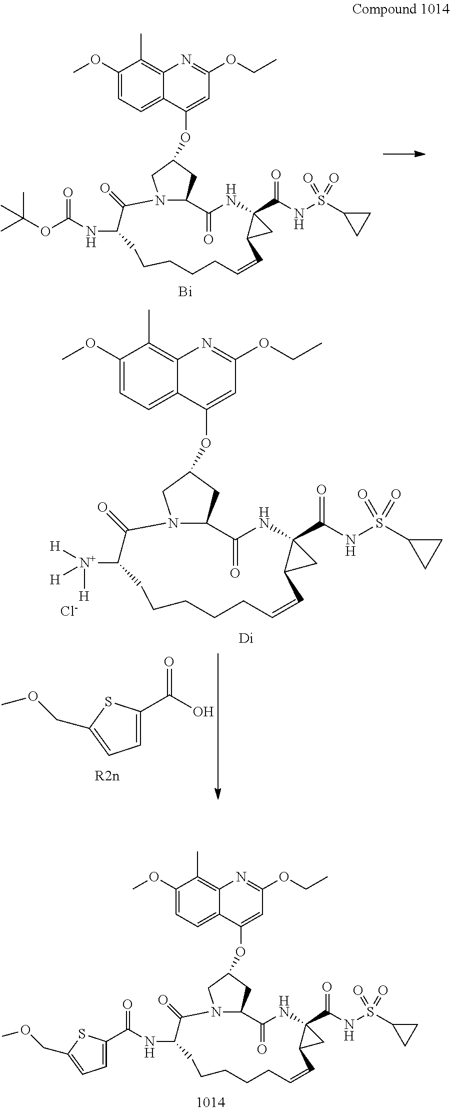

Compound 1014 is synthesized analogously to the procedure described for compound 1030 starting from the macrocyclic intermediate Bi (50 mg, 0.069 mmol) and using R2n (0.016 g, 0.090 mmol) as the coupling partner.

FIA M.S. (electrospray): 838.3 (M+H)⁺

Retention time (min): 5.6 min

¹H NMR (400 MHz, DMSO-d₆): δ 11.03 (s, 1H), 8.76 (s, 1H), 8.69 (d, 1H, J=6.5 Hz), 7.97 (d, 1H, J=9.1 Hz), 7.75 (d, 1H, J=3.7 Hz), 7.08 (d, 1H, J=9.1 Hz), 7.03 (d, 1H, J=3.7 Hz), 6.39 (s, 1H), 5.62 (q, 1H, J=9.8 Hz), 5.44 (bs, 1H), 5.11 (dd, 1H, J=9.3, 8.8 Hz), 4.72 (d, 1H, J=11.4 Hz), 4.58 (s, 2H), 4.52-4.45 (m, 3H), 4.32 (dd, 1H, J=9.8, 6.9 Hz), 3.94 (dd, 1H, J=11.3, 3.4 Hz), 3.88 (s, 3H), 3.29 (s, 3H), 2.93-2.86 (m, 1H), 2.69-2.57 (m, 2H), 2.44 (s, 3H), 2.40-2.29 (m, 2H) 2.06-1.95 (m, 1H), 1.78-1.69 (m, 1H), 1.57 (dd, 1H, J=8.2, 4.9 Hz), 1.51 (dd, 1H, J=9.3, 4.6 Hz), 1.50-1.35 (m, 5H), 1.39 (t, 3H, J=7.0 Hz), 1.32-1.20 (m, 2H), 1.11-0.97 (m, 4H).

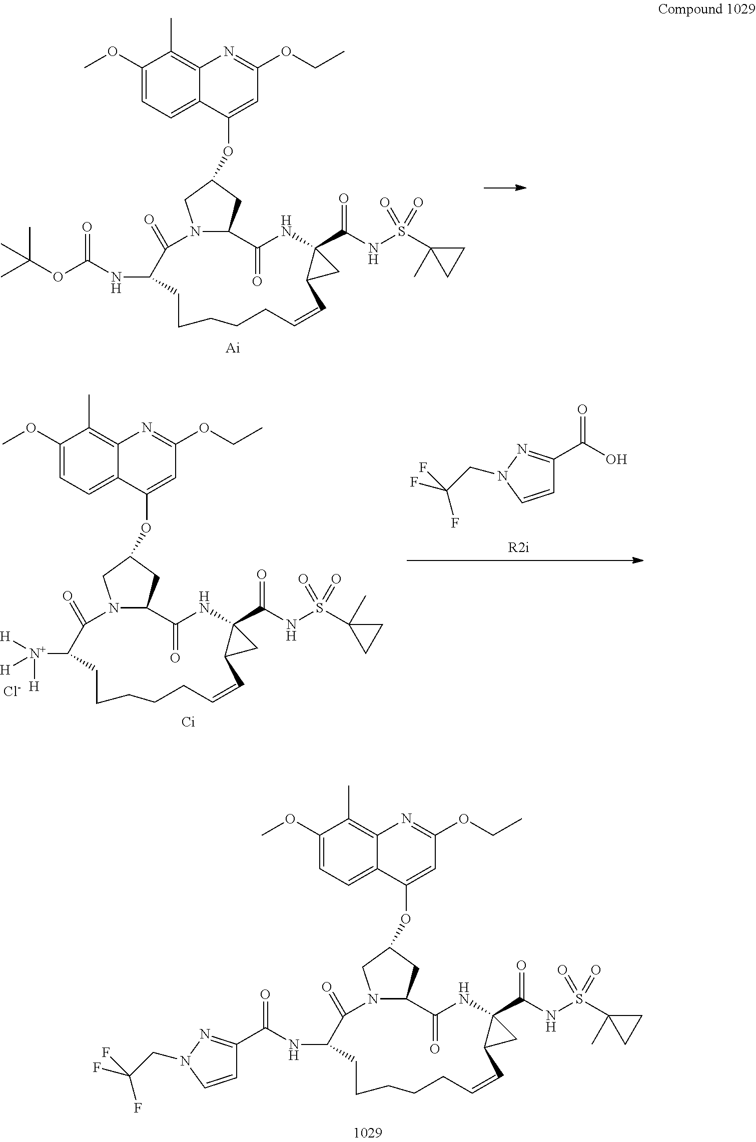

Starting material Ai (45 mg, 0.056 mmol) is dissolved in 4 N HCl/dioxane and the reaction mixture is for 1 h at RT. The reaction mixture is concentrated in vacuo to give crude Ci. The acid R2i is dissolved in DMF (2.0 mL), DIPEA (0.059 mL, 0.338 mmol) and TBTU (21.7 mg, 0.068 mmol) are added. The reaction mixture is stirred for 15 mins. The crude Ci is added and the reaction mixture is stirred at RT for 16 h. The reaction mixture is purified by prep HPLC to provide compound 1029.

FIA M.S. (electrospray): 874.4 (M+H)⁺

Retention time (min)=6.0 min

¹H NMR (400 MHz, DMSO-d₆): δ 10.83 (s, 1H), 8.92 (s, 1H), 7.95 (b, 1H), 7.93 (d, 1H, J=2.3 Hz), 7.86 (d, 1H, J=9.0 Hz), 7.08 (d, 1H, J=9.4 Hz), 6.74 (d, 1H, J=2.3 Hz), 6.40 (s, 1H), 5.60 (b, 1H), 5.44 (m, 1H), 5.23 (q, 2H, J=9.3 Hz), 5.09 (b, 1H), 4.63 (b, 1H), 4.47 (q, 2H, J=6.9 Hz), 4.43 (b, 2H), 4.04 (b, 1H), 3.87 (s, 3H), 2.67-2.65 (m, 1H), 2.43 (s, 3H), 2.38-2.32 (m, 3H), 2.01-1.80 (m, 2H), 1.58-1.51 (m, 3H), 1.44-1.23 (m, 11H), 1.39 (t, 3H, J=7.0 Hz), 0.85 (b, 2H).

### Synthesis of Compounds from Tables 1 and 2 Using Intermediates Aj and Bj

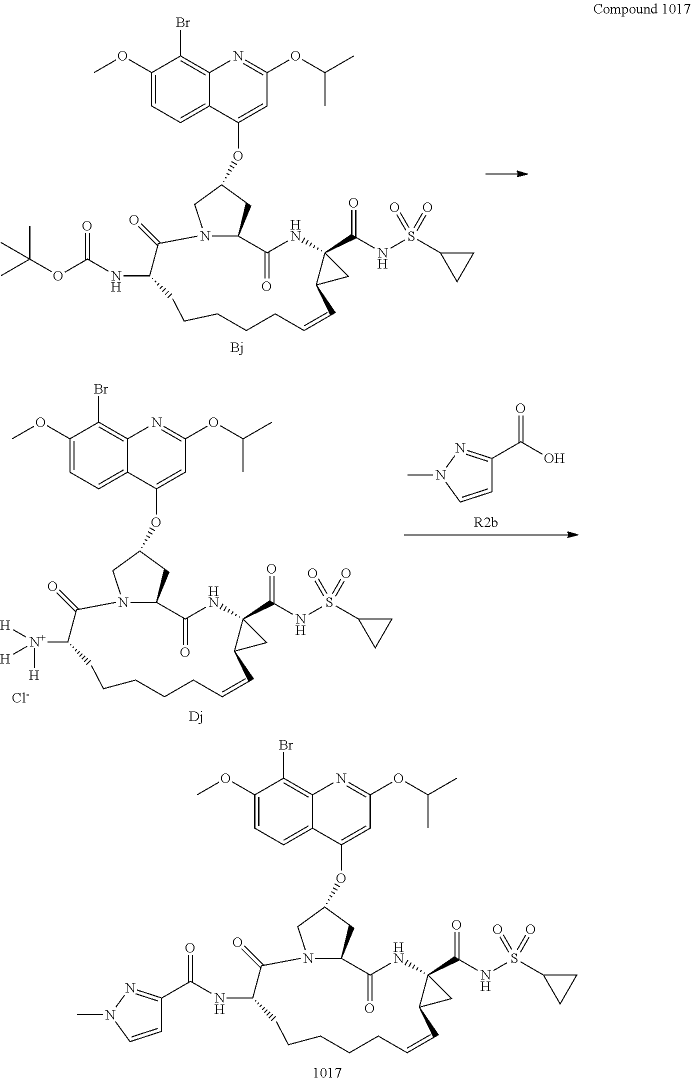

Boc protected macrocyclic amine Bj (40 mg, 0.047 mmole) is charged in a vial with a 4 M solution of HCl in dioxane (3 mL). The solution is stirred at RT for 1 h, after which the solution is evaporated to dryness to give crude Dj. 1-methyl-1H-pyrazole-3-carboxylic acid R2b (7.1 mg; 0.057 mmol) is dissolved in DMF (1.5 mL) and DIPEA (49 μL; 0.283 mmol) and HATU (21.5 mg; 0.057 mmol) are added and the mixture stirred for 15 mins. The Boc deprotected macrocyclic amine hydrochloride Dj is dissolved in DMF (1.5 mL) and added to the reaction mixture which is subsequently stirred at RT overnight. The resulting solution is filtered through a Millex filter and purified by prep HPLC (Sunfire column; 0.1% TFA/CH₃CN: 0.1% TFA/H₂O). The pure fractions are combined, concentrated, frozen and lyophilized to provide compound 1017.

FIA M.S. (electrospray): 854/856.3 (M−H)−, 856/858.3 (M+H)⁺

Retention Time (min)=6.5 min

¹H NMR (400 MHz, DMSO-d₆): δ 11.05 (s, 1H), 8.80 (s, 1H), 8.00 (d, 1H, J=9.0 Hz), 7.79 (d, 1H, J=6.7 Hz), 7.75 (d, 1H, J=2.4 Hz), 7.18 (d, 1H, J=9.4 Hz), 6.57 (d, 1H, J=2.4 Hz), 6.52 (s, 1H), 5.66-5.57 (m, 1H), 5.48 (bs, 1H), 5.16-5.10 (m, 1H), 4.60-4.48 (m, 4H), 4.40-4.34 (m, 1H), 4.02-3.95 (m, 1H), 3.95 (s, 3H), 3.88 (s, 3H), 2.94-2.88 (m, 1H), 2.65-2.58 (m, 1H), 2.39-2.26 (m, 3H), 2.00-1.87 (m, 1H), 1.87-1.73 (m, 1H), 1.62-1.50 (m, 3H), 1.48-1.32 (m, 4H), 1.40 (t, 3H, J=7.1, 14.1 Hz), 1.30-1.19 (m, 2H), 1.12-0.95 (m, 4H).

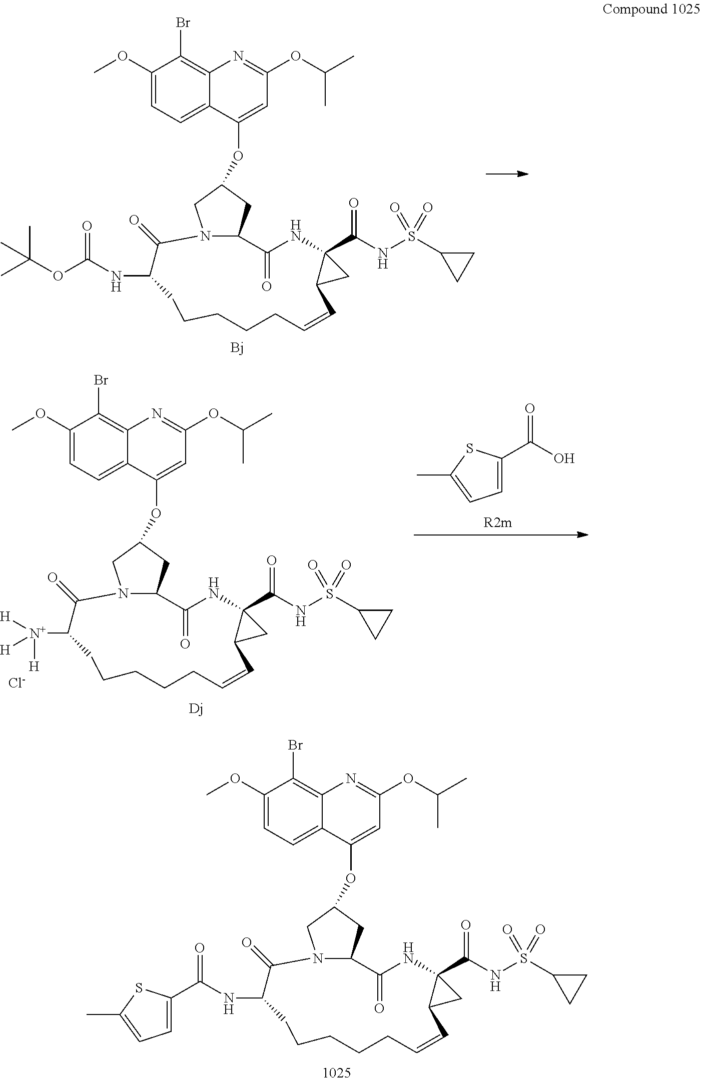

Starting material Bj (40 mg, 0.047 mmol) is dissolved in 4 N HCl/dioxane and the reaction mixture is stirred for 1 h at RT. The reaction mixture is concentrated in vacuo to give crude Ci. The acid R2m (15 mg, 0.060 mmol) is dissolved in DMF (2.0 mL) and DIPEA (0.059 mL, 0.338 mmol) and TBTU (21.7 mg, 0.068 mmol) are added. The reaction mixture is stirred for 15 mins. The crude Ci is added and the reaction mixture is stirred at RT for 16 h. The reaction mixture is purified by prep HPLC to provide compound 1025.

FIA M.S. (electrospray): 872.3 (M+H)⁺, 874.3 (M+2H)⁺

Retention time (min)=6.8 min

¹H NMR (400 MHz, DMSO-d₆): δ 11.07 (s, 1H), 8.78 (s, 1H), 8.58 (d, 1H, J=6.6 Hz), 8.07 (d, 1H, J=9.0 Hz), 7.65 (d, 1H, J=3.5 Hz), 7.15 (d, 1H, J=9.4 Hz), 6.81 (d, 1H, J=6.8 Hz), 6.51 (s, 1H), 5.65-5.58 (m, 1H), 5.47 (m, 1H), 5.12 (dd, 1H, J=10.2, 8.6 Hz), 4.72 (d, 1H, J=10.9 Hz), 4.55-4.50 (m, 2H), 4.46-4.40 (m, 1H), 4.33 (dd, 1H, J=10.2, 6.7 Hz), 4.01-3.90 (m, 2H), 3.96 (s, 3H), 2.93-2.87 (m, 1H), 2.68-2.58 (m, 2H), 2.45 (s, 3H), 2.40-2.30 (m, 2H), 2.04-1.94 (m, 1H), 1.76-1.67 (m, 1H), 1.58-1.35 (m, 7H), 1.40 (t, 3H, J=7.0 Hz), 1.30-1.22 (m, 1H), 1.11-0.97 (m, 4H).

### Synthesis of Compounds from Tables 1 and 2 Using Intermediate Ak

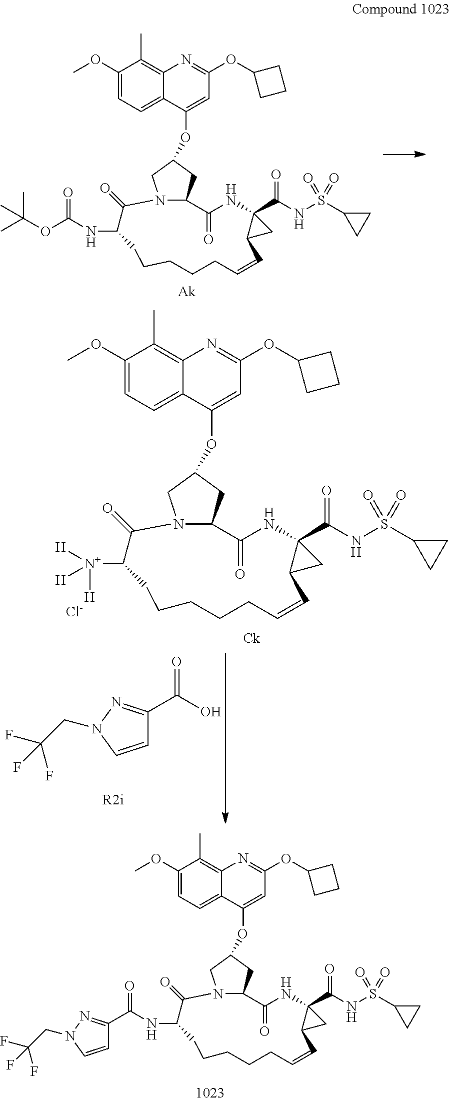

Intermediate Ck is prepared from Ak by treatment with 4 N HCl/dioxane for 1 h followed by concentration in vacuo. Acid R2i (12.2 mg, 0.063 mmol) is dissolved in DMF (1 mL), then TEA (29 μL, 0.210 mmol) is added followed by TBTU (19.4 mg, 0.060 mmol). The solution is stirred for 15 mins, after which the amine hydrochloride Ck (40 mg, 0.053 mmol) is added in DMF (1 mL). The solution is stirred at RT for 16 h. AcOH is added and the resulting solution is filtered through a Millex filter and purified by prep HPLC (Sunfire column, 0.1% TFA). The pure fractions are combined, frozen and lyophilized to provide compound 1023.

FIA M.S. (electrospray): 900.4 (M+H)⁺

Retention time (min)=6.4 min

¹H NMR (400 MHz, DMSO-d₆): δ 10.82 (s, 1H), 8.94 (s, 1H), 7.96 (d, 1H, J=7.1 Hz), 7.93 (d, 1H, J=2.3 Hz), 7.84 (d, 1H, J=9 Hz), 7.07 (d, 1H, J=9.4 Hz), 6.74 (d, 1H, J=2.3 Hz), 6.40 (s, 1H), 5.65-5.58 (m 1H), 5.48-5.43 (m, 1H), 5.32 (qn, 1H J=7.8 Hz), 5.22 (q, 2H, J=9 Hz), 5.06 (dd, 1H, J=9.8, 9.4 Hz), 4.63-4.57 (m, 1H), 4.54 (d, 1H, J=11.8), 4.41 (dd, 1H, J=7.4, 7.0 Hz), 4.04-4.00 (m, 1H), 3.87 (s, 3H), 2.64-2.58 (m, 2H), 2.43 (s, 3H), 2.38-2.31 (m, 3H), 2.15-2.08 (m, 2H), 2.01-1.98 (m, 1H), 1.85-1.67 (m, 3H), 1.59-1.23 (m, 15H), 0.90-0.86 (m, 2H).

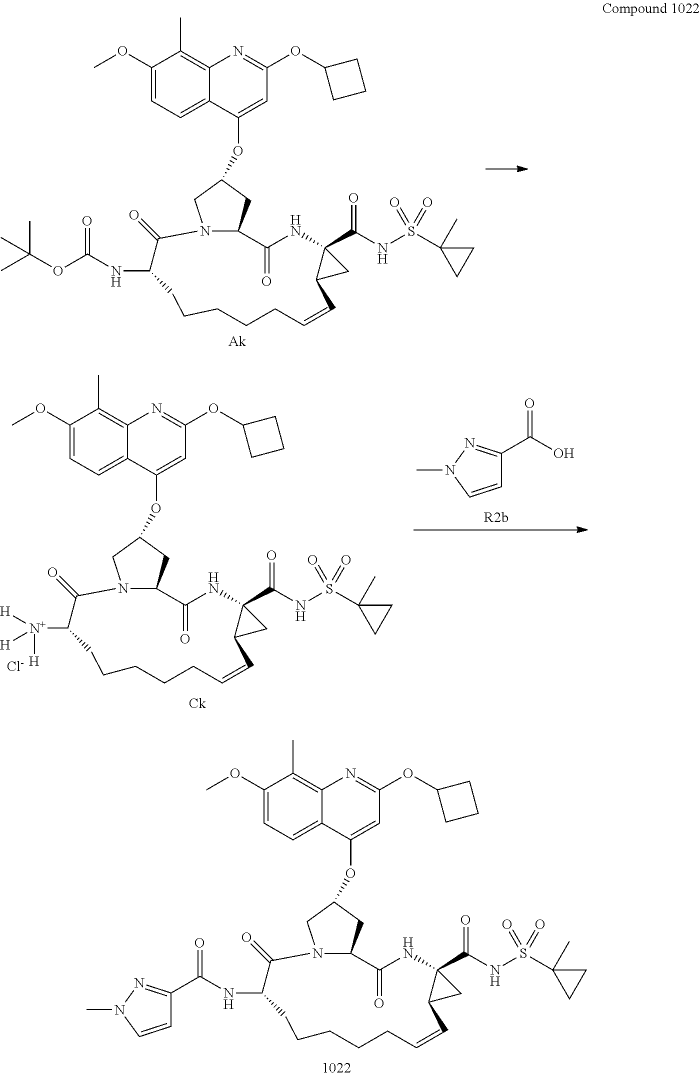

1-methyl-1H-pyrazole-3-carboxylic Acid R2b (5.9 mg, 0.047 mmol) is dissolved in DMF (1 mL), then TEA (27 μL, 0.197 mmol) is added followed by TBTU (14.6 mg, 0.045 mmol). The solution is stirred for 15 mins, after which the amine hydrochloride Ck (30 mg, 0.039 mmol) is added in DMF (0.5 mL). The solution is stirred at RT for 16 h. AcOH is added and the resulting solution is filtered through a Millex filter and purified by prep HPLC (Sunfire column, ammonium formate and MeOH). The pure fractions are combined, concentrated, redissolved in MeCN and water, frozen and lyophilized to provide compound 1022.

FIA M.S. (electrospray): 832.4 (M+H)⁺

Retention time (min)=6.0 min

¹H NMR (400 MHz, DMSO-d₆): δ 10.83 (s, 1H), 8.94 (s, 1H), 7.83 (d, 1H, J=9.4 Hz), 7.78 (d, 1H, J=7.1 Hz), 7.76 (d, 1H, J=2.3 Hz), 7.08 (d, 1H, J=9.4 Hz), 6.59 (d, 1H, J=2.4 Hz), 6.39 (s, 1H), 5.65-5.58 (m 1H), 5.47-5.42 (m, 1H), 5.32 (qn, 1H J=8.0 Hz), 5.06 (dd, 1H, J=9.8, 9.4 Hz), 4.61-4.57 (m, 1H), 4.51 (d, 1H, J=11.8), 4.41 (dd, 1H, J=9.4, 7.4 Hz), 4.06-3.99 (m, 1H), 3.88 (s, 3H), 3.87 (s, 3H), 2.61-2.58 (m, 1H), 2.43 (s, 3H), 2.38-2.27 (m, 2H), 2.15-2.08 (m, 2H), 1.99-1.65 (m, 5H), 1.59-1.18 (m, 16H), 0.92-0.83 (m, 2H).

### Synthesis of Compounds from Tables 1 and 2 Using Intermediates Al and Bl

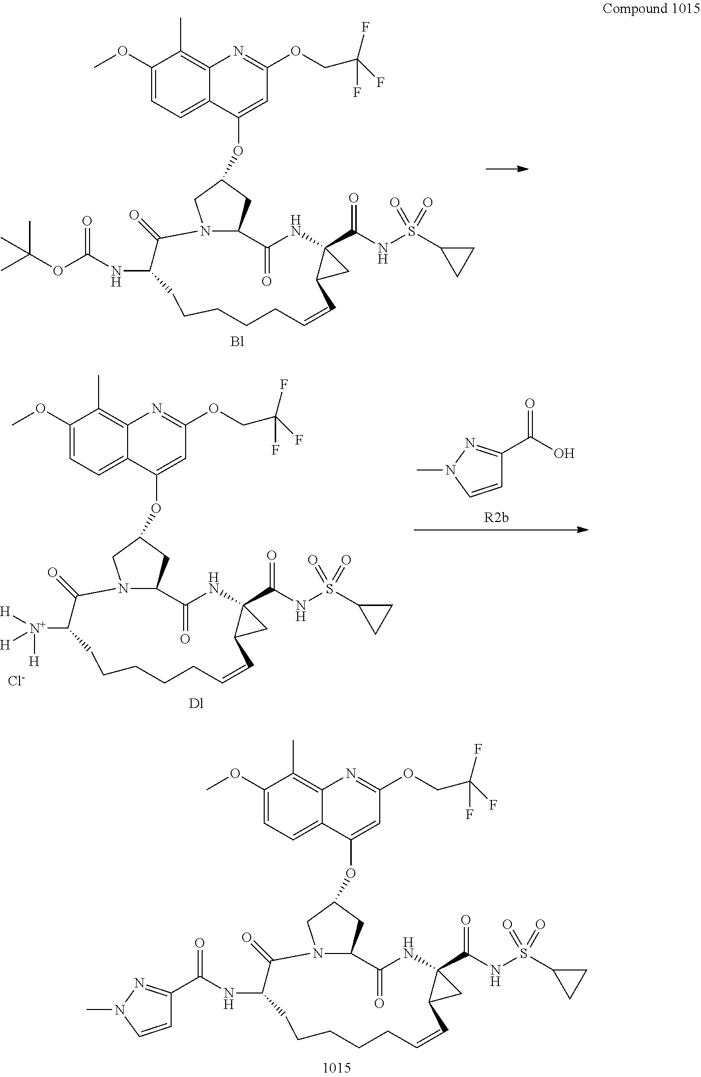

Boc protected amine macrocycle Bl (45 mg, 0.054 mmol) is charged in a vial, then a 4 M solution of HCl in dioxane (2 mL, 8 mmol) is added. The solution is stirred at RT for 1 h, after which a precipitate forms. The solution is evaporated to dryness to provide Dl. 1-methyl-1H-pyrazole-3-carboxylic Acid R2b (8.1 mg, 0.064 mmol, 1.2 equiv) is dissolved in DMF (3 mL), then TEA (30 μL, 0.215 mmol) is added followed by HATU (24.5 mg, 0.064 mmol, 1.2 equiv). The solution is stirred for 15 mins, after which the amine hydrochloride Dl is added in DMF (1 mL) and the solution is stirred at RT for 16 h. The resulting solution is filtered through a Millex filter and purified by prep HPLC (Sunfire column, 0.1% TFA). The pure fractions are combined, concentrated, frozen and lyophilized to provide compound 1015.

FIA M.S. (electrospray): 846.1 (M+H)⁺

Retention time (min)=6.8 min

¹H NMR (400 MHz, DMSO-d₆): δ 11.06 (s, 1H), 8.80 (s, 1H), 7.90 (d, 1H, J=9.0 Hz), 7.79 (d, 1H, J=7 Hz), 7.75 (d, 1H, J=2.3 Hz), 7.16 (d, 1H, J=9.4 Hz), 6.61 (s, 1H), 6.58 (d, 1H, J=2.4 Hz), 5.66-5.58 (m 1H), 5.51 (m, 1H), 5.23-5.07 (m, 3H), 4.60-4.54 (m, 2H), 4.37 (dd, 1H, J=9.7, 7.1 Hz), 4.02 (m, 1H), 3.89 (s, 3H), 3.88 (s, 3H), 2.94-2.88 (m, 1H), 2.64-2.59 (m, 1H), 2.45 (s, 3H), 2.43 (s, 3H), 2.39-2.28 (m, 2H), 1.99-1.90 (m, 1H), 1.88-1.77 (m, 1H), 1.58-1.52 (m, 1H), 1.40 (m, 4H), 1.25 (m, 2H) 1.11-0.97 (m, 4H).

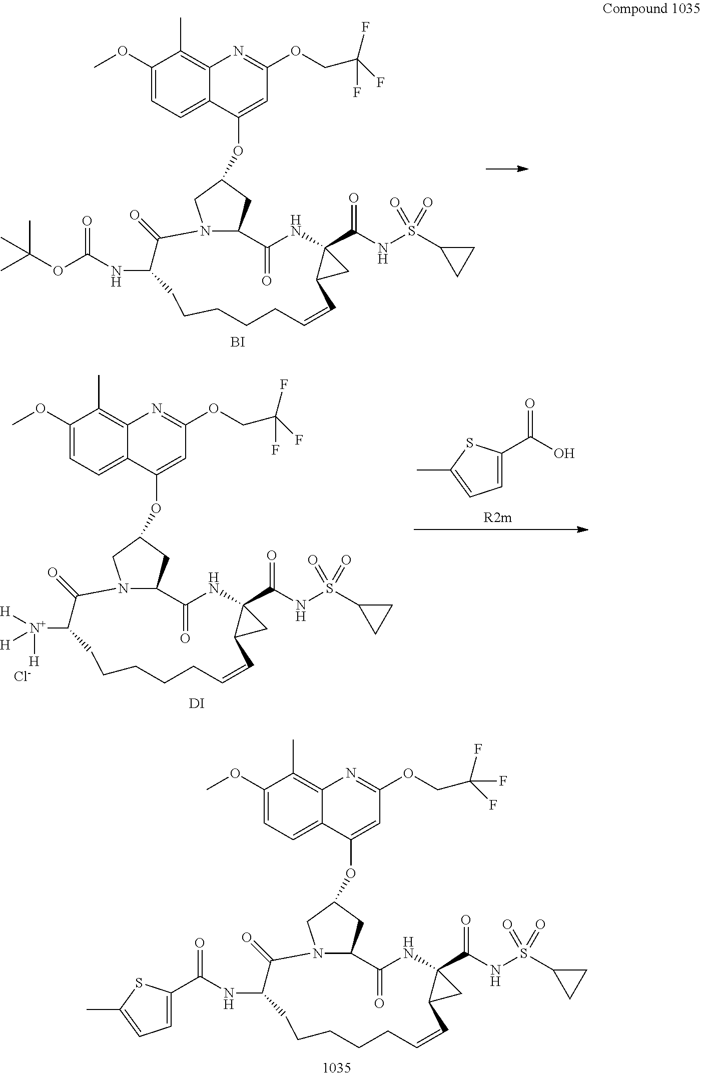

The Boc protected amine macrocycle Bl (40 mg, 0.048 mmol) is charged in a vial, then a 4 M solution of HCl in dioxane (3 mL, 12 mmol) is added. The solution is stirred at RT for 1 h. The solution is evaporated to dryness to give Dl.

The 5-methyl-2-thiophene carboxylic acid R2m (8.145 mg, 0.057 mmol, 1.2 equiv) is dissolved in DMF (3 mL), then TEA (27 μL, 0.191 mmol, 4 equiv) followed by HATU (21.8 mg, 0.057 mmol, 1.2 equiv). The reaction mixture is stirred for 15 mins, after which the amine hydrochloride Dl is added in DMF (1 mL). The solution is stirred at RT for 16 h. The resulting solution is filtered through a Millex filter and purified by prep HPLC (Sunfire column, 0.1% TFA). The pure fractions are combined, concentrated, frozen and lyophilized to provide compound 1035.

FIA M.S. (electrospray): 860.3 (M−H)⁻, 862.2 (M+H)⁺

Retention time (min)=7.1 min

¹H NMR (400 MHz, DMSO-d₆): δ 11.07 (s, 1H), 8.77 (s, 1H), 8.60 (d, 1H, J=6.7 Hz), 7.99 (d, 1H, J=9 Hz), 7.68 (d, 1H, J=3.3 Hz), 7.14 (d, 1H, J=9.3 Hz), 6.82 (d, 1H, J=2.7 Hz), 6.61 (s, 1H), 5.65-5.58 (m 1H), 5.50 (m, 1H), 5.24-5.06 (m, 3H), 4.74-4.71 (m, 2H), 4.48-4.43 (m, 2H), 4.34-4.29 (m, 1H), 3.95 (m, 1H), 3.91 (s, 3H), 2.93-2.87 (m, 1H), 2.68-2.59 (m, 2H), 2.45 (s, 3H), 2.41-2.29 (m, 3H), 2.02-1.97 (m, 1H), 1.75-1.70 (m, 1H), 1.58-1.38 (m, 7H), 1.26-1.23 (m, 2H), 1.10-0.97 (m, 4H).

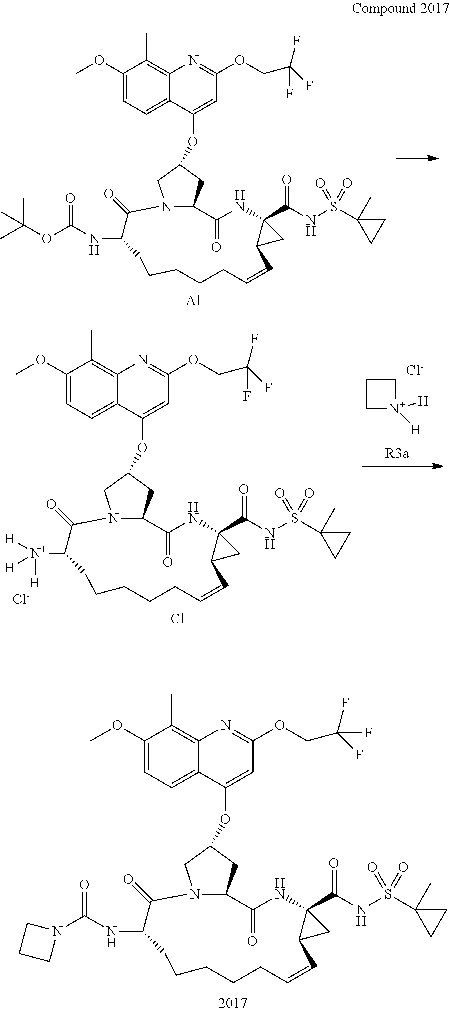

The Boc protected macrocycle Al (65 mg, 0.076 mmol) is dissolved in 4 N HCl/dioxane (3.0 mL) and the reaction is stirred for 1 h at RT to give the amine hydrochloride Cl. To the crude deprotected macrocyclic amine Cl (64 mg, 0.76 mmol) in DCM (1.9 mL) is added carbonyl diimidazole (15 mg, 0.092 mmol) and TEA (0.032 mL, 0.23 mmol). The reaction mixture is stirred at RT overnight. The crude reaction mixture is concentrated in vacuo. Azetidine hydrochloride salt R3a (11 mg, 0.114 mmol) in solution in DMF (3 mL) is added to the concentrated product from above. The reaction mixture is stirred at RT for 4 h and then at 70° C. overnight. The resulting solution is purified by prep HPLC (Sunfire column, ammonium formate and MeOH). The pure fractions are combined concentrated, frozen and lyophilized to provide compound 2017.

FIA M.S. (electrospray): 835.4 (M+H)⁺

Retention time (min)=6.8 min

¹H NMR (400 MHz, DMSO-d₆): δ 10.85 (s, 1H), 8.88 (s, 1H), 7.96 (d, 1H, J=9.0 Hz), 7.19 (d, 1H, J=9.4 Hz), 6.59 (s, 1H), 6.43 (d, 1H, J=7.4 Hz), 5.64-5.57 (m, 1H), 5.47 (m, 1H), 5.20-2.09 (m, 3H), 5.04 (dd, 1H, J=9.7, 9.0 Hz), 4.59 (d, 1H, J=10.9 Hz), 4.36 (dd, 1H, J=10.2, 7.0 Hz), 4.19-4.15 (m, 1H), 3.96-3.86 (m, 1H), 3.91 (s, 3H), 3.72-3.63 (m, 2H), 2.67-2.65 (m, 1H), 2.95 (s, 3H), 2.41-2.28 (m, 3H), 2.07-1.99 (m, 2H), 1.85-1.82 (m, 1H), 1.75-1.71 (m, 1H), 1.57 (dd, 1H, J=8.2, 5.1 Hz), 1.52-1.48 (m, 1H), 1.42-1.19 (m, 10H), 1.38 (s, 3H), 0.92-0.83 (m, 2H).

### Synthesis of Compounds from Tables 1 & 2 Using Intermediates Am and Bm

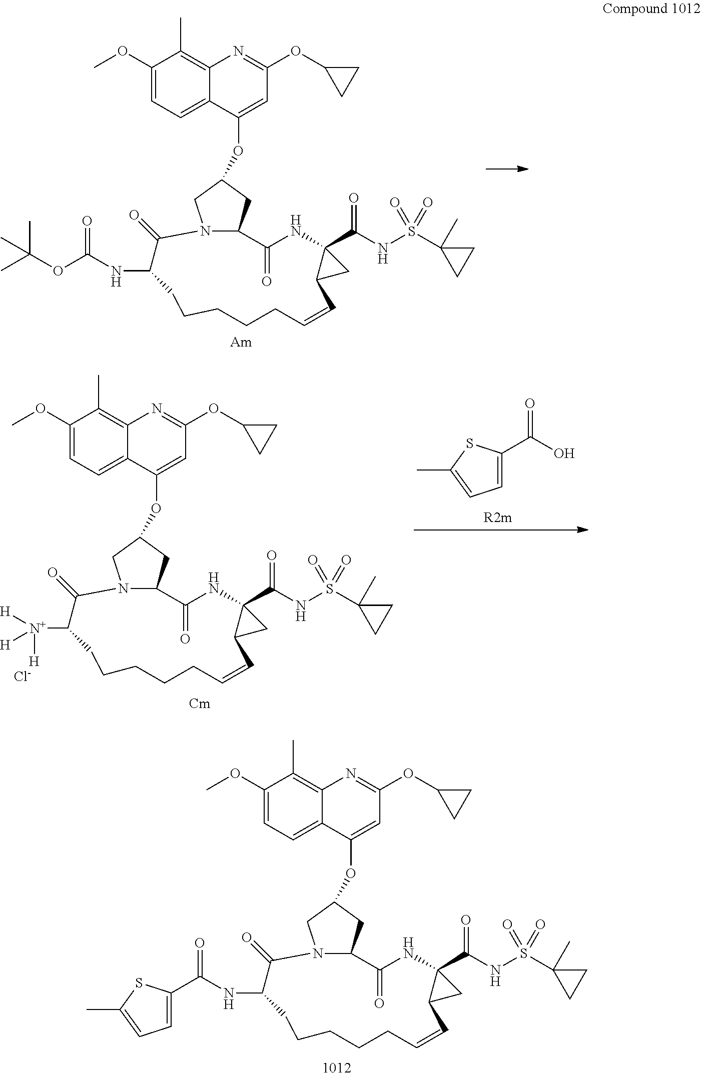

Intermediate Cm is prepared by dissolving Am in 4 N HCl/dioxane, stirring for 1 h and concentrating in vacuo.

5-methyl-2-thiophene carboxylic Acid R2m (11.4 mg, 0.080 mmol) is dissolved in DCM (2 mL), then TEA (47 μL, 0.335 mmol) is added followed by TBTU (24.7 mg, 0.077 mmol). The solution is stirred for 15 mins, after which the amine hydrochloride Cm (50 mg, 0.067 mmol) is added. The solution is stirred at RT for 16 h, concentrated and then the residual is dissolved in DMSO. The resulting solution is filtered through a Millex filter and purified by prep HPLC (Sunfire column, ammonium formate and MeOH). The pure fractions are combined, concentrated, redissolved in MeCN and water, frozen and lyophilized to provide compound 1012.

FIA M.S. (electrospray): 834.3 (M+H)⁺

Retention time (min)=6.0 min

¹H NMR (400 MHz, DMSO-d₆): δ 10.79 (s, 1H), 8.84 (s, 1H), 8.57-8.50 (m, 1 Hz), 7.93 (d, 1H, J=9.2 Hz), 7.68 (d, 1H, J=3.5 Hz), 7.08 (d, 1H, J=9.2 Hz), 6.82 (dd, 1H, J=3.7, 3.5 Hz), 6.37 (s, 1H), 5.66-5.52 (m, 1H), 5.45-4.40 (m, 1H), 5.15-5.04 (m, 1H), 4.69-4.62 (m, 1H), 4.53-4.48 (m, 2H), 4.38 (dd, 1H, J=7.4, 7.2 Hz), 4.03-3.95 (m, 1H), 3.89 (s, 3H), 2.63-2.60 (m, 1H), 2.48 (s, 3H), 2.46 (s, 3H), 2.41-2.30 (m, 2H), 2.05-1.95 (m, 1H), 1.87-1.69 (m, 1H), 1.58-1.19 (m, 15H), 0.85-0.79 (m, 4H), 0.75-0.68 (m, 2H).

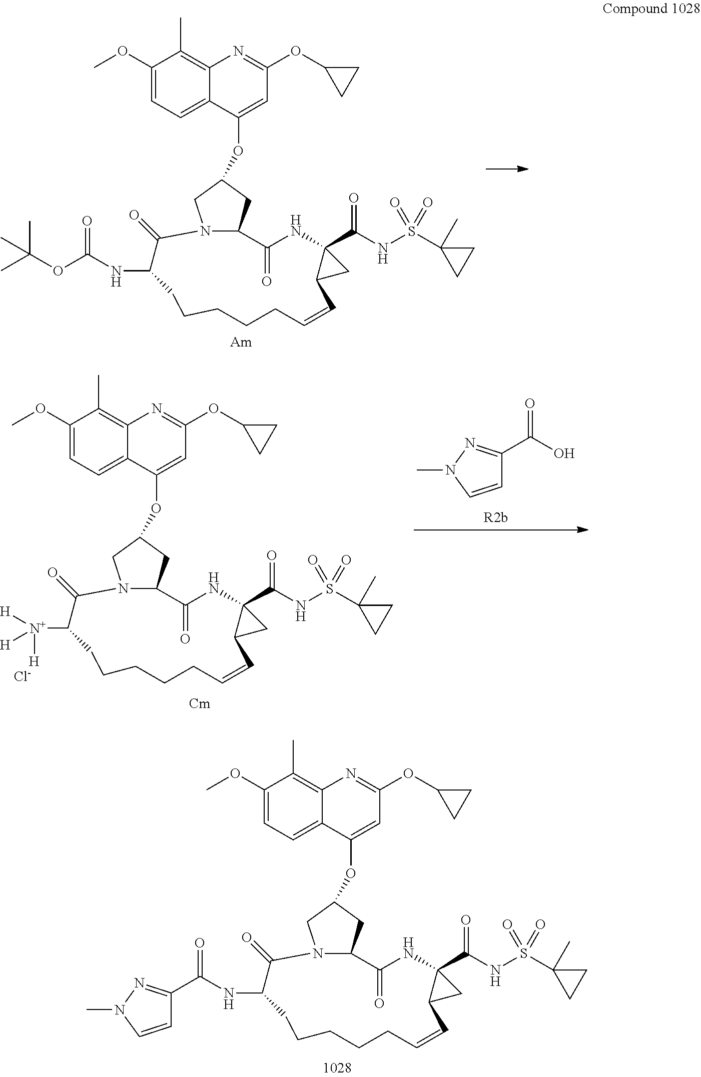

C2-Ocyclopropyl Boc protected amine macrocycle Am (75 mg, 0.093 mmol) is charged in a vial, then a 4 M solution of HCl in dioxane (1 mL, 4 mmol) is added. The solution is stirred at RT for 1.5 h, after which a precipitate forms. The solution is evaporated to dryness. 1-methyl-1H-pyrazole-3-carboxylic acid R2b (14.0 mg, 0.111 mmol) is dissolved in DCM (2 mL), then TEA (51.6 μL, 0.370 mmol) is added followed by TBTU (35.7 mg, 0.111 mmol). The solution is stirred for 15 mins, after which it is added to the amine hydrochloride in solution in DCM (1 mL). The solution is stirred at RT for 16 h. The reaction is not complete so additional 1-methyl-1H pyrazole-3-carboxylic Acid R2b (3.5 mg, 0.028 mmol), TEA (13.0 μL, 0.092 mmol) followed by TBTU (8.9 mg, 0.028 mmol) are added. The solution is stirred at RT for 5 h, concentrated and then the residual is dissolved in DMSO. The resulting solution is filtered through a Millex filter and purified by prep HPLC (Sunfire column, ammonium formate and MeOH). The pure fractions are combined, concentrated, redissolved in MeCN and water, frozen and lyophilized to provide compound 1028.

FIA M.S. (electrospray): 818.4 (M+H)⁺

Retention time (min)=5.5 min

¹H NMR (400 MHz, DMSO-d₆): δ 10.80 (bs, 1H), 8.93 (s, 1H), 7.84 (d, 1H, J=8.9 Hz), 7.76 (d, 1H, J=2.4 Hz), 6.74-7.70 (m, 1 Hz), 7.10 (d, 1H, J=9.2 Hz), 6.59 (d, 1H, J=2.1 Hz), 6.38 (s, 1H), 5.68-5.54 (m, 1H), 5.47-5.40 (m, 1H), 5.10-5.00 (m, 1H), 4.68-4.56 (m 1H), 4.50 (qn, 1H, J=3.2 Hz) 4.47-4.38 (m, 2H), 4.08-3.96 (m, 1H), 3.89 (s, 3H), 3.88 (s, 3H), 2.66-2.58 (m, 1H), 2.47 (s, 3H), 2.40-2.28 (m, 2H), 2.01-1.74 (m, 2H), 1.64-1.49 (m, 3H), 1.48-1.13 (m, 12H), 0.93-0.79 (m, 4H), 0.76-0.69 (m, 2H).

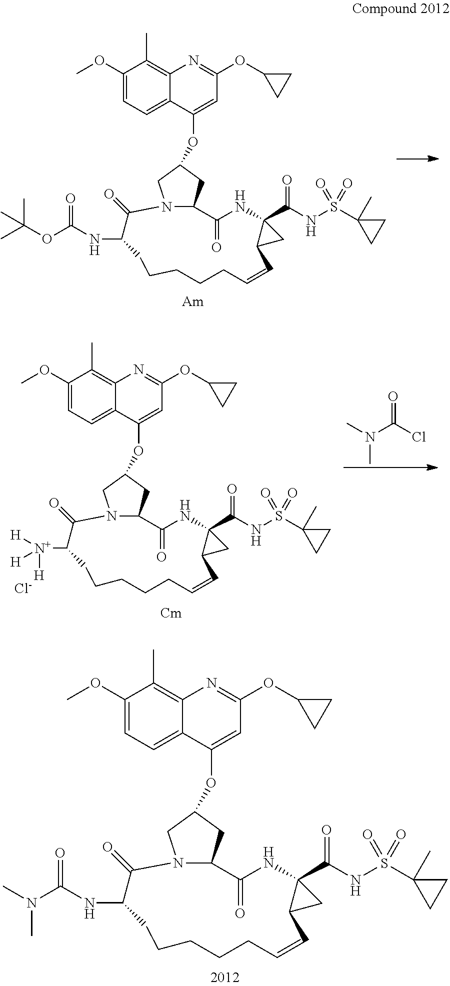

To the crude deprotected macrocyclic amine Cm (60 mg, 0.080 mmol) in DCM (2 mL) is added TEA (0.045 mL, 0.322 mmol) followed by dimethylcarbamyl chloride (14.8 μL, 0.161 mmol). The resulting solution is stirred at RT for 16 h, concentrated and then the residual is dissolved in DMSO. The resulting solution is filtered through a Millex filter and purified by prep HPLC (Sunfire column, ammonium formate and MeOH). The pure fractions are combined, concentrated, redissolved in MeCN and water, frozen and lyophilized to provide compound 2012.

FIA M.S. (electrospray): 781.3 (M+H)⁺

Retention time (min)=5.3 min

¹H NMR (400 MHz, DMSO-d₆): δ 10.81 (bs, 1H), 8.85 (bs, 1H), 7.98 (d, 1H, J=9.0 Hz), 7.10 (d, 1H, J=9.2 Hz), 6.36 (s, 1H), 6.25 (d, 1H, J=7.0 Hz), 5.65-5.55 (m, 1H), 5.42-5.37 (m, 1H), 5.09-5.01 (m, 1H), 4.65 (d, 1H, J=11.5),4.50 (tt, 1H, J=6.2, 6.2 Hz), 4.37 (dd, 1H, J=7.0, 7.0 Hz), 4.22-4.13 (m, 1H), 3.94-3.87 (m, 1H), 3.89 (s, 3H), 2.73 (s, 6H), 2.58-2.52 (m, 2H), 2.47 (s, 3H), 2.34-2.27 (m, 2H), 188-1.68 (m, 2H), 1.59. 1.56 (m, 1H), 1.52-1.47 (m, 1H), 1.43-1.34 (m, 9H), 1.30-1.17 (m, 3H), 0.89-0.79 (m, 4H), 0.74-0.69 (m, 2H)

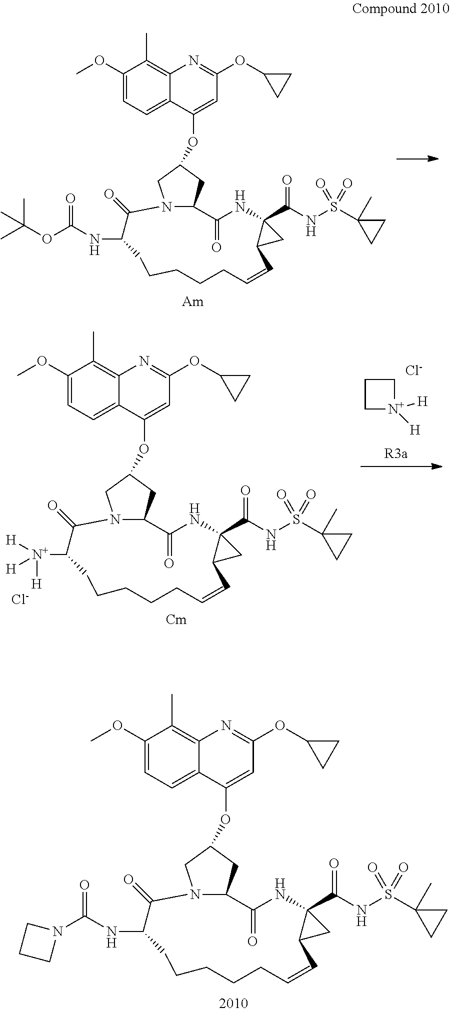

To the crude deprotected macrocyclic amine Cm (70 mg, 0.094 mmol) in DMF (1 mL) is added carbonyl diimidazole (18.2 mg, 0.113 mmol) and TEA (105 μl, 0.750 mmol). The reaction mixture is stirred at RT for 60 min. Azetidine hydrochloride salt (43.7 mg, 0.469 mmol) in solution in DMF (1 mL) is added. The reaction mixture is stirred at RT for 16 h. The reaction is incomplete so azetidine hydrochloride salt (14.5 mg, 0.156 mmol) and TEA (26 μl, 0.187 mmol) is added. The resulting solution is stirred at RT for 24 h and then concentrated. To the solid is added DMSO and a few drops of acetic acid. The resulting solution is filtered through a Millex filter and purified by prep HPLC (Sunfire column, ammonium formate and MeOH). The pure fractions are combined concentrated, frozen and lyophilized to provide compound 2010.

FIA M.S. (electrospray): 793.4 (M+H)⁺

Retention time (min)=5.3 min

¹H NMR (400 MHz, DMSO-d₆): δ 10.99 (bs, 1H), 8.73 (bs, 1H), 7.90 (d, 1H, J=9.0

Hz), 7.15 (d, 1H, J=9.1 Hz), 6.36 (s, 1H), 6.33-6.28 (m 1H), 5.58-5.50 (m, 1H), 5.42-5.47 (m, 1H), 5.16-5.07 (m, 1H), 4.50 (tt, 1H, J=6.3, 6.2 Hz), 4.47-4.43 (m, 1H), 4.39 (dd, 1H, J=7.4, 7.0 Hz), 4.27-4.19 (m, 1H), 4.00-3.92 (m, 1H), 3.90 (s, 3H), 3.67 (t, 4H, J=7.6 Hz), 2.61-2.55 (m, 3H), 2.47 (s, 3H), 2.02 (qn, 2H, J=7.6 Hz), 1.89-1.77 (m, 2H), 1.59-1.53 (m, 2H), 1.45-1.13 (m, 13H), 0.95-0.76 (m, 4H), 0.745-0.69 (m, 2H).

### Synthesis of Compounds in Table 1 Using Scheme 2

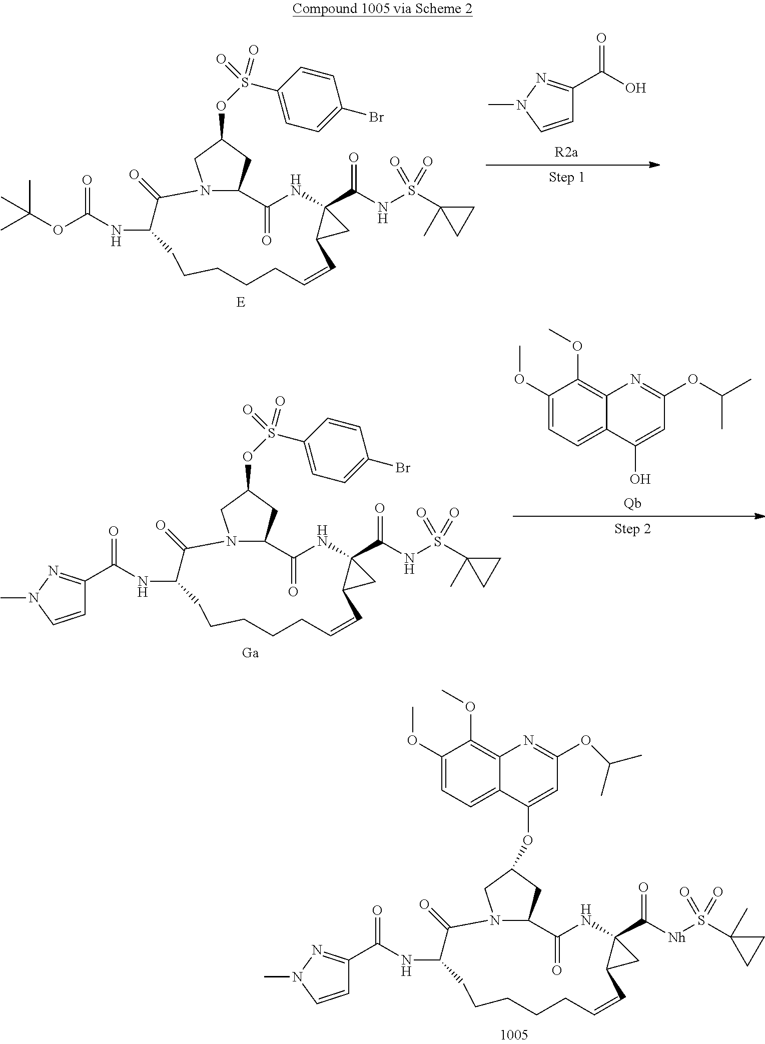

Step 1: Deprotection and Coupling of Intermediate E with R2a

Macrocyclic brosylate E (1.01 g, 1.26 mmol) is dissolved in 4 N HCl/dioxane (5 mL) and stirred for 45 mins, then concentrated in vacuo. The residue is redissolved in DCM (10 mL), TEA (0.90 mL, 6.5 mmol), TBTU (485 mg, 1.51 mmol) and 1-methyl-1H-pyrazole-3-carboxylic acid R2a (206.5 mg, 1.64 mmol) are added and the reaction is stirred for 4 h at RT. The reaction mixture is concentrated in vacuo and the resulting material purified by flash chromatography using DCM/MeOH (0-10%). The pure fractions are combined and concentrated in vacuo to give intermediate Ga.

Step 2: Brosylate Displacement with Hydroxy Quinoline Qb

Intermediate Ga (64 mg, 0.079 mmol) and hydroxy quinoline Qb (23 mg, 0.087 mmol) are dissolved in NMP (2 mL). Cs₂CO₃ (77 mg, 0.237 mmol) is added and the mixture is heated to 80° C. for 16 h. The material is purified by prep HPLC (ammonium formate/MeOH). The product containing fractions are combined, concentrated in vacuo, redissolved in MeCN/H₂O, frozen and lyophilized to give compound 1005.

FIA M.S. (electrospray): 836.5 (M+H)⁺

Retention time (min)=5.2 min

¹H NMR (400 MHz, DMSO-d₆): δ 11.0 (br.s, 1H), 8.61 (br.s, 1H), 7.70 (d, 1H, J=2.4 Hz), 7.68 (d, 1H, J=4.0 Hz), 7.62 (d, 1H, J=9.0 Hz), 6.30 (s, 1H), 5.45 (S, 1H, J=5.9 Hz) 5.34-5.38 (m, 1H), 5.15 (br.s, 1H), 4.67-4.55 (m, 1H), 4.37-4.33 (m, 2H), 4.07-3.98 (m, 1H), 3.84 (s, 3H), 3.82 (s, 3H), 3.80 (s, 3H), 2.36-2.24 (m, 1H), 2.13-2.09 (m, 1H), 1.92-1.83 (m, 3H), 1.64-1.54 (m, 1H), 1.50-1.46 (m, 3H), 1.32-1.29 (m, 14H), 1.23-1.12 (m, 6H), 0.75-0.57 (m, 2H).

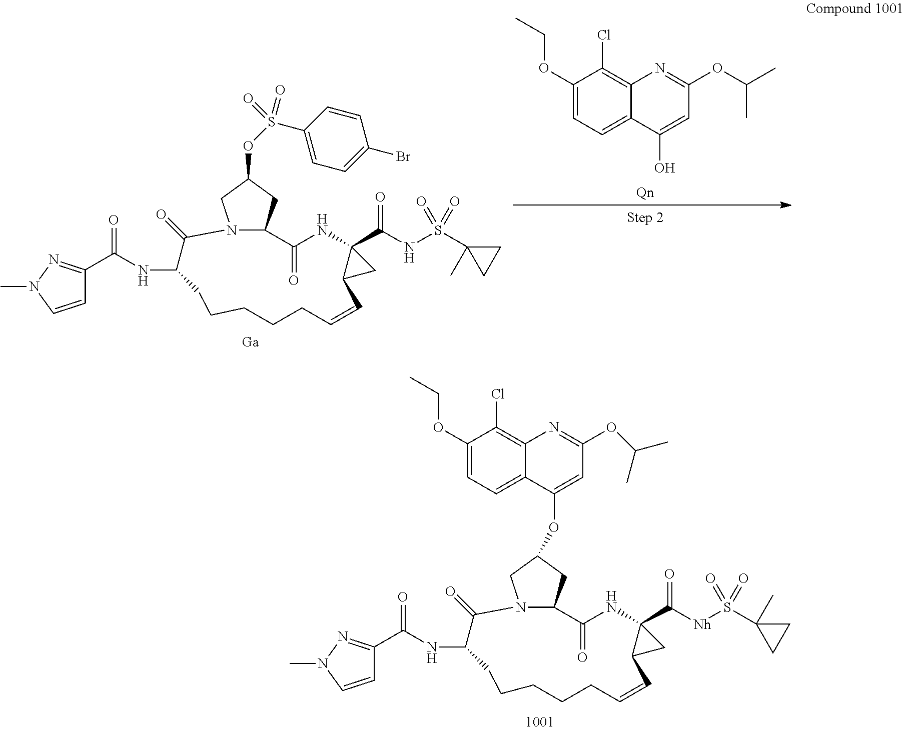

The brosylated macrocycle component Ga (75 mg; 0.093 mmol) is dissolved in NMP (2 mL) and the quinoline Qn (28.7 mg; 0.102 mmol) and cesium carbonate (90.5 mg; 0.278 mmol) are added and the mixture is heated at 80° C. overnight. The mixture is dissolved in MeOH, filtered through a Millex filter and purified by preparative HPLC (Sunfire column,10 mM Ammonium Formate pH3.8: MeOH) The pure fractions aere combined, concentrated, frozen and lyophilized to provide compound 1001.

FIA M.S. (electrospray): 852.4 (M−H)−, 854.3 (M+H)⁺

Retention time (min)=6.6 min

¹H NMR (400 MHz, DMSO-d₆): δ 10.82 (bs, 1H), 8.91 (s, 1H), 7.91 (d, 1H, J=9 Hz), 7.78 (d, 1H, J=7.0 Hz), 7.76 (d, 1H, J=2.3 Hz), 7.18 (d, 1H, J=9.0 Hz), 6.57 (d, 1H, J=2.3 Hz), 6.46 (s, 1H), 5.66-5.49 (m, 2H), 5.47 (bs, 1H), 5.17-5.02 (m, 1H), 4.65-4.47 (m, 2H), 4.47-4.37 (m, 1H), 4.26-4.18 (m, 2H), 4.11-3.97 (m, 1H), 3.88 (s, 3H), 2.66-2.56 (m, 1H), 2.41-2.23 (m, 2H), 2.00-1.89 (m, 1H), 1.86-1.74 (m, 1H), 1.61-1.51 (m, 3H), 1.51-1.16 (m, 21H), 0.93-0.79 (m, 2H).

### Synthesis of Compounds 2016 and 2019 Using Scheme 5

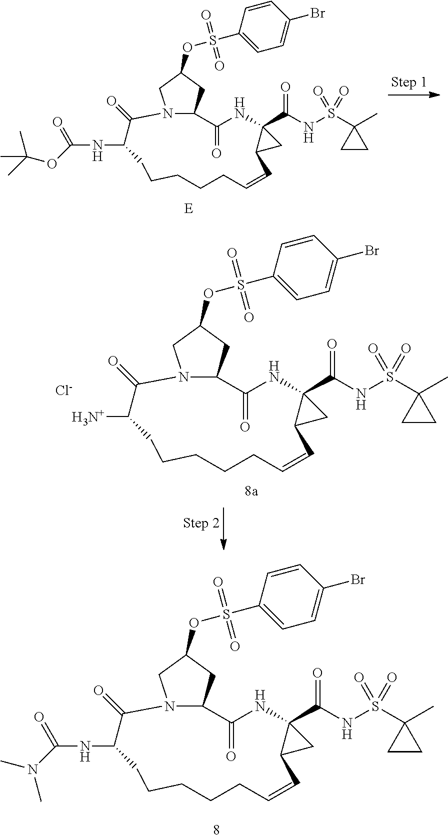

### Step 1: Deprotection of Intermediate E

Intermediate E (700 mg, 0.873 mmol) is dissolved in 4 N HCl/dioxane (4 mL) and stirred for 1 h at RT then concentrated in vacuo to provide crude amine hydrochloride 8a which is used as such.

### Step 2: Formation of 8b

To the amine hydrochloride 8a (183 mg, 0.25 mmol) in DCM (2.0 mL) is added carbonyl diimidazole (60.6 mg, 0.375 mmol) and DIPEA (0.043 mL, 0.249 mmol). The reaction mixture is stirred for 1 h at RT. Dimethylamine (0.374 mL of 2.0 M solution in THF, 0.747 mmol) is added and the reaction is capped and stirred overnight at RT. The reaction mixture is concentrated in vacuo and the crude 8b (185 mg, 100%) is used as such in subsequent reactions.

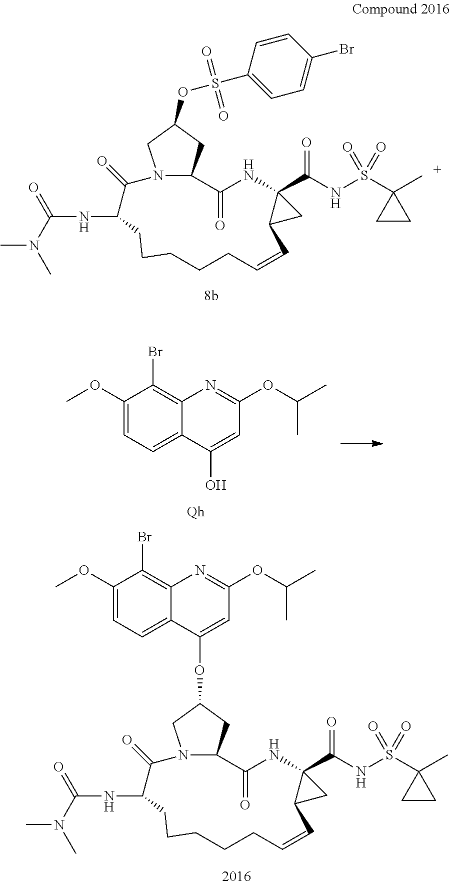

To the crude macrocyclic brosylate 8b (96.3 mg, 0.125 mmol) in NMP (2 mL) is added the quinoline Qh (66.1 mg, 0.212 mmol) and cesium carbonate (81.2 mg, 0.249 mmol). The solution is heated at 80° C. overnight. The resulting solution is filtered through a Millex filter and purified by prep HPLC. The pure fractions are combined, concentrated, frozen and lyophilized to provide compound 2016.

FIA M.S. (electrospray): 847.3 (M+H)⁺

Retention time (min)=6.6 min

¹H NMR (400 MHz, DMSO-d₆): δ 10.77 (s, 1H), 8.79 (s, 1H), 8.05 (d, 1H, J=9.0 Hz), 7.10 (d, 1H, J=9.0 Hz), 6.38 (s, 1H), 6.25 (d, 1H, J=6.7 Hz), 5.57-5.51 (m, 1H), 5.49 (S, 1H, J=5.9 Hz), 5.39-5.35 (m, 1H), 5.01 (dd, 1H, J=12.0, 8.1 Hz), 4.65 (d, 1H, J=11.7 Hz), 4.29 (dd, 1H, J=10.1, 7.1 Hz), 4.08-4.03 (m, 1H), 3.89 (s, 3H), 3.84-3.78 (m. 1H), 2.65 (s, 6H), 2.57-2.51 (m, 2H), 2.31-2.21 (m, 2H), 1.83-1.77 (m, 1H), 1.68-1.59 (m, 1H), 1.51-1.41 (m, 2H), 1.36-1.27 (m, 14H), 1.24-1.15 (m, 4H), 0.84-0.76 (m, 2H).

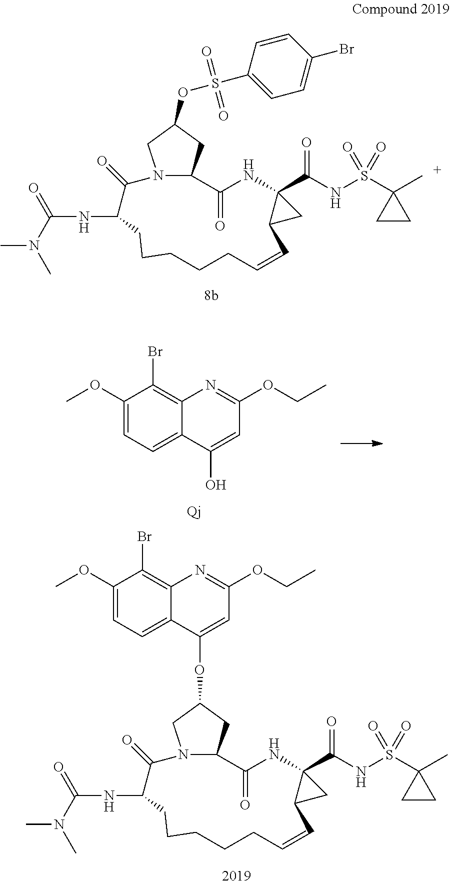

To the crude macrocyclic brosylate 8b (96.3 mg, 0.125 mmol) in NMP (2 mL) is added the quinoline Qj (40.9 mg, 0.137 mmol) and cesium carbonate (81.2 mg, 0.249 mmol). The solution is heated at 80° C. overnight. The resulting solution is filtered through a Millex filter and purified by prepHPLC. The pure fractions are combined, concentrated, frozen and lyophilized to provide compound 2019.

FIA M.S. (electrospray): 833.3 (M+H)⁺

Retention time (min)=6.5 min

¹H NMR (400 MHz, DMSO-d₆): δ 10.79 (s, 1H), 8.79 (s, 1H), 8.04 (d, 1H, J=9.0 Hz), 7.12 (d, 1H, J=9.4 Hz), 6.42 (s, 1H), 6.25-6.16 (m, 1H), 5.59-5.45 (m, 1H), 5.40-5.34 (m, 1H), 5.10-4.94 (m, 1H), 4.68-4.56 (m, 1H), 4.47 (q, 2H, J=6.9 Hz), 4.31 (dd, 1H, J=9.2, 7.2 Hz), 4.14-4.04 (m, 1H), 3.89 (s, 3H), 3.86-3.76 (m. 1H), 2.63 (s, 6H), 2.26-2.25 (m, 2H), 1.84-1.61 (m, 2H), 1.51-1.43 (m, 2H), 1.35-1.30 (m, 13H), 1.21-1.08 (m, 4H), 0.84-0.71 (m, 2H).

### HCV Replicon RNA Replication Assay (NS3 Protease Variants)

HCV replicons:

HCVPV1a and HCVPV1b are subgenomic replicons. HCVPV1a is genotype 1a (strain H77); HCVPV1b is genotype b (Con-1), see Science 1999, 285: 110-113. Both subgenomic replicons contain a hybrid HCV-poliovirus (PV) 5′UTR, a modified luciferase reporter gene expressed as a luciferase-FMDV2A-neomycin phosphotransferase gene fusion and a NS2-NS5B subgenomic fragment with its 3′UTR. The replication of both HCV NS2-NS5B subgenomic replicons is enhanced by cell-culture adaptive mutations in the NS3 and the NS4B coding regions for the genotype 1a replicon and in the NS3, NS4A and NS5A coding regions for the genotype 1b, as described below. Stable replicon cell lines are established as described, for example, in Science 1999, 285: 110-113. The amount of luciferase expressed by selected cells directly correlates with the level of HCV replication, as measured by real-time PCR.

SEQ ID NO: 1 is a nucleotide sequence representing the HCV genotype 1a subgenomic fragment NS2-NS3-NS4A-NS4B-NS5A-NS5B. SEQ ID NO: 1 is 6609 bases wherein nucleotide bases 1-651 of SEQ ID NO: 1 encode NS2, nucleotide bases 652-2544 encode NS3, nucleotide bases 2545-2706 encode NS4A, nucleotide bases 2707-3489 encode NS4B, nucleotide bases 3490-4833 encode NSSA, and nucleotide bases 4834-6606 encode NS5B. NS3 resistance mutation R155K is encoded by the codon of bases 1114-1116 of SEQ ID NO: 1. SEQ ID NO: 2 is the corresponding polypeptide. SEQ ID NO: 2 includes adaptive mutations over reference sequence (GenBank accession number AF009606, residues 811 to 3011 where 811 corresponds to residue 2 in SEQ ID NO: 2) in the NS3 and NS4B coding regions, namely at residues 471, 549, 622, 1000 and 1030 of SEQ ID NO: 2. SEQ ID NO: 2 further includes NS3 resistance mutation R155K which is residue 372.

SEQ ID NO: 3 is a nucleotide sequence representing HCV genotype 1b subgenomic fragment NS2-NS3-NS4A-NS4B-NSSA-NS5B. SEQ ID NO: 3 is 6615 bases wherein nucleotide bases 1-651 of SEQ ID NO: 3 encode NS2, nucleotide bases 652-2544 encode NS3, nucleotide bases 2545-2706 encode NS4A, nucleotide bases 2707-3489 encode NS4B, nucleotide bases 3490-4839 encode NSSA, and nucleotide bases 4840-6612 encode NS5B. NS3 resistance mutation D168V is encoded by the codon of bases 1153-1155 of SEQ ID NO: 3. SEQ ID NO: 4 is the corresponding polypeptide. SEQ ID NO: 4 includes adaptive mutations over reference sequence CON-1 (GenBank accession number AJ238799, residues 811 to 3010) in the NS3, NS4A and NSSA coding regions, namely at residues 326, 751, 882, 1184, 1233, 1346 and 1357 of SEQ ID NO: 4. SEQ ID NO: 4 further includes NS3 resistance mutation D168V which is residue 385 of SEQ ID NO: 4.

All amino acid substitutions were generated by site-directed mutagenesis using Quick change (Stratagene, La Jolla, Calif.) according to the manufacturer's instructions.

HCV replicon RNA replication assay: To generate cell lines harboring the replicon containing the NS3 substitutions, Huh-7 cells are electroporated with 1-10 μg of purified in vitro transcripts and stable cell lines are selected in the presence of G418 (0.25 mg/ml).

The stable HCV replicon cells are maintained in Dulbecco's Modified Eagle Medium (DMEM) supplemented with 10% FBS and 0.25 mg/ml G418 (standard medium).

During the assay, DMEM supplemented with 10% FBS, containing 0.5% DMSO and lacking neomycin are used as assay medium.

For the assay, the cell stocks are trypsinized and diluted in assay medium to distribute 70 μl (8,000 ells) in black 96-well plates. The plates are then incubated at 37° until compound addition. The test compound in 100% DMSO is first diluted in assay medium to a final DMSO concentration of 0.5%. Serial dilutions are prepared in assay medium to generate nine-concentration dose response curves. A fixed volume from each well of the compound dilution plate is transferred to a corresponding well of the cell culture plate. The cell culture plate is incubated at 37° C. with 5% CO₂ for 72 hours. Following the 72h incubation period, the medium is aspirated from the 96-well assay plate and a volume of 50 μl of 1× Glo Lysis Buffer (Promega) is added to each well. The luciferase activity is determined using Bright-Glo luciferase substrate (Promega) according to the manufacturers instructions and the luminescence is detected on a Packard Topcount instrument. The luminescence (CPS) in each well of the culture plate is a measure of the amount of HCV RNA replication in the presence of various concentrations of inhibitor. The % inhibition is calculated for each inhibitor concentration and used to determine the concentration that results in 50% inhibition of HCV replication (EC₅₀).

Table 3 shows the EC₅₀ (nM) for the compounds of the invention when tested in the HCV replicon RNA replication assay for the genotype 1a R155K and genotype 1b D168V resistance mutations (using SEQ ID NOS: 1 and 3 respectively).

TABLE 3

| | R155K 1a | D168V 1b |
|---|---|---|
| Cmpd # | EC₅₀ (nM) | EC₅₀ (nM) |
| 1001 | 2.6 | 0.33 |
| 1002 | 2.6 | 3.0 |
| 1003 | 4.3 | 0.29 |
| 1004 | 2.8 | 0.22 |
| 1005 | 2.5 | 2.0 |
| 1006 | 7.0 | 0.33 |
| 1007 | 3.3 | 0.25 |
| 1008 | 2.9 | 0.35 |
| 1009 | 1.9 | 0.49 |
| 1010 | 7.0 | 0.33 |
| 1011 | 8.4 | 0.63 |
| 1012 | 5.6 | 0.26 |
| 1013 | 7.8 | 0.52 |
| 1014 | 8.3 | 0.31 |
| 1015 | 8.2 | 0.57 |
| 1016 | 8.6 | 0.92 |
| 1017 | 9.5 | 1.1 |
| 1018 | 2.4 | 0.14 |
| 1019 | 0.88 | 0.16 |
| 1020 | 3.1 | 0.23 |
| 1021 | 4.3 | 0.24 |
| 1022 | 6.8 | 0.24 |
| 1023 | 8.2 | 0.25 |
| 1024 | 3.9 | 0.31 |
| 1025 | 4.1 | 0.32 |
| 1026 | 3.3 | 0.32 |
| 1027 | 8.4 | 0.35 |
| 1028 | 3.7 | 0.37 |
| 1029 | 6.0 | 0.37 |
| 1030 | 6.3 | 0.38 |
| 1031 | 4.2 | 0.39 |
| 1032 | 7.8 | 0.58 |
| 1033 | 0.77 | 0.58 |
| 1034 | 7.7 | 0.67 |
| 1035 | 8.2 | 0.77 |
| 1036 | 3.2 | 1.3 |
| 1037 | 4.3 | 3.7 |
| 2001 | 2.3 | 0.48 |
| 2002 | 4.7 | 0.41 |
| 2003 | 9.1 | 0.47 |
| 2004 | 8.2 | 1.6 |
| 2005 | 4.8 | 0.82 |
| 2006 | 9.9 | 2.1 |
| 2007 | 9.0 | 0.71 |
| 2008 | 6.7 | 1.5 |
| 2009 | 6.8 | 2.9 |
| 2010 | 6.4 | 0.66 |
| 2011 | 6.1 | 3.9 |
| 2012 | 9.9 | 1.8 |
| 2013 | 6.5 | 3.3 |
| 2014 | 8.4 | 0.53 |
| 2015 | 5.4 | 0.67 |
| 2016 | 2.0 | 1.0 |
| 2017 | 6.3 | 1.1 |
| 2018 | 5.7 | 1.2 |
| 2019 | 5.2 | 1.4 |
| 2020 | 4.0 | 1.4 |
| 2021 | 3.0 | 1.6 |
| 2022 | 4.9 | 1.8 |

Table 4 shows the EC₅₀ (nM) of three compounds that belong to the second class currently in clinical trials, namely MK-7009, ITMN-191 and TMC435, when tested in the HCV replicon RNA replication assay described above for activity in each of genotype 1a R155K and genotype 1b D168V resistance mutations using SEQ ID NOS: 1 and 3 respectively.

TABLE 4

| | R155K 1a | D168V 1b |
|---|---|---|
| Compound | Ec₅₀ (nM) | Ec₅₀ (nM) |
| 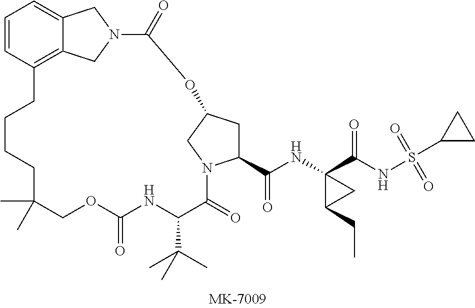 | 1200 | 4400 |
| 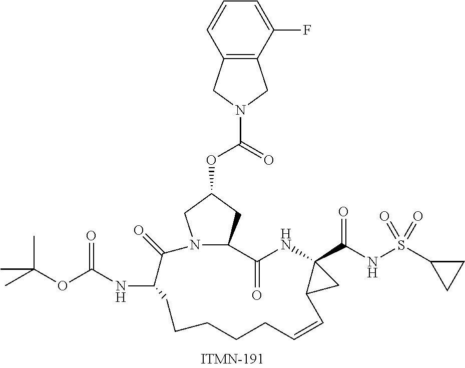 | 690 | 120 |
| 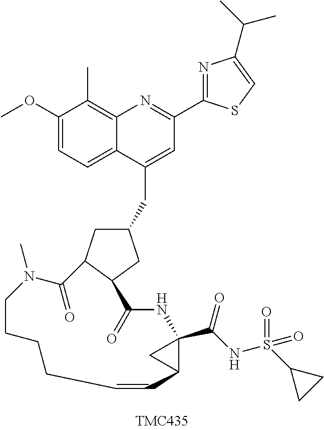 | 300 | 4400 |

Each reference, including all patents, patent applications, and publications cited in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application.

What is claimed is:

1. A compound of Formula (I) or a racemate, diastereoisomer, optical isomer or salt thereof: 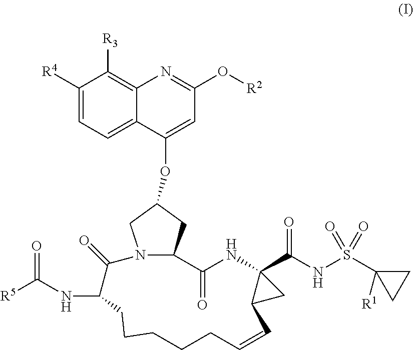
wherein:
R¹ is H or (C₁₋₆)alkyl;
R² is (C₁₋₆)alkyl optionally substituted 1-3 times with halo or (C₃₋₇)cycloalkyl;
R³ is (C₁₋₆)alkyl, —O—(C₁₋₆)alkyl or halo;
R⁴ is —O—(C₁₋₆)alkyl, —OH or halo;
R⁵ is Het optionally substituted 1-3 times with (C₁₋₆)alkyl, —(C₁₋₆)alkyl-C(═O)—N((C₁₋₆)alkyl)₂, —(C₁₋₆)alkyl-O—(C₁₋₆)alkyl or (C₁₋₆)haloalkyl; or
R⁵ is —N(R^{A})(R^{B}) wherein R^{A} and R^{B} are (C₁₋₆)alkyl or R^{A} and R^{B} are linked together with the N to which they are attached to form a 4- to 7-membered saturated ring, wherein said ring is optionally substituted 1-3 times with (C₁₋₆)alkyl, —O—-(C₁₋₆)alkyl, —OH or halo.

2. A compound of Formula (I) or a racemate, diastereoisomer, optical isomer or salt thereof: 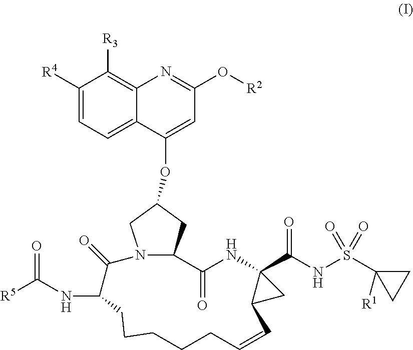
wherein:
R¹ is H or (C₁₋₆)alkyl;
R² is (C₁₋₆)alkyl optionally substituted 1-3 times with halo or (C₃₋₇)cycloalkyl;
R³ is (C₁₋₆)alkyl, —O—(C₁₋₆)alkyl or halo;
R⁴ is —O—(C₁₋₆)alkyl, —OH or halo;
R⁵ is Het optionally substituted 1-3 times with (C₁₋₆)alkyl, (C₁₋₆)alkyl-C(═O)—N((C₁₋₆)alkyl)₂, —(C₁₋₆)alkyl-O—(C₁₋₆)alkyl or (C₁₋₆)haloalkyl; or
R⁵ is —N(R^{A})(R^{B}) wherein R^{A} and R^{B} are (C₁₋₆)alkyl or R^{A} and R^{B} are linked together with the N to which they are attached to form a 4- to 7-membered saturated ring, wherein said ring is optionally substituted 1-3 times with (C₁₋₆)alkyl, —O—(C₁₋₆)alkyl, —OH or halo;
with the proviso that the following compounds are excluded:
(2R,6S,12Z,13aS,14aR,16aS)-6-({[1-(2,2-difluoroethyl)-1H-pyrazol-3-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-6-({[1-(2-methoxyethyl)-1H-pyrazol-3-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[7-chloro-8-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-6-({[1-(2-fluoroethyl)-1H-pyrazol-3-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[7-methoxy-8-methyl-2-(propan-2-yloxy)quinolin-4-yl]oxy}-N-[(1-methylcyclopropyl)sulfonyl]-5,16-dioxo-6-({[1-(2,2,2-trifluoroethyl)-1H-pyrazol-3-yl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[7-methoxy-8-methyl-2-(propan-2-yloxy)quinolin-4-yl]oxy}-N-[(1-methylcyclopropyl)sulfonyl]-6-{[(2-methyl-2H-1,2,3-thazol-4-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[7-methoxy-8-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[7-fluoro-8-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)—N-(cyclopropylsulfonyl)-2-{[7-methoxy-8-methyl-2-(propan-2-yloxy)quinolin-4-yl]oxy}-6-({[5-(methoxymethyl)thiophen-2-yl]carbonyl}amino)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-6-({[2-(2,2-difluoroethyl)-2H-1,2,3-thazol-4-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)—N-(cyclopropylsulfonyl)-6-({[1-(2,2-difluoroethyl)-1H-pyrazol-3-yl]carbonyl}amino)-2-{[7-methoxy-8-methyl-2-(propan-2-yloxy)quinolin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)—N-(cyclopropylsulfonyl)-2-{[7-methoxy-8-methyl-2-(propan-2-yloxy)quinolin-4-yl]oxy}-5,16-dioxo-6-({[1-(2,2,2-trifluoroethyl)-1H-pyrazol-3-yl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[7-fluoro-8-methoxy-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)—N-(cyclopropylsulfonyl)-6-({[5-(2-hydroxypropan-2-yl)thiophen-2-yl]carbonyl}amino)-2-{[7-methoxy-8-methyl-2-(propan-2-yloxy)quinolin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[7-hydroxy-8-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)—N-(cyclopropylsulfonyl)-2-{[7-fluoro-8-methoxy-2-(propan-2-yloxy)quinolin-4-yl]oxy}-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-6-[({1-[2-(dimethylamino)-2-oxoethyl]-1H-pyrazol-3-yl}carbonyl)amino]-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)—N-(cyclopropylsulfonyl)-2-{[7-methoxy-8-methyl-2-(propan-2-yloxy)quinolin-4-yl]oxy}-6-[({1-[2-(methylamino)-2-oxoethyl]-1H-pyrazol-3-yl}carbonyl)amino]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[7-methoxy-8-methyl-2-(propan-2-yloxy)quinolin-4-yl]oxy}-6-[({1-[2-(methylamino)-2-oxoethyl]-1H-pyrazol-3-yl}carbonyl)amino]-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-6-({[5-(2-hydroxypropan-2-yl)thiophen-2-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-6-({[1-(2-fluoroethyl)-1H-pyrazol-3-yl]carbonyl}amino)-2-{[7-fluoro-8-methoxy-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-6-({[1-(2,2-difluoroethyl)-1H-pyrazol-3-yl]carbonyl}amino)-2-{[7-fluoro-8-methoxy-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[7-fluoro-8-methoxy-2-(propan-2-yloxy)quinolin-4-yl]oxy}-N-[(1-methylcyclopropyl)sulfonyl]-5,16-dioxo-6-({[1-(propan-2-yl)-1H-pyrazol-3-yl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[8-bromo-7-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[7,8-dichloro-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[7-fluoro-8-methoxy-2-(propan-2-yloxy)quinolin-4-yl]oxy}-N-[(1-methylcyclopropyl)sulfonyl]-5,16-dioxo-6-({[1-(2,2,2-trifluoroethyl)-1H-pyrazol-3-yl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-6-({[1-difluoromethyl)-1H-pyrazol-3-yl]carbonyl}amino)-2-{[7-fluoro-8-methoxy-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,12Z,13aS,14aR,16aS)-2-{[7-fluoro-8-methoxy-2-(propan-2-yloxy)quinolin-4-yl]oxy}-N-[(1-methylcyclopropyl)sulfonyl]-6-{[(5-methylthiophen-2-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide; and
(2R,6S,12Z,13aS,14aR,16aS)-2-{[7-methoxy-8-methyl-2-(2,2,2-trifluoroethoxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide.

3. The compound according to claim 1 or 2, or a pharmaceutically acceptable salt thereof, wherein R¹ is H or (C₁₋₃)alkyl.

4. The compound according to claim 1 or 2, or a pharmaceutically acceptable salt thereof, wherein R² is (C₁₋₃)alkyl optionally substituted 1-3 times with halo or (C₃₋₄)cycloalkyl.

5. The compound according to claim 4, or a pharmaceutically acceptable salt thereof, wherein R² is

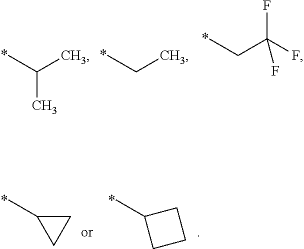

6. The compound according to claim 1 or 2, or a pharmaceutically acceptable salt thereof, wherein R³ is CH₃, —OCH₃, Cl, Br or F.

7. The compound according to claim 1 or 2, or a pharmaceutically acceptable salt thereof, wherein R⁴ is —OCH₃, —OCH₂CH₃, —OH, F or Cl.

8. The compound according to claim 1 or 2, or a pharmaceutically acceptable salt thereof, wherein R⁵ is a 5-membered aromatic Het optionally substituted 1-3 times with (C₁₋₄)alkyl, (C₁₋₃)alkyl-C(═O)—N((C₁₋₃)alkyl)₂, —(C₁₋₃)alkyl-O—(C₁₋₃)alkyl or (C₁₋₃)haloalkyl; or
R⁵ is —N(R^{A})(R^{B}) wherein R^{A} and R^{B} are (C₁₋₃)alkyl or R^{A} and R^{B} are linked together with the N to which they are attached to form a 4- to 5-membered saturated ring, wherein said ring is optionally substituted 1-3 times with (C₁₋₃)alkyl, —O—(C₁₋₃)alkyl, —OH or halo.

9. The compound according to claim 8, or a pharmaceutically acceptable salt thereof, wherein R⁵ is 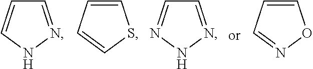
optionally substituted 1-3 times with (C₁₋₄)alkyl, (C₁₋₃)alkyl-C(═O)—N((C₁₋₃)alkyl)₂, —(C₁₋₃)alkyl-O—(C₁₋₃)alkyl or (C₁₋₃)haloalkyl; or
R⁵ is —N(CH₃)₂, 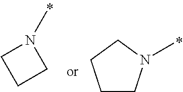
wherein said rings are optionally substituted 1-3 times with (C₁₋₃)alkyl, —O—(C₁₋₃)alkyl, —OH or halo.

10. A compound selected from the group consisting of:

| Cmpd # | Structure |
|---|---|
| 1001 | 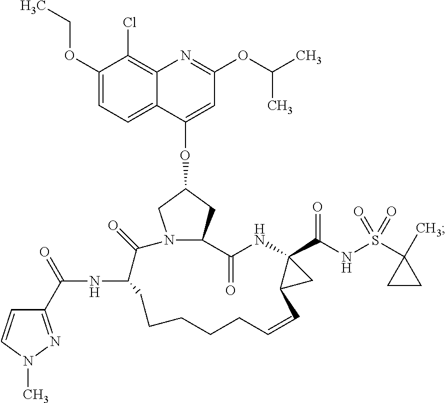 |
| 1002 | 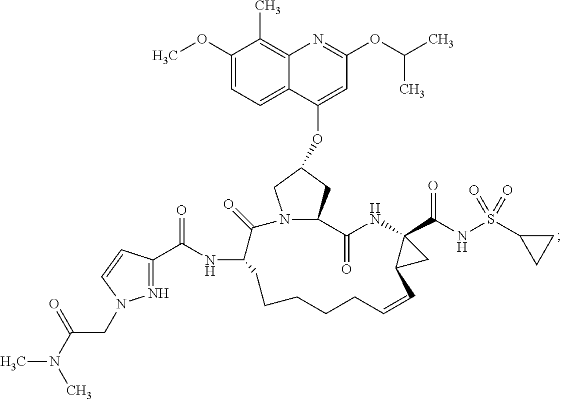 |
| 1003 | 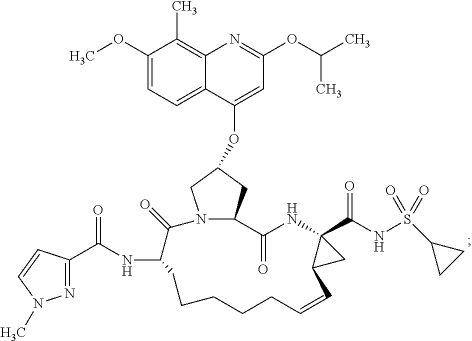 |
| 1004 | 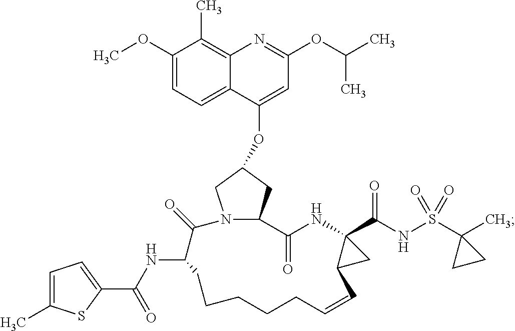 |
| 1005 | 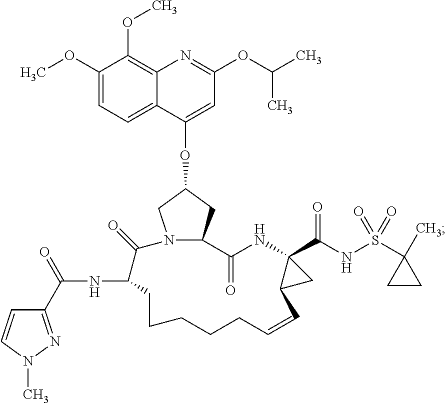 |
| 1006 | 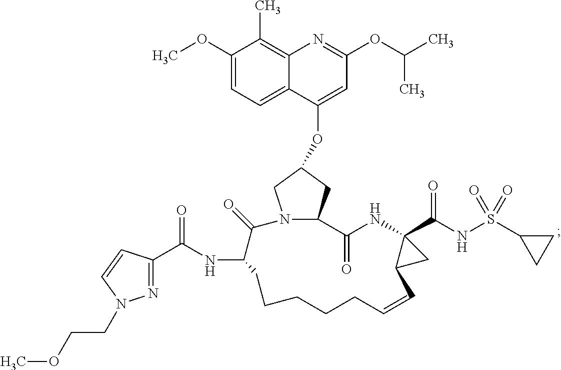 |
| 1007 | 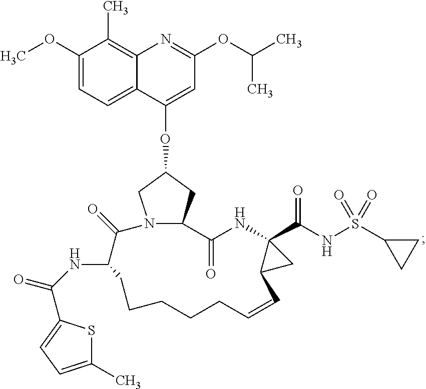 |
| 1008 | 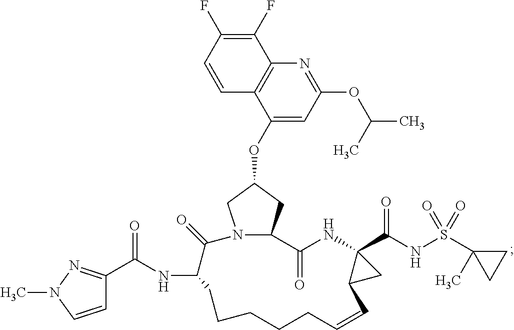 |
| 1009 | 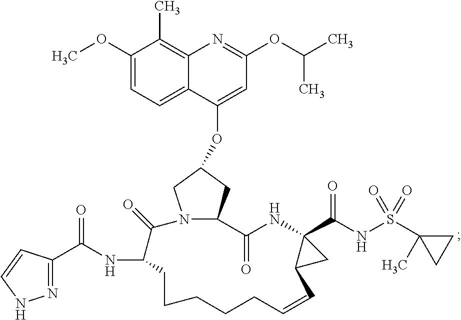 |
| 1010 | 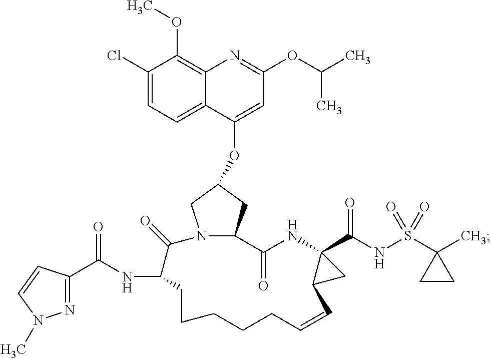 |
| 1011 | 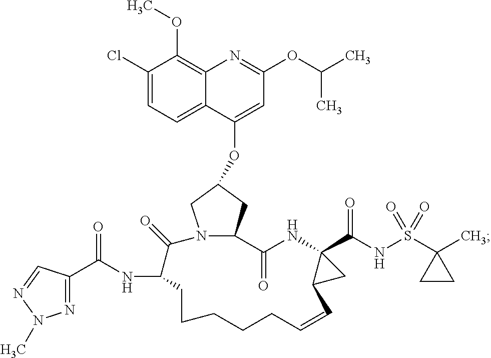 |
| 1012 | 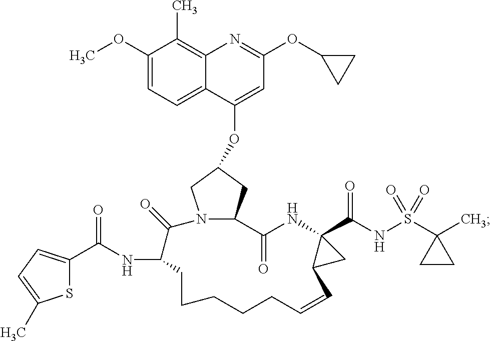 |
| 1013 | 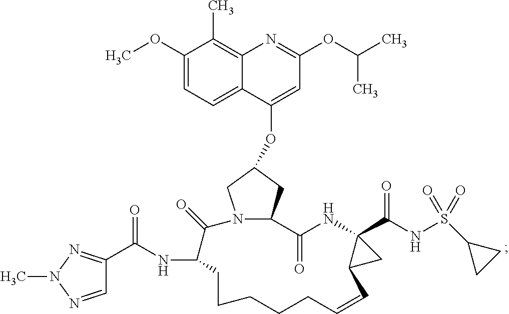 |
| 1014 | 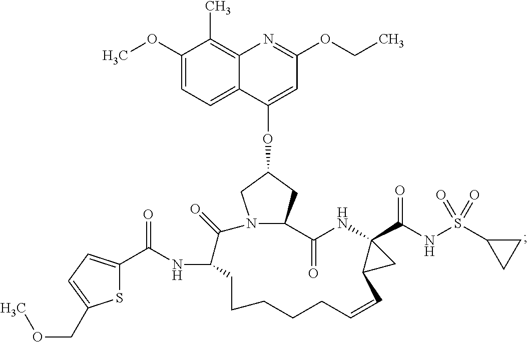 |
| 1015 | 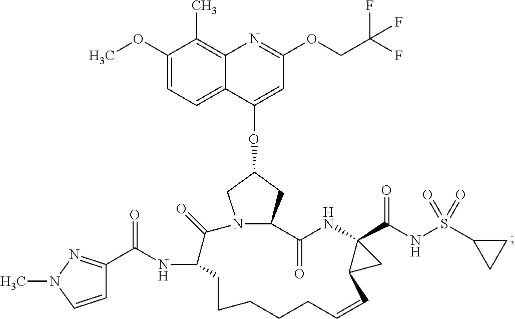 |
| 1016 | 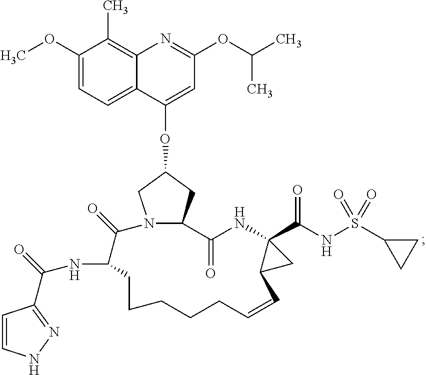 |
| 1017 | 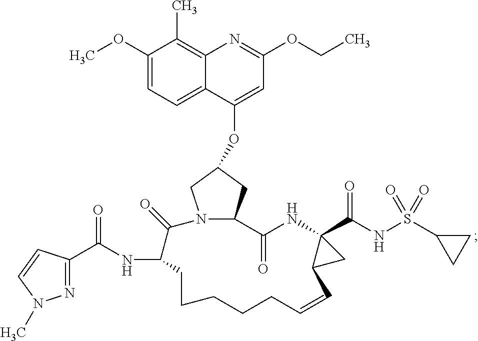 |
| 1018 | 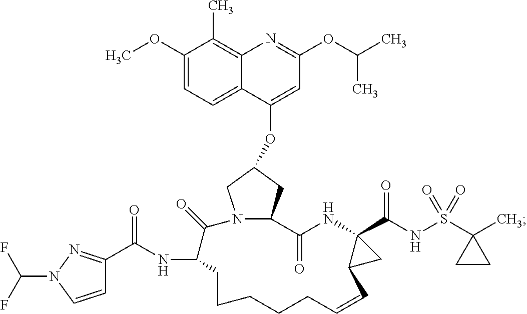 |
| 1019 | 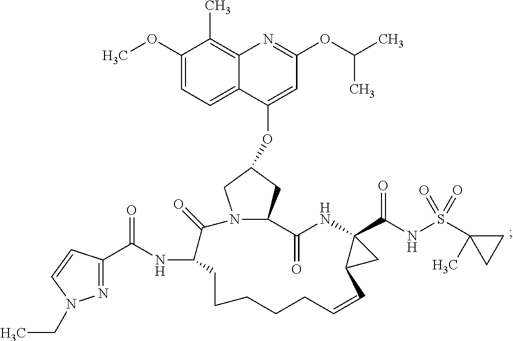 |
| 1020 | 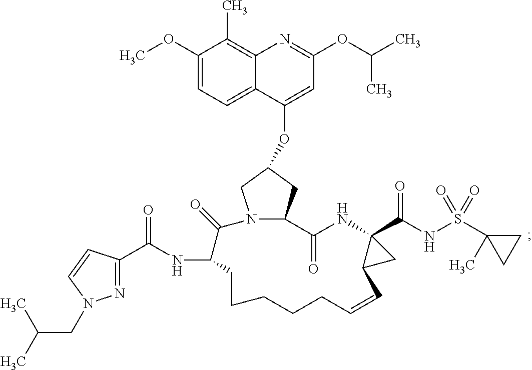 |
| 1021 | 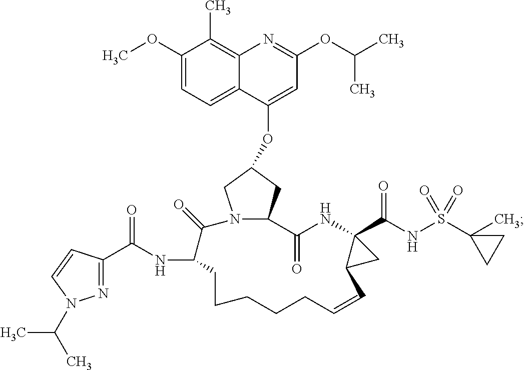 |
| 1022 | 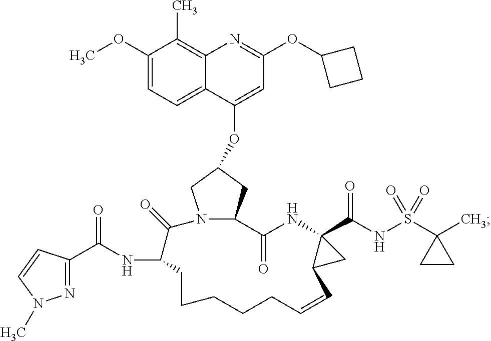 |
| 1023 | 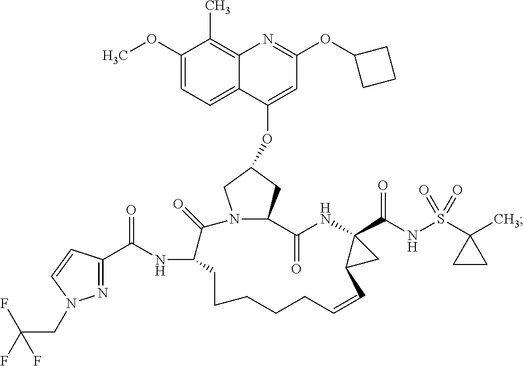 |
| 1024 | 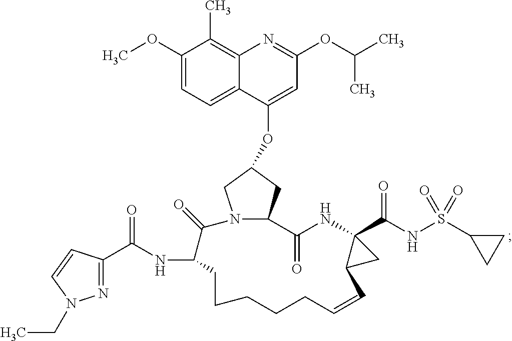 |
| 1025 | 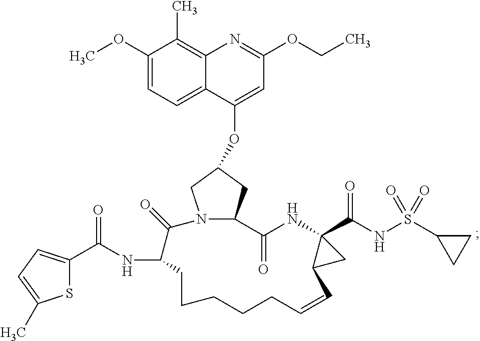 |
| 1026 | 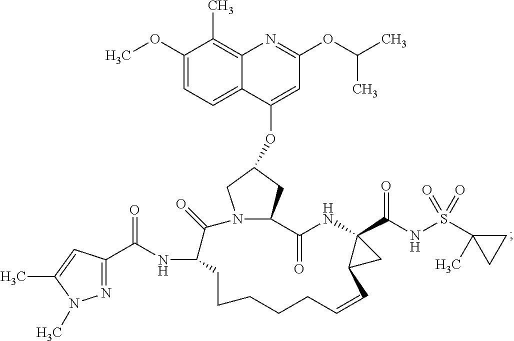 |
| 1027 | 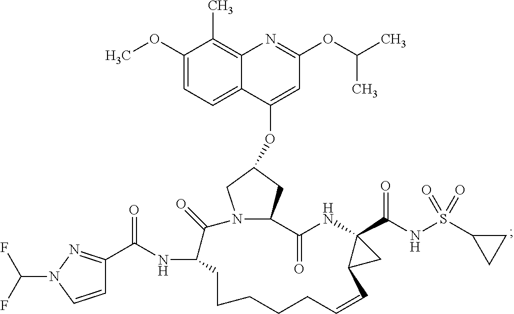 |
| 1028 | 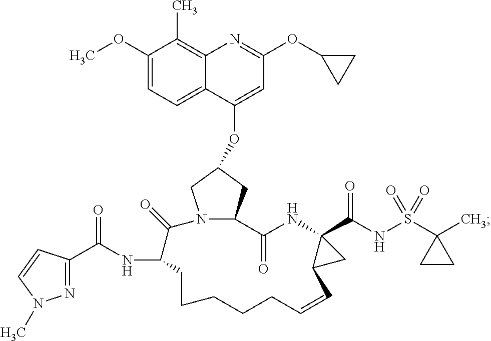 |
| 1029 | 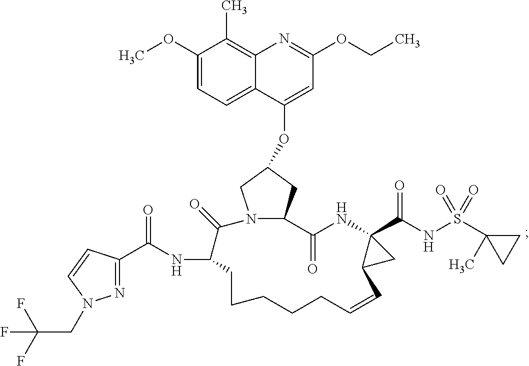 |
| 1030 | 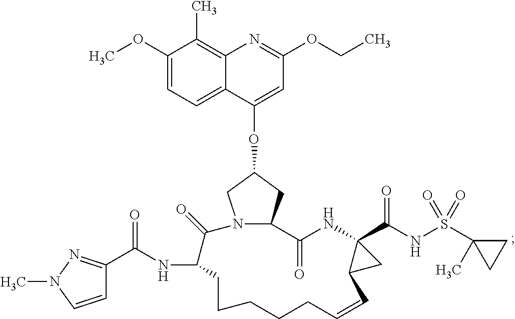 |
| 1031 | 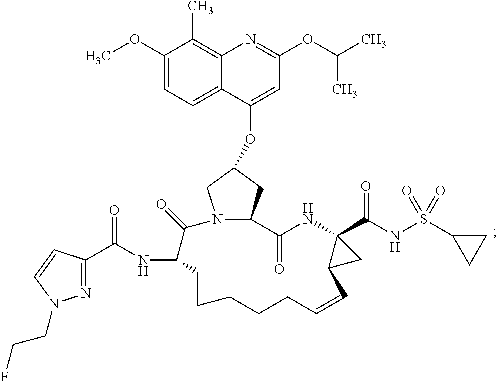 |
| 1032 | 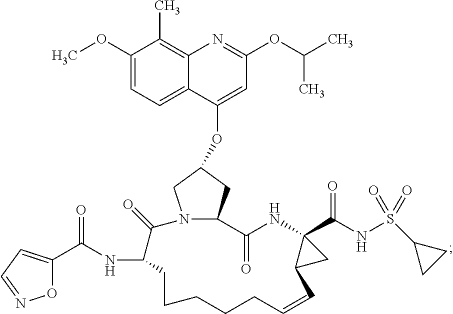 |
| 1033 | 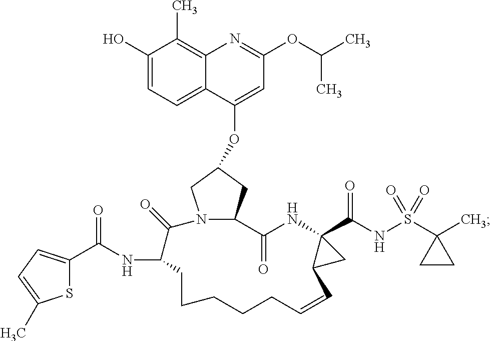 |
| 1034 | 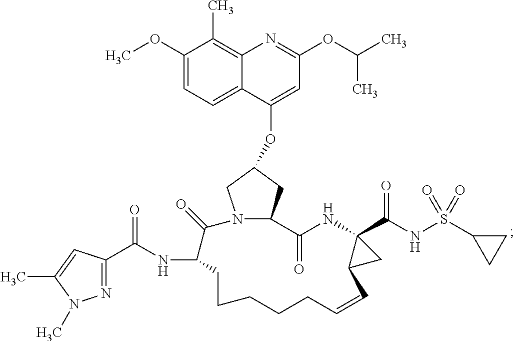 |
| 1035 | 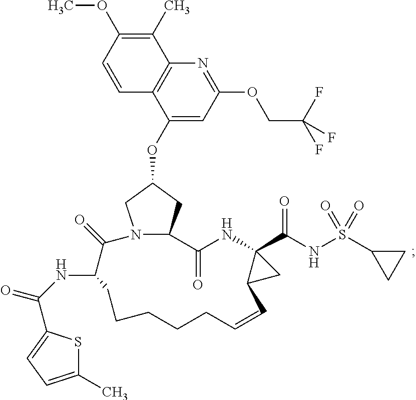 |
| 1036 | 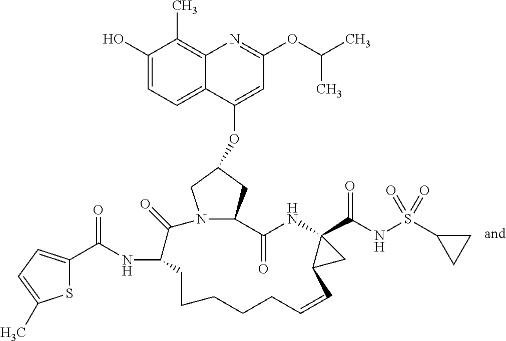 |
| 1037 | 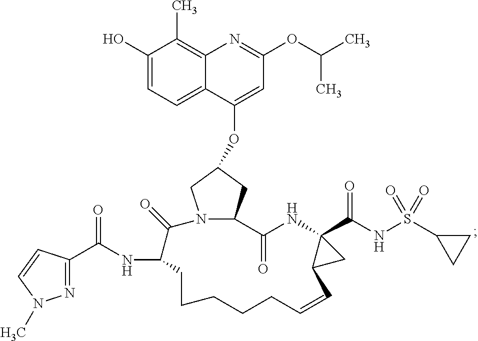 |

or a salt thereof.

11. A compound selected from the group consisting of:

| Cmpd # | Structure |
|---|---|
| 2001 | 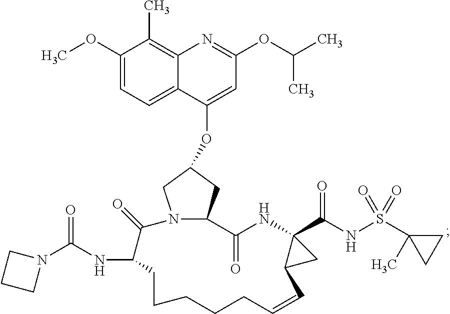 |
| 2002 | 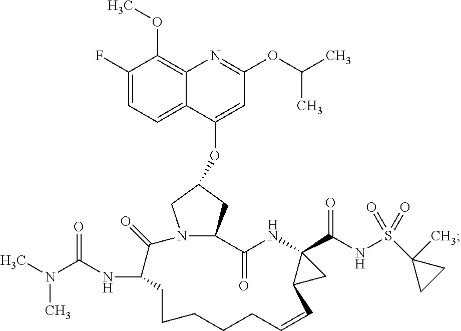 |
| 2003 | 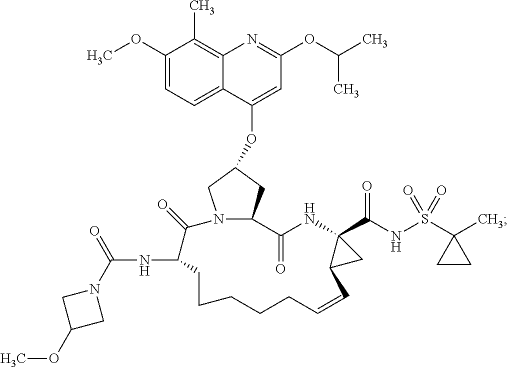 |
| 2004 | 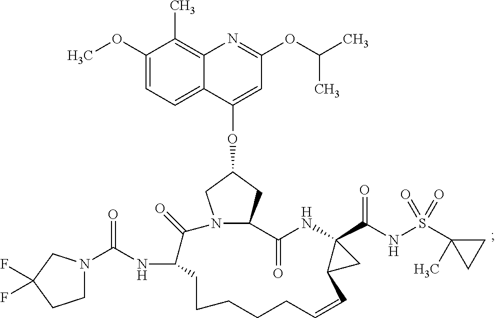 |
| 2005 | 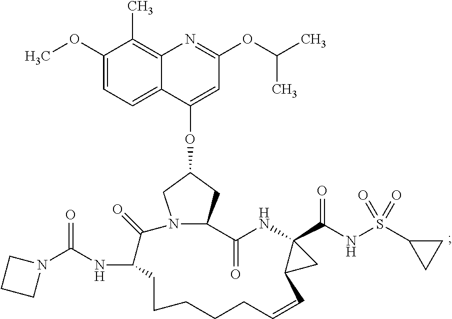 |
| 2006 | 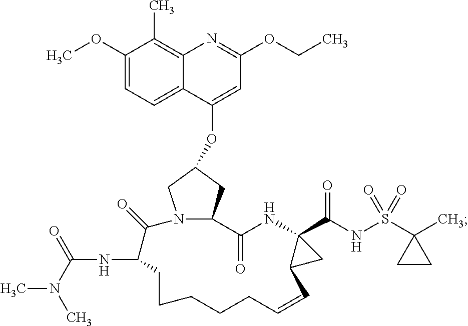 |
| 2007 | 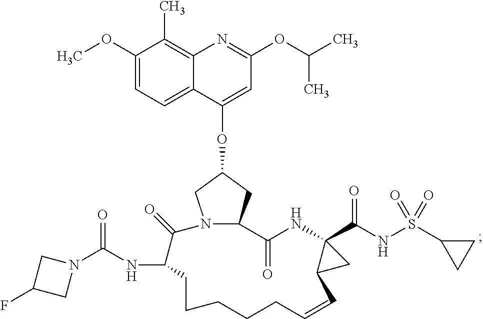 |
| 2008 | 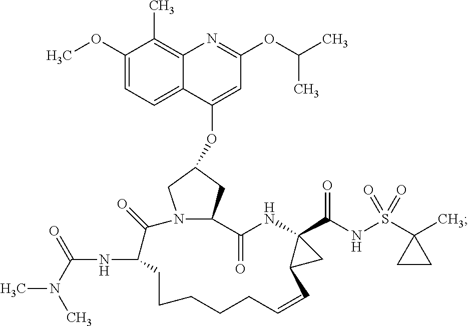 |
| 2009 | 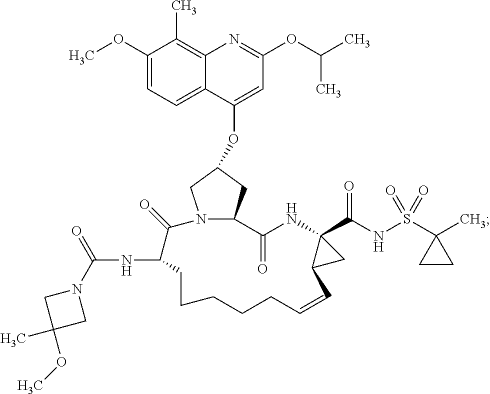 |
| 2010 | 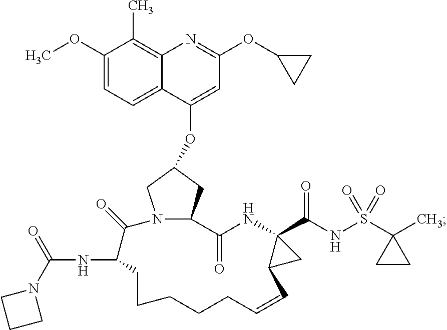 |
| 2011 | 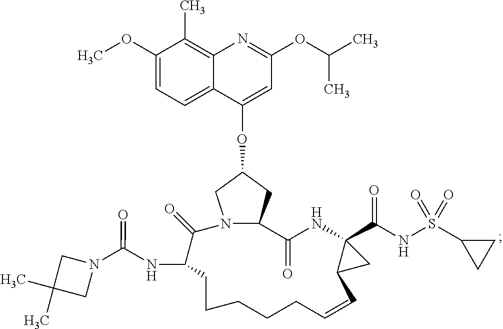 |
| 2012 | 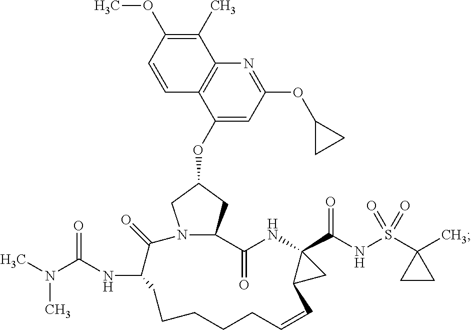 |
| 2013 | 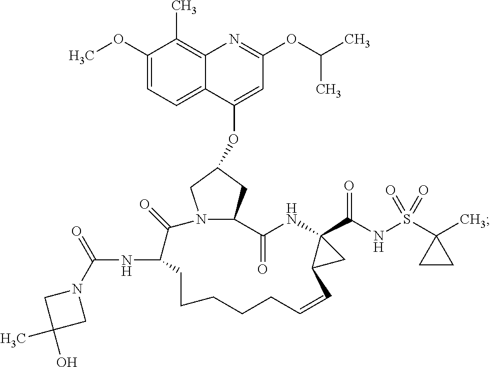 |
| 2014 | 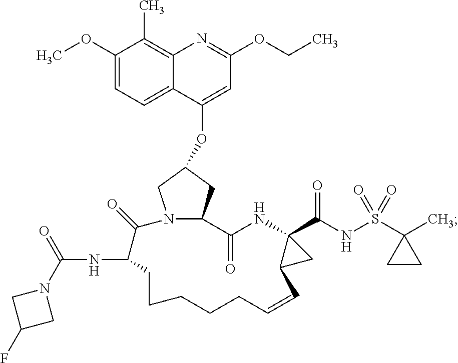 |
| 2015 | 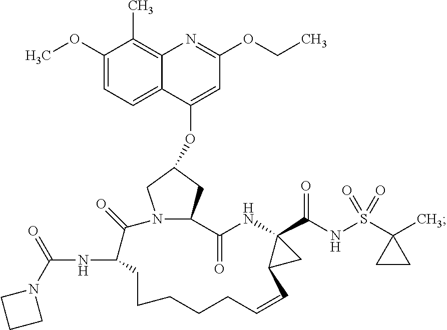 |
| 2016 | 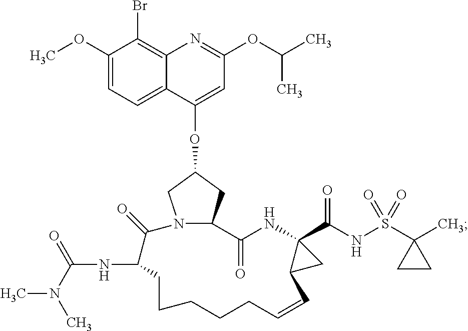 |
| 2017 | 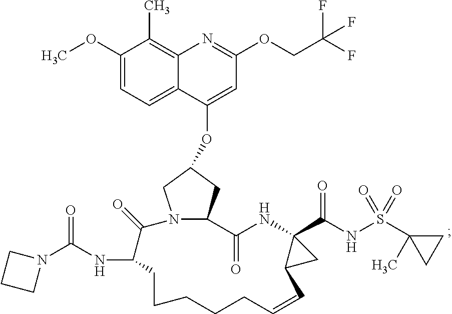 |
| 2018 | 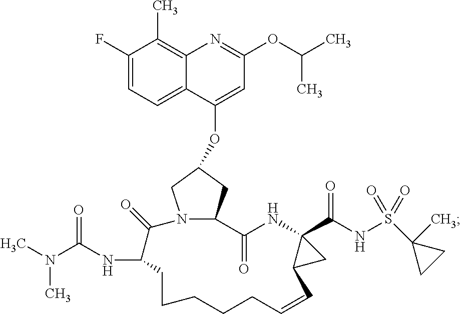 |
| 2019 | 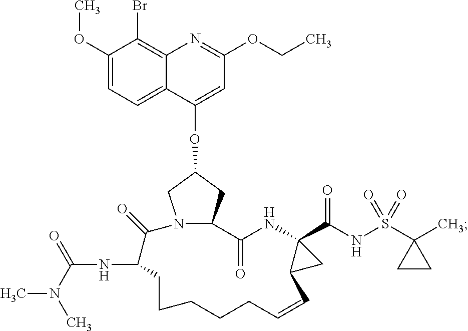 |
| 2020 | 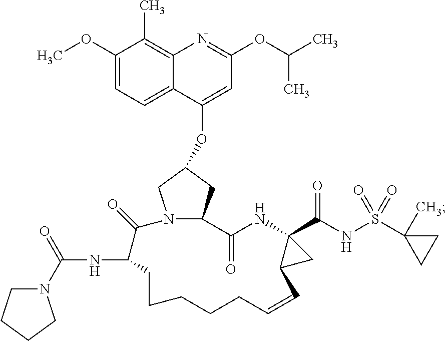 |
| 2021 | 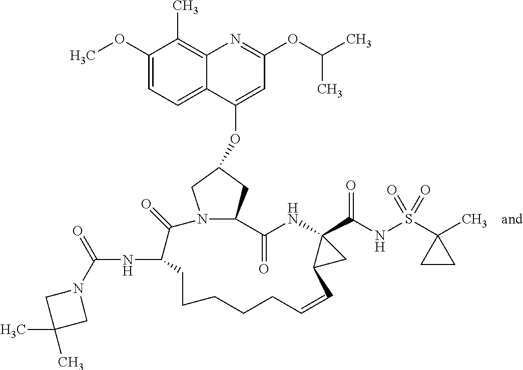 |
| 2022 | 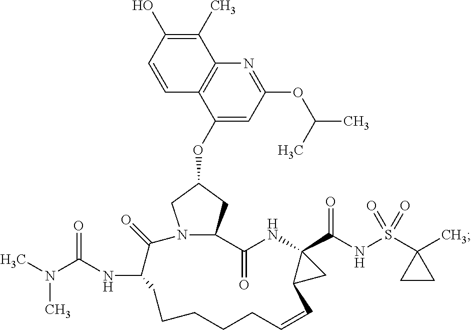 |

or a salt thereof.

12. A pharmaceutical composition comprising an anti-hepatitis C virally effective amount of a compound according to claim 1 or 2, or a pharmaceutically acceptable salt thereof, in admixture with at least one pharmaceutically acceptable carrier medium or auxiliary agent.

13. The pharmaceutical composition according to claim 12 further comprising a therapeutically effective amount of at least one other antiviral agent.

14. A method for the treatment or prevention of hepatitis C viral infection in a human being comprising administering to said human being an effective amount of a compound according to claim 1 or 2 or a pharmaceutically acceptable salt thereof.


SEQUENCE LISTING (4 sequences)

SEQ ID NO: 1 - DNA, 6609 bp, HCV
  CDS (1)...(6609)
atggacacggaggtggccgcgtcgtgtggcggcgttgttcttgtcgggttaatggcgctgactctgtcgccatattacaagcgctatatcagctggtgcatgtggtggcttcagtattttctgaccagagtagaagcgcaactgcacgtgtgggttccccccctcaacgtccggggggggcgcgatgccgtcatcttactcatgtgtgtagtacacccgaccctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcaaggccgcggtgtgcacccgtggagtggctaaagcggtggactttatccctgtggagaacctagggactaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcagggccggggtgagaacaattaccactggcagccccatcacgtactccacctacggcaagttccttgccgacggcgggtgctcagggggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgccccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtaatcccgaccagcggcaatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtttttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactgaccactggccaaaccctcctcctcaacatattgggggggtgggtggctgcccagctcgccgcccccggtgccgctactgcctttgtgggtgctggcctagctggcgccgccgtcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacgtgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatga

SEQ ID NO: 2 - protein, 2202 aa, HCV
MDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFKAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRAGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGNVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRHAEVITPAVQTNWQKLEVFWAKHMWNFISGIQYLAGLSTLPGNPAIASLMAFTAAVTSPLTTGQTLLLNILGGWVAAQLAAPGAATAFVGAGLAGAAVGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR

SEQ ID NO: 3 - DNA, 6615 bp, HCV
  CDS (1)...(6615)
atggaccgggagatggcagcatcgtgcggaggcgcggttttcgtaggtctgatactcttgaccttgtcaccgcactataagctgttcctcgctaggctcatatggtggttacaatattttatcaccagggccgaggcacacttgcaagtgtggatcccccccctcaacgttcgggggggccgcgatgccgtcatcctcctcacgtgcgcgatccacccagagctaatctttaccatcaccaaaatcttgctcgccatactcggtccactcatggtgctccaggctggtataaccaaagtgccgtacttcgtgcgcgcacacgggctcattcgtgcatgcatgctggtgcggaaggttgctgggggtcattatgtccaaatggctctcatgaagttggccgcactgacaggtacgtacgtttatgaccatctcaccccactgcgggactgggcccacgcgggcctacgagaccttgcggtggcagttgagcccgtcgtcttctctgatatggagaccaaggttatcacctggggggcagacaccgcggcgtgtggggacatcatcttgggcctgcccgtctccgcccgcagggggagggagatacatctgggaccggcagacagccttgaagggcaggggtggcgactcctcgcgcctattacggcctactcccaacagacgcgaggcctacttggctgcatcatcaccagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaagcatgccgatgtcattccggtgcgccggcggggcgacagcagggggagcctactctccccccggcccgtctcctacttgaagggctcttcgggcggtccactgctctgcccctcggggcacgctgtgggcatctttcgggctgccgtgtgcacccgaggggttgcgaaggcggtggtctttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgcgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagggcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggctgcgtattgcctgacaacaggcagcgtggtcattgtgggcaggatcatcttgtccggaaggccggccatcattcccgacagggaagtcctttaccgggagttcgatgagatggaagagtgtgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttccgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaaggcctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaactgttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgagtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacccggttgggtcacagctcccatgcgagcccgaactggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagagaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggagttaattaacgctagtgaggatatcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccacggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatga

SEQ ID NO: 4 - protein, 2204 aa, HCV
MDREMAASCGGAVFVGLILLTLSPHYKLFLARLIWWLQYFITRAEAHLQVWIPPLNVRGGRDAVILLTCAIHPELIFTITKILLAILGPLMVLQAGITKVPYFVRAHGLIRACMLVRKVAGGHYVQMALMKLAALTGTYVYDHLTPLRDWAHAGLRDLAVAVEPVVFSDMETKVITWGADTAACGDIILGLPVSARRGREIHLGPADSLEGQGWRLLAPITAYSQQTRGLLGCIITSLTGRDRNQVEGEVQVVSTATQSFLATCVNGVCWTVYHGAGSKTLAGPKGPITQMYTNVDQDLVGWQAPPGARSLTPCTCGSSDLYLVTKHADVIPVRRRGDSRGSLLSPRPVSYLKGSSGGPLLCPSGHAVGIFRAAVCTRGVAKAVVFVPVESMETTMRSPVFTDNSSPPAVPQTFQVAHLHAPTGSGKSTKVPAAYAAQGYKVLVLNPSVAATLGFGAYMSKAHGIDPNIRTGVRTITTGAPITYSTYGKFLADGGCSGGAYDIIICDECHSTDSTTILGIGTVLDQAETAGARLVVLATATPPGSVTVPHPNIEEVALSSTGEIPFYGKAIPIETIKGGRHLIFCHSKKKCDELAAKLSGLGLNAVAYYRGLDVSVIPTSGDVIVVATDALMTGFTGDFDSVIDCNTCVTQTVDFSLDPTFTIETTTVPQDAVSRSQRRGRTGRGRMGIYRFVTPGERPSGMFDSSVLCECYDAGCAWYELTPAETSVRLRAYLNTPGLPVCQDHLEFWEGVFTGLTHIDAHFLSQTKQAGDNFPYLVAYQATVCARAQAPPPSWDQMWKCLIRLKPTLHGPTPLLYRLGAVQNEVTTTHPITKYIMACMSADLEVVTSTWVLVGGVLAALAAYCLTTGSVVIVGRIILSGRPAIIPDREVLYREFDEMEECASHLPYIEQGMQLAEQFKQKAIGLLQTATKQAEAAAPVVESKWRTLEAFWAKHMWNFISGIQYLAGLSTLPGNPAIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKAWLQSKLLPRLPGVPFFSCQRGYKGVWRGDGIMQTTCPCGAQITGHVKNCSMRIVGPRTCSNTWHGTFPINAYTTGPCTPSPAPNYSRALWRVAAEEYVEVTRVGDFHYVTGMTTDNVKCPCQVPAPEFFTEVDGVRLHRYAPACKPLLREEVTFLVGLNQYPVGSQLPCEPELDVAVLTSMLTDPSHITAETAKRRLARGSPPSLASSSASQLSAPSLKATCTTRHDSPDADLIEANLLWRQEMGGNITRVESENKVVILDSFEPLQAEEDEREVSVPAEILRRSRKFPRAMPIWARPDYNPPLLESWKDPDYVPPVVHGCPLPPAKAPPIPPPRRKRTVVLSESTVSSALAELATKTFGSSESSAVDSGTATASPDQPSDDGDAGSDVESYSSMPPLEGEPGDPDLSDGSWSTVSEELINASEDIVCCSMSYTWTGALITPCAAEETKLPINALSNSLLRHHNLVYATTSRSASLRQKKVTFDRLQVLDDHYRDVLKEMKAKASTVKAKLLSVEEACKLTPPHSARSKFGYGAKDVRNLSSKAVNHIRSVWKDLLEDTETPIDTTIMAKNEVFCVQPEKGGRKPARLIVFPDLGVRVCEKMALYDVVSTLPQAVMGSSYGFQYSPGQRVEFLVNAWKAKKCPMGFAYDTRCFDSTVTENDIRVEESIYQCCDLAPEARQAIRSLTERLYIGGPLTNSKGQNCGYRRCRASGVLTTSCGNTLTCYLKAAAACRAAKLQDCTMLVCGDDLVVICESAGTQEDEASLRAFTEAMTRYSAPPGDPPKPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIQRLHGLSAFSLHSYSPGEINRVASCLRKLGVPPLRVWRHRARSVRARLLSQGGRAATCGKYLFNWAVRTKLKLTPIPAASQLDLSSWFVAGYSGGDIYHSLSRARPRWFMWCLLLLSVGVGIYLLPNR